(19) United States
(12) Patent Application Publication
(10) Pub. No.: US 2022/0389092 A1
(43) Pub. Date: Dec. 8, 2022

(54) METHODS AND COMPOSITIONS INVOLVING CHIMERIC BINDING POLYPEPTIDES

(71) Applicant: The Regents of the University of California, Oakland, CA (US)

(72) Inventors: Meng-Yin LIN, Los Angeles, CA (US); Yvonne Yu-Hsuan CHEN, Los Angeles, CA (US)

(73) Assignee: The Regents of the University of California, Oakland, CA (US)

(21) Appl. No.: 17/600,095

(22) PCT Filed: Apr. 2, 2020
(86) PCT No.: PCT/US2020/026428

§ 371 Date: Sep. 29, 2021

Related U.S. Application Data
(60) Provisional application No. 62/829,905, filed on Apr. 5, 2019.

(51) Int. Cl. C07K 16/24; C07K 16/28; A61P 37/06; A61K 35/17; A61K 45/06; A61K 39/395

(52) U.S. Cl. CPC C07K 16/249; C07K 16/241; C07K 16/2803; C07K 16/2887; A61P 37/06; A61K 35/17; A61K 45/06; A61K 39/3955; C07K 16/248; C07K 2317/565; A61K 2039/505

(57) ABSTRACT

The current disclosure provides polypeptide, nucleic acid, compositions, and methods for treating or preventing CRS in patients in need thereof, particularly for those receiving an immunotherapy, such as a cancer immunotherapy, that may provoke a CRS response. Accordingly, aspects of the disclosure relate to a chimeric binding polypeptides comprising a heavy chain variable region comprising CDR1, CDR2, and CDR3 attached by a heterologous linker to a light chain variable region comprising CDR4, CDR5, and CDR6.

This application claims priority of U.S. Provisional Application No. 62/829,905 filed on Apr. 5, 2019, the entirety of which is incorporated by reference.

### BACKGROUND OF THE INVENTION

The prospects of using antibodies that interact with T-cells for employing new and effective therapies for the treatment of a variety of diseases and conditions, including cancer, microbial infections, and autoimmune diseases, continue to develop. However, there are side effects and toxicities that can result, including cytokine release syndrome (CRS). CRS can cause a patient to experience hypotension, pyrexia, and rigors (a sudden feeling of cold with shivering accompanied by a rise in temperature) similar to the onset of a high fever. Treatments are needed that will prevent or alleviate the side effects and toxicities from T-cell engaging therapies.

### SUMMARY OF THE INVENTION

The current disclosure provides polypeptide, nucleic acid, compositions, and methods for treating or preventing CRS in patients in need thereof, particularly for those receiving an immunotherapy, such as a cancer immunotherapy, that may provoke a CRS response. Accordingly, aspects of the disclosure relate to a chimeric tumor necrosis factor alpha (TNF-α) binding polypeptide comprising a heavy chain variable region comprising CDR1, CDR2, and CDR3 attached by a heterologous linker to a light chain variable region comprising CDR4, CDR5, and CDR6; wherein the polypeptide comprises CDR1, CDR2, and CDR3 of the heavy chain variable region of SEQ ID NO:4 and CDR4, CDR5, and CDR6 of the light chain variable region of SEQ ID NO:3; CDR1, CDR2, and CDR3 of the heavy chain variable region of SEQ ID NO:6 and CDR4, CDR5, and CDR6 of the light chain variable region of SEQ ID NO:5; CDR1, CDR2, and CDR3 of the heavy chain variable region of SEQ ID NO:8 and CDR4, CDR5, and CDR6 of the light chain variable region of SEQ ID NO:7; or CDR1, CDR2, and CDR3 of the heavy chain variable region of SEQ ID NO:10 and CDR4, CDR5, and CDR6 of the light chain variable region of SEQ ID NO:9.

Further aspects relate to a chimeric binding polypeptide comprising a heavy chain variable region comprising CDR1, CDR2, and CDR3 attached by a heterologous linker to a light chain variable region comprising CDR4, CDR5, and CDR6; wherein the polypeptide comprises CDR1, CDR2, and CDR3 of the heavy chain variable region of SEQ ID NO:2 and CDR4, CDR5, and CDR6 of the light chain variable region of SEQ ID NO:1.

Further aspects relate to a chimeric interferon gamma (IFN-γ) binding polypeptide comprising a heavy chain variable region comprising CDR1, CDR2, and CDR3 attached by a heterologous linker to a light chain variable region comprising CDR4, CDR5, and CDR6; wherein the polypeptide comprises CDR1, CDR2, and CDR3 of the heavy chain variable region of SEQ ID NO:12 and CDR4, CDR5, and CDR6 of the light chain variable region of SEQ ID NO:11.

Further aspects of the disclosure relate to a chimeric binding protein comprising an IL-1 receptor binding polypeptide of SEQ ID NO:13 and a CAR.

Further aspects relate to T cells expressing the chimeric binding polypeptide embodiments of the disclosure. Further aspects relate to a nucleic acid molecule encoding the chimeric binding polypeptide embodiments of the disclosure. Further aspects relate to an expression construct comprising a nucleic acid molecule embodiment of the disclosure.

Further aspects relate to a recombinant T cell comprising a nucleic acid embodiment of the disclosure.

Further aspects relate to a method for reducing the risk of cytokine release syndrome comprising administering to a patient at risk for cytokine release syndrome a composition comprising a chimeric binding polypeptide embodiment of the disclosure, a nucleic acid of the disclosure, or a T cell of the disclosure.

Further aspects relate to a method for reducing toxicity in a patient comprising administering a composition comprising a chimeric binding polypeptide embodiment of the disclosure, a nucleic acid of the disclosure, or a T cell of the disclosure; wherein the patient has been administered an immunotherapy.

Further aspects relate to a method for reducing toxicity in a patient comprising administering a composition comprising a chimeric binding polypeptide embodiment of the disclosure, a nucleic acid of the disclosure, or a T cell of the disclosure; wherein the method further comprises administration of a cancer immunotherapy.

Further aspects relate to a method for reducing the toxicity of adoptive cell therapy in a cancer patient comprising administering to the patient a composition comprising a chimeric binding polypeptide, nucleic acid, or T cell embodiment of the disclosure. Further aspects relate to a method for treating an autoimmune disease or cancer in a patient comprising administering to the patient a composition comprising a composition comprising a chimeric binding polypeptide, nucleic acid, or T cell embodiment of the disclosure.

In some embodiments, the polypeptide comprises: a CDR1, CDR2, CDR3, CDR4, CDR5, and/or CDR6 with at least 80% sequence identity to SEQ ID NOS:91-96, respectively. In some embodiments, the polypeptide comprises: a CDR1, CDR2, CDR3, CDR4, CDR5, and/or CDR6 with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NOS:91-96, respectively. In some embodiments, the polypeptide comprises: a CDR1, CDR2, CDR3, CDR4, CDR5, and/or CDR6 with at least 80% sequence identity to SEQ ID NOS:97-102, respectively. In some embodiments, the polypeptide comprises: a CDR1, CDR2, CDR3, CDR4, CDR5, and/or CDR6 with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NOS:97-102, respectively. In some embodiments, the polypeptide comprises: a CDR1, CDR2, CDR3, CDR4, CDR5, and/or CDR6 with at least 80% sequence identity to SEQ ID NOS:103-108, respectively. In some embodiments, the polypeptide comprises: a CDR1, CDR2, CDR3, CDR4, CDR5, and/or CDR6 with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NOS:103-108, respectively. In some embodiments, the polypeptide comprises: a CDR1, CDR2, CDR3, CDR4, CDR5, and/or CDR6 with at least 80% sequence identity to SEQ ID NOS:109-114, respectively. In some embodiments, the polypeptide comprises: a CDR1, CDR2, CDR3, CDR4, CDR5, and/or CDR6 with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NOS:109-114, respectively. In some embodiments, the polypeptide comprises: a CDR1, CDR2, CDR3, CDR4, CDR5, and/or CDR6 with at least 80% sequence identity to SEQ ID NOS:115-120, respectively. In some embodiments, the polypeptide comprises: a CDR1, CDR2, CDR3, CDR4, CDR5, and/or CDR6 with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NOS:115-120, respectively. In some embodiments, the polypeptide comprises: a CDR1, CDR2, CDR3, CDR4, CDR5, and/or CDR6 with at least 80% sequence identity to SEQ ID NOS:121-126, respectively. In some embodiments, the polypeptide comprises: a CDR1, CDR2, CDR3, CDR4, CDR5, and/or CDR6 with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NOS:121-126, respectively.

In some embodiments, the polypeptide comprises: a CDR1, CDR2, CDR3, CDR4, CDR5, and CDR6 having a sequence corresponding to SEQ ID NOS:91-96, respectively. In some embodiments, the polypeptide comprises: a CDR1, CDR2, CDR3, CDR4, CDR5, and CDR6 having a sequence corresponding to SEQ ID NOS:97-102, respectively. In some embodiments, the polypeptide comprises: a CDR1, CDR2, CDR3, CDR4, CDR5, and CDR6 having a sequence corresponding to SEQ ID NOS:103-108, respectively. In some embodiments, the polypeptide comprises: a CDR1, CDR2, CDR3, CDR4, CDR5, and CDR6 having a sequence corresponding to SEQ ID NOS:109-114, respectively. In some embodiments, the polypeptide comprises: a CDR1, CDR2, CDR3, CDR4, CDR5, and CDR6 having a sequence corresponding to SEQ ID NOS:115-120, respectively. In some embodiments, the polypeptide comprises: a CDR1, CDR2, CDR3, CDR4, CDR5, and CDR6 having a sequence corresponding to SEQ ID NOS:121-126, respectively.

A CDR may also comprise 1, 2, 3, 4, 5, 6, 7, 8, 9, 10, 11, 12, 13, 14, 15, 16, 16, 18, 19, 20, 21, 22, 23, or more contiguous amino acid residues (or any range derivable therein) flanking one or both sides of a particular CDR sequence; therefore, there may be one or more additional amino acids at the N-terminal or C-terminal end of a particular CDR sequence, such as those shown in SEQ ID NOS:91-126. A CDR may have at least or at most 1, 2, 3, 4, 5, 6, 7, 8, 9, 10, 11, 12, 13, 14, 15, 16, 16, 18, 19, 20, 21, 22, or 23 amino acid substitutions, insertions, or deletions that may correspond to the amino acid at position 1, 2, 3, 4, 5, 6, 7, 8, 9, 10, 11, 12, 13, 14, 15, 16, 16, 18, 19, 20, 21, 22, or 23 of any one of SEQ ID NO:91-126. A CDR may be a fragment of at least 3, 4, 5, 6, 7, 8, 9, 10, 11, 12, 13, 14, 15, 16, 16, 18, 19, 20, 21, 22, or 23 contiguous amino acids of any one of SEQ ID NO:91-126 and may have at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NOS:91-126.

In some embodiments, chimeric binding polypeptide is an scFv, (scFv)2, scFvFc, Fab, Fab′, F(ab)2, or a single chain antibody. In some embodiments, the binding polypeptide is one polypeptide. In some embodiments, the one polypeptide is a single chain variable fragment (scFv). In embodiments involving a single polypeptide containing both a heavy chain variable region and a light chain variable region, both orientations of of these variable regions are contemplated. In some cases, the heavy chain variable region is on the N-terminal side of the light chain variable region, which means the heavy chain variable region is closer to the N-terminus of the polypeptide. In other cases, the light chain variable region is on the N-terminal side of the heavy chain variable region, which means the light chain variable region is closer to the N-terminus of the polypeptide than the heavy chain variable region. In some embodiments, the light chain variable region is on the N-terminal side of the heavy chain variable region.

In some embodiments, the linker comprises the amino acid sequence GSTSGSGKPGSGEGSTKG (SEQ ID NO:14). In some embodiments, the chimeric binding polypeptide further comprises a leader peptide. In some embodiments, the chimeric binding polypeptide further comprises an isolation tag.

In some embodiments, the polypeptide comprises: the heavy chain variable region of SEQ ID NO:4 and light chain variable region of SEQ ID NO:3; the heavy chain variable region of SEQ ID NO:6 and the light chain variable region of SEQ ID NO:5; the heavy chain variable region of SEQ ID NO:8 and the light chain variable region of SEQ ID NO:7; or the heavy chain variable region of SEQ ID NO:10 and the light chain variable region of SEQ ID NO:9. In some embodiments, the polypeptide comprises: the heavy chain variable region of SEQ ID NO:2 and light chain variable region of SEQ ID NO:1. In some embodiments, the polypeptide comprises the heavy chain variable region of SEQ ID NO:12 and the light chain variable region of SEQ ID NO:11.

In some embodiments, the polypeptide further comprises a chimeric antigen receptor (CAR). In some embodiments, the chimeric antigen receptor is expressed from the same nucleic acid as the chimeric binding polypeptide. In some embodiments, the chimeric antigen receptor is covalently linked to the chimeric binding polypeptide. In some embodiments, the chimeric antigen receptor is covalently linked to the CAR through a peptide bond. In some embodiments, a T cell comprises a first nucleotide comprising a chimeric binding polypeptide of the disclosure and a second nucleotide comprising a CAR embodiment of the disclosure. In some embodiments, the first and second nucleotide are different molecules (not covalently linked). In some embodiments, the chimeric binding polypeptide and CAR are expressed from different promoters. In some embodiments, the different promoters provide for different expression levels of each of the molecules. In some embodiments, the ratio of expressed protein per cell, on average, of the chimeric binding polypeptide to the CAR polypeptide is 1:1. In some embodiments, the ratio of the expressed protein of the chimeric binding polypeptide to the CAR polypeptide is at least, at most, or about 0.25:1, 0.5:1, 0.75:1 1:1, 1.25:1, 1.5:1, 1.75:1, 2:1, 2.25:1, 2.5:1, 2.75:1, 3:1, 3.25:1, 3.5:1, 3.75:1, 4:1, 4.25:1, 4.5:1, 4.75:1, 5:1, 5.25:1, 5.5:1, 5.75:1, 6:1, 6.25:1, 6.5:1, 6.75:1 7:1, 7.25:1, 7.5:1, 7.75:1, 8:1, 8.25:1, 8.5:1, 8.75:1, 9:1, 9.25:1, 9.5:1, 9.75:1, 10:1, 11:1, 12:1, 13:1, 14:1, 15:1, 20:1, 25:1, 30:1, 40:1, or 50:1 (or any range derivable therein).

In some embodiments, the CAR comprises a CD19 CAR. In some embodiments, the CAR comprises a CD20 CAR. In some embodiments, the CAR is a monospecific CAR. In some embodiments, the CAR is a bispecific CAR. In some embodiments, the CAR comprises a CD19/CD20 bispecific CAR. and/or CD20 monospecific or bispecific CAR. In some embodiments, the CAR comprises a TGF-β CAR. In some embodiments, the CAR comprises a BCMA CAR. In some embodiments, the CAR comprises a CS1 CAR. In some embodiments, the CAR comprises a BCMA/CAR1 bispecific CAR. In some embodiments, the CAR comprises a CD22 CAR. In some embodiments, the CAR comprises a HER2 CAR. In some embodiments, the CAR comprises a mesothelin CAR.

In some embodiments, the T cell is reduced in expression of TRAC and/or B2M gene products. In some embodiments, the endogenous TRAC and/or B2M genes are mutated to reduce or eliminate expression of the TRAC and/or B2M gene products.

In some embodiments, the CAR comprises a CD19/CD22 bispecific CAR. In some embodiments, the chimeric binding polypeptide comprises or further comprises a cleavage site between i) the CAR and ii) the heavy and light chain variable regions of the chimeric binding polypeptide. In some embodiments, the cleavage site comprises a self-cleaving 2A polypeptide. In some embodiments, the cleavage site comprises a protease cleavage site. In some embodiments, the cleavage site is site-specific. In some embodiments, the CAR comprises or further comprises a CD34 epitope. The CD34 epitope may be used to sort T cells expressing polypeptides of the disclosure.

In some embodiments, the nucleic acid comprises or further comprises a promoter controlling expression of the chimeric binding polypeptide. In some embodiments, the promoter is constitutive. In some embodiments, the promoter is conditional. In some embodiments, the promoter responds positively to at least one cytokine or to T-cell activation. In some embodiments, the at least one cytokine is IL-6, TNF-α, IFN-γ, IL-1β, IL-2, IL-8, IL-1, or IL-10 or the promoter responds positively to NFAT-1 or NF-κB. In some embodiments, the promoter is inducible. In some embodiments, the inducible promoter comprises an inducible response element and/or a minimal promoter. In some embodiments, the response element comprises one or more of TRE, JRE-IL-6, JEBS-VIPCRE, APRE, and IRF1-IL-6RE. In some embodiments, the inducible promoter comprises the minimal promoter: pJB42CAT5, miniTK, YB TATA, minCMV, minSV40, CMV53 or MLP. In some embodiments, the inducible promoter comprises an NFAT-inducible promoter.

In some embodiments, the expression construct is a viral vector. In some embodiments, the viral vector comprises a lenti viral vector or is a viral vector derived from lentivirus. In some embodiments, the viral vector comprises a retroviral vector. In some embodiments, the vector is derived from a retroviral vector. In some embodiments, the expression construct is a plasmid.

In some embodiments, a T cell of the disclosure comprises an expression construction comprising a cytokine responsive promoter or promoter that increases expression when T cells are activated. In some embodiments, the promoter responds positively to one or more of the following: NFAT-1, NF-κB, IL-6, TNF-α, IFN-γ, IL-1β, IL-2, IL-8, IL-1, and IL-10.

In some embodiments, the chimeric binding polypeptide is secreted by a T cell of the disclosure. In some embodiments, the secretion is in vivo. In some embodiments, administration of a T cell of the disclosure provides for a reduction in the effective concentration of the chimeric binding polypeptide compared to administration of the same or similar polypeptide constructs. In some embodiments, administration of a T cell of the disclosure provides for a reduction in the effective concentration of the chimeric binding polypeptide compared to systemic administration of the same or similar polypeptide constructs. In some embodiments, the reduction is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein).

In some embodiments, the cancer immunotherapy is CAR-T cell therapy. In some embodiments, the immunotherapy is administered at the same time as the chimeric binding polypeptide, nucleic acid, or T cell of the disclosure. In some embodiments, the patient has cancer. In some embodiments, the patient has or will receive adoptive T-cell therapy. In some embodiments, the method further comprises treating the cancer. In some embodiments, the cancer comprises lymphoma. In some embodiments, the cancer comprises Burkitt's lymphoma. In some embodiments, the patient has or will receive lymphodepletion. In some embodiments, the patient has an autoimmune disease. In some embodiments, the T cells administered to the patient are autologous. In some embodiments, the T cells administered to the patient are non-autologous.

In some embodiments, the patient is administered an additional therapy. In some embodiments, the additional therapy comprises a cancer therapy. In some embodiments, the additional therapy comprises an immunotherapy. In some embodiments, the additional therapy comprises a therapy described herein. In some embodiments, the methods further comprise administering to the patient an antihistamine, a corticosteroid, a steroid, acetaminophen, furosemide, and/or intravenous fluids.

In some embodiments, the patient has one or more symptoms of cytokine release syndrome. In some embodiments, the patient does not have symptoms of cytokine release syndrome.

In one embodiment of the methods described herein, the subject is a human subject. The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Other embodiments are described in WO2018042385, which is herein incorporated by references.

It is specifically contemplated that embodiments described herein may be excluded. It is further contemplated that, when a range is described, certain ranges may be excluded.

As used herein the specification, “a” or “an” may mean one or more. As used herein in the claim(s), when used in conjunction with the word “comprising”, the words “a” or “an” may mean one or more than one.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” Is is specifically contemplated that x, y, or z may be specifically excluded from an embodiment.

Throughout this application, the term “about” is used according to its plain and ordinary meaning in the area of cell biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-D. Antagonists derived from commercial drugs can be expressed by human cells. Western blot on the supernatant of HEK293T cells transfected with (A) IL-6Rα antagonists, (B) TNF-α antagonists and (C) IFN-γ antagonists. (D) Intracellular staining result showed expression of antagonist derived from anakinra in transfected HEK293T cells.

FIG. 2. Both orientations of tocilizumab scFvs efficiently inhibit IL-6 signaling in human cells. Western blot on the lysate of primary human CD4⁺ T cells treated with different concentrations of scFvs and indicated concentration of IL-6. pSTAT3, phosphorylated STAT3. “Normalized STAT3” values indicate the pSTAT3 band intensity normalized by the GAPDH band intensity.

FIG. 3. Efficacy of different TNF-α antagonists in inhibiting NFκB signaling in Jurkat cells. The NFκB signaling triggered by TNF-α addition is inhibited by TNF-α antagonists in a dose-dependent manner. A Jurkat cell line that stably expresses an NFκB reporter driving the expression of EGFP was stimulated by the addition of 10 ng/ml TNF-α, which triggers NFκB signaling. Various amounts of scFvs were added to inhibit TNF-α binding. The intensity of NF□B signaling was evaluated by EGFP expression 24-hours after experiment setup. Values shown are the means of three biologically independent cell cultures, with error bars indicating ±1 standard deviation (s.d.). Each group of three bars represents 10, 20, and 50 μg/ml when going from left to right. The bars in the controls represent 10 μg/ml.

FIG. 4A-D. Human cells can be engineered to express cytokine antagonists while maintaining high CAR surface expression levels. (A) Schematic of a self-regulating CAR construct. (B) The CD19 CAR was efficiently expressed on the T-cell surface as detected by HA staining. (C) The CD19 CAR expression level was unaffected by co-expression with cytokine antagonists. (D) Intracellular staining for myc or FLAG tags attached on the N-terminus of cytokine antagonists confirms protein expression, albeit with low efficiency for anakinra. EGFRt: truncated epidermal growth factor receptor that serves as a transduction marker for the CD19 CAR construct.

FIG. 5A-B. Secretion of cytokine antagonists by primary human T cells does not affect the expression of CD19 CAR or the CD4:CD8 ratio in T-cell subtype distribution. (A) EGFRt vs. CD19 CAR expression in primary human T cells 7-days after activation. (B) Ratio of CD4⁺ and CD8⁺ T cells in primary human T cells expressing different cytokine antagonists. All the constructs contain CD19 CAR and the expression of CD19 CAR is assessed by HA expression. CAR, chimeric antigen receptor. scFv, single-chain variable fragment.

FIG. 6A-B. Cytotoxicity and proliferation of primary human CD19 CAR-T cells were not compromised by the expression of different cytokine antagonists. CD19 CAR-T cells with expression of EGFRt or different cytokine antagonists were co-incubated with wildtype Raji cells at 1:1 effector-to-target ratio. To evaluate the functionality of T cells after repeated antigen challenge, additional Raji cells were added into the culture every two days. Viable cell counts of (A) T cells and (B) target cells were quantified by flow cytometry every two days, before addition of Raji cells. Cell numbers at challenge 1 day 2 (C1D2) is defined as fold change=1. CAR-T cells with expression of different cytokine antagonists showed comparable trends of T-cell proliferation and target cell lysis compare to their EGFRt counterpart. Values shown are the means of three biologically independent cell cultures, with error bars indicating ±1 standard deviation (s.d.). Values shown are the means of three biologically independent cell cultures from the same donor, with error bars indicating ±1 standard deviation (s.d.). Statistical comparisons were performed on challenge 5 day 2 (C5D2) values by comparing scFv-expressing or anakinra-expressing T cells with CD19 CAR-EGFRt T cells using a two-tailed Student's t-test. *, p≤0.05; **, p≤0.01. Unmarked pairs do not show statistically significant difference (p>0.05).

FIG. 7A-B. CD34 epitope enables detection and isolation of transduced CAR-T cells. (A) Schematic of CD34 epitope-incorporated CAR. Color-shaded boxes indicate modified portions compare to regular CAR constructs. (B) FACS analysis of the panel of different CD34 epitope-incorporated CARs for their CAR and CD34 epitope expression. Primary human T cells were sorted by CD34 expression 15 days after activation. Incorporation of CD34 epitope provides a strategy to enrich for CAR-T cells.

FIG. 8. Cytokine antagonists-expressing CD19 CAR-T cells can be enriched by CD34 epitope. Flow cytometry analysis of the panel of CAR and CD34 epitope expression. Primary human T cells were sorted by CD34 expression 13-days after activation. The strategy of using CD34 epitope to enrich CAR-T cells is applicable to CAR-T cells with expression of cytokine antagonists. However, cells with lower CD34-epitope expression may be lost after the enrichment.

FIG. 9. Schematic of humanized NSG-SGM3 mouse model with high tumor burden. Six- to 8-weeks old NSG-SGM3 mice were sub-lethally irradiated followed by retro-orbital injection of 3×10⁵ HSPCs for humanization. Once animals have been confirmed to be humanized with >20% of human cells in peripheral blood. Humanized mice were given 4.5×10⁶ ffluc-expressing WT Raji cells via tail-vein injection. Tumor progression was monitored by bioluminescence imaging. Upon tumor engraftment, animals were treated with 8×10⁶ CARP naïve/memory T cells via tail-vein injection. n=4 for all test groups except for PBS condition which had n=2.

FIG. 10. Mean weight change following CAR-T cell injection. Weight of animals at the time of T-cell injection were defined as 100% initial weight. Animals were monitored at a minimum frequency of once per day after T-cell injection and the average weight for each condition was plotted in this figure. The weight change of individual animals are shown in the next figure.

FIG. 11. Weight change of individual animals. Animal weights at the time of T-cell injection were defined as 100% initial weight. Each line represents one animal. Weight of individual animals were monitored at least once a day. PBS condition has n=2 and all the other conditions have n=4.

FIG. 12. Mean clinical scores following CAR-T cell injection. Clinical scores of animals were given based on the health condition of animals with 5 being the maximum and 0 being the minimum. Score: 5=healthy animal; 4=normal activity, ruffled hair; 3=ruffled hair, hunched posture, reduced activity but still active when agitated; 2=ruffled hair, hunched posture, low activity; 1=very low activity, not responsive upon agitation, or moribund; 0=end point. The clinical score of individual animals are shown in the next figure.

FIG. 13. Clinical score of individual animals in different conditions. Clinical score is defined as in FIG. 4.10 and in Method section. Each line represents one animal. Animals were monitored at least once per day and a score was given based on the health condition of animal at the time of monitoring. PBS condition has n=2 and all the other conditions have n=4.

FIG. 14. Kaplan-Meier analysis of the mice survival. Survival of animals treated with CAR-T cells with different self-modulators. PBS condition has n=2 and all the other conditions have n=4. Log-rank Mantel-Cox test was used to calculate statistical significance comparing the difference between CD19 CAR and the other test groups. *, p≤0.05; **, p≤0.01. Unmarked pairs do not show statistical difference (p>0.05).

FIG. 15. Engineered T cells were capable of eliminating tumors in vivo. Tumor burden was evaluated by measuring radiance intensity via bioluminescence imaging. Each line represents one animal. PBS condition has n=2 and all the other conditions have n=4.

FIG. 16. Cytokine measurement in peripheral blood 3 days post T-cell injection. Peripheral blood samples from animals were collected via submandibular bleeding 3 days post T-cell injection. Cytokine levels were quantified by bead-based flow cytometry assay. Serum levels of human IL-2, IL-4, IL-6, IL-10, IFN-γ, TNF-α and GM-CSF were quantified by bead-based flow cytometry. IL-4 was undetectable for all the conditions. Serum level of IL-10, IL-6 and IL-2 were down-regulated when T cells were expressing TNF-α antagonists. On the other hand, tocilizumab scFv has similar cytokine expression level as CD19 CAR condition. PBS condition has n=2 and all the other conditions have n=4.

FIG. 17. End-point observations log (Day 0=day of tumor injection; Day 6=day of T cell injection).

FIG. 18. Self-regulating T cells to prevent CRS.

FIG. 19. sToci (Tocilizumab scFv) can inhibit IL-6 signaling.

FIG. 20. sToci-T cells: cytokine production test.

FIG. 21. sToci reduces the production of inflammatory cytokines.

FIG. 22. sToci-T cells: proliferation, cytotoxicity, differentiation, and exhaustion.

FIG. 23. sToci does not inhibit T-cell proliferation and cytotoxicity.

FIG. 24. Secretion of sToci does not alter the differentiation and exhaustion marker expression of primary human CD4⁺ T cells. Constitutive secretion of sToci does not alter the subtype differentiation in primary human CD4⁺ T cells. CD19 CAR-T cells were co-incubated with Raji target cells. T-cell subtype differentiation was evaluated every two days by flow cytometry. data from a total of four experiments are shown.

FIG. 25. sToci does not alter exhaustion marker expression. Constitutive secretion of sToci does not alter the exhaustion-marker expression in primary human CD4⁺ T cells. CD19 CAR-T cells were co-incubated with Raji target cells. Exhaustion-marker expression was evaluated every two days by flow cytometry. Each dot represents the average value of triplicate samples from each independent experiment; data from a total of four experiments are shown. Black bars represent the average value of the four experiments performed with cells from two healthy donors. T_{E/EM}, CCR7⁻/CD45RA⁻/CD57⁻; T_{E/Exh}, CCR7⁻/CD45RA⁻/CD57⁺; TEMRA, CCR7⁻/CD45RA⁺, T_{cm}, CCR7⁺/CD45RA⁻; Tₙₐᵢᵥₑ, CCR7⁺/CD45RA⁺.

FIG. 26. sToci-CAR-T cells maintain anti-tumor efficacy in vivo.

FIG. 27. In vivo model for CAR-T cell-mediated CRS.

FIG. 28. PBMC-humanized NSG model triggers cytokine production in antigen-specific manner.

FIG. 29A-B. Schematic of self-regulating T-cell system. (A) Uncontrolled CAR-T-cell activation may lead to immune over-stimulation and result in CRS. (B) Self-regulating CAR-T cells secrete anti-IL-6Ra sToci to inhibit IL-6 signaling, thus preventing immune over-stimulation.

FIG. 30A-B. Tocilizumab-derived sToci is efficiently produced and secreted by human cells. (A) HEK293T cells were transfected with DNA plasmids encoding the constructs shown in Figure. 2.2A and serum-starved for 24 h beginning 24 h after transfection. Culture supernatant was subsequently collected for western blotting with an anti-FLAG antibody. (B) Lentivirally transduced primary CD4⁺ T cells expressing a second-generation CD19 CAR with or without sToci expression were serum-starved for 72 h. Culture supernatant was subsequently collected for western blotting with an anti-FLAG antibody.

FIG. 31A-C. Tocilizumab-derived single-chain antibody efficiently inhibits IL-6 signaling in human cells. Western blot on the lysate of (A) HepG2 cells, (B) primary human CD4⁺ and CD8⁺ T cells, and (C) primary human CD4⁺ T cells expressing second-generation CD19 CAR with or without sToci and stimulated with indicated concentrations of IL-6. pSTAT3, phosphorylated STAT3. “Normalized pSTAT3” values indicate the pSTAT3 band intensity normalized by the GAPDH band intensity.

FIG. 32A-B. Anti-IL-6Ra sToci reduces the production of inflammatory cytokines in co-cultures of antigen-stimulated primary human T cells and donor-matched PBMCs.

FIG. 33A-B. IFN-γ and IL-10 production was highly variable across donors. (A) CD4⁺ T cells expressing the CD19 CAR with or without sToci were co-cultured with donor-matched PBMCs and CD19⁺ Raji lymphoma cells at a 1:1:1 ratio, with or without the addition of 90 μg/ml tocilizumab. Cytokine concentration in culture supernatant was quantified by bead-based flow cytometry assay after a 12-hours co-incubation period. (B) Cell co-cultures were set up as described in (A), and culture supernatant was harvested after 12 hours for IFN-γ analysis by ELISA. Values shown in all plots are the means of three biologically independent cell cultures from the same donor, with error bars indicating ±1 standard deviation (s.d.). Statistical comparisons were performed using a two-tailed Student's t-test. *, p≤0.05; **, p≤0.01; ***, p≤0.001; unmarked pairs do not show statistically significant difference (p>0.05).

FIG. 34A-C. Constitutively expressed sToci does not inhibit the proliferation and cytotoxicity of primary human T cells. (A) Timeline of co-incubation experiment. CD19 CAR-expressing sToci- and no-sToci-T cells were co-incubated with either wildtype (WT; CD19⁺) or CD19⁻ mutant Raji target cells at a 2:1 effector-to-target (E:T) ratio. Viable cell counts were quantified by flow cytometry every two days. CD4⁺ T cells were harvested 8 days after initial antigen exposure and re-challenged with fresh target cells at a 2:1 E:T ratio. No re-challenge was performed on CD8⁺ T cells due to the limited number of viable T cells remaining at the end of the first challenge. (B, C) Expression of sToci does not hinder the cytotoxicity and proliferation of (B) CD4⁺ and (C) CD8⁺ T cells. Values shown are the means of three biologically independent cell cultures from the same donor, with error bars indicating ±1 s.d. The results for CD4⁺ T cells are representative of four independent experiments performed with cells from two healthy donors; results for CD8⁺ T cells are representative of three independent experiments performed with cells from two healthy donors.

FIG. 35A-B. Secretion of sToci does not alter the differentiation and exhaustion marker expression in primary human CD8⁺ T cells. Constitutive secretion of sToci does not alter (A) subtype differentiation or (B) exhaustion-marker expression in primary human CD8⁺ T cells. CD19 CAR-T cells were co-incubated with Raji target cells as described in FIG. 2.4. T-cell subtype differentiation and exhaustion-marker expression were evaluated every two days by flow cytometry. Each dot in (B) represents the average value of triplicate samples from each independent experiment; data from a total of three experiments are shown. Black bars represent the average value of the three experiments performed with cells from two healthy donors. T_{E/EM}, CCR7⁻/CD45RA⁻/CD57⁻; T_{E/Exh}, CCR7⁻/CD45RA⁻/CD57⁺; TEMRA, CCR7⁻/CD45RA⁺, T_{cm}, CCR7⁺/CD45RA⁻; Tₙₐᵢᵥₑ, CCR7⁺/CD45RA⁺.

FIG. 36. CD19 CAR-T cells that constitutively secrete sToci retain robust anti-tumor cytotoxicity in vivo. NSG mice were engrafted with 2×10⁶ human PBMC and 0.5×10⁶ CD19⁺, firefly luciferase-expressing Raji lymphoma 7 days before treatment with 2×10⁶ donor-matched CD4⁺ T cells. Three groups of mice (n=5) were treated with either (i) no-sToci-T cells, (ii) sToci-T cells, or (iii) T cells expressing only the transduction marker EGFRt. Tumor progression was monitored by bioluminescence imaging. T cells were injected on day 7. Statistical comparisons were performed using a two-tailed Student's t-test at each time point. Results showed no statistically significant difference between animals in the sToci- and no-sToci-T cell groups at any time point.

FIG. 37A-C. CD19 CAR-T cells that constitutively secrete sToci retain robust anti-tumor cytotoxicity and T-cell persistency in vivo. Animals engrafted with human PBMCs and firefly luciferase-expressing Raji tumors were treated with T cells as described in FIG. 2.6. (A) Animals treated with CAR-T cells (with or without sToci production) showed distinct weight loss corresponding to tumor regression after T-cell injection on day 7. No substantial difference was observed in the weight of animals treated with sToci- versus no-sToci-T cells. (B) Kaplan-Meier survival analysis. All surviving animals were sacrificed on day 35 (28 days post T-cell injection) as the experimental end point. There were no observable differences in the behavior or health conditions of the animals at the time of sacrifice. (C) Prevalence of CAR-T cells (CD45⁺/HA⁺) in the bone marrow and blood at the time of sacrifice. Statistical comparisons were performed using a two-tailed Student's t-test. *, p≤0.05; **, p≤0.01; n.s., not significant.

FIG. 38. TNF-α antagonists can decrease the intensity of NFκB signaling in Jurkat cells. Jurkat cells stably expressing an NFκB reporter were stimulated with TNF-α and treated with various scFvs as descried in FIG. 3.2. The median fluorescent intensity of EGFP in Jurkat cells was evaluated by flow cytometry. Values shown are the means of three biologically independent cell cultures, with error bars indicating ±1 standard deviation (s.d). Each group of three bars represents 10, 20, and 50 μg/ml when going from left to right. The bars in the controls represent 10 μg/ml.

FIG. 39A-B. Incorporation of CD34 epitope dampen the effector function of CD19 CAR. Three T cell lines were included in this experiment: (1) CD19 CAR T cells with EGFRt, unsorted; (2) CD19 CAR with CD8a hinge and CD34 epitope, unsorted and (3) CD19 CAR with CD8a hinge and CD34 epitope, sorted. T cells were co-incubated with wildtype Raji cells at 1:1 effector-to-target ratio based on number of CARP T cells. To evaluate the functionality of T cells after repeated antigen challenge, additional Raji cells were added into the culture every 3 days. Viable cell counts of (A) T cells and (B) target cells were quantified by flow cytometry every 3 days, before addition of Raji cells. CD19 CAR with CD34 epitope showed inferior cytotoxicity and proliferation compare to the original CD19 CAR. However, because of the inconsistency between two donors, it is unclear whether the sorting process contributes to the inferior performance of CAR with CD34 epitope. Values shown are the means of three biologically independent cell cultures, with error bars indicating ±1 s.d.

FIG. 40A-C. T cells expressing CD19 CAR incorporated with CD34 epitope and CD8□ hinge showed lower expression of exhaustion markers upon antigen stimulation compared to T cells expressing the original CD19 CAR. Three T cell lines were included in this experiment: (1) CD19 CAR T cells with EGFRt, unsorted; (2) CD19 CAR with CD8a hinge and CD34 epitope, unsorted and (3) CD19 CAR with CD8a hinge and CD34 epitope, sorted. T cells were co-incubated with wildtype or CD19-knockout Raji cells at 1:1 effector-to-target ratio. The expression of (A) PD-1 (B) LAG-3 and (C)Tim-3 were quantified by flow cytometry after a 2-day incubation. CD19 CAR with CD34 epitope showed lower expression of all 3 exhaustion markers when co-incubated with wildtype Raji cells.

FIG. 41A-B. Cytokine antagonists can modulate cytokine production by both engineered T cells and nearby, unmodified immune cells. (A) Polyfunctionality and (B) polyfunctional strength index (PSI) results generated by Isoplexis analysis. Co-culture of (i) CD4⁺ CAR-T cells, (ii) CD8⁺ CAR-T cells, (iii) target cells, (iv) PBMC-derived CD4⁺ T cells and (v) monocytes were mixed in 1.5:0.5:2:1:1 ratio and incubated for 21 hours before Isoplexis assay. Cells were co-incubated with off-target K562 cells as negative control. All cell lines express CD19 CAR and co-express EGFRt or cytokine antagonist. Cell lines were labeled with EGFRt or cytokine antagonists in this figure. Adali-, adalimumab scFv. Certo-, certolizumab scFv. Emapa-, emapalumab scFv. Tocili-, tocilizumab scFv. This experiment was performed in collaboration with Isoplexis. Cells were generated in the Chen Lab, and the experiment was performed by Isolaexis. Data and figure were generated by Isoplexis using Isospeak software.

FIG. 42. PBMC-humanized NSG mice showed potential CRS weight loss pattern. Three NSG mice were injected with 2×10⁷ of human PBMCs and 5×10⁵ of CD19⁺, firefly luciferase-expressing Raji lymphoma cells via tail-vein injection on day 0. Once the tumor engraftment was confirmed by bioluminescence imaging, 1×10⁷ of donor-matched T cells expressing CD19 CAR were injected into animals via tail-vein injection. Weight of animals were monitored daily and the weight on the day of PBMC and Raji cell injection is defined as 100% initial weight. Units of radiance: p/sec/cm²/sr.

FIG. 43A-B. Incorporation of VIVIT peptide by using lentiviral supernatant leads to highly transduced cells with low viability. EGFP NFAT reporter Jurkat cells were transduced with the panel of VIVIT peptide by spinfection. 20-hours after transduction, Jurkat cells were then transferred to a new plate and cultured in complete RPMI. (A) Viability of cells, (B) transfection efficiency.

FIG. 44A-B. Human CAR-T cells trigger IFN-γ and GM-CSF production but the expression of sToci does not alter the cytokine expression levels in a PBMC-humanized NSG mouse model. Experiment was set up as indicated in FIG. 27. Peripheral blood was collected 8 days post T-cell injection and again at the time of euthanasia. Serum levels of human IL-2, IL-4, IL-6, IL-10, IFN-γ, TNF-α and GM-CSF were quantified by bead-based flow cytometry. Only (A) IFN-γ and (B) GM-CSF were present at detectable levels. Statistical comparisons were performed using a two-tailed Student's t-test. *, p≤0.05; **, p≤0.01; ***, p≤0.001; n.s., not significant. Units of radiance: p/sec/cm²/sr. Each group of three bars represents Original CD19 CAR, Unsorted CD19 CAR-CD34 epitope, and Sorted CD19 CAR-CD34 epitope, when going from left to right.

FIG. 45. T cells generated from cord blood showed efficacy in proliferation and cytotoxicity. CD19 CAR T cells and untransduced (UT) T cells generated from cord blood were co-incubated with wildtype or CD19⁻ Raji cells at 1:1 effector-to-target ratio. To evaluate the functionality of T cells after repeated antigen challenge, additional Raji cells were added into the culture every two days. Viable cell counts of (left) target cells and (right) T cells were quantified by flow cytometry every two days, before addition of Raji cells. CD19 CAR T cells generated from cord blood were able to clear WT Raji cells for up to three challenges and did not have off-target toxicity. Values shown are the means of three biologically independent cell cultures, with error bars indicating ±1 standard deviation (s.d.).

FIG. 46A-B. Alternative humanized mouse model utilizing HSPC-humanized NSG-SGM3 mice and TRAC/B2M knockout T cells. (A) Schematic of HSPC-humanized NSG-SGM3 mouse model. Seven 12-weeks-old NSG-SGM3 mice were sub-lethally irradiated (200 rads) with a Cesium-137 irradiator followed by tail-vein injection of 3×10⁵ of human hematopoietic stem and progenitor cells generated from fetal liver tissue. Humanization was confirmed 4-weeks after injection of HSPCs by observing the total percentage of human cells to be higher than 20% in the peripheral blood. Upon humanization, animals were injected with 6×10⁶ of either (1) edited CD19 CART cells (n=3), (2) edited EGFRt T cells (n=3), or (3) unedited CD19 CAR T cells (n=1). (B) TRAC and B2M expression in the injected T cells. The percentage of knockout populations were confirmed and adjusted to similar levels with TRAC single-knockout cells or unedited cells before injection.

FIG. 47A-B. TRAC/B2M-knockout CD19 CAR-T cells kill B cells in HSPC-humanized NSG-SGM3 mice but do not persist in vivo. Experiment was set up as described in FIG. 4.5. Blood samples were collected 1 day before T-cell injection, 5 days after T-cell injection, or at end point. (A) Frequency of human B cells in peripheral blood were quantified based on human CD19 expression. Animals receiving CD19 CAR-T cells showed decreased B-cell population in peripheral blood, confirming the ability of CD19 CAR-T cells to target CD19⁺ B cells. (B) HA-tag expression is used to evaluate CAR expression on T cells. CAR expression levels were measured before T-cell injection and at the end point. CD45⁺ T cells recovered from animals treated with TRAC/B2M-knockout (i.e., “edited”) cells showed reduced % CAR⁺ compared to T cells recovered from the “unedited” are, suggesting that CARP cells lacking T-cell receptor and/or MHC-I expression may have inferior persistence in vivo. Error bar is only included in one sample as all other conditions had n=1 in this staining experiment.

FIG. 48. Edited (TRAC/B2M-knockout) T cells postponed the occurrence of GvHD-associated weight loss in HSPC-humanized NSG-SGM3 mice. Experiment was set up as indicated in FIG. 4.4. Animal weights were recorded daily, with the weight on the day of irradiation and HSPCs cell injection defined as 100% initial weight. There was no significant difference in weight changes among the different test groups until 190 hours post T-cell injection, at which point the animal treated with unedited CD19 CAR-T cells began showing significant weight loss. The absence of weight loss from animals treated with edited T cells suggests that TRAC and B2M knockout may have delayed the occurrence of GvHD.

FIG. 49. Limited CD3⁺ cells in both CD34-enriched and CD34-depleted cells from fetal liver. Human CD34-enriched and CD34-depleted cells were isolated from fetal liver tissue. Both populations of cells were then stained with CD3 antibody to identify the CD3⁺ population within the cells.

FIG. 50. CD3⁺ T cells can be cultured and transduced from the CD34-depleted cells generated from fetal liver. Both CD34-enriched and CD34-depleted cells generated from human fetal liver were stimulated with CD3/CD28 dynabeads and transduced with untitered retrovirus that denotes the expression of a HA-tagged CD19 CAR. 8-days post stimulation, dynabeads were removed from culture and the CD3 expression and transduction efficiency were evaluated. A distinct CD3⁺ population with HA expression was observed in CD34-depleted culture, indicating successful transduction in T cells.

FIG. 51. CD3⁺ cells could be enriched from CD34-depleted fetal liver cells before stimulation, but the population was not viable. CD34-depleted cells generated from human fetal liver were enriched for human CD3 expression by using bead-based magnetic enrichment. CD3 expression was evaluated by antibody staining after enrichment. Percentage of CD3⁺ cells was significantly increased but viable cell population was not distinct after the enrichment.

FIG. 52A-B. Human fetal spleen tissue contains CD3⁺ cells and mixed cell types. (A) Mononuclear cells isolated from human fetal spleen were evaluated for CD45 and CD3 expression via antibody staining. Upon isolation, cells were stimulated with CD3/CD28 dynabeads and transduced with untitered retrovirus encoding HA-tagged CD19 CAR 2- and 3-days after stimulation. (B) Dyanbeads were removed 7 days after stimulation. Transduction efficiencies from untransduced and transduced cells were evaluated by antibody staining of CD3 and HA tag.

FIG. 53A-B. Cord blood can be a source for CD3⁺ T cells. (A) CD3 expression of cells generated from cord blood were evaluated after isolation and 7-days post dynabeads stimulation. Pan-T cell enriched cells showed distinct CD3 expression that coincides with primary human T-cell control. (B) Cells generated from cord blood were stimulated with CD3/CD28 dynabeads upon isolation and transduced with untitered retrovirus encoding HA-tagged CD19 CAR 2 and 3 days after stimulation. Transduced efficiency was evaluated on day 7, upon dynabead removal. Pan-T cell enriched cells were mostly CD3⁺ and had a transduction efficiency of about 83%.

FIG. 54A-B. Transduced and electroporated primary human T cells showed adequate editing and transduction efficiencies. (A) TRAC and B2M expression in edited and unedited T cells. The editing efficiencies were similar between T cells transduced with different constructs, whereas unedited cells maintained high TRAC and B2M expression. (B) HA tag expression was used to evaluate the expression of CD19 CAR on transduced cells. The transduction efficiency was not affected by electroporation of Cas9 RNP, and both edited and unedited cells showed transduction efficiency of about 57%-59%.

FIG. 55A-B. Injected T cells have similar levels of CAR expression and CTV labeling. Primary human naïve/memory T cells were stimulated with CD3/CD28 dynabeads and transduced with untitered retrovirus. Dynabeads were removed from culture on day 7 and T cells were labeled with CellTrace Violet (CTV) on day 11 before being injected into animals. (A) HA tag expression was used to evaluate the expression of CD19 CAR on transduced cells. All conditions have similar expression levels of CD19 CAR. (B) All conditions showed uniform CTV expression levels after CTV labeling on the day of T-cell injection.

FIG. 56A-C. Weight change, clinical score and tumor burden of individual animals in CD19 CAR-tocilizumab scFv conditions. (A) weight change, (B) Clinical score and (C) tumor burden of animals treated with CD19 CAR-tocilizumab scFv. Animal weight at the time of T-cell injection was defined as 100% initial weight. Clinical score was defined as described in FIG. 4.10 and in the Methods section. Tumor burden was evaluated by measuring radiance intensity via bioluminescence imaging. Each line represents one animal. Animals were monitored at least once per day and a score was given based on the health condition of animal at the time of monitoring.

FIG. 57A-B. Schematic of IL-6 inducible system. (A) IL-6 inducible system in CAR-T cells, the production of self-modulators would be up-regulated once IL-6 signaling in T cells is triggered. (B) a basic IL-6 inducible promoter contains an IL-6 response element and a minimal promoter. The list of response elements and minimal promoters included in this study is shown in the pink and black boxes, respectively.

FIG. 58A-D. Screening of responsive elements and minimal promoters in HEK293T cells. Screening of (A, B) Different IL-6 responsive elements were coupled with miniTK as minimal promoter to screen responsive elements. (C, D) Different minimal promoters were coupled with JRE-IL-6 as responsive element to screen minimal promoters. (A, C) Gluc enzymatic activity in culture supernatant was quantified as a measure of gene expression output. (B, D) Fold change was obtained by comparing the difference in Gluc signal between samples with and without IL-6. Values shown are the means of three biologically independent cell cultures, with error bars indicating ±1 standard deviation (s.d.). Units of radiance: p/sec/cm²/sr. In A, the left bar of each set of two bars represents no IL-6 and the right bar represents 50 ng/mL IL-6. In C, the left bar of each set of two bars represents no IL-6 and the right bar represents 10 ng/mL IL-6.

FIG. 59A-B. Firefly luciferase (ffluc) assay in primary human T cells showed limited inducibility of IL-6 inducible promoter. (A) Primary human T cells were transduced with the best-performing IL-6 inducible promoter (JRE-IL-6 as response element and YB TATA as minimal promoter) driving the expression of ffluc. Cells were incubated with 0 to 50 ng/ml of IL-6 for 24 hours, and luciferase activity in the culture supernatant was quantified. (B) Fold change of ffluc activity upon IL-6 induction for the experiment shown in (A). Values shown are the means of three biologically independent cell cultures, with error bars indicating ±1 s.d.

FIG. 60A-C. Incorporation of ZF-STAT3 increases fold change but decreases expression level. (A) Schematic of ZF-STAT3 strategy to increase fold induction. (B, C) HEK293T cells were transfected with plasmids encoding Gluc expressed from IL-6 inducible promoter (JRE-IL-6 coupled to YB TATA) with or without ZF-binding site. Two orientations of ZF-STAT3 constructs were transfected into designated cells at the same time. Cells were incubated with 4 ng/ml IL-6 for 24 hours, and the (B) Gluc activity in the culture was quantified. In B, the left bar of each set of two bars represents no IL-6 and the right bar represents 4 ng/mL IL-6. (C) Fold changes of Gluc activity upon IL-6 induction are reported. Values shown are the means of three biologically independent cell cultures, with error bars indicating ±1 s.d.

FIG. 61A-E. NFAT-responsive promoter driving the expression of single-chain tocilizumab (sToci) cytokine expression Jurkat cells. (A) Schematics of NFAT-responsive promoter driving the expression of sToci or ffluc. Constitutively expressed EGFRt is included as transfection control. Proper expression of NFAT-responsive constructs was observed in Jurkat cells with CD19 CAR transduced with lentivirus encoding NFAT-responsive promoter driving the expression of sToci or ffluc (B). NFAT promoter contains 3 copies (3×) or 6 copies (6×) of NFAT binding domain were tested. Cells were incubated with wildtype or CD19 knock-out Raji cells for 24 hours. Cytokine expression was evaluated by intracellular staining of (C) IFN-γ, (D) IL-2 and (E) TNF-α. Values shown are the means of three biologically independent cell cultures, with error bars indicating ±1 s.d. Statistical comparisons were performed using a two-tailed Student' t-test. *, p≤0.05. Unmarked pairs do not show statistically significant difference (p>0.05). In C-E, each set of three bars represents, from left to right, 6×pNFAT-ffluc; 6×pNFAT-sToci; and 3×pNFAT-sToci.

FIG. 62. Low percentage of double-positive population in primary human T cells after dual-transduction of CD19 CAR and NFAT-responsive reporter. Primary human T cells were transduced with two different lentiviruses encoding (1) NFAT reporter driving expression of ffluc or sToci and (2) CD19 CAR on day 1 and day 3, respectively. No lentivirus was added into mock transduced cells. Expression of CD19 CAR was evaluated by HA-tag staining. Expression of NFAT reporter was evaluated by the constitutively expressed EGFRt.

FIG. 63A-D. IL-6-inducible expression of VIVIT or RCAN peptide to inhibit NFAT signaling as alternative strategy to mediate CRS. (A) Schematics of IL-6-inducible expression of VIVIT or RCAN peptides to mediate CRS. VIVIT and RCAN peptides can inhibit NFAT signaling and control T-cell activation. (B, C, D) Verification of endogenous IL-6-inducible genes. Primary human T cells were incubated with IL-6 from 0 to 10 ng/ml for 24 or 48 hours followed by intracellular staining of (B) IL-17A or (C) IL-21; or surface staining of (D) IL-23R. For B-D, each set of 3 bars represents, from left to right, 0, 5, and 10 ng/ml.

FIG. 64A-D. Electroporation of VIVIT and RCAN peptides damages the fitness of Jurkat cells. (A) List of VIVIT and RCAN peptides tested. EGFP NFAT reporter Jurkat cells were electroporated with the panel of VIVIT peptides. 24-hours after electroporation, PMA/ionomycin were added into designated cells to induce NFAT signaling. (B) transfection efficiency, (C) viability of cells and (D) EGFP expression were evaluated by flow cytometry 24-hours after PMA/ionomycin addition. UT, untransfected. For B-D, the left bar of each set of two bars represents w/o PMA/ionomycin and the right bar represents w/ PMA/ionomycin.

FIG. 65. Minimal promoters provide a range of background expression levels. HEK293T cells were transfected with a panel of different minimal promoters driving the expression of Gluc to evaluate the basal expression from minimal promoters. Cells were incubated in complete or serum-free media for 24 hours before the measurement of Gluc enzymatic activity in culture supernatant. Values shown are the means of three biologically independent cell cultures, with error bars indicating ±1 standard deviation (s.d.). The left bar of each set of bars represents complete media and the right bar represents serum-free media.

FIG. 66A-B. IL-6 inducible promoter showed limited inducibility in Jurkat cells. Jurkat cells were transfected with plasmids encoding Gluc expressed from various minimal promoters coupled with JRE-IL-6 response element. Cells were incubated with 0 or 10 ng/ml IL-6 for 24 hours, and the (A) Gluc activity in the culture was quantified. The left bar of each set of 2 bars represents no IL-6 and the right bar represents 10 ng/ml IL-6. (B) Fold changes of Gluc activity upon IL-6 induction were calculated by dividing Gluc radiance of samples treated with IL-6 by the radiance of those not treated with IL-6. Values shown are the means of three biologically independent cell cultures, with error bars indicating ±1 s.d.

FIG. 67A-B. IL-6 inducible promoter can be expressed in primary human T cells. Primary human CD4⁺ T cells were transduced with lentivirus encoding an IL-6 inducible promoter driving the expression of firefly luciferase (ffLuc) in addition to a constitutive EF1α promoter driving the expression of a truncated epidermal growth factor receptor (EGFRt), which serves as a transduction marker (A). (B) Transduced T cells were enriched by EGFRt expression prior to use in experiments.

FIG. 68. B2M locus can be edited by CRISPR-HDR strategy for constitutive expression of gene of interest. Primary human CD4⁺ and CD8⁺ T cells were edited with CRISPR-HDR to knock-out B2M gene and incorporate plasmid encoding EGFP. FACS analysis was conducted 3 days post editing and flow plots showed efficient editing of B2M locus. Blue population: unedited cells, red population: edited cells. VG, viral genome.

FIG. 69A-C. Decreasing DNA input in electroporation reaction reduces toxicity but leads to poor transfection efficiency. EGFP NFAT reporter Jurkat cells were electroporated with the panel of VIVIT peptide constructs. The amount of DNA used in this experiment was half of that used in typical electroporation reactions. Twenty-four hours after electroporation, PMA/ionomycin were added into designated cells to induce NFAT signaling. (A) Viability of cells, (B) transfection efficiency and (C) EGFP expression were evaluated by flow cytometry 24 hours after PMA/ionomycin addition. UT, untransfected. The left bar of each set of two bars represents w/o PMA/ionomycin and the right bar represents w/ PMA/ionomycin.

FIG. 70A-B. Effects of cytokine modulators on tumor-bearing humanized mice treated with CD19 CAR-T cells. NSG-SGM3 mice humanized with fetal liver CD34⁺ T cells were engrafted with 4.5 million Raji tumor cells that stably express firefly luciferase. Nine days post tumor injection, each animal was treated with 8 million CD19 CAR-expressing T cells that either does not produce a cytokine modulator (CD19 CAR-only) or secretes the cytokine modulator denoted above each plot (n=10 per group for emapalumab scFv, certolizumab scFv, and CD19 CAR only; n=9 for tocilizumab scFv and anti-IL1Ra). The CAR-T cells originate from a healthy adult human donor (i.e., a different donor than the fetal liver donor). (A) Animal health was monitored daily, and weight measurements reveal the rate and manner of decline in each animal. T-cell injection triggers rapid weight loss within the first 4 days in all test groups, but animals treated with T cells that secrete cytokine modulators show improved recovery relative to those treated with T cells that do not produce cytokine modulators. (B) Tumor progression was monitored through bioluminescence imaging. Results show all CAR-T cells were able to eradicate the engrafted tumor, indicating the secretion of cytokine modulators did not compromise the anti-tumor efficacy of engineered cells.

FIG. 71A-C. Confirmation of cytokine modulator activity in humanized mouse model. NSG-SGM3 mice humanized with fetal liver CD34+ T cells were engrafted with 5 million Raji tumor cells that stably express firefly luciferase. Five days post tumor injection, each animal was treated with 8 million CD19 CAR-expressing T cells that either does not produce a cytokine modulator (CD19 CAR-only) or secretes the cytokine modulator denoted above each plot (n=10 per group). “Untreated” animals were engrafted with tumor but not treated with CAR-T cells. “Mock-Transduced T Cells” were cells from the same healthy human donor but transduced with a virus that does not encode for any CAR. (A) Animal health was monitored daily, and weight measurements reveal the rate and manner of decline in each animal. Results confirmed that animals treated with T cells that secrete cytokine modulators show improved recovery relative to those treated with T cells that do not produce cytokine modulators. Animals that were untreated or treated with mock-transduced T cells did not experience the initial weight decline seen in animals treated with CD19 CAR-T cells, confirming this model's ability to reveal toxicity related to antigen-specific T-cell stimulation. Animals treated with mock-transduced T cells eventually exhibited weight decline and symptoms consistent with graft-versus host disease, which was expected given that the adoptively transferred T cells came from a different donor than the CD34⁺ cells used for mouse humanization. (B) Tumor progression was monitored through bioluminescence imaging. Results confirm untreated animals could not control tumor progression. Mock-transduced T cells showed initial tumor decline that can be attributed to allogeneic effects of the adoptively transferred T cells against Raji tumors. However, these animals eventually experience tumor resurgence. Results confirmed that all CAR-T cell-treated groups were able to eradicate the engrafted tumor, indicating the secretion of cytokine modulators did not compromise the anti-tumor efficacy of engineered cells. (C) Survival data indicate prolonged survival of animals treated with CAR-T cells that co-express tocilizumab scFv and, to a lesser extent, emapalumab scFv relative to animals treated with CD19 CAR-T cells without cytokine modulators.

### DESCRIPTION OF ILLUSTRATIVE EMBODIMENTS

Embodiments disclosed herein include polypeptides that modulate the inflammatory effects of cytokines such as TNF-α, INF-gamma, and IL-1 receptor. Further embodiments include cells that express the polypeptides, as well as methods of using the cells and polypeptides in cancer therapy, autoimmune therapy, and anti-inflammatory therapy, among others. These and other non-limiting aspects of the present invention are discussed in further detail in the following sections.

### I. Definitions

Embodiments disclosed herein may include chimeric antigen receptors (CARs), which may be expressed by T cells or other immune cell types. CARs are engineered receptors, which graft an arbitrary specificity onto an immune effector cell. Typically, these receptors are used to graft the specificity of a monoclonal antibody onto a T cell. The receptors are called chimeric because they are composed of parts from different sources.

The terms “protein,” “polypeptide,” and “peptide” are used interchangeably herein when referring to a gene product.

“Homology,” “identity,” or “similarity” refers to sequence similarity between two peptides or between two nucleic acid molecules. Identity can be determined by comparing a position in each sequence which may be aligned for purposes of comparison. When a position in the compared sequence is occupied by the same base or amino acid, then the molecules share sequence identity at that position. A degree of identity between sequences is a function of the number of matching or homologous positions shared by the sequences. An “unrelated” or “non-homologous” sequence shares less than 40% identity, or less than 25% identity, with one of the sequences of the current disclosure.

The terms “amino portion,” “N-terminus,” “amino terminus,” and the like as used herein are used to refer to order of the regions of the polypeptide. Furthermore, when something is N-terminal to a region it is not necessarily at the terminus (or end) of the entire polypeptide, but just at the terminus of the region or domain. Similarly, the terms “carboxy portion,” “C-terminus,” “carboxy terminus,” and the like as used herein is used to refer to order of the regions of the polypeptide, and when something is C-terminal to a region it is not necessarily at the terminus (or end) of the entire polypeptide, but just at the terminus of the region or domain.

The terms “subject,” “individual,” or “patient” are used interchangeably herein and refer to a vertebrate, for example a primate, a mammal or preferably a human. Mammals include, but are not limited to equines, canines, bovines, ovines, murines, rats, simians, humans, farm animals, sport animals and pets.

The term “xeno-free (XF)” or “animal component-free (ACF)” or “animal free,” when used in relation to a medium, an extracellular matrix, or a culture condition, refers to a medium, an extracellular matrix, or a culture condition which is essentially free from heterogeneous animal-derived components. For culturing human cells, any proteins of a non-human animal, such as mouse, would be xeno components. In certain aspects, the xeno-free matrix may be essentially free of any non-human animal-derived components, therefore excluding mouse feeder cells or Matrigel™. Matrigel™ is a solubilized basement membrane preparation extracted from the Engelbreth-Holm-Swarm (EHS) mouse sarcoma, a tumor rich in extracellular matrix proteins to include laminin (a major component), collagen IV, heparin sulfate proteoglycans, and entactin/nidogen. In some embodiments, the compositions described herein or cells of the disclosure are cultured in and/or prepared in/with xeno-free or animal component-free or animal free medium.

Cells are “substantially free” of certain reagents or elements, such as serum, signaling inhibitors, animal components or feeder cells, exogenous genetic elements or vector elements, as used herein, when they have less than 10% of the element(s), and are “essentially free” of certain reagents or elements when they have less than 1% of the element(s). However, even more desirable are cell populations wherein less than 0.5% or less than 0.1% of the total cell population comprise exogenous genetic elements or vector elements.

A culture, matrix or medium are “essentially free” of certain reagents or elements, such as serum, signaling inhibitors, animal components or feeder cells, when the culture, matrix or medium respectively have a level of these reagents lower than a detectable level using conventional detection methods known to a person of ordinary skill in the art or these agents have not been extrinsically added to the culture, matrix or medium. The serum-free medium may be essentially free of serum.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “cell” is herein used in its broadest sense in the art and refers to a living body which is a structural unit of tissue of a multicellular organism, is surrounded by a membrane structure which isolates it from the outside, has the capability of self-replicating, and has genetic information and a mechanism for expressing it. Cells used herein may be naturally-occurring cells or artificially modified cells (e.g., fusion cells, genetically modified cells, etc.).

As used herein, the term “stem cell” refers to a cell capable of self-replication and pluripotency or multipotency. Typically, stem cells can regenerate an injured tissue. Stem cells herein may be, but are not limited to, embryonic stem (ES) cells, induced pluripotent stem cells or tissue stem cells (also called tissue-specific stem cell, or somatic stem cell).

“Embryonic stem (ES) cells” are pluripotent stem cells derived from early embryos. An ES cell was first established in 1981, which has also been applied to production of knockout mice since 1989. In 1998, a human ES cell was established, which is currently becoming available for regenerative medicine.

Unlike ES cells, tissue stem cells have a limited differentiation potential. Tissue stem cells are present at particular locations in tissues and have an undifferentiated intracellular structure. Therefore, the pluripotency of tissue stem cells is typically low. Tissue stem cells have a higher nucleus/cytoplasm ratio and have few intracellular organelles. Most tissue stem cells have low pluripotency, a long cell cycle, and proliferative ability beyond the life of the individual. Tissue stem cells are separated into categories, based on the sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreatic (common) stem cells, liver stem cells, and the like. Tissue stem cells in the bone marrow system include hematopoietic stem cells, mesenchymal stem cells, and the like. Tissue stem cells in the nervous system include neural stem cells, retinal stem cells, and the like.

“Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by introducing certain factors, referred to as reprogramming factors.

“Pluripotency” refers to a stem cell that has the potential to differentiate into all cells constituting one or more tissues or organs, or particularly, any of the three germ layers: endoderm (interior stomach lining, gastrointestinal tract, the lungs), mesoderm (muscle, bone, blood, urogenital), or ectoderm (epidermal tissues and nervous system). “Pluripotent stem cells” used herein refer to cells that can differentiate into cells derived from any of the three germ layers, for example, direct descendants of totipotent cells or induced pluripotent cells.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

In some embodiments, the methods are useful for reducing the size and/or cell number of a solid tumor. In some embodiments, the method of the disclosure are useful for inhibiting the growth of tumors, such as solid tumors, in a subject.

The term “antigen” refers to any substance that causes an immune system to produce antibodies against it, or to which a T cell responds. In some embodiments, an antigen is a peptide that is 5-50 amino acids in length or is at least, at most, or exactly 5, 10, 15, 20, 25, 30, 35, 40, 45, 50, 55, 60, 65, 70, 75, 80, 85, 90, 95, 100, 125, 150, 175, 200, 250, or 300 amino acids, or any derivable range therein.

The term “antibody” includes monoclonal antibodies, polyclonal antibodies, dimers, multimers, multispecific antibodies and antibody fragments that may be human, mouse, humanized, chimeric, or derived from another species. A “monoclonal antibody” is an antibody obtained from a population of substantially homogeneous antibodies that is being directed against a specific antigenic site.

“Antibody or functional fragment thereof” means an immunoglobulin molecule that specifically binds to, or is immunologically reactive with a particular antigen or epitope, and includes both polyclonal and monoclonal antibodies. The term antibody includes genetically engineered or otherwise modified forms of immunoglobulins, such as intrabodies, peptibodies, chimeric antibodies, fully human antibodies, humanized antibodies, and heteroconjugate antibodies (e.g., bispecific antibodies, diabodies, triabodies, and tetrabodies). The term functional antibody fragment includes antigen binding fragments of antibodies, including e.g., Fab′, F(ab′)2, Fab, Fv, rlgG, and scFv fragments. The term scFv refers to a single chain Fv antibody in which the variable domains of the heavy chain and of the light chain of a traditional two chain antibody have been joined to form one chain.

The use of a single chain variable fragment (scFv) is of particular interest. scFvs are recombinant molecules in which the variable regions of light and heavy immunoglobulin chains encoding antigen-binding domains are engineered into a single polypeptide. Generally, the VH and VL sequences are joined by a linker sequence. See, for example, Ahmad (2012) Clinical and Developmental Immunology Article ID 980250, herein specifically incorporated by reference.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

### II. Polypeptides

A. Chimeric Binding Polypeptides

Besides scFvs, the chimeric binding polypeptides can take a variety of forms that may incorporate one or more of the VH, VL, or CDR sequences disclosed herein or derivatives or variations thereof. For example, the binding protein can be an antibody-like molecule that has an antigen binding region, including antibody fragments such as Fab′, Fab, F(ab′)2, F(ab)2, (scFv)2, single domain antibodies (DABs), Fv, and polypeptides with antibody CDRs, scaffolding domains that display the CDRs (e.g., anticalins) or a nanobody. For example, the nanobody can be antigen-specific VHH (e.g., a recombinant VHH) from a camelid IgG2 or IgG3, or a CDR-displaying frame from such camelid Ig. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

“Mini-antibodies” or “minibodies” are also contemplated for use with embodiments. Minibodies are sFv polypeptide chains which include oligomerization domains at their C-termini, separated from the sFv by a hinge region. Pack et al. (1992). The oligomerization domain comprises self-associating α-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art. See, e.g., Pack et al. (1992); Cumber et al. (1992).

Antibody-like binding peptidomimetics are also contemplated in embodiments. Liu et al. (2003) describe “antibody like binding peptidomimetics” (ABiPs), which are peptides that act as pared-down antibodies and have certain advantages of longer serum half-life as well as less cumbersome synthesis methods.

Alternative scaffolds for antigen binding peptides, such as CDRs are also available and can be used to generate chimeric binding polypeptides in accordance with the embodiments. Generally, a person skilled in the art knows how to determine the type of protein scaffold on which to graft at least one of the CDRs arising from the original antibody. More particularly, it is known that to be selected such scaffolds must meet the greatest number of criteria as follows (Skerra, 2000): good phylogenetic conservation; known three-dimensional structure (as, for example, by crystallography, NMR spectroscopy or any other technique known to a person skilled in the art); small size; few or no post-transcriptional modifications; and/or easy to produce, express and purify.

The origin of such protein scaffolds can be, but is not limited to, the structures selected among: fibronectin and preferentially fibronectin type III domain 10, lipocalin, anticalin (Skerra, 2001), protein Z arising from domain B of protein A of Staphylococcus aureus, thioredoxin A or proteins with a repeated motif such as the “ankyrin repeat” (Kohl et al., 2003), the “armadillo repeat”, the “leucine-rich repeat” and the “tetratricopeptide repeat”. For example, anticalins or lipocalin derivatives are a type of binding proteins that have affinities and specificities for various target molecules and can be used as target binding molecules. Such proteins are described in US Patent Publication Nos. 20100285564, 20060058510, 20060088908, 20050106660, and PCT Publication No. WO2006/056464, incorporated herein by reference.

Scaffolds derived from toxins such as, for example, toxins from scorpions, insects, plants, mollusks, etc., and the protein inhibiters of neuronal NO synthase (PIN) may also be used in certain aspects.

Monoclonal antibodies (MAbs) are recognized to have certain advantages, e.g., reproducibility and large-scale production. Embodiments include monoclonal antibodies of the human, murine, monkey, rat, hamster, rabbit and chicken origin.

“Humanized” antibodies are also contemplated, as are chimeric antibodies from mouse, rat, or other species, bearing human constant and/or variable region domains, bispecific antibodies, recombinant and engineered antibodies and fragments thereof. As used herein, the term “humanized” immunoglobulin refers to an immunoglobulin comprising a human framework region and one or more CDR's from a non-human (usually a mouse or rat) immunoglobulin. The non-human immunoglobulin providing the CDR's is called the “donor” and the human immunoglobulin providing the framework is called the “acceptor”. A “humanized antibody” is an antibody comprising a humanized light chain and a humanized heavy chain immunoglobulin. In order to describe antibodies of some embodiments, the strength with which an antibody molecule binds an epitope, known as affinity, can be measured. The affinity of an antibody may be determined by measuring an association constant (Ka) or dissociation constant (Kd). Antibodies deemed useful in certain embodiments may have an association constant of about, at least about, or at most about 10⁶, 10⁷, 10⁸, 10⁹ or 10¹⁰ M or any range derivable therein. Similarly, in some embodiments antibodies may have a dissociation constant of about, at least about or at most about 10⁻⁶, 10⁻⁷, 10⁻⁸, 10⁻⁹ or 10⁻¹⁰ M or any range derivable therein.

### B. CDR Embodiments

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR4, CDR5, and CDR6. The L3 (CDR-6) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as H1, H2 and H3 or CDR1, CDR2, and CDR3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope; 2) Hydrogen-deuterium exchange and mass spectroscopy; 3) Polypeptide fragmentation and peptide mapping approaches in which one generates multiple overlapping peptide fragments from the full length of the polypeptide and evaluates the binding affinity of these peptides for the epitope; 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate. In certain embodiments, CDR1, CDR2, CDR3, CDR4, CDR5, and/or CDR6 correspond to CDRs determined by the Kabat method based on a VH and VL region described herein. In certain embodiments, CDR1, CDR2, CDR3, CDR4, CDR5, and/or CDR6 correspond to CDRs determined by the Chothia method based on a VH and VL region described herein.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

### C. Modifications to Chimeric Binding Polypeptides

1. Signal Peptides

Any binding polypeptide can be tagged with the leader (i.e. signal) peptide. A “signal peptide” refers to a peptide sequence that directs the transport and localization of the protein within a cell, e.g. to a certain cell organelle (such as the endoplasmic reticulum) and/or the cell surface. A signal peptide directs the nascent protein into the endoplasmic reticulum. Generally, the signal peptide natively attached to the amino-terminal most component is used (e.g. in a scFv with orientation light chain-linker-heavy chain, the native signal of the light-chain is used). In some embodiments the signal peptide from the murine kappa light chain is used so that the proteins are secreted from the expressing cells. This also allows the expressed protein to be directly collected from the media in which the producer cells are cultured. Other leader/signal peptides known to those in the art can also be used.

In some embodiments, the signal peptide is cleaved after passage of the endoplasmic reticulum (ER), i.e., is a cleavable signal peptide. In some embodiments, a restriction site is at the carboxy end of the signal peptide to facilitate cleavage.

2. Markers and Epitope Tags

The binding polypeptides can also be tagged with markers such as the DYKDDDDK (SEQ ID NO:71) (FLAG) epitope, HA tag, or cMyc tag or other epitope tags, markers, and affinity tags known in the art. This can facilitate isolation, purification, localization, and targeted binding of the chimeric binding polypeptides. In some embodiments, the marker is flanked by GGS linkers.

3. Linkers

In some embodiments, the polypeptides of the disclosure include peptide linkers (sometimes referred to as a linker). A peptide linker may be separating any of the peptide domain/regions described herein. As an example, a linker may be between the signal peptide and the antigen binding domain, between a VH and VL of an antigen binding domain, between an antigen binding domain and a peptide spacer, between a peptide spacer and a transmembrane domain. A peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)n, glycine-serine polymers (including, for example, (GS)n, (GSGGS)n (SEQ ID NO:73) and (GGGS)n (SEQ ID NO:74), where n is an integer of at least one, glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. Exemplary spacers can comprise amino acid sequences including, but not limited to, GGSG (SEQ ID NO:75), GGSGG (SEQ ID NO:76), GSGSG (SEQ ID NO:77), GSGGG (SEQ ID NO:78), GGGSG (SEQ ID NO:79), GSSSG (SEQ ID NO:80), and the like.

4. Modifications to Antigen-Binding Domains

The variable regions of antigen-binding domains of the chimeric binding polypeptides of the disclosure can be modified by mutating amino acid residues within VH and/or VL CDR regions to improve one or more binding properties (e.g., affinity) of the protein. The term “CDR” refers to a complementarity-determining region that is based on a part of the variable chains in immunoglobulins (antibodies) and T-cell receptors, generated by B cells and T cells respectively, where these molecules bind to their specific antigen. Since most sequence variation associated with immunoglobulins and T-cell receptors are found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to chimeric binding polypeptides to decrease immunogenicity, for example, by “backmutating” one or more framework residues to a corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more (or any derivable range therein), than the affinity of an antibody for unrelated amino acid sequences. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges.

5. Chemical Modifications

Additionally, the polypeptides of the disclosure may be chemically modified. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861).

The polypeptides of the invention can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive watersoluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. Methods for pegylating proteins are known in the art and can be applied to the polypeptides of the invention (EP 0 154 316 and EP 0 401 384).

Additionally, polypeptides may be chemically modified by conjugating or fusing the polypeptide to serum protein, such as human serum albumin, to increase half-life of the resulting molecule. Such approach is for example described in EP 0322094 and EP 0 486 525.

The polypeptides of the disclosure may be conjugated to a diagnostic or therapeutic agent and used diagnostically, for example, to monitor the development or progression of a disease and determine the efficacy of a given treatment regimen. The polypeptides may also be conjugated to a therapeutic agent to provide a therapy in combination with the therapeutic effect of the polypeptide. Examples of diagnostic agents include enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the polypeptide, or indirectly, through a linker using techniques known in the art. Examples of suitable enzymes include horseradish peroxidase, alkaline phosphatase, beta-galactosidase, or acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerythrin. An example of a luminescent material includes luminol. Examples of bioluminescent materials include luciferase, luciferin, and aequorin. Examples of suitable radioactive material include .sup.125I, .sup.131I, Indium-111, Lutetium-171, Bismuth-212, Bismuth-213, Astatine-211, Copper-62, Copper-64, Copper-67, Yttrium-90, Iodine-125, Iodine-131, Phosphorus-32, Phosphorus-33, Scandium-47, Silver-111, Gallium-67, Praseodymium-142, Samarium-153, Terbium-161, Dysprosium-166, Holmium-166, Rhenium-186, Rhenium-188, Rhenium-189, Lead-212, Radium-223, Actinium-225, Iron-59, Selenium-75, Arsenic-77, Strontium-89, Molybdenum-99, Rhodium-1105, Palladium-109, Praseodymium-143, Promethium-149, Erbium-169, Iridium-194, Gold-198, Gold-199, and Lead-211. Chelating agents may be attached through amities (Meares et al., 1984 Anal. Biochem. 142: 68-78); sulfhydral groups (Koyama 1994 Chem. Abstr. 120: 217262t) of amino acid residues and carbohydrate groups (Rodwell et al. 1986 PNAS USA 83: 2632-2636; Quadri et al. 1993 Nucl. Med. Biol. 20: 559-570).

The polypeptides may also be conjugated to a therapeutic agent to provide a therapy in combination with the therapeutic effect of the polypeptide.

Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. Aptamers are small nucleic acids ranging from 15-50 bases in length that fold into defined secondary and tertiary structures, such as stern-loops or G-quartets, and can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Ribozymes are nucleic acid molecules that are capable of catalyzing a chemical reaction, either intramolecularly or intermolecularly. Ribozymes typically cleave nucleic acid substrates through recognition and binding of the target substrate with subsequent cleavage. Triplex forming function nucleic acid molecules can interact with double-stranded or single-stranded nucleic acid by forming a triplex, in which three strands of DNA form a complex dependent on both Watson-Crick and Hoogsteen base-pairing. Triplex molecules can bind target regions with high affinity and specificity.

The functional nucleic acid molecules may act as effectors, inhibitors, modulators, and stimulators of a specific activity possessed by a target molecule, or the functional nucleic acid molecules may possess a de novo activity independent of any other molecules.

D. Expression of Chimeric Binding Polypeptides

Chimeric binding polypeptides can be expressed in a variety of cell types. An expression construct encoding a chimeric binding polypeptide can be transfected into cells according to a variety of methods known in the art. In some embodiments, the chimeric binding polypeptide expression construct can be under control of a constitutive promoter. In some embodiments, it can be under control of a regulatable promoter that drives expression only under certain conditions. This can provide for expression of a chimeric binding polypeptide only when necessary to respond to a potentially harmful situation or a situation in which it is desirable to reduce cytokine signaling, such as, for example, when there are increased levels of cytokines present. To accomplish this, the chimeric binding polypeptide expression construct may be placed under control of a promoter that is responsive to cytokines such as IL-6, TNF-α, IFN-γ, IL-1β, IL-2, IL-8, and IL-10. The IL-6Ra-binding protein expression construct can also be placed under control of a promoter that is linked to T-cell activation, such as one that is controlled by NFAT-1 or NF-κB, both of which are transcription factors that can be activated upon T-cell activation. Control of protein expression allows T cells, such as tumor-targeting T cells, to sense their surroundings and perform real-time modulation of cytokine signaling, both in the T cells themselves and in surrounding endogenous immune cells.

E. Polypeptide Compositions

The polypeptides or polynucleotides of the disclosure, such as the VH or VL regions of SEQ ID NOS:1-12 or the polypeptide of SEQ ID NO:13, or the linker of SEQ ID NO:14, or other polypeptide or nucleic acid embodiments described throughout, may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOs:1-14 or SEQ ID NO:1-90.

The polypeptides or polynucleotides of the disclosure, such as the V_{H} or V_{L} regions of SEQ ID NOS:1-12 or the polypeptide of SEQ ID NO:13, or the linker of SEQ ID NO:14, or other polypeptide or nucleic acid embodiments described throughout, may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein, of SEQ ID NO:1-14 or of 1-90.

In some embodiments, a polypeptide of the disclosure comprises amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) of SEQ ID NOs:1-14 or 1-90.

In some embodiments, the chimeric binding polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, or 160 (or any derivable range therein) contiguous amino acids of SEQ ID NOs:1-14.

In some embodiments, the chimeric binding polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, or 160 (or any derivable range therein) contiguous amino acids of SEQ ID NOs:1-14 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of SEQ ID NOS:1-14.

In other embodiments, a V_{L} region comprises amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, or 160 (or any derivable range therein) of SEQ ID NO:1, 3, 5, 7, 9, or 11.

In other embodiments, a V_{H} region comprises amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, or 160 (or any derivable range therein) of SEQ ID NO:2, 4, 6, 8, 10, or 12.

In some embodiments, an IL-1 receptor binding polypeptide comprises 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, or 153 (or any derivable range therein) of SEQ ID NO:13.

In other embodiments, a V_{L} region comprises amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, or 160 (or any derivable range therein) contiguous amino acids of SEQ ID NO: 1, 3, 5, 7, 9, or 11 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of SEQ ID NO: 1, 3, 5, 7, 9, or 11.

In other embodiments, a V_{H} region comprises amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, or 160 (or any derivable range therein) contiguous amino acids of SEQ ID NO: 2, 4, 6, 8, 10, or 12 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of SEQ ID NO: 2, 4, 6, 8, 10, or 12.

In some embodiments, an IL-1 receptor binding polypeptide comprises 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, or 153 (or any derivable range therein) contiguous amino acids of SEQ ID NO:13 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of SEQ ID NO:13.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In other embodiments, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific embodiments, all or part of proteins described herein can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One embodiment includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

### III. Nucleic Acids

In certain embodiments, there are recombinant polynucleotides encoding the proteins, polypeptides, or peptides described herein. Polynucleotide sequences contemplated include those encoding chimeric-binding proteins and/or CARs.

As used in this application, the term “polynucleotide” refers to a nucleic acid molecule that either is recombinant or has been isolated free of total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In particular embodiments, there are isolated nucleic acid segments and recombinant vectors incorporating nucleic acid sequences that encode a polypeptide (e.g., an antibody or fragment thereof) that binds to a cytokine or antigen. The term “recombinant” may be used in conjunction with a polypeptide or the name of a specific polypeptide, and this generally refers to a polypeptide produced from a nucleic acid molecule that has been manipulated in vitro or that is a replication product of such a molecule.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

A. Vectors

Polypeptides may be encoded by a nucleic acid molecule. The nucleic acid molecule can be in the form of a nucleic acid vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). Vectors may be used in a host cell to produce an antibody that binds to a cytokine.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.)

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, incorporated herein by reference.)

The vectors or constructs will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels. In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message.

In expression, particularly eukaryotic expression, one will typically include a polyadenylation signal to effect proper polyadenylation of the transcript.

In order to propagate a vector in a host cell, it may contain one or more origins of replication sites (often termed “ori”), which is a specific nucleic acid sequence at which replication is initiated. Alternatively, an autonomously replicating sequence (ARS) can be employed if the host cell is yeast.

B. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

C. Expression Systems

Numerous expression systems exist that comprise at least a part or all of the compositions discussed above. Prokaryote- and/or eukaryote-based systems can be employed for use with an embodiment to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems, other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

D. Methods of Gene Transfer

Suitable methods for nucleic acid delivery to effect expression of compositions are believed to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Through the application of techniques such as these, organelle(s), cell(s), tissue(s) or organism(s) may be stably or transiently transformed. Other methods include viral transduction, such as gene transfer by lentiviral or retroviral transduction.

### IV. Cells

A. Cell Types

Certain embodiments relate to cells comprising polypeptides or nucleic acids of the disclosure. In some embodiments the cell is an immune cell or a T cell. “T cell” includes all types of immune cells expressing CD3 including T-helper cells (CD4+ cells), cytotoxic T-cells (CD8+ cells), T-regulatory cells (Treg), suppressor T cells, and gamma-delta T cells. A “cytotoxic cell” includes CD8+ T cells, natural-killer (NK) cells, and neutrophils, which cells are capable of mediating cytotoxicity responses. In some embodiments, the cell is a progenitor cell or stem cell. In some embodiments, the progenitor or stem cell is in vitro differentiated into an immune cell. In some embodiments, the cell is ex vivo. The term immune cells includes cells of the immune system that are involved in defending the body against both infectious disease and foreign materials. Immune cells may include, for example, neutrophils, eosinophils, basophils, natural killer cells, lymphocytes such as B cells and T cells, and monocytes.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell or progenitor cell obtained from an individual.

B. Adoptive Cell Therapy

In some embodiments, cells disclosed herein are used in adoptive cell therapy or adoptive cell transfer, which involves administration of therapeutic cells to subjects. This therapy can involve administration of tumor-infiltrating lymphocytes, cytotoxic T lymphotytes (CTLs), helper T (Th) cells, and Treg cells, and other immune cells and other types of cells. The therapeutic cells may originate from a patient or from a donor. In some embodiments, cells are extracted from a patient, genetically modified and cultured in vitro and returned to the same patient.

Cells disclosed herein can be administered to a subject to treat, for example, cancer, autoimmune diseases, infections, and inflammatory disorders. In some embodiments, expression of chimeric binding polypeptides by cells used in adoptive T cell therapy can reduce or eliminate side effects that may occur as a result of adoptive T cell therapy, such as cytokine release syndrome and other inflammatory or autoimmune conditions that involve cytokine signaling. In addition, administration of chimeric-binding polypeptides in conjunction with therapeutic T cells can eliminate, reduce, or mitigate the side effects of adoptive T cell therapy.

1. Chimeric Antigen Receptors

a. Use in Adoptive Cell Therapies

In some embodiments, therapeutic cells used in adoptive cell therapies express chimeric antigen receptors (CARs). CARs are fusion proteins that are commonly composed of an extracellular antigen-binding domain (which may be an scFv), an extracellular spacer, a transmembrane domain, costimulatory signaling regions (the number of which varies depending on the specific CAR design), and a CD3-zeta signaling domain/endodomain. Immune cells, including T cells and natural killer (NK) cells, can be engineered to express CARs by a variety of methods known in the art, including viral transduction, DNA nucleofection, and RNA nucleofection. CAR binding to the antigen target can activate human T cells expressing the CAR, which may result in killing of the cell bearing the antigen or some other immunological response.

In some embodiments, the cells comprise a cancer-specific CAR. The term “cancer-specific” in the context of CARs refers to CARs that have an antigen binding specificity for a cancer-specific molecule, such as a cancer-specific antigen. In some embodiments, the cancer specific CAR is in a cell with a CAR specific for another target, such as a TGF-β CAR. In some embodiments, the cancer-specific CAR and another CAR are on separate polypeptides. In some embodiments, the CAR is a bi-specific CAR that has antigen binding for a cancer-specific molecule and for another antigen, such as TGF-β. For example, a bi-specific CAR may have a signaling peptide, a cancer molecule-specific scFv, optionally a peptide linker/spacer, followed by an scFv that binds another antigen, followed by a spacer, a transmembrane domain, and a costimulatory domain. In some embodiments, the bi-specific CAR comprises one or more additional peptide segments described herein.

In some embodiments, CARs of the disclosure may comprise a CD20 scFv. An exemplary CD20 scFv comprises the following:

(SEQ ID NO: 15)
DIVLTQSPAILSASPGEKVTMTCRASSSVNYMDWY
QKKPGSSPKPWIYATSNLASGVPARFSGSGSGTSY
SLTISRVEAEDAATYYCQQWSFNPPTFGGGTKLEI
KGSTSGGGSGGGSGGGGSSEVQLQQSGAELVKPGA
SVKMSCKASGYTFTSYNMHVWKQTPGQGLEWIGAI
YPGNGDTSYNQKFKGKATLTADKSSSTAYMQLSSL
TSEDSADYYCARSNYYGSSYWFFDVWGAGTTVTVS
S.

In some embodiments, CARs of the disclosure may comprise a CD19 scFv. An exemplary CD19 scFv comprises the following:

(SEQ ID NO: 16)
DIQMTQTTSSLSASLGDRVTISCRASQDISKYLNW
YQQKPDGTVKLLIYHTSRLHSGVPSRFSGSGSGTD
YSLTISNLEQEDIATYFCQQGNTLPYTFGGGTKLE
ITGSTSGSGKPGSGEGSTKGEVKLQESGPGLVAPS
QSLSVTCTVSGVSLPDYGVSWIRQPPRKGLEWLGV
IWGSETTYYNSALKSRLTIIKDNSKSQVFLKMNSL
QTDDTAIYYCAKHYYYGGSYAMD YWGQGTSVTVSS.

In some embodiments, an anti-CD19 CAR is encoded by a DNA construct with the following structure: GM-CSF signal peptide-HA tag-CD19 scFv-IgG4 hinge-CD28 tm-4-1BB co-stimulatory domain-CD3 zeta. This construct has the following sequence:

(SEQ ID NO: 17)
ATGCTGCTGCTGGTGACCAGCCTGCTGCTGTGCGA
GCTGCCCCACCCCGCCTTTCTGCTGATCCCCGGCG
GAAGTTACCCATATGACGTTCCCGACTACGCTGGC
GACATCCAGATGACCCAGACCACCTCCAGCCTGAG
CGCCAGCCTGGGCGACCGGGTGACCATCAGCTGCC
GGGCCAGCCAGGACATCAGCAAGTACCTGAACTGG
TATCAGCAGAAGCCCGACGGCACCGTCAAGCTGCT
GATCTACCACACCAGCCGGCTGCACAGCGGCGTGC
CCAGCCGGTTTAGCGGCAGCGGCTCCGGCACCGAC
TACAGCCTGACCATCTCCAACCTGGAACAGGAAGA
TATCGCCACCTACTTTTGCCAGCAGGGCAACACAC
TGCCCTACACCTTTGGCGGCGGAACAAAGCTGGAA
ATCACCGGCAGCACCTCCGGCAGCGGCAAGCCTGG
CAGCGGCGAGGGCAGCACCAAGGGCGAGGTGAAGC
TGCAGGAAAGCGGCCCTGGCCTGGTGGCCCCCAGC
CAGAGCCTGAGCGTGACCTGCACCGTGAGCGGCGT
GAGCCTGCCCGACTACGGCGTGAGCTGGATCCGGC
AGCCCCCCAGGAAGGGCCTGGAATGGCTGGGCGTG
ATCTGGGGCAGCGAGACCACCTACTACAACAGCGC
CCTGAAGAGCCGGCTGACCATCATCAAGGACAACA
GCAAGAGCCAGGTGTTCCTGAAGATGAACAGCCTG
CAGACCGACGACACCGCCATCTACTACTGCGCCAA
GCACTACTACTACGGCGGCAGCTACGCCATGGACT
ACTGGGGCCAGGGCACCAGCGTGACCGTGAGCAGC
GAATCTAAGTACGGACCGCCCTGCCCCCCTTGCCC
TATGTTCTGGGTGCTGGTGGTGGTCGGAGGCGTGC
TGGCCTGCTACAGCCTGCTGGTCACCGTGGCCTTC
ATCATCTTTTGGGTGAAACGGGGCAGAAAGAAACT
CCTGTATATATTCAAACAACCATTTATGAGACCAG
TACAAACTACTCAAGAGGAAGATGGCTGTAGCTGC
CGATTTCCAGAAGAAGAAGAAGGAGGATGTGAACT
GCGGGTGAAGTTCAGCAGAAGCGCCGACGCCCCTG
CCTACCAGCAGGGCCAGAATCAGCTGTACAACGAG
CTGAACCTGGGCAGAAGGGAAGAGTACGACGTCCT
GGATAAGCGGAGAGGCCGGGACCCTGAGATGGGCG
GCAAGCCTCGGCGGAAGAACCCCCAGGAAGGCCTG
TATAACGAACTGCAGAAAGACAAGATGGCCGAGGC
CTACAGCGAGATCGGCATGAAGGGCGAGCGGAGGC
GGGGCAAGGGCCACGACGGCCTGTATCAGGGCCTG
TCCACCGCCACCAAGGATACCTACGACGCCCTGCA
CATGCAGGCCCTGCCCCCAAGG.

Other cancer-specific molecules (in addition to CD19 and CD20) that can be targeted by CARs can include CAIX, CD33, CD44v7/8, CEA, EGP-2, EGP-40, erb-B2, erb-B3, erb-B4, FBP, fetal acetycholine receptor, GD2, GD3, Her2/neu, IL-13R-a2, KDR, k-light chain, LeY, L1 cell adhesion molecule, MAGE-A1, mesothelin, MUC1, NKG2D ligands, oncofetal antigen (h5T4), PSCA, PSMA, TAA targeted by mAb IgE, TAG-72, and VEGF-R2. In some embodiments, the cancer-specific molecule comprises Her2.

b. Signal Peptides

In some embodiments, CARs include a signal peptide directs the transport and localization of the protein within a cell, e.g. to a certain cell organelle (such as the endoplasmic reticulum) and/or the cell surface. A signal peptide directs the nascent protein into the endoplasmic reticulum. This is essential if the receptor is to be glycosylated and anchored in the cell membrane. Generally, the signal peptide natively attached to the amino-terminal most component is used (e.g. in a scFv with orientation light chain-linker-heavy chain, the native signal of the light-chain is used). In some embodiments the signal peptide has the sequence METDTLLLWVLLLWVPGSTG (SEQ ID NO:18), MLLVTSLLLCELPHPAFLLIPDT (SEQ ID NO:19), or MGTSLLCWMALCLLGADHADG (SEQ ID NO:20). In some embodiments, the signal peptide is cleaved after passage of the endoplasmic reticulum (ER), i.e. is a cleavable signal peptide. In some embodiments, a restriction site is at the carboxy end of the signal peptide to facilitate cleavage.

c. Antigen-Binding Domain

The antigen-binding domain of a CAR is an antigen-binding protein, such as a single-chain variable fragment (scFv) based on antibodies against the target antigen. Framework modifications can be made to the antigen-binding domain to decrease immunogenicity, for example, by “backmutating” one or more framework residues to the corresponding germline sequence. It is also contemplated that the antigen binding domain may be multi-specific or multivalent by multimerizing the antigen binding domain with VH and VL region pairs that bind either the same antigen (multi-valent) or a different antigen (multi-specific).

d. Peptide Spacer

A spacer region links the antigen-binding domain to the transmembrane domain of a CAR. It should be flexible enough to allow the antigen-binding domain to orient in different directions to facilitate antigen recognition. The simplest form is the hinge region from IgG. Alternatives include the CH2CH3 region of immunoglobulin and portions of CD3. In some embodiments, the CH2CH3 region may have L235E/N297Q or L235D/N297Q modifications, or at least 75%, at least 80%, at least 85%, at least 90%, at least 95%, at least 98%, or 100% amino acid sequence identity of the CH2CH3 region. For most scFv-based constructs, the IgG hinge suffices. However the best spacer often has to be determined empirically. In some embodiments, the spacer is from IgG4.

As used herein, the term “hinge” refers to a flexible polypeptide connector region (also referred to herein as “hinge region” or “spacer”) providing structural flexibility and spacing to flanking polypeptide regions and can consist of natural or synthetic polypeptides. A “hinge” derived from an immunoglobulin (e.g., IgG1) is generally defined as stretching from Glu216 to Pro230 of human IgG1 (Burton (1985) Molec. Immunol., 22: 161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425. The hinge region can include complete hinge region derived from an antibody of a different class or subclass from that of the CH1 domain. The term “hinge” can also include regions derived from CD8 and other receptors that provide a similar function in providing flexibility and spacing to flanking regions.

The peptide spacer can have a length of at least, at most, or exactly 4, 5, 6, 7, 8, 9, 10, 15, 16, 17, 18, 19, 20, 20, 25, 30, 35, 40, 45, 50, 75, 100, 110, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). In some embodiments, the peptide spacer consists of or comprises a hinge region from an immunoglobulin. Immunoglobulin hinge region amino acid sequences are known in the art; see, e.g., Tan et al. (1990) Proc. Natl. Acad. Sci. USA 87: 162; and Huck et al. (1986) Nucl. Acids Res.

The length of a peptide spacer may have effects on the response to the target antigen and/or expansion properties. In some embodiments, a shorter spacer such as less than 50, 45, 40, 30, 35, 30, 25, 20, 15, or 10 amino acids may have the advantage of a decrease in the concentration of target antigen required for an effective activation response. In some embodiments, a longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids may have the advantage of increased expansion in vivo or in vitro.

As non-limiting examples, an immunoglobulin hinge region can include one of the following amino acid sequences: DKTHT (SEQ ID NO:21); CPPC (SEQ ID NO:22); CPEPKSCDTPPPCPR (SEQ ID NO:23); ELKTPLGDTTHT (SEQ ID NO:24); KSCDKTHTCP (SEQ ID NO:25); KCCVDCP (SEQ ID NO:26); KYGPPCP (SEQ ID NO:27); EPKSCDKTHTCPPCP (SEQ ID NO:28) (human IgG1 hinge); ERKCCVECPPCP (SEQ ID NO:29) (human IgG2 hinge); ELKTPLGDTTHTCPRCP (SEQ ID NO:30) (human IgG3 hinge); SPNMVPHAHHAQ (SEQ ID NO:31); ESKYGPPCPPCP (SEQ ID NO:32) or ESKYGPPCPSCP (SEQ ID NO:33) (human IgG4 hinge-based) and the like.

The hinge region can comprise an amino acid sequence of a human IgG1, IgG2, IgG3, or IgG4, hinge region. The hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO:34).

The hinge region can comprise an amino acid sequence derived from human CD8; e.g., the hinge region can comprise the amino acid sequence:

(SEQ ID NO: 35)
TTTPAPRPPTPAPTIASQPLSLRPEACRPAAGGAVHTRGLDFACD,

or a variant thereof.

e. Transmembrane Domain

The transmembrane domain of the CAR is a hydrophobic alpha helix that spans the membrane. Generally, the transmembrane domain from the most membrane proximal component of the endodomain is used. Different transmembrane domains result in different receptor stability.

The transmembrane domain is interposed between the peptide spacer and the endodomain. In some embodiments, the transmembrane domain is interposed between the peptide spacer and a co-stimulatory region. In some embodiments, a linker is between the transmembrane domain and a co-stimulatory region or endodomain.

Any transmembrane domain that provides for insertion of a polypeptide into the cell membrane of a eukaryotic (e.g., mammalian) cell is suitable for use. As one non-limiting example, the transmembrane sequence IYIWAPLAGTCGVLLLSLVITLYC (SEQ ID NO:36) can be used. In some embodiments, the transmembrane domain is

CD8 beta derived:
(SEQ ID NO: 37)
LGLLVAGVLVLLVSLGVAIHLCC;
CD4 derived:
(SEQ ID NO: 38)
ALIVLGGVAGLLLFIGLGIFFCVRC;
CD3 zeta derived:
(SEQ ID NO: 39)
LCYLLDGILFIYGVIL
TALFLRV;
CD28 derived:
(SEQ ID NO: 40)
WVLVVVGGVLACYSLLVTVAFIIFWV;
CD134 (OX40) derived:
(SEQ ID NO: 41)
VAAILGLGLVLGLLGPLAILLALYLL;
or
CD7 derived:
(SEQ ID NO: 42)
ALPAALAVISFLLGLGLGVACVLA.

f. Endodomain

After antigen recognition, receptors cluster and a signal is transmitted to the cell through the endodomain and/or co-stimulatory domain. In some embodiments, the co-stimulatory domains described herein are part of the endodomain. The most commonly used endodomain component is CD3-zeta, which contains 3 ITAMs. This transmits an activation signal to the T cell after antigen is bound. CD3-zeta may not provide a fully competent activation signal and additional co-stimulatory signaling is needed. For example, chimeric CD28 and OX40 can be used with CD3-Zeta to transmit a proliferative/survival signal, or all three can be used together.

Further endodomains suitable for use in CARs include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some embodiments, the endodomain includes at least one (e.g., one, two, three, four, five, six, etc.) ITAM motif as described herein. In some embodiments, the endodomain includes DAP10/CD28 type signaling chains.

Endodomains suitable for use in CARs include immunoreceptor tyrosine-based activation motif (ITAM)-containing intracellular signaling polypeptides. An ITAM motif is YX1X2(L/I), where X1 and X2 are independently any amino acid. In some cases, the endodomain comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX1X2(L/I))(X3)n(YX1X2(L/I)), where n is an integer from 6 to 8, and each of the 6-8 X3 can be any amino acid.

A suitable endodomain may be an FFAM motif-containing portion that is derived from a polypeptide that contains an ITAM motif. For example, a suitable endodomain can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12; FCER1G (Fc epsilon receptor I gamma chain); CD3D (CD3 delta); CD3E (CD3 epsilon); CD3G (CD3 gamma); CD3Z (CD3 zeta); and CD79A (antigen receptor complex-associated protein alpha chain).

In some cases, the endodomain is derived from DAP12 (also known as TYROBP; TYRO protein tyrosine kinase binding protein; KARAP; PLOSL; DN AX-activation protein 12; KAR-associated protein; TYRO protein tyrosine kinase-binding protein; killer activating receptor associated protein; killer-activating receptor-associated protein; etc.). For example, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 43)
MGGLEPCSRLLLLPLLLAVSGLRPVQAQAQSDCSC
STVSPGVLAGIVMGDLVLTVLIALAVYFLGRLVPR
GRGAAEAATRKQRITETESPYQELQGQRSDVYSDL
NTQRPYYK;
(SEQ ID NO: 44)
MGGLEPCSRLLLLPLLLAVSGLRPVQAQAQSDCSC
STVSPGVLAGIVMGDLVLTVLIALAVYFLGRLVPR
GRGAAEATRKQRITETESPYQELQGQRSDVYSDLN
TQRPYYK;
(SEQ ID NO: 45)
MGGLEPCSRLLLLPLLLAVSDCSCSTVSPGVLAGI
VMGDLVLTVLIALAVYFLGRLVPRGRGAAEAATRK
QRITETESPYQELQGQRSDVYSDLNTQRPYYK;
or
(SEQ ID NO: 46)
MGGLEPCSRLLLLPLLLAVSDCSCSTVSPGVLAGI
VMGDLVLTVLIALAVYFLGRLVPRGRGAAEATRKQ
RITETESPYQELQGQRSDVYSDLNTQRPYYK.

In some embodiments, a suitable endodomain polypeptide can comprise an ITAM motif-containing portion of the full length DAP12 amino acid sequence. Thus, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 47)
ESPYQELQGQRSDVYSDLNTQ

In some embodiments, the endodomain is derived from FCER1G (also known as FCRG; Fc epsilon receptor I gamma chain; Fc receptor gamma-chain; fc-epsilon R1-gamma; fcRgamma; fceRI gamma; high affinity immunoglobulin epsilon receptor subunit gamma; immunoglobulin E receptor, high affinity, gamma chain; etc.). For example, a suitable endodomain polypeptide can comprise an amino acid sequence having at least 75%, at least 80%, at least 85%, at least 90%, at least 95%, at least 98%, or 100% amino acid sequence identity to

(SEQ ID NO: 48)
MIPAVVLLLLLLVEQAAALGEPQLCYILDAILFLY
GIVLTLLYCRLKIQVRKAAITSYEKSDGVYTGLST
RNQETYETLKHEKPPQ.

In some embodiments, a suitable endodomain polypeptide can comprise an ITAM motif-containing portion of the full length FCER1G amino acid sequence. Thus, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 49)
DGVYTGLSTRNQETYETLKHE.

In some embodiments, the endodomain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). For example, a suitable endodomain polypeptide can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 170 aa, of either of the following amino acid sequences (2 isoforms):

(SEQ ID NO: 50)
MEHSTFLSGLVLATLLSQVSPFKIPIEELEDRVFV
NCNTSITWVEGTVGTLLSDITRLDLGKRILDPRGI
YRCNGTDIYKDKESTVQVHYRMCQSCVELDPATVA
GIIVTDVIATLLLALGVFCFAGHETGRLSGAADTQ
ALLRNDQVYQPLRDRDDAQYSHLGGNWARNK
or
(SEQ ID NO: 51)
MEHSTFLSGLVLATLLSQVSPFKIPIEELEDRVFV
NCNTSITWVEGTVGTLLSDITRLDLGKRILDPRGI
YRCNGTDIYKDKESTVQVHYRTADTQALLRNDQVY
QPLRDRDDAQYSHLGGNWARNK.

In some embodiments, a suitable endodomain polypeptide can comprise an ITAM motif-containing portion of the full length CD3 delta amino acid sequence. Thus, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 52)
DQVYQPLRDRDDAQYSHLGGN.

In some embodiments, the endodomain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). For example, a suitable endodomain polypeptide can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 205 aa, of the following amino acid sequence:

(SEQ ID NO: 53)
MQSGTHWRVLGLCLLSVGVWGQDGNEEMGGITQTP
YKVSISGTTVILTCPQYPGSEILWQHNDKNIGGDE
DDKNIGSDEDHLSLKEFSELEQSGYYVCYPRGSKP
EDANFYLYLRARVCENCMEMDVMSVATIVIVDICI
TGGLLLLVYYWSKNRKAKAKPVTRGAGAGGRQRGQ
NKERPPPVPNPDYEPIRKGQRDLYSGLNQRRI.

In some embodiments, a suitable endodomain polypeptide can comprise an ITAM motif-containing portion of the full length CD3 epsilon amino acid sequence. Thus, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 54)
NPDYEPIRKGQRDLYSGLNQR.

In some embodiments, the endodomain is derived from T-cell surface glycoprotein CD3 gamma chain (also known as CD3G, T-cell receptor T3 gamma chain, CD3-GAMMA, T3G, gamma polypeptide (TiT3 complex), etc.). For example, a suitable endodomain polypeptide can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 180 aa, of the following amino acid sequence:

(SEQ ID NO: 55)
MEQGKGLAVLILAIILLQGTLAQSIKGNHLVKVYDYQEDGSVLLTCDAEA
KNITWFKDGKMIGFLTEDKKKWNLGSNAKDPRGMYQCKGSQNKSKPLQVY
YRMCQNCIELNAATISGFLFAEIVSIFVLAVGVYFIAGQDGVRQSRASDK
QTLLPNDQLYQPLKDREDDQYSHLQGNQLRRN.

In some embodiments, a suitable endodomain polypeptide can comprise an ITAM motif-containing portion of the full length CD3 gamma amino acid sequence. Thus, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 56)
DQLYQPLKDREDDQYSHLQGN.

In some embodiments, the endodomain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). For example, a suitable intracellular signaling domain polypeptide can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of either of the following amino acid sequences (2 isoforms):

(SEQ ID NO: 57)
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR
or
(SEQ ID NO: 58)
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR.

In some embodiments, a suitable endodomain polypeptide can comprise an ITAM motif-containing portion of the full length CD3 zeta amino acid sequence. Thus, a suitable endodomain polypeptide can comprise an amino acid sequence having at least 75%, at least 80%, at least 85%, at least 90%, at least 95%, at least 98%, or 100%, amino acid sequence identity to any of the following amino acid sequences:

(SEQ ID NO: 59)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR;
(SEQ ID NO: 60)
NQLYNELNLGRREEYDVLDKR;
(SEQ ID NO: 90)
EGLYNELQKDKMAEAYSEIGMK;
or
(SEQ ID NO: 61)
DGLYQGLSTATKDTYDALHMQ.

In some embodiments, the endodomain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). For example, a suitable endodomain polypeptide can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 150 aa, from about 150 aa to about 200 aa, or from about 200 aa to about 220 aa, of either of the following amino acid sequences (2 isoforms):

(SEQ ID NO: 62)
MPGGPGVLQALPATIFLLFLLSAVYLGPGCQALWMHKVPASLMVSLGEDA
HFQCPHNSSNNANVTWWRVLHGNYTWPPEFLGPGEDPNGTLIIQNVNKSH
GGIYVCRVQEGNESYQQSCGTYLRVRQPPPRPFLDMGEGTKNRIITAEGI
ILLFCAVVPGTLLLFRKRWQNEKLGLDAGDEYEDENLYEGLNLDDCSMYE
DISRGLQGTYQDVGSLNIGDVQLEKP;
or
(SEQ ID NO: 63)
MPGGPGVLQALPATIFLLFLLSAVYLGPGCQALWMHKVPASLMVSLGEDA
HFQCPHNSSNNANVTWWRVLHGNYTWPPEFLGPGEDPNEPPPRPFLDMGE
GTKNRIITAEGIILLFCAVVPGTLLLFRKRWQNEKLGLDAGDEYEDENLY
EGLNLDDCSMYEDISRGLQGTYQDVGSLNIGDVQLEKP.

In some embodiments, a suitable endodomain polypeptide can comprise an ITAM motif-containing portion of the full length CD79A amino acid sequence. Thus, a suitable endodomain polypeptide can comprise an amino acid sequence having at least 75%, at least 80%, at least 85%, at least 90%, at least 95%, at least 98%, or 100%, amino acid sequence identity to the following amino acid sequence:

(SEQ ID NO: 64)
ENLYEGLNLDDCSMYEDISRG.

In some embodiments, suitable endodomains can comprise a DAP10/CD28 type signaling chain. An example of a DAP 10 signaling chain is the amino acid sequence: RPRRSPAQDGKVYINMPGRG (SEQ ID NO:65). In some embodiments, a suitable endodomain comprises an amino acid sequence having at least 85%, at least 90%, at least 95%, at least 98%, or at least 99%, amino acid sequence identity to the entire length of the amino acid sequence

(SEQ ID NO: 66)
RPRRSPAQDGKVYINMPGRG.

An example of a CD28 signaling chain is the amino acid sequence

(SEQ ID NO: 67)
FWVLVVVGGVLACYSLLVTVAFIIFWVRSKRSRLLHSDYMNMTPRRPGP
TRKHYQPYAPPRDFAAYRS.

In some embodiments, a suitable endodomain comprises an amino acid sequence having at least 85%, at least 90%, at least 95%, at least 98%, or at least 99%, amino acid sequence identity to the entire length of the amino acid sequence

(SEQ ID NO: 68)
FWVLVVVGGVLACYSLLVTVAFIIFWVRSKRSRLLHSDYMNMTPRRPGP
TRKHYQPYAPPRDFAAYRS.

Further endodomains suitable for use in the polypeptides of the disclosure include a ZAP70 polypeptide, e.g., a polypeptide comprising an amino acid sequence having at least 85%, at least 90%, at least 95%, at least 98%, at least 99%, or 100%, amino acid sequence identity to a contiguous stretch of from about 300 amino acids to about 400 amino acids, from about 400 amino acids to about 500 amino acids, or from about 500 amino acids to 619 amino acids, of the following amino acid sequence:

(SEQ ID NO: 69)
MPDPAAHLPFFYGSISRAEAEEHLKLAGMADGLFLLRQCLRSLGGYVLS
LVHDVRFHHFPIERQLNGTYAIAGGKAHCGPAELCEFYSRDPDGLPCNL
RKPCNRPSGLEPQPGVFDCLRDAMVRDYVRQTWKLEGEALEQAIISQAP
QVEKLIATTAHERMPWYHSSLTREEAERKLYSGAQTDGKFLLRPRKEQG
TYALSLIYGKTVYHYLISQDKAGKYCIPEGTKFDTLWQLVEYLKLKADG
LIYCLKEACPNSSASNASGAAAPTLPAHPSTLTHPQRRIDTLNSDGYTP
EPARITSPDKPRPMPMDTSVYESPYSDPEELKDKKLFLKRDNLLIADIE
LGCGNFGSVRQGVYRMRKKQIDVAIKVLKQGTEKADTEEMMREAQIMHQ
LDNPYIVRLIGVCQAEALMLVMEMAGGGPLHKFLVGKREEIPVSNVAEL
LHQVSMGMKYLEEKNFVHRDLAARNVLLVNRHYAKISDFGLSKALGADD
SYYTARSAGKWPLKWYAPECINFRKFSSRSDVWSYGVTMWEALSYGQKP
YKKMKGPEVMAFIEQGKRMECPPECPPELYALMSDCWIYKWEDRPDFLT
VEQRMRACYYSLASKVEGPPGSTQKAEAACA.

g. Detection Peptides

CARs can include detection peptides. Suitable detection peptides include hemagglutinin (HA; e.g., YPYDVPDYA (SEQ ID NO:70); FLAG (e.g., DYKDDDDK (SEQ ID NO:71); c-myc (e.g., EQKLISEEDL; SEQ ID NO:72), and the like. Other suitable detection peptides are known in the art.

h. Peptide Linkers

In some embodiments, CARs may also include peptide linkers (sometimes referred to as a linker). A peptide linker may be separating any of the peptide domain/regions described herein. As an example, a linker may be between the signal peptide and the antigen binding domain, between the VH and VL of the antigen binding domain, between the antigen binding domain and the peptide spacer, between the peptide spacer and the transmembrane domain, flanking the co-stimulatory region or on the N- or C-region of the co-stimulatory region, and/or between the transmembrane domain and the endodomain. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)n, glycine-serine polymers (including, for example, (GS)n, (GSGGS)n (SEQ ID NO:73) and (GGGS)n (SEQ ID NO:74), where n is an integer of at least one, glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. Exemplary spacers can comprise amino acid sequences including, but not limited to, GGSG (SEQ ID NO:75), GGSGG (SEQ ID NO:76), GSGSG (SEQ ID NO:77), GSGGG (SEQ ID NO:78), GGGSG (SEQ ID NO:79), GSSSG (SEQ ID NO:80), and the like.

i. Co-stimulatory Region

Non-limiting examples of suitable co-stimulatory regions in CARs include, but are not limited to, polypeptides from 4-1BB (CD137), CD28, ICOS, OX-40, BTLA, CD27, CD30, GITR, and HVEM.

A co-stimulatory region may have a length of at least, at most, or exactly 20, 25, 30, 35, 40, 50, 60, 70, 80, 90, 100, 150, 200, or 300 amino acids or any range derivable therein. In some embodiments, the co-stimulatory region is derived from an intracellular portion of the transmembrane protein 4-1BB (also known as TNFRSF9; CD137; 4-1BB; CDw137; ILA; etc.). For example, a suitable co-stimulatory region can comprise an amino acid sequence having at least 75%, at least 80%, at least 85%, at least 90%, at least 95%, at least 98%, or 100% amino acid sequence identity to

(SEQ ID NO: 81)
KRGRKKLLYIFKQPFMRPVQTTQEEDGCSCRFPEEEEGGCEL.

In some embodiments, the co-stimulatory region is derived from an intracellular portion of the transmembrane protein CD28 (also known as Tp44). For example, a suitable co-stimulatory region can comprise an amino acid sequence having at least 75%, at least 80%, at least 85%, at least 90%, at least 95%, at least 98%, or 100% amino acid sequence identity to

(SEQ ID NO: 82)
FWVRSKRSRLLHSDYMNMTPRRPGPTRKHYQPYAPPRDFAAYRS.

In some embodiments, the co-stimulatory region is derived from an intracellular portion of the transmembrane protein ICOS (also known as AILIM, CD278, and CVID1). For example, a suitable co-stimulatory region can comprise an amino acid sequence having at least 75%, at least 80%, at least 85%, at least 90%, at least 95%, at least 98%, or 100% amino acid sequence identity to

(SEQ ID NO: 83)
TKKKYSSSVHDPNGEYMFMRAVNTAKKSRLTDVTL.

In some embodiments, the co-stimulatory region is derived from an intracellular portion of the transmembrane protein OX-40 (also known as TNFRSF4, RP5-902P8.3, ACT35, CD134, OX40, TXGP1L). For example, a suitable co-stimulatory region can comprise an amino acid sequence having at least 75%, at least 80%, at least 85%, at least 90%, at least 95%, at least 98%, or 100% amino acid sequence identity to

(SEQ ID NO: 84)
RRDQRLPPDAHKPPGGGSFRTPIQEEQADAHSTLAKI

In some embodiments, the co-stimulatory region is derived from an intracellular portion of the transmembrane protein BTLA (also known as BTLA1 and CD272). For example, a suitable co-stimulatory region can comprise an amino acid sequence having at least 75%, at least 80%, at least 85%, at least 90%, at least 95%, at least 98%, or 100% amino acid sequence identity to

(SEQ ID NO: 85)
CCLRRHQGKQNELSDTAGREINLVDAHLKSEQTEASTRQNSQVLLSET
GIYDNDPDLCFRMQEGSEVYSNPCLEENKPGIVYASLNHSVIGPNSRL
ARNVKEAPTEYASICVRS.

In some embodiments, the co-stimulatory region is derived from an intracellular portion of the transmembrane protein CD27 (also known as S 152, T14, TNFRSF7, and Tp55). For example, a suitable co-stimulatory region can comprise an amino acid sequence having at least 75%, at least 80%, at least 85%, at least 90%, at least 95%, at least 98%, or 100% amino acid sequence identity to

(SEQ ID NO: 86)
HQRRKYRSNKGESPVEPAEPCRYSCPREEEGSTIPIQEDYRKPEPACS
P.

In some embodiments, the co-stimulatory region is derived from an intracellular portion of the transmembrane protein CD30 (also known as TNFRSF8, D1S166E, and Ki-1). For example, a suitable co-stimulatory region can comprise an amino acid sequence having at least 75%, at least 80%, at least 85%, at least 90%, at least 95%, at least 98%, or 100% amino acid sequence identity to

(SEQ ID NO: 87)
RRACRKRIRQKLHLCYPVQTSQPKLELVDSRPRRSSTQLRSGASVTEP
VAEERGLMSQPLMETCHSVGAAYLESLPLQDASPAGGPSSPRDLPEPR
VSTEHTNNKIEKIYIMKADTVIVGTVKAELPEGRGLAGPAEPELEEEL
EADHTPHYPEQETEPPLGSCSDVMLSVEEEGKEDPLPTAASGK.

In some embodiments, the co-stimulatory region is derived from an intracellular portion of the transmembrane protein GITR (also known as TNFRSF18, RP5-902P8.2, AITR, CD357, and GITR-D). For example, a suitable co-stimulatory region can comprise an amino acid sequence having at least 75%, at least 80%, at least 85%, at least 90%, at least 95%, at least 98%, or 100% amino acid sequence identity to

(SEQ ID NO: 88)
HIWQLRSQCMWPRETQLLLEVPPSTEDARSCQFPEEERGERSAEEKGR
LGDLWV.

In some embodiments, the co-stimulatory region derived from an intracellular portion of the transmembrane protein HVEM (also known as TNFRSF14, RP3-395M20.6, ATAR, CD270, HVEA, HVEM, LIGHTR, and TR2). For example, a suitable co-stimulatory region can comprise an amino acid sequence having at least 75%, at least 80%, at least 85%, at least 90%, at least 95%, at least 98%, or 100% amino acid sequence identity to

(SEQ ID NO: 89)
CVKRRKPRGDVVKVIVSVQRKRQEAEGEATVIEALQAPPDVTTVAVEE
TIPSFTGRSPNH.

### V. Methods

Aspects of the current disclosure relate to methods of treating a condition, for stimulating an immune response, for regulating an immune response, for suppressing an immune response, and for reducing the risk of an adverse immune response, such as cytokine release syndrome. These actions may be done in vitro, in vivo, or ex vivo. In some embodiments, the methods relate to cells capable of stimulating an immune response and/or suppressing a cytokine response in the presence of a cancer cell. The method generally involves genetically modifying a mammalian cell with an expression vector, or an RNA (e.g., in vitro transcribed RNA), comprising nucleotide sequences encoding a polypeptide of the disclosure or directly transferring the polypeptide to the cell. The cell can be an immune cell (e.g., a T lymphocyte or NK cell), a stem cell, a progenitor cell, etc. In some embodiments, the cell is a cell described herein.

In some embodiments, the genetic modification is carried out ex vivo. For example, a T lymphocyte, a stem cell, or an NK cell (or cell described herein) is obtained from an individual; and the cell obtained from the individual is genetically modified to express a polypeptide of the disclosure. In some cases, the genetically modified cell is activated ex vivo (i.e., a target antigen is contacted with the cells ex vivo). In other cases, the genetically modified cell is introduced into an individual (e.g., the individual from whom the cell was obtained); and the genetically modified cell is activated in vivo (i.e., by endogenously produced target antigen).

In some embodiments, the methods further comprise the administration of additional therapeutic agents, such as bi-specific T cell engagers (BiTE). Such therapeutic agents may be administered in peptide form to the patient or expressed in cells of the disclosure, such as those that that comprise the chimeric binding polypeptide. The BiTE may have antigen specificity for a cancer antigen/cancer molecule known in the art and/or described herein and may also have antigen specificity for a T cell molecule such as CD3. In some embodiments, the additional therapeutic agents are expressed in the same T cell as the chimeric binding polypeptide. In some embodiments, the additional therapeutic agents are expressed from the same nucleic acid as the chimeric binding polypeptide.

In some embodiments, the methods relate to administration of the cells or peptides described herein for the treatment of a cancer or administration to a person with a cancer. In some embodiments the cancer is adrenal cancer, anal cancer, bile duct cancer, bladder cancer, bone cancer, brain/CNS tumors in children or adults, breast cancer, cervical cancer, colon/rectum cancer, endometrial cancer, esophagus cancer, Ewing family of tumors, eye cancer, gallbladder cancer, gastrointestinal carcinoid tumors, gastrointestinal stromal tumor (GIST), gestation trophoblastic disease, hodgkin disease, kaposi sarcoma, kidney cancer, laryngeal and hypopharyngeal cancer, leukemia, acute lymphocytic leuckemia, acute myeloid leukemia, chronic lymphocytic leukemia, chronic myeloid leukemia, chronic myelomonocytic leukemia, liver cancer, lung cancer, non-small cell lung cancer, small cell lung cancer, lung carcinoid tumor, lymphoma, malignant mesothelioma, multiple myeloma, myelodysplastic syndrome, nasal cavity and paranasal sinum cancer, nasopharyngeal cancer, neuroblastoma, non-hodgkin lymphoma, oral cavity or oropharyngeal cancer, osteosarcoa, ovarian cancer, pancreatic cancer, penile cancer, pituitary tumors, prostate cancer, retinoblastoma, rhabdomyosarcoma, salivary gland cancer, skin cancer, sarcoma, basal skin cancer, squamous cell skin cancer, melanoma, merkel cell skin cancer, small intestine cancer, stomach cancer, testicular cancer, thymus cancer, thyroid cancer, uterine sarcoma, vaginal cancer, vulvar cancer, waldenstrom macroglobulinemia, or wilms tumor.

Embodiments can be used to treat or ameliorate a number of immune-mediated, inflammatory, or autoimmune-inflammatory diseases, e.g., allergies, asthma, diabetes (e.g. type 1 diabetes), graft rejection, etc. Examples of such diseases or disorders also include, but are not limited to arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and systemic juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes) and autoimmune diabetes. Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), experimental autoimmune encephalomyelitis, myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, graft versus host disease, contact hypersensitivity, asthmatic airway hyperreaction, and endometriosis.

In some embodiments, the patient is one that does not have a particular indication described above. For example, in some embodiments, the patient does not have and/or has not been diagnosed with rheumatoid arthritis. In some embodiments, the patient does not have a chronic autoimmune condition. The term chronic refers to an illness that is persisting for a long time or constantly recurring.

### VI. Additional Therapies

In some embodiments, the methods of the disclosure comprise administration of an additional therapy to a subject. In some embodiments, the additional therapy comprises an immunotherapy. These immunotherapies are further described below.

A. Immunotherapies

1. Checkpoint Inhibitors and Combination Treatment

Embodiments of the disclosure may include administration of immune checkpoint inhibitors, which are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WOO 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

2. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.[60]

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

B. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune responses for long-term immunotherapy

C. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

D. Neoantigens

In some embodiments, the additional therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

E. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments. In some embodiments, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

F. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

H. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VII. Pharmaceutical Compositions

The present disclosure includes methods for modulating immune responses in a subject in need thereof. The disclosure includes cells and proteins that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, intramuscular, intraperitoneal, intranasal, or intravenous injection.

Typically, compositions of the invention are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2-day to 12-week intervals, more usually from one to two week intervals. The course of the administrations may be followed by assays for alloreactive immune responses and T cell activity.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (i.e. cells of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

### VIII. Sequence Listing

Amino acid sequence of cytokine antagonists

| | | SEQ ID |
|---|---|---|
| Description | Sequence | NO: |
| Linker | GSTSGSGKPGSGEGSTKG | 14 |
| Tocilizumab V_{L} | DIQMTQSPSSLSASVGDRVTITCRASQDISSYLN | 1 |
| | WYQQKPGKAPKLLIYYTSRLHSGVPSRFSGSGS | |
| | GTDFTFTISSLQPEDIATYYCQQGNTLPYTFGQG | |
| | TKVEIK | |
| CDR4 | RASQDISSYLN | 94 |
| CDR5 | YTSRLHS | 95 |
| CDR6 | QQGNTLPYT | 96 |
| Tocilizumab V_{H} | EVQLQESGPGLVRPSQTLSLTCTVSGYSITSDH | 2 |
| | AWSWVRQPPGRGLEWIGYISYSGITTYNPSLKS | |
| | RVTMLRDTSKNQFSLRLSSVTAADTAVYYCAR | |
| | SLARTTAMDYWGQGSLVTVSS | |
| CDR1 | GYSITSDHA | 91 |
| CDR2 | ISYSGIT | 92 |
| CDR3 | ARSLARTTAMDY | 93 |
| Infliximab V_{L} | DILLTQSPAILSVSPGERVSFSCRASQFVGSSIH | 3 |
| | WYQQRTNGSPRLLIKYASESMSGIPSRFSGSGS | |
| | GTDFTLSINTVESEDIADYYCQQSHSWPFTFGS | |
| | GTNLEVK | |
| CDR4 | RASQFVGSSIH | 100 |
| CDR5 | YASESMS | 101 |
| CDR6 | QQSHSWPFT | 102 |
| Infliximab V_{H} | EVKLEESGGGLVQPGGSMKLSCVASGFIFSNH | 4 |
| | WMNWVRQSPEKGLEWVAEIRSKSINSATHYAE | |
| | SVKGRFTISRDDSKSAVYLQMTDLRTEDTGVY | |
| | YCSRNYYGSTYDYWGQGTTLTVSS | |
| CDR1 | GFIFSNHWMN | 97 |
| CDR2 | EIRSKSINSATHYAESVKG | 98 |
| CDR3 | NYYGSTYDY | 99 |
| Adalimumab V_{L} | DIQMTQSPSSLSASVGDRVTITCRASQGIRNYL | 5 |
| | AWYQQKPGKAPKLLIYAASTLQSGVPSRFSGS | |
| | GSGTDFTLTISSLQPEDVATYYCQRYNRAPYTF | |
| | GQGTKVEIK | |
| CDR4 | RASQGIRNYLA | 106 |
| CDR5 | AASTLQS | 107 |
| CDR6 | QRYNRAPYT | 108 |
| Adalimumab V_{H} | EVQLVESGGGLVQPGRSLRLSCAASGFTFDDY | 6 |
| | AMHWVRQAPGKGLEWVSAITWNSGHIDYADS | |
| | VEGRFTISRDNAKNSLYLQMNSLRAEDTAVYY | |
| | CAKVSYLSTASSLDYWGQGTLVTVSS | |
| CDR1 | GFTFDDYAMH | 103 |
| CDR2 | AITWNSGHIDYADSVEG | 104 |
| CDR3 | VSYLSTASS | 105 |
| Golimumab V_{L} | EIVLTQSPATLSLSPGERATLSCRASQSVYSYLA | 7 |
| | WYQQKPGQAPRLLIYDASNRATGIPARFSGSGS | |
| | GTDFTLTISSLEPEDFAVYYCQQRSNWPPFTFGP | |
| | GTKVDIK | |
| CDR4 | RASQSVYSYLA | 112 |
| CDR5 | DASNRAT | 113 |
| CDR6 | QQRSNWPPFT | 114 |
| Golimumab V_{H} | QVQLVESGGGVVQPGRSLRLSCAASGFIFSSYA | 8 |
| | MHWVRQAPGNGLEWVAFMSYDGSNKKYADS | |
| | VKGRFTISRDNSKNTLYLQMNSLRAEDTAVYY | |
| | CARDRGIAAGGNYYYYGMDVWGQGTTVTVSS | |
| CDR1 | GFIFSSYAMH | 109 |
| CDR2 | FMSYDGSNKKYADSVKG | 110 |
| CDR3 | DRGIAAGGNYYYYGMDV | 111 |
| Certolizumab V_{L} | DIQMTQSPSSLSASVGDRVTITCKASQNVGTNV | 9 |
| | AWYQQKPGKAPKALIYSASFLYSGVPYRFSGS | |
| | GSGTDFTLTISSLQPEDFATYYCQQYNIYPLTFG | |
| | QGTKVEIK | |
| CDR4 | KASQNVGTNVA | 118 |
| CDR5 | SASFLYS | 119 |
| CDR6 | QQYNIYPLT | 120 |
| Certolizumab V_{H} | EVQLVESGGGLVQPGGSLRLSCAASGYVFTDY | 10 |
| | GMNWVRQAPGKGLEWMGWINTYIGEPIYADS | |
| | VKGRFTFSLDTSKSTAYLQMNSLRAEDTAVYY | |
| | CARGYRSYAMDYWGQGTLVTVSS | |
| CDR1 | GYVFTDYGMN | 115 |
| CDR2 | WINTYIGEPIYADSVKG | 116 |
| CDR3 | GYRSYAMDY | 117 |
| Emapalumab V_{L} | NFMLTQPHSVSESPGKTVTISCTRSSGSIASNYV | 11 |
| | QWYQQRPGSSPTTVIYEDNQRPSGVPDRFSGSI | |
| | DSSSNSASLTISGLKTEDEADYYCQSYDGSNRW | |
| | MFGGGTKLTVL | |
| CDR4 | SGSIASNY | 124 |
| CDR5 | EDN | 125 |
| CDR6 | QSYDGSNRWM | 126 |
| Emapalumab V_{H} | EVQLLESGGGLVQPGGSLRLSCAASGFTFSSYA | 12 |
| | MSWVRQAPGKGLEWVSAISGSGGSTYYADSV | |
| | KGRFTISRDNSKNTLYLQMNSLRAEDTAVYYC | |
| | AKDGSSGWYVPHWFDPWGQGTLVTVSS | |
| CDR1 | GFTFSSYA | 121 |
| CDR2 | ISGSGGST | 122 |
| CDR3 | AKDGSSGWYVPHWFDP | 123 |
| Anakinra | MRPSGRKSSKMQAFRIWDVNQKTFYLRNNQL | 13 |
| | VAGYLQGPNVNLEEKIDVVPIEPHALFLGIHGG | |
| | KMCLSCVKSGDETRLQLEAVNITDLSENRKQD | |
| | KRFAFIRSDSGPTTSFESAACPGWFLCTAMEAD | |
| | QPVSLTNMPDEGVMVTKFYFQEDE | |
| Tociilzumab scFv | GATATACAAATGACTCAATCACCTTCCTCACT | 150 |
| (Vh-linker (lower | TTCCGCCAGCGTGGGTGATCGAGTGACCATC | |
| case)-VI) | ACATGCCGCGCTTCACAGGACATCTCCTCTTA | |
| | CCTGAACTGGTACCAGCAGAAACCCGGGAAA | |
| | GCCCCAAAGCTGTTGATCTACTATACATCAA | |
| | GACTGCACTCTGGCGTCCCCTCTAGGTTCAGT | |
| | GGATCTGGCTCAGGTACTGACTTCACGTTCA | |
| | CTATTTCCAGCCTGCAGCCGGAAGATATTGC | |
| | TACCTACTATTGTCAGCAGGGAAATACCCTC | |
| | CCATATACCTTTGGGCAGGGGACAAAGGTGG | |
| | AAATCAAG [symbol] | |
| | [symbol] | |
| | [symbol] | |
| | GAGGTCCAACTGCAGGAA | |
| | AGCGGTCCAGGCTTGGTGAGACCATCCCAGA | |
| | CCCTGAGCCTCACCTGTACCGTGTCCGGGTA | |
| | CAGTATCACCTCCGACCATGCATGGTCCTGG | |
| | GTGCGCCAGCCTCCCGGAAGAGGTCTGGAAT | |
| | GGATCGGGTATATCTCCTACTCAGGAATCAC | |
| | CACCTACAATCCCTCCCTTAAGTCAAGGGTG | |
| | ACTATGCTCCGCGATACATCCAAAAATCAGT | |
| | TCTCCCTTCGGTTGTCAAGTGTTACAGCCGCC | |
| | GACACCGCAGTCTACTACTGTGCAAGGAGCC | |
| | TCGCCAGGACGACTGCAATGGATTATTGGGG | |
| | CCAGGGCTCCCTGGTGACTGTCAGCAGC | |
| | DIQMTQSPSSLSASVGDRVTITCRASQDISSYLN | 151 |
| | WYQQKPGKAPKLLIYYTSRLHSGVPSRFSGSGS | |
| | GTDFTFTISSLQPEDIATYYCQQGNTLPYTFGQG | |
| | TKVEIK [symbol] | |
| | EVQLQESGPGLVRPS | |
| | QTLSLTCTVSGYSITSDHAWSWVRQPPGRGLE | |
| | WIGYISYSGITTYNPSLKSRVTMLRDTSKNQFSL | |
| | RLSSVTAADTAVYYCARSLARTTAMDYWGQG | |
| | SLVTVSS | |
| Emapalumab scFv | GAAGTCCAACTCCTGGAAAGTGGGGGCGGCC | 152 |
| (Vh-linker (lower | TCGTGCAACCTGGAGGTTCCCTGAGGTTGTC | |
| case)-VI) | CTGCGCTGCTAGCGGGTTCACGTTTAGCAGC | |
| | TATGCAATGTCATGGGTTAGGCAGGCACCTG | |
| | GCAAGGGCCTGGAATGGGTTTCAGCTATCTC | |
| | AGGGTCAGGGGGTAGCACCTACTATGCTGAT | |
| | AGTGTGAAAGGAAGGTTTACGATCTCCAGAG | |
| | ATAATTCCAAGAATACCCTGTACCTTCAAAT | |
| | GAACAGTCTCAGGGCGGAGGATACGGCAGT | |
| | ATACTACTGCGCCAAGGACGGTTCTAGCGGT | |
| | TGGTACGTGCCGCATTGGTTCGACCCGTGGG | |
| | GCCAAGGGACTTTGGTCACTGTGTCCTCT [symbol] | |
| | [symbol] | |
| | [symbol] | |
| | [symbol] AACTTTATGCTTACTCAGCCGCATAGCGT | |
| | ATCCGAAAGCCCAGGCAAAACGGTTACTATT | |
| | TCATGTACCAGATCCAGCGGAAGCATAGCTA | |
| | GTAATTACGTGCAGTGGTACCAACAGCGACC | |
| | AGGTTCAAGTCCAACTACTGTGATTTATGAA | |
| | GATAATCAGAGGCCCAGTGGGGTCCCAGATC | |
| | GCTTCTCTGGCTCAATAGATTCTTCTTCTAAT | |
| | TCCGCAAGTCTTACAATCTCTGGGTTGAAGA | |
| | CAGAAGATGAGGCGGATTATTACTGTCAATC | |
| | TTATGACGGTTCTAACCGCTGGATGTTTGGG | |
| | GGCGGAACGAAGCTGACAGTCCTT | |
| | EVQLLESGGGLVQPGGSLRLSCAASGFTFSSYA | 153 |
| | MSWVRQAPGKGLEWVSAISGSGGSTYYADSV | |
| | KGRFTISRDNSKNTLYLQMNSLRAEDTAVYYC | |
| | AKDGSSGWYVPHWFDPWGQGTLVTVSS | |
| | [symbol] [symbol] | |
| | NFMLTQPHSVSESPGKTVTISCTRS | |
| | SGSIASNYVQWYQQRPGSSPTTVIYEDNQRPSG | |
| | VPDRFSGSIDSSSNSASLTISGLKTEDEADYYCQ | |
| | SYDGSNRWMFGGGTKLTVL | |
| Anti-IL1Rα | [symbol] | 154 |
| ([signal sequence- | [symbol] | |
| lower case]- | [symbol] | |
| protein) | AGGCCGTCCGGACGGAAA | |
| *Note: “Anakinra” is | TCTTCAAAAATGCAAGCTTTTCGGATATGGG | |
| the “protein” portion | ATGTAAACCAAAAGACGTTCTACCTTCGCAA | |
| of the amino acid | TAATCAACTCGTGGCGGGGTATCTTCAGGGT | |
| shown below, | CCTAATGTTAACCTTGAGGAGAAGATAGACG | |
| preceded with an N- | TCGTACCAATAGAGCCACATGCCCTCTTTCTT | |
| terminal methionine | GGGATTCATGGGGGTAAAATGTGTCTTTCAT | |
| | GTGTAAAGTCTGGCGATGAAACACGGCTTCA | |
| | GCTTGAAGCTGTAAACATTACTGACCTTAGT | |
| | GAGAACCGAAAACAGGATAAGCGATTTGCGT | |
| | TCATCCGGTCTGACAGCGGCCCTACTACCTC | |
| | ATTTGAGTCAGCGGCGTGTCCAGGGTGGTTT | |
| | TTGTGTACGGCTATGGAAGCAGATCAGCCCG | |
| | TCAGTCTTACCAATATGCCGGATGAGGGAGT | |
| | CATGGTAACCAAATTCTACTTCCAGGAGGAT | |
| | GAG | |
| | [symbol] | 155 |
| | RPSGRKSSKMQAFRIWDV | |
| | NQKTFYLRNNQLVAGYLQGPNVNLEEKIDVVP | |
| | IEPHALFLGIHGGKMCLSCVKSGDETRLQLEAV | |
| | NITDLSENRKQDKRFAFIRSDSGPTTSFESAACP | |
| | GWFLCTAMEADQPVSLTNMPDEGVMVTKFYF | |
| | QEDE | |

### IX. EXAMPLES

The following examples are included to demonstrate embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Mediating Cytokine Release Syndrome with Self-Regulating T Cells that Modulate IL-6 Signaling

The adoptive transfer of CD19-specific chimeric antigen receptor (CAR)-T cells has shown remarkable efficacy against B-cell malignancies. However, cytokine release syndrome (CRS) is a frequently observed side effect. Although CRS can often be managed by immunosuppression, multiple patient deaths highlight the need for more effective strategies to prevent and ameliorate CRS. Here, the inventors report a strategy to modulate CRS by engineering self-regulating T cells that secret a single-chain variant of the antibody tocilizumab (sToci), which inhibits interleukin-6 receptor alpha (IL-6Ra). CD19 CAR-T cells engineered to secrete sToci show significantly reduced IL-6 signaling and cytokine production levels. Importantly, sToci secretion does not hinder CAR-T cells' ability to lyse CD19⁺ target cells in vitro or in vivo, nor does it affect T-cell differentiation or proliferation upon antigen stimulation. Engineering CAR-T cells to self-regulate inflammatory cytokine production has the potential to prevent CRS without inhibiting the therapeutic efficacy of CAR-T cells

Adoptive T-cell therapy—i.e., the infusion of ex-vivo-expanded T cells with specificity against disease-associated antigens—has demonstrated promising therapeutic efficacy in multiple clinical trials in recent years (1, 2). In particular, the adoptive transfer of T cells engineered to express chimeric antigen receptors (CARs) that target the pan-B-cell marker CD19 has achieved >85% complete remission rate in treating refractory acute lymphocytic leukemia across multiple trials (3-7). These promising results led the United States Food and Drug Administration (FDA) to approve CD19 CAR-T cells in 2017 as the first genetically modified cell therapy for use in humans (8, 9), with growing recognition of cell-based immunotherapy as a potentially transformative treatment paradigm for cancer. However, accumulating clinical experience has also highlighted significant challenges associated with the clinical implementation of CAR-T cell therapy. In particular, cytokine release syndrome (CRS) is a complication characterized by sudden and dramatic elevations of inflammatory cytokines and immunomodulatory proteins (10-12). CRS of varying severity is commonly observed in patients treated with CD19 CAR-T cell therapy (12-16), and it has also been reported for CAR-T cell therapy targeting HER2, BCMA, and mesothelin (17-19). Severe CRS has been identified as the root cause of multiple patient deaths (14, 18), and the severity of CRS has also been found to be associated with an increased incidence of infectious complications 20, 21), highlighting an urgent need to develop effective preventive, diagnostic, and therapeutic methods for CRS management.

At the present time, corticosteroids and tocilizumab are the most common treatments for CRS, with additional agents such as etanercept and infliximab serving as less frequently used alternatives (22, 23). Corticosteroids have pleotropic effects on the immune system, which may compromise T-cell treatment efficacy by non-specifically inhibiting the anti-tumor functions of engineered T cells (12, 24). Therefore, there is a need to identify more targeted therapeutic that exhibit less side-effects.

As a precaution against the possibility of runaway immune responses and fatal CRS symptoms, numerous systems incorporating suicide genes have been proposed (28-30). However, such strategies necessitate the termination of the T-cell therapy, and it remains unclear whether triggering the suicide option would lead to sufficiently rapid reversal of life-threatening CRS and improve patient outcomes.

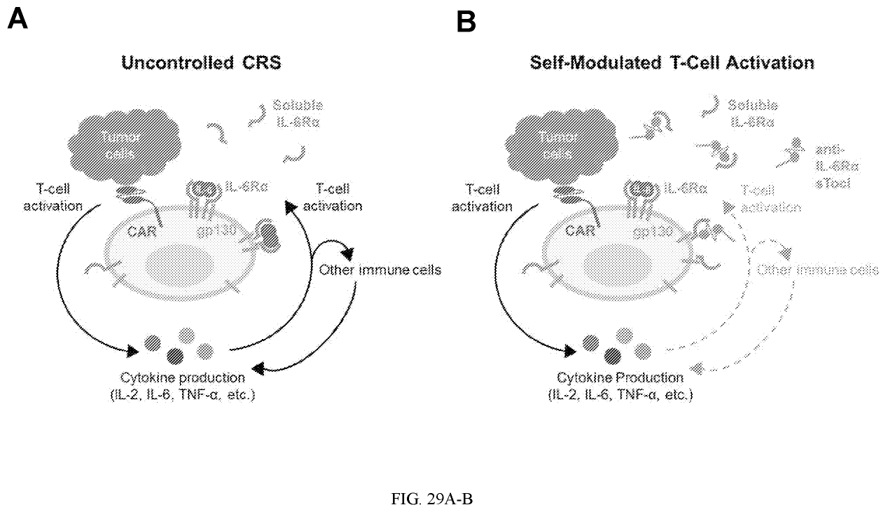

The inventors hypothesized that—instead of relying on indirect diagnostic measures and externally administered immunomodulatory drugs whose proper dosage and timing of application are difficult to determine—it would be beneficial to endow tumor-reactive T cells with the ability to self-modulate their cytokine signaling activities. Here, the inventors report the engineering of primary human CAR-T cells to secrete a single-chain derivative of tocilizumab (sToci) that efficiently inhibits IL-6 signaling, thereby buffering the cytokine production levels of CAR-T cells as well as nearby immune cells (FIG. 29). CD19-CART cells engineered to secrete sToci show reduced IL-6 signaling and significantly lower production of inflammatory cytokines upon antigen stimulation in vitro. Importantly, sToci secretion and the resultant decrease in cytokine production does not hinder the cytotoxicity of CD19 CAR-T cells, nor does it affect T-cell proliferation, subtype differentiation, or exhaustion. CD19 CAR-T cells that constitutively secrete sToci retain the ability to effectively control tumor progression in vivo, demonstrating feasibility of this engineering strategy as a means to regulate cytokine production while maintaining anti-tumor effector function. Future work in an animal model that adequately recapitulates symptoms of CRS would be necessary to fully demonstrate the ability of sToci-expressing T cells to regulate CRS in vivo. The self-regulating T cells reported here represent a new approach to increasing the safety of adoptive T-cell therapy by providing a cell-autonomous means to prevent and control CRS.

A. Results

1. Tocilizumab-Derived Single-Chain Antibody Efficiently Inhibits IL-6 Signaling

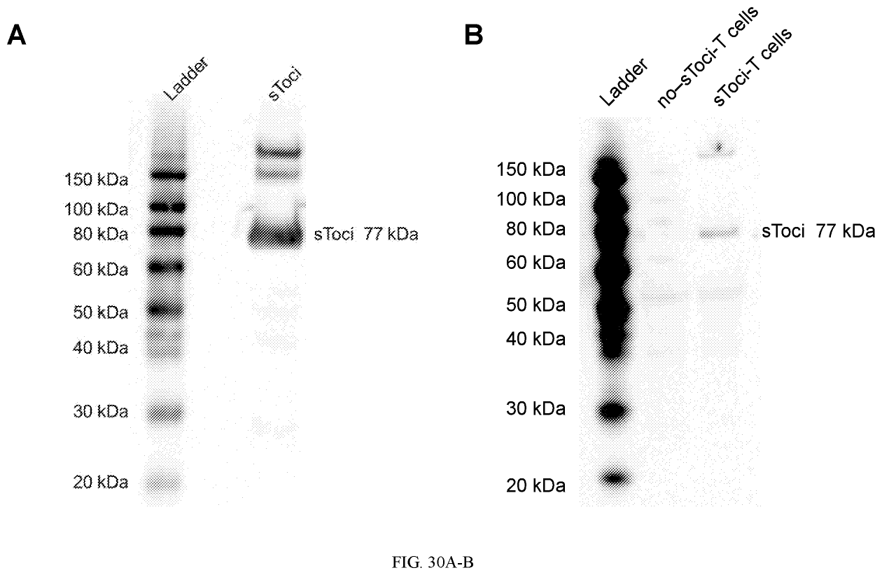

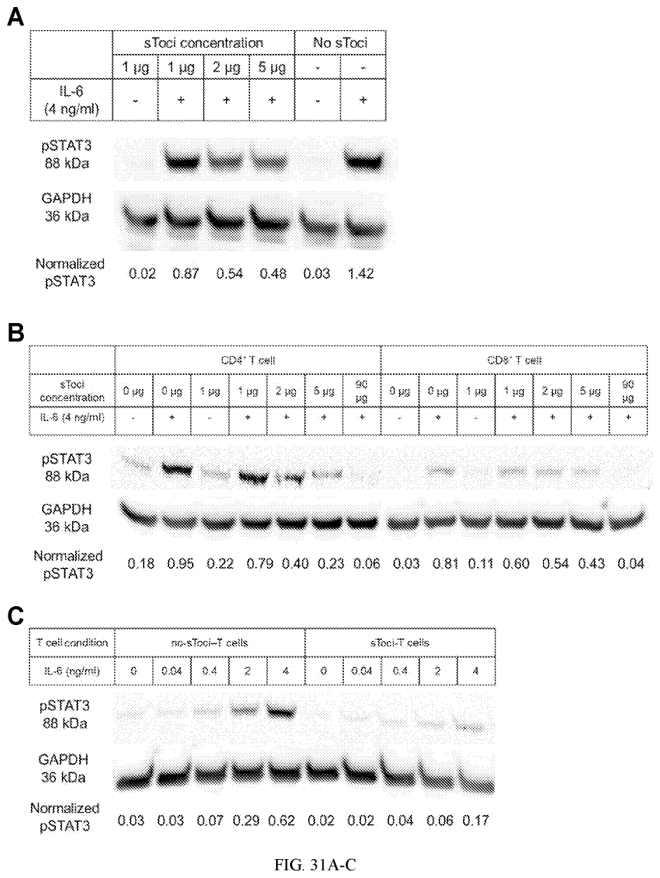

Given the clinical efficacy of tocilizumab administration as a treatment for CRS, the inventors reasoned that engineering T cells to produce an IL-6Ra inhibitor could enable cell-autonomous regulation of cytokine signaling and potentially prevent or ameliorate CRS. A single-chain variant of tocilizumab, termed sToci, was confirmed to be efficiently expressed and secreted by HEK293T cells as well as primary human T cells (FIG. 30). HepG2 cells, which express IL-6 receptors and phosphorylate STAT3 in response to IL-6 signaling, showed a reduction of STAT3 phosphorylation upon sToci addition, demonstrating sToci-mediated inhibition of IL-6 signaling in a dose-dependent manner (FIG. 31A). Similarly, both CD4⁺ and CD8⁺ primary human T cells showed dose-dependent reduction in STAT3 phosphorylation in response to sToci addition (FIG. 31B).

Having observed concentrated sToci's ability to inhibit endogenous IL-6 signaling in primary human T cells, the inventors next evaluated whether T cells can be engineered to secrete sufficient amounts of sToci to influence IL-6 signaling in a cell-intrinsic manner. CD4⁺ primary human T cells were lentivirally transduced to express a second-generation CD19 CAR with or without sToci, and subsequently stimulated with 4 ng/ml IL-6. Western blot results indicate substantially reduced STAT3 phosphorylation among CAR-T cells that express sToci compared to those that do not (FIG. 31C), confirming that engineered T cells can self-regulate IL-6 signaling via sToci secretion.

2. sToci Reduces the Production of Inflammatory Cytokines by Primary Human T Cells

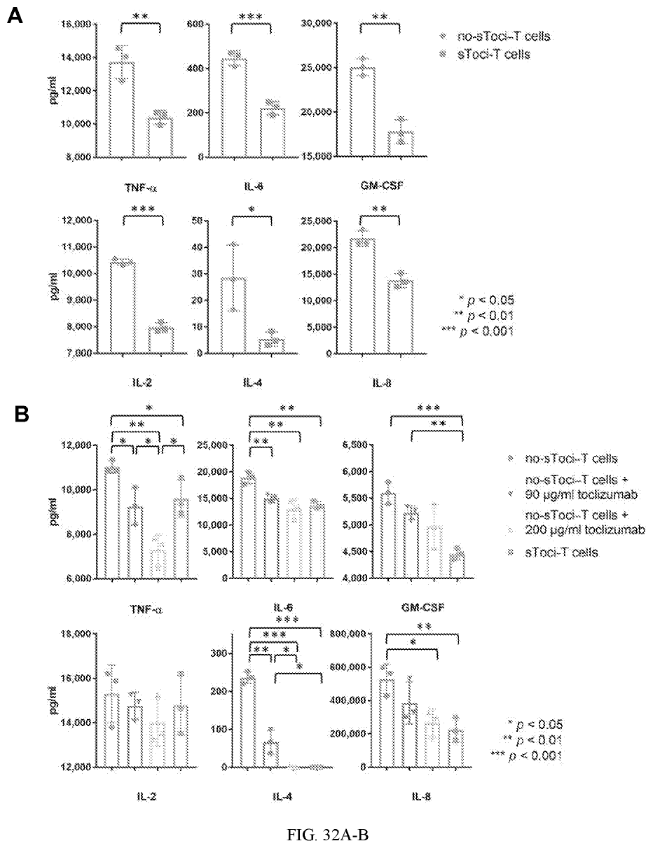

The inventors next evaluated whether constitutive sToci production by CAR-T cells could affect cytokine production by T cells and nearby immune cells in the presence of antigen stimulation. Although the connection between CRS onset and T-cell infusion is well established (10, 13, 22, 25), recent studies have also revealed that many of the cytokines that contribute to CRS are not directly produced by T cells (31-33). In particular, it has been shown that IL-6, a critical CRS modulator, is primarily produced by monocytes and monocyte-derived antigen-presenting cells (APCs) (32). To capture this multifaceted nature of CRS onset and progression, a co-culture system involving both primary human T cells and donor-matched PBMCs was developed to study the effect of T-cell-mediated sToci production on overall cytokine levels. Primary human T cells that stably express a second-generation CD19 CAR with or without sToci were co-incubated with donor-matched PBMCs plus CD19⁺ Raji lymphoma cells, and cytokine secretion into culture media was measured via a bead-based flow cytometry assay. Upon antigen stimulation, CD19 CAR-T cells that constitutively expressed sToci (termed sToci-T cells hereafter) produced significantly lower levels of inflammatory cytokines-including IL-2, IL-4, IL-6, IL-8, TNF-α, and GM-CSF-compared to CD19 CAR-T cells that did not express the sToci (termed no-sToci-T cells hereafter) (FIG. 32A). These results suggest that T-cell-secreted sToci can significantly reduce the overall cytokine level in a mixed immune cell population.

The inventors next evaluated the effects of sToci secretion on cytokine production compared to direct treatment with tocilizumab. Co-cultures containing PBMCs and no-sToci-T cells showed reduced cytokine production when treated with 90 μg/ml tocilizumab, which corresponds to a 4 mg/kg clinical dose (FIG. 32B). Increasing tocilizumab to 200 μg/ml led to further reduced cytokine production, indicating a dose-dependent effect of the anti-IL-6Rα antibody. Even compared to 200 μg/ml tocilizumab, which is slightly above the 8 mg/kg clinical dose now commonly used for CRS treatment, T-cell-secreted sToci resulted in comparable cytokine levels (FIG. 32B). These results suggest constitutive sToci production by CAR-T cells has the potential to effectively modulate cytokine production.

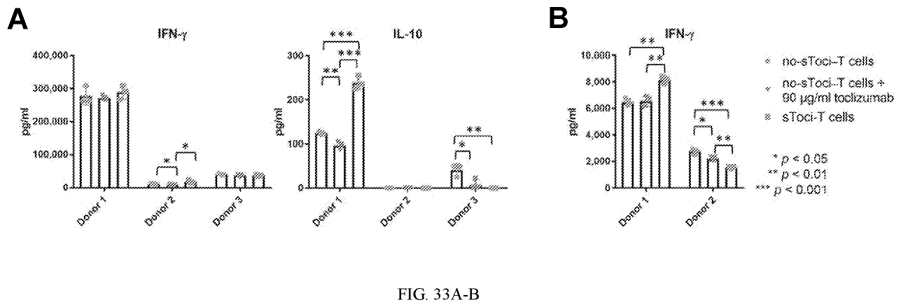

In addition to the cytokines shown in FIG. 31, IL-10 and IFN-γ were also quantified by bead-based flow cytometry. However, the overall levels of these two cytokines proved highly variable from donor to donor (FIG. 33A). In particular, IFN-γ levels appeared unresponsive to both tocilizumab and sToci production based on bead-based flow cytometry results, which was contrary to expectations given tocilizumab's clinical efficacy. To further investigate, ELISA was performed as an alternative method for IFN-γ quantification. However, the results again proved inconsistent across donors (FIG. 33B). The inventors therefore conclude that IFN-γ and IL-10 production levels are highly variable in the CAR-T cell/PBMC/Raji lymphoma co-culture setting, precluding quantitative evaluation of sToci's effect on the production of these cytokines.

3. Constitutive sToci Production does not Inhibit T-Cell Proliferation or Cytotoxicity

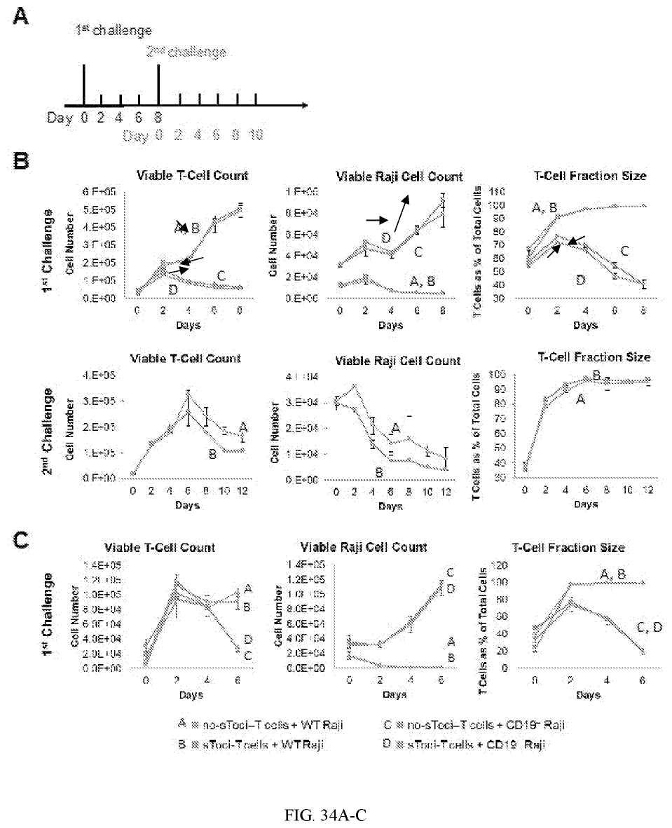

A potential concern of engineering T cells to constitutively secrete sToci is that T-cell effector functions may be compromised, either due to the metabolic burden exerted by sToci production or due to potential inhibitory effects of sToci on T-cell activation. To evaluate the impact of sToci production on T-cell fitness and function, the proliferation and cytotoxicity of sToci- and no-sToci-T cells were quantified after stimulation with CD19⁺ Raji cells (FIG. 34A).

Results indicated similar T-cell proliferation and antigen-specific target-cell lysis for both sToci- and no-sToci-T cells upon antigen stimulation, with consistent behavior in both CD4⁺ (FIG. 34B) and CD8⁺ T cells (FIG. 34C). Importantly, sToci-T cells' ability to lyse target cells was maintained upon repeated antigen challenge (FIG. 34B), confirming sToci production does not result in premature loss of T-cell effector function.

Upon antigen re-challenge, sToci-T cells were observed to proliferate at a slightly lower rate than no-sToci-T cells. However, sToci-T cells also eliminated Raji tumor cells at a faster rate than no-sToci-T cells. Since T-cell expansion is dependent on antigen stimulation, and antigen availability is contingent upon the survival of tumor cells, it is likely that the reduced T-cell expansion observed in sToci-T cells was due to the early depletion of antigen-presenting Raji cells. This is supported by the observation that the T-cell fraction size (i.e., T cells as a percent of all cells present in the co-culture) was essentially identical for both sToci- and no-sToci-T cells throughout the time-course study (FIG. 34B). Taken together, these results indicate that constitutive sToci production does not inhibit the antigen-stimulated proliferation and cytotoxicity of primary human T cells.

4. Constitutive sToci Production does not Alter T-Cell Subtype Differentiation or Exhaustion Pattern

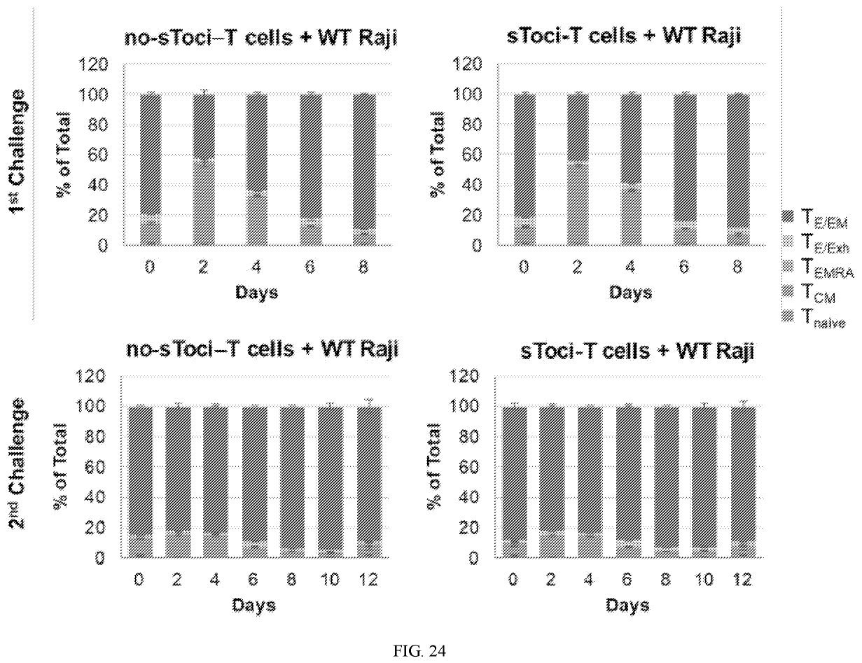

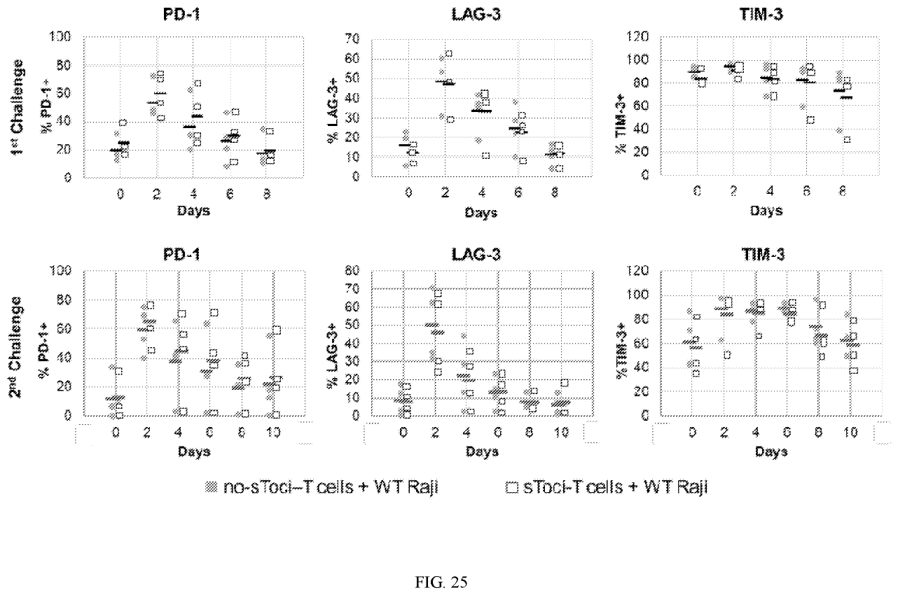

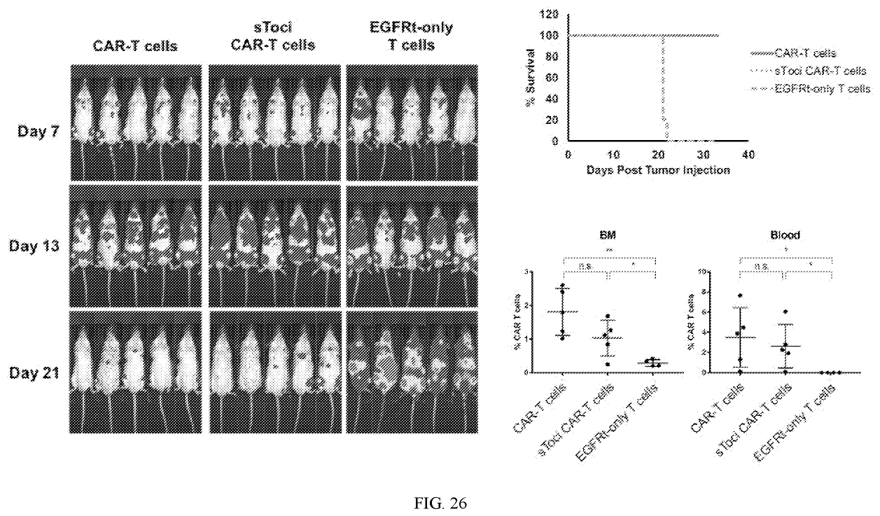

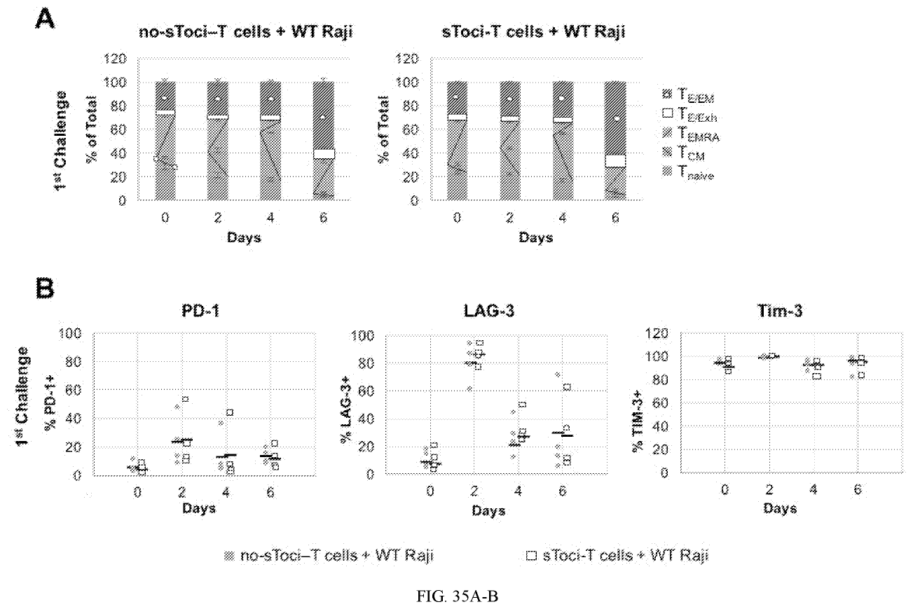

To further confirm that constitutive sToci secretion does not alter aspects of T-cell biology that are critical to anti-tumor effector functions, T-cell subtype differentiation and exhaustion-marker expression were evaluated by flow cytometry. Time-course results demonstrate that antigen stimulation triggers dynamic changes in T-cell subtype distribution (FIGS. 24-25 and FIG. 35A) and exhaustion-marker expression (FIG. 25B and FIG. 35B). However, sToci- and no-sToci-T cells showed nearly identical behaviors at each time point, confirming that sToci production does not alter the dynamics of T-cell subtype differentiation nor the exhaustion state of engineered T cells.

5. Constitutive sToci Production does not Hinder Cytotoxicity of CD19 CAR-T Cells In Vivo

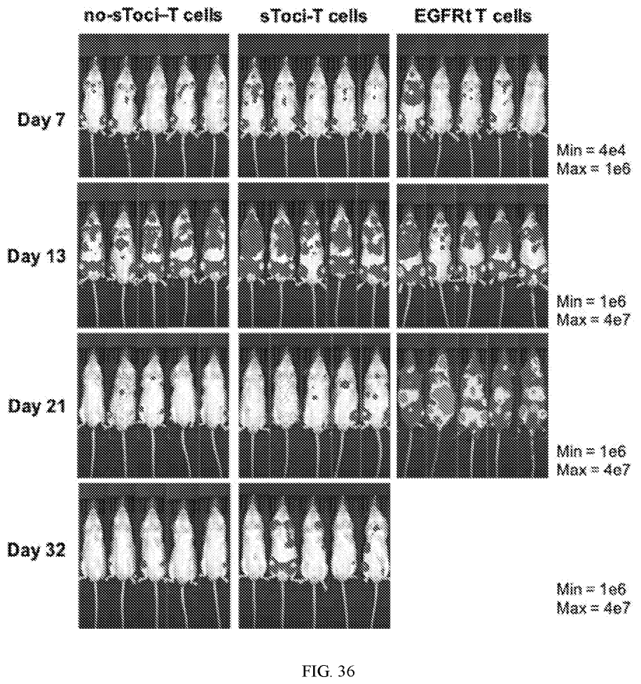

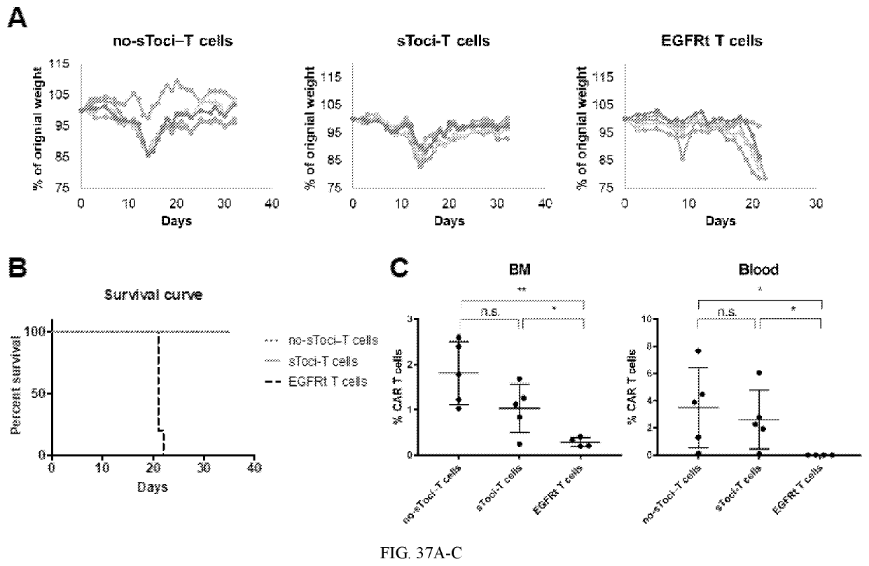

To evaluate the performance of sToci-T cells in vivo, NSG mice were engrafted with 2×10⁶ healthy donor PBMC and 0.5×10⁶ firefly luciferase-expressing CD19⁺ Raji lymphoma cells, and subsequently treated with 2×10⁶ donor-matched CD4⁺ T cells expressing either a truncated epidermal growth factor receptor (EGFRt), which served as a negative control, or an anti-CD19 CAR with or without sToci. The inclusion of PBMC is to simulate the complexity of immune cells in human because CRS is a systemic immune response that associated with multiple cell types. However, there is no observable CRS symptoms in any of the animals, thus the CRS-mediating effect of sToci cannot be evaluated. Despite the lack of CRS occurrence, this in vivo study confirmed that both sToci- and no-sToci-T cells effectively controlled tumor growth, resulting in no significant difference in tumor burden (FIG. 36), weight loss (FIG. 37A), or overall survival (FIG. 37B). Both sToci- and no-sToic-T cells were detectable in the blood and bone marrow of the animals at the time of sacrifice, with no significant difference in the persistence of the two T-cell types (FIG. 37C). Taken together, these results indicate that sToci production does not compromise the in vivo anti-tumor capability or proliferative potential of CAR-T cells.

B. Discussion

CRS is a potentially fatal side effect of immunotherapy that has been widely observed in patients treated with CD19 CAR-T cells (13, 14, 16, 25, 34, 35). The anti-IL-6Rα antibody tocilizumab has shown clinical efficacy in the treatment of CRS, but CRS diagnosis and the timing of CRS treatment remains a highly imprecise process. In this work, the inventors aimed to develop an alternative approach to CRS management by engineering T cells that can lower cytokine production without compromising anti-tumor effector functions, with the goal of generating a therapeutic cell product with significantly reduced risk of CRS. The inventors reasoned that engineering T cells to secrete effector molecules that inhibit IL-6 signaling should be able to exert effective control over CRS without affecting CAR-T cell functions that are essential for anti-tumor efficacy.

Results from this study demonstrate that tocilizumab can be converted into a single-chain format and retain potent inhibitory function against IL-6 signaling. Primary human T cells that stably express sToci show substantially reduced STAT3 phosphorylation upon IL-6 treatment, indicating effective self-regulation of IL-6 signaling. In co-cultures of CD19 CAR-T cells and donor-matched PBMCs, constitutive sToci production by T cells resulted in significantly reduced production of inflammatory cytokines, including TNF-α, IL-2, IL-4, IL-6, IL-8, and GM-CSF. Importantly, this was achieved without affecting antigen-specific cytotoxicity or T-cell proliferation, differentiation, and exhaustion, even upon repeated antigen stimulation. Furthermore, sToci secretion by CAR-T cells yielded similar or better control of cytokine production compared to clinical doses of tocilizumab, highlighting the therapeutic potential of this engineering strategy.

The inventors observed that T-cell-secreted sToci was able to reduce cytokine production to a comparable degree as direct addition of tocilizumab at concentrations equivalent to clinical doses commonly used for CRS treatment. It is unlikely that T cells are able to secrete sToci at concentrations that approach 90 or 200 μg/ml. Indeed, attempts at quantifying sToci concentration in T-cell supernatants yielded undetectable signals by ELISA (data not shown). However, T-cell secreted sToci is detectable by western blot (FIG. 30B) and clearly results in IL-6 signaling inhibition in sToci-producing T cells (FIG. 31C). It is possible that the close physical proximity of T-cell-secreted sToci to its intended target (i.e., the T cells themselves and surrounding immune cells) enables a relatively modest dose of sToci to achieve functionally relevant outcomes. The ability to exert control over cytokine production while greatly reducing the amount of therapeutic agent required has the potential advantage of lowering the risk of toxicities associated with tocilizumab (37-40).

Using a PBMC-humanized mouse model, the inventors the inventors further demonstrated that CD19 CAR-T cells that constitutively express sToci retain potent tumor-lysis capability in vivo, achieving comparable levels of tumor control as CD19 CAR-T cells that do not produce sToci. Taken together, these results demonstrate that CAR-T cells engineered to secrete sToci has the potential to modulate cytokine production levels without compromising anti-tumor effector functions.

The inventors have demonstrated that constitutive sToci production by CD19 CAR-T cells can lead to significant reductions in inflammatory cytokines without compromising antigen-specific T-cell cytotoxicity, proliferation, or subtype differentiation in cell culture. The inventors further demonstrated that despite reduced cytokine production, sToci-producing CAR-T cells retain robust anti-tumor functionality upon repeated antigen challenge in vitro. Further work is needed to fully evaluate the ability of sToci-producing CAR-T cells to prevent or ameliorate CRS in vivo, with a critical challenge being the development of an animal model that can adequately recapitulate the onset and symptoms of CRS upon adoptive T-cell transfer. This work establishes the basic feasibility of engineering T cells with self-autonomous mechanisms to regulate cytokine production. A fully optimized system would have the potential to eliminate the need for indirect monitoring and external interventions and offer a new approach to increasing the safety profile of adoptive T-cell therapy.

C. Methods

1. Plasmid Construction

Anti-IL-6Ra sToci comprised of the light and heavy chains of tocilizumab (41) connected via an 18-amino acid linker (Supplementary Table 2.S1) was synthesized as gBlocks (Integrated DNA) and cloned into the epHIV7 vector (42, 43) downstream of the EF1α promoter by standard molecular cloning methods. A second-generation CD19 CAR containing a 4-1BB co-stimulatory domain was constructed as previously described (44), and a bicistronic plasmid expressing an HA-tagged CD19 CAR fused to sToci via a T2A self-cleaving peptide was constructed in the epHIV7 backbone by isothermal DNA assembly. For the purpose of retrovirus production, both CD19 CAR and CD19 CAR-T2A-sToci constructs were also transferred to the MSCV vector by standard molecular cloning methods. The MSCV vector was a generous gift from Dr. Steven Feldman (National Cancer Institute).

2. Cell Line Maintenance

Wild-type Raji cells were obtained from ATCC in 2003 and CD19⁻ Raji cells were generated as previously described (44). TM-LCL cells were a generous gift from Dr. Michael C. Jensen (Seattle Children's Research Institute). TM-LCL and Raji cells were cultured in RPMI-1640 (Lonza) with 10% heat-inactivated FBS (HI-FBS; Life Technologies). HepG2 human hepatocarcinoma cells were obtained from ATCC in 2014 and cultured in EMEM (Lonza) supplemented with 10% HI-FBS, 1× non-essential amino acid and 1× sodium butyrate (all components from Life Technologies). Human embryonic kidney (HEK)293T cells were obtained from ATCC in 2011 and cultured in DMEM (HyClone) with 10% HI-FBS. Cell lines were routinely checked for Mycoplasma contamination using MycoAlert PLUS Mycoplasma Detection Kit (Lonza). The TM-LCL cell line had been confirmed by flow cytometry to express CD19 and CD20. Cell lines received from ATCC were certified by ATCC and no further authentication was performed on these cell lines.

3. Primary Human T Cell Generation

Primary human CD4⁺ and CD8⁺ T cells as well as peripheral blood mononuclear cells (PBMCs) were isolated from healthy donor blood obtained from the UCLA Blood and Platelet Center using antibody cocktails (RosetteSep kits) followed by Ficoll-Paque (GE Healthcare Life Sciences) density-gradient separation. Isolated cells were stimulated with CD3/CD28 Dynabeads (Life Technologies) at a 1:1 or 1:3 bead:cell ratio. Two days after Dynabeads stimulation, cells were transduced with lentivirus (MOT=3) or untitered retrovirus. Dynabeads were removed on day 9 or 10. Transduced cells were sorted by magnetic bead-based sorting (Miltenyi Biotec) and expanded in the presence of irradiated TM-LCL feeder cells (T cell:TM-LCL=1:7). T cells used in in vivo studies did not go through expansion with TM-LCL feeder cells. All T cells were cultured in RPMI-1640 supplemented with 10% HI-FBS and fed with 50 Um′ IL-2 (Life Technologies) and 1 ng/ml IL-15 (Miltenyi BIotec) every two days. 100 U/ml penicillin and 100 μg/ml streptomycin (Life Technologies) was supplemented once to T-cell expansion cultures at the time of TM-LCL addition.

4. Lentivirus and Retrovirus Production

For lentivirus production, HEK293T cells were seeded in 10 cm² dishes and transfected with lentiviral packaging constructs and expression vector using polyethyleneimine (PEI) (Polysciences). Viral supernatant was harvested 48 and 72 h after transfection. Concentrated lentivirus was generated by PEG-8000 (Bioexpress) treatment and ultracentrifugation and stored at −80° C. Retroviruses were produced by transfecting HEK293T cells with retroviral packaging constructs and expression vector using PEI. Viral supernatant was harvested 48 and 72 h after transfection. Supernatant was filtered through 45 μm filter (VWR) to remove cell debris and either used for transduction directly or stored at −80° C.

5. sToci Production in HEK293T Cells

HEK293T cells were seeded in 10 cm² dishes and transiently transfected with plasmids encoding sToci using PEI. Cells were cultured in serum-free media for 24 h starting 24 h after transfection. Culture supernatant was harvested and concentrated using Amicon Centrifugal filter units (10,000 NMWL, EMD Millipore).

6. Western Blot

For pSTAT3 western blots of untransduced cells, HepG2 or primary human T cells were seeded in 12-well plates, and incubated with indicated amounts of sToci for 3 h. Indicated wells were subsequently treated with 4 ng/ml of human recombinant IL-6 (Biolegend) for another 30 min before cell harvest. Cell pellets were lysed in 1×RIPA buffer (1% Igepal CA-630, 0.1% SDS, 0.5% sodium Deoxycholate) supplemented with protease and phosphatase inhibitor cocktail tablets (Fisher Scientific). Samples were run on Bolt 4-12% Bis-Tris Plus gels (Life Technologies) then transferred to nitrocellulous membranes (Life Technologies). Membranes were blocked with 5% BSA (Amresco) in TBS-T buffer (50 mM Tris, 150 mM NaCl, 0.05% Tween 20) for 1 h at room temperature, then incubated in TBS-T buffer containing pSTAT3 (Y705) antibody (Biolegend; cat: 651002) and 5% BSA overnight at 4° C. GAPDH (Sigma Aldrich; cat: G9292) was stained as loading control for all the samples. For pSTAT3 western blot of lentivirally transduced T cells, both sToci- and no-sToci-T cells were cultured in 24-well plates and followed the same sample preparation as the pSTAT3 western blot indicated above.

For HEK293T cell-secreted sToci western blots, sToci samples were prepared as described in the sToci production section. Membranes for sToci western blots were processed with SNAP i.d. protein detection system (EMD Millipore) following manufacturer's protocol. For T-cell-secreted sToci western blots, lentivirally transduced T cells were serum-starved for 72 hours, and culture supernatant was prepared as described in the sToci production section. Sample-containing membrane was blocked with 0.5% non-fat milk in TBS-T buffer, then incubated in TBS-T buffer containing anti-FLAG antibody (Sigma Aldrich; cat: F1804-200UG). Anti-mouse secondary antibody conjugated to horseradish peroxidase (HRP; Jackson ImmunoResearch; cat: 115-035-062) was used for both western blots. Images of western blots were visualized using SuperSignal West Pico Chemiliminescent Substrate (Thermo Scientific).

7. Cytokine Production Assay

Primary CD4⁺ T cells expressing CD19 CAR with or without sToci expression were co-incubated with PBMCs from the same donor plus wild-type Raji cells in a 1:1:1 ratio for 12 h. CD19 CAR-T cells that did not express sToci were treated with 0, 90, or 200 μg/ml of tocilizumab (Genentech) for comparison. The tocilizumab dosage was calculated from the recommended clinical dose of 4 mg/kg tocilizumab, which converts to 90 μg/ml based on the assumption that a 70-kg adult has 3L of blood plasma. To account for the higher, 8 mg/kg, dose of tocilizumab that has become more common in recent years, a 200 μg/ml concentration was also tested. Culture supernatant was harvested by centrifuging cells at 1200 rpm for 2 min after a 12-h co-incubation. Interleukin (IL)-2, IL-4, IL-6, IL-8, IL-10, interferon (IFN)-γ, tumor necrosis factor (TNF)-α, and granulocyte-macrophage colony-stimulating factor (GM-CSF) levels in culture supernatants were quantified with the BD Cytometric Bead Array Human Th1/Th2 Cytokine Kit II and BD CBA Flex Sets (BD Biosciences, San Jose, Calif., USA) following manufacturer's protocols. With the same co-culture setup, the concentration of IFN-γ was also measured by enzyme-linked immunosorbent assay (ELISA) following manufacturer's protocols (Human IL-6 ELISA MAX Deluxe kit, Biolegend).

8. T-Cell Proliferation, Cytotoxicity, Subtype Differentiation, and Exhaustion-Marker Expression Assays

Primary CD4⁺ or CD8⁺ T cells expressing anti-CD19 CAR with or without sToci expression were co-incubated with wild-type (WT; CD19⁺) or CD19⁻ mutant Raji cells at a 2:1 effector-to-target (E:T) ratio. Both Raji cell lines were engineered to stably express EGFP to enable distinction between T cells and Raji cells during flow-cytometry analysis. Samples were harvested every 2 days and analyzed for viable T-cell count, viable target-cell count, as well as differentiation and exhaustion-marker expression. T-cell proliferation and cytotoxicity were quantified by counting viable T cells and Raji cells on a MACSQuant VYB flow cytometer (Miltenyi BIotec). T-cell differentiation was monitored by staining the cells with CD45RA-Viogreen (cat: 130-096-921), CCR7-PE-vio770 (cat: 130-108-287), and CD57-APC (cat: 130-092-141) (all from Miltenyi Biotec) to determine the T-cell subtype. Exhaustion-marker expression was quantified by surface antibody staining of PD-1-PE-vio770 (Miltenyi Blotec; cat: 130-099-878), LAG-3-APC (eBioscience; cat: 17-2239-42) and Tim-3-BV421 (Biolegend; cat: 345008). For primary CD4⁺ T cells, T cells co-incubated with WT Raji cells were harvested after 8 days and re-challenged with fresh WT Raji cells at a 2:1 E:T ratio. Proliferation and differentiation were monitored for another 12 days during the second challenge. CD8⁺ T cells could not be re-challenged due to the limited number of viable T cells remaining at the end of the first challenge.

9. In Vivo Study

In vivo experiment was approved by the UCLA Institutional Animal Care and Use Committee. Six- to eight-week-old NOD/SCID/□c^{-/-} (NSG) mice were purchased from UCLA Department of Radiation and Oncology. Two million peripheral blood mononuclear cells (PBMCs) and 0.5×10⁶ EGFP+, firefly luciferase (ffluc)-expressing Raji cells were injected into NSG mice via tail-vein injection on day 0. On day 7, animals with engrafted tumors were treated via tail-vein injection with 2×10⁶ donor-matched CD4⁺ T cells that were transduced with a truncated epidermal growth factor receptor (EGFRt; negative control) or a CD19 CAR with or without co-expression of sToci. Peripheral blood was collected by retro-orbital or submandibular bleeding on day 15 (8 days post T-cell injection). Animals were sacrificed at the humane endpoint or on day 35 (28 days post T-cell injection), whichever was earlier. At the time of animal sacrifice, a final blood sample was obtained by cardiac puncture and bone marrow was collected from the femur and tibia. Blood cellular contents were stained with human CD45-PacBlue (Biolegend; cat: 304022) and HA-biotin (Miltenyi Biotec; cat: 120-002-691) antibodies followed by streptavidin-PE (Jackson ImmunoResearch; cat: 016-110-084), and analyzed on a MACSQuant VYB flow cytometer (Miltenyi Biotec) to determine CAR-T cell content. Tumor progression was monitored by bioluminescence imaging using an IVIS Lumina III LT Imaging System (PerkinElmer, Waltham, Mass., USA). Animals were weighed daily per standard animal care. The sample size of n=5 was designed to acquire statistically meaningful data. There was no randomization or blinding in the animal studies reported.

10. Statistical Analysis

A minimum of triplicates was chosen to allow for calculation of statistics. Statistical significance was analyzed by using two-tailed, unpaired, homoscedastic Student's t-test.

D. Table

SUPPLEMENTARY TABLE 2.S1
Amino acid sequence of components in sToci

Tocilizumab light chain
DIQMTQSPSSLSASVGDRVTITCRASQDISSYLNWYQQKPGKAPK
LLIYYTSRLHSGVPSRFSGSGSGTDFTFTISSLQPEDIATYYCQQ
GNTLPYTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
(SEQ ID NO: 127)
Linker sequence
GSTSGSGKPGSGEGSTKG (SEQ ID NO: 14)
Tocilizumab heavy chain
EVQLQESGPGLVRPSQTLSLTCTVSGYSITSDHAWSWVRQPPGRG
LEWIGYISYSGITTYNPSLKSRVTMLRDTSKNQFSLRLSSVTAAD
TAVYYCARSLARTTAMDYWGQGSLVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPELLGGPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSRDEL
TKNQVSLTCLVKGFYPSDIAVEWESNGQPENNYKTTPPVLDSDGS
FFLYSKLTVDKSRWQQGNVFSCSVMHEALHNHYTQKSLSLSPG
(SEQ ID NO: 128)

E. Reference

The following references and the publications referred to throughout the specification, to the extent that they provide exemplary procedural or other details
supplementary to those set forth herein, are specifically incorporated herein by reference.
1. Rosenberg, S. A. & Restifo, N. P. Adoptive cell transfer as personalized immunotherapy for human cancer. Science. 348, 62-68 (2015).
2. June, C. H. Adoptive T cell therapy for cancer in the clinic. J. Clin. Invest. 117, 1466-1476 (2007).
3. Brentjens, R. J. et al. Safety and persistence of adoptively transferred autologous CD19-targeted T cells in patients with relapsed or chemotherapy refractory B-cell leukemias. Blood 118, 4817-4828 (2011).
4. Kochenderfer, J. N. et al. Chemotherapy-refractory diffuse large B-cell lymphoma and indolent B-cell malignancies can be effectively treated with autologous T cells expressing an anti-CD19 chimeric antigen receptor. J. Clin. Oncol. 33, 540-549 (2015).
5. Garfall, A. L. et al. Chimeric Antigen Receptor T Cells against CD19 for Multiple Myeloma. N. Engl. J. Med. 373, 1040-1047 (2015).
6. Wang, X. et al. Phase 1 studies of central memory-derived CD19 CAR T-cell therapy following autologous HSCT in patients with B-cell NHL. Blood 127, 2980-2991 (2016).
7. Turtle, C. J. et al. CD19 CAR-T cells of defined CD4+: CD8+ composition in adult B cell ALL patients. J. Clin. Invest. 126, 2123-2138 (2016).
8. FDA approves tisagenlecleucel for B-cell ALL and tocilizumab for cytokine release syndrome. (2017). at <https://www.fda.gov/Drugs/InformationOnDrugs/ApprovedDrugs/ucm574154.htm>
9. FDA approves CAR-T cell therapy to treat adults with certain types of large B-cell lymphoma. (2017). at <https://www.fda.gov/NewsEvents/Newsroom/PressAnnouncements/ucm581216.htm>
10. Porter, D. L. et al. Chimeric antigen receptor T cells persist and induce sustained remissions in relapsed refractory chronic lymphocytic leukemia. Sci. Transl. Med. 7, 1-12 (2015).
11. Lee, D. W. et al. T cells expressing CD19 chimeric antigen receptors for acute lymphoblastic leukaemia in children and young adults: A phase 1 dose-escalation trial. Lancet 385, 517-528 (2015).
12. Davila, M. L. et al. Efficacy and toxicity management of 19-28z CAR T cell therapy in B cell acute lymphoblastic leukemia. Sci. Transl. Med. 6, 1-13 (2014).
13. Maude, S. L. et al. Chimeric Antigen Receptor T Cells for Sustained Remissions in Leukemia. N. Engl. J. Med. 371, 1507-1517 (2014).
14. Brentjens, R., Yeh, R., Bernal, Y., Riviere, I. & Sadelain, M. Treatment of chronic lymphocytic leukemia with genetically targeted autologous T cells: case report of an unforeseen adverse event in a phase I clinical trial. Mol. Ther. 18, 666-668 (2010).
15. Locke, F. L. et al. Phase 1 Results of ZUMA-1: A Multicenter Study of KTE-C19 Anti-CD19 CAR T Cell Therapy in Refractory Aggressive Lymphoma. Mol. Ther. 25, 285-295 (2017).
16. Kochenderfer, J. N. et al. B-cell depletion and remissions of malignancy along with cytokine-associated toxicity in a clinical trial of anti-CD19 chimeric-antigen-receptor-transduced T cells. Blood 119, 2709-2721 (2012).
17. Tanyi, J. L. et al. Possible compartmental cytokine release syndrome in a patient with recurrent ovarian cancer after treatment with mesothelin-targeted CAR-T cells. J. Immunother. 40, 104-107 (2017).
18. Morgan, R. A. et al. Case report of a serious adverse event following the administration of T cells transduced with a chimeric antigen receptor recognizing ERBB2. Mol. Ther. 18, 843-851 (2010).
19. Ali, S. A. et al. T cells expressing an anti-B-cell maturation antigen chimeric antigen receptor cause remissions of multiple myeloma. Blood 128, 1688-1701 (2016).
20. Hill, J. A. et al. Infectious complications of CD19-targeted chimeric antigen receptor-modified T cell immunotherapy. Blood 131, 121-130 (2017).
21. Park, J. H. et al. Cytokine Release Syndrome Grade is a Predictive Marker for Infections in Relapsed or Refractory B-cell All Patients Treated with CAR T Cells. Clin. Infect. Dis. 1-8 (2018). doi:10.1093/cid/ciy152
22. Lee, D. W. et al. Current concepts in the diagnosis and management of cytokine release syndrome. Blood 124, 188-195 (2014).
23. Brudno, J. N. & Kochenderfer, J. N. Toxicities of chimeric antigen receptor T cells: recognition and management. Blood 127, 3321-3331 (2016).
24. Maude, S. L., Barrett, D., Teachey, D. T. & Grupp, S. A. Managing Cytokine Release Syndrome Associated With Novel T Cell-Engaging Therapies. Cancer J. 20, 119-122 (2014).
25. Grupp, S. A. et al. Chimeric antigen receptor-modified T cells for acute lymphoid leukemia. N. Engl. J. Med. 368, 1509-18 (2013).
26. Teachey, D. T. et al. Identification of Predictive Biomarkers for Cytokine Release Syndrome after Chimeric Antigen Receptor T-cell Therapy for Acute Lymphoblastic Leukemia Clinical Description of Patients. Cancer Discov. 6, 664-679 (2016).
27. Hay, K. A. et al. Kinetics and biomarkers of severe cytokine release syndrome after CD19 chimeric antigen receptor-modified T-cell therapy. Blood 130, 2295-2306 (2017).
28. Diaconu, I. et al. Inducible Caspase-9 Selectively Modulates the Toxicities of CD19-Specific Chimeric Antigen Receptor-Modified T Cells. Mol. Ther. 25, 1-13 (2017).
29. Di Stasi, A. et al. Inducible Apoptosis as a Safety Switch for Adoptive Cell Therapy. N. Engl. J. Med. 365, 1673-1683 (2011).
30. Wang, X. et al. A transgene-encoded cell surface polypeptide for selection, in vivo tracking, and ablation of engineered cells. Blood 118, 1255-1263 (2011).
31. Obstfeld, A. E. et al. Cytokine release syndrome associated with chimeric-antigen receptor T-cell therapy; clinicopathological insights. Blood prepublished online Oct. 26 2017 (2017). doi:10.1182/blood-2017-08-802413
32. Singh, N. et al. Monocyte lineage-derived IL-6 does not affect chimeric antigen receptor T-cell function. Cytotherapy 19, 867-880 (2017).
33. Barrett, D. M., Singh, N., Hofmann, T. J., Gershenson, Z. & Grupp, S. A. Interleukin 6 Is Not Made By Chimeric Antigen Receptor T Cells and Does Not Impact Their Function. Blood 128, 654 LP-654 (2016).
34. Porter, D. L., Levine, B. L., Kalos, M., Bagg, A. & June, C. H. Chimeric Antigen Receptor-Modified T Cells in Chronic Lymphoid Leukemia. N. Engl. J. Med. 365, 725-733 (2011).
35. Kalos, M. et al. T Cells with Chimeric Antigen Receptors Have Potent Antitumor Effects and Can Establish Memory in Patients with Advanced Leukemia. Sci. Transl. Med. 73, 1-11 (2011).
36. Adachi, K. et al. IL-7 and CCL19 expression in CAR-T cells improves immune cell infiltration and CAR-T cell survival in the tumor. Nat. Biotechnol. 36, 346 (2018).
37. Jones, G. & Ding, C. Tocilizumab: A review of its safety and efficacy in rheumatoid arthritis. Clin. Med. Insights Arthritis Musculoskelet. Disord. 3, 81-89 (2010).
38. Koike, T. et al. Postmarketing surveillance of tocilizumab for rheumatoid arthritis in Japan: Interim analysis of 3881 patients. Ann. Rheum. Dis. 70, 2148-2151 (2011).
39. Hiura, M. et al. Case of severe liver damage after the induction of tocilizumab therapy for rheumatoid vasculitis. Hepatol. Res. 41, 492-496 (2011).
40. Mahamid, M., Paz, K., Reuven, M. & et al. Hepatotoxicity due to tocilizumab and anakinra in rheumatoid arthritis: two case reports. Int J Gen Med 657-660 (2011).
41. Zhang, X., Bao, M., Jahreis, A. M. & Terao, K. U.S. Pat. No. 8,580,264B2. 1, (2013).
42. Yam, P. Y. et al. Design of HIV Vectors for Efficient Gene Delivery into Human Hematopoietic Cells. Mol. Ther. 5, 479-484 (2002).
43. Wang, X. et al. A transgene-encoded cell surface polypeptide for selection, in vivo tracking, and ablation of engineered cells. Blood 118, 1255-1263 (2011).
44. Zah, E., Lin, M., Silva-benedict, A., Jensen, M. C. & Chen, Y. Y. T Cells Expressing CD19/CD20 Bispecific Chimeric Antigen Receptors Prevent Antigen Escape by Malignant B Cells. Cancer Immunol. Res. 4, 498-508 (2016).

### Example 2: Identifying Alternative Treatment Agents for CRS

The frequent occurrence of cytokine release syndrome (CRS) in adoptive-T cell therapy has prompted numerous researches on controlling immune response through synthetic biology tools such as incorporating suicide genes into CAR-T cells. However, very limited development of new CRS treatment agents has been reported. Although the current treatments, corticosteroids and tocilizumab, are effective for most patients, the lack of new treatment agents poses a risk on patients who do not respond to the current treatments. Here, the inventors developed a panel of cytokine antagonists as candidates for new treatment agents of CRS. CD19 CAR-T cells co-expressing the panel of cytokine antagonists retain robust cytotoxicity and proliferation upon antigen stimulation in vitro, while enabling modulation of their own cytokine production as well as that of surrounding non-CAR-T cells and monocytes. This work identifies potential CRS treatment agents and verifies their efficacy in modulating cytokine production without hampering critical anti-tumor T-cell effector functions.

Cytokine release syndrome (CRS) is one of the most common adverse events in adoptive-T cell therapy. In recent clinical trials, around 80% of the patients were reported to develop different degrees of CRS after adoptive transfer of CD19 CAR-T cells (1-6). Severe cases of CRS can be life-threatening, which makes the management of CRS critical for the implementation of CAR-T cell therapy. CRS is characterized by pathogenically high levels of cytokine expression, but cytokine production also correlates with the efficacy of CAR-T cell therapy. The conflicted nature of cytokine expression underscores the delicate balance that much be achieved in CAR-T cell-induced immune response and makes the management of CRS very difficult.

The common treatments for CRS include administering corticosteroids or tocilizumab (7, 8), which work by suppressing the immune response or inhibiting Interleukin-6 receptor alpha (IL-6Ra), respectively. However, corticosteroids and tocilizumab fail to treat some patients with severe CRS, which highlights the need to develop a better approach for CRS management. Our knowledge about cell-therapy-induced CRS has grown broader and deeper with the accumulated clinical observations, and there is a concerted effort to develop a way to predict CRS based on biomarker expression^{1,11,12}. However, relatively few new CRS treatment agents have been explored.

Multiple cytokines (IL-1β, IL-2, IL-6, IL-10, interferon (IFN)-γ, tumor necrosis factor (TNF)-α, etc.) and acute-phase protein (C-reactive protein (CRP) and ferritin) have been observed to be up-regulated in patients with CRS (1, 9-11). A number of studies reported on the use of IL-1β or GM-CSF antagonist as treatments for CRS (13-15). In spite of the fact that the efficacy of IL-1β and GM-CSF antagonist have only been verified with murine models, those studies demonstrated the possibility of mediating CRS with different cytokine antagonists.

In this study, the inventors aim to identify additional treatment agents for CRS based on the broad cytokine expression pattern in CRS patients. Although multiple cytokines are up-regulated in CRS, the choice of which cytokine to inhibit is crucial because of the tight association between cytokine and immune response. Based on previously reported cytokine expression patterns in CRS patients, the inventors chose to dampen the signaling of four different cytokines and evaluate their potential as CRS treatment. Because our ultimate goal is to incorporate CRS treatments into previously-developed ‘self-modulating T cells’ system, where engineered T cells safeguard against immune over-stimulation by secreting ‘self-modulators’, antagonists for cytokine signaling were cloned in single-chain variable fragment (scFv) form to obtain better plasmid incorporation and gene expression in cells.

First, an IL-6Rα antagonist was derived from tocilizumab, a monoclonal antibody that has received FDA approval as CRS treatment. IL-6 has been considered as a key cytokine that was up-regulated in patients with CRS and the treatment of tocilizumab has been successful in most of the CRS patients. However, the treatment of tocilizumab requires systemic high-dose administration, which may lead to unwanted side effect and the treatment of tocilizumab could not help every patient with severe CRS. Accordingly, additional therapeutic molecules are needed.

The inventors cloned, tested, and studied a panel of anti-TNF-α scFvs derived from four different monoclonal antibodies-Infliximab, Adalimumab, Golimumab, and Certolizumab.

IL-1 is another cytokine that is up-regulated in CRS patients. It has been reported that IL-1 is up-regulated prior to IL-6 up-regulation (13) and the efficacy of IL-1 signaling antagonist as CRS treatment in murine model has been demonstrated in recent publications (13, 14). Therefore, an IL-1 antagonist derived from anakinra, a recombinant version of human IL-1 receptor antagonist protein, was also included in this study.

Lastly, in addition to the above-mentioned cytokines, the possibility of interfering IFN-γ signaling was also considered due to the fact that the up-regulation of IFN-γ is always observed in patients with CRS. Furthermore, IFN-γ is associated with neurotoxicity, another common toxicity in CAR-T cell therapy that cannot be effectively addressed by tocilizumab^{18,19}.

The panel of antagonists were assessed for their ability to inhibit cytokine signaling as well as their impact on anti-tumor effector function. Antagonists secreted by engineered T cells were observed to lower cytokine production by both engineered T cells and nearby, unmodified immune cells. In particular, when co-expressed with CD19 CAR in primary human T cells, scFvs derived from tocilizumab and the anti-TNF-α antibody certolizumab were shown to 1 modulate cytokine production without compromising antigen-stimulated T-cell proliferation and target-cell killing, confirming the feasibility of productively incorporating the panel of antagonists into a self-modulating T-cell system for cancer therapy.

A. Results

1. The Panel of Cytokine Signaling Antagonists can be Expressed in Mammalian Cells

To generate clinically applicable antagonists for different cytokines, the inventors searched for the sequence of multiple commercially available biologics and derived corresponding scFvs from them (Table 3.1, Supplementary Table 3.S1). Specifically, scFv was derived by connecting variable heavy chain (V_{H}) and variable light chain (V_{L}) through an scFv linker sequence. Both orientations of scFv (V_{H}-V_{L} or V_{L}-V_{H}) were generated for each antibody of interest to evaluate whether orientation affects scFv function.

TABLE 3.1
List of antagonists incorporated in this study.

| Original antagonist | Type | Source | Target |
|---|---|---|---|
| Tocilizumab | Antibody | Humanized | IL-6 receptor □ |
| Infliximab | Antibody | Chimeric (murine variable and | TNF-□ |
| | | human constant region) | |
| Adalimumab | Antibody | Human | TNF-□ |
| Golimumab | Antibody | Human | TNF-□ |
| Certolizumab pegol | Fab’ fragment | Humanized | TNF-□ |
| Emapalumab | Antibody | Human | IFN-□ |
| Anakinra | Recombinant protein | Human | IL-1 receptor |

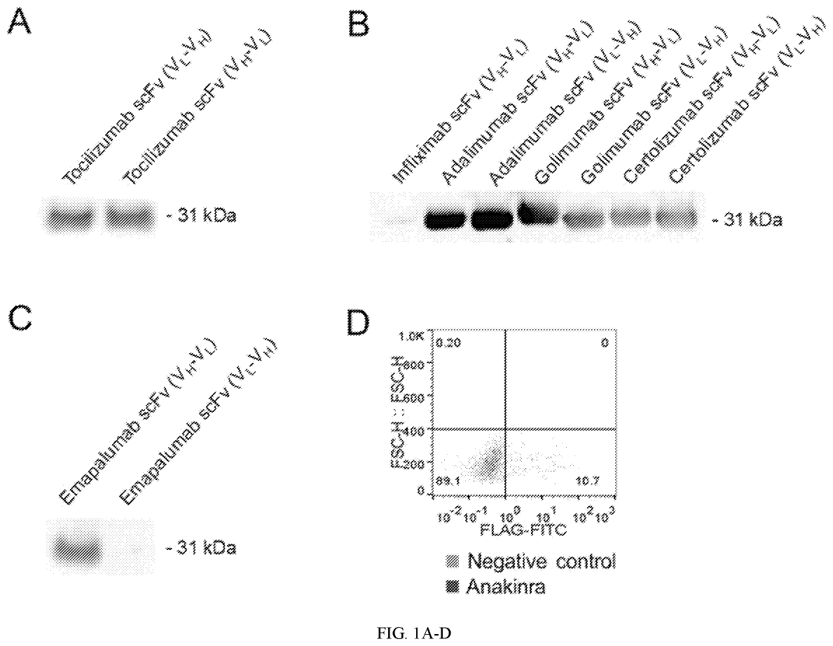

Both versions of tocilizumab-derived scFvs were confirmed to be efficiently expressed and secreted by HEK293T cells (FIG. 1A). The expression and secretion of most of the anti-TNF-α scFvs were verified in a similar manner as tocilizumab scFv. However, the V_{H}-V_{L} scFv derived from infliximab was detected at very low levels compared to other TNF-α scFvs (FIG. 1B), and the V_{L}-V_{H} version of this antibody could not be successfully cloned. Anti-IFN-7 scFvs derived from emapalumab was more efficiently expressed in V_{H}-V_{L} orientation even though both orientations were successfully cloned (FIG. 1C). Expression of anakinra in HEK293T cells was confirmed by flow analysis of intracellularly stained sample, albeit at low expression levels (FIG. 1D). Based on all the results, emapalumab V_{L}-V_{H} were excluded from future experiments due to poor expression. Anakinra was retained because of its unique target (IL-1 receptor) compare to other scFvs.

2. IL-6Rα and TNF-α scFvs have Varying Efficacy in Dampening Cytokine Signaling Despite Proper Expression and Secretion

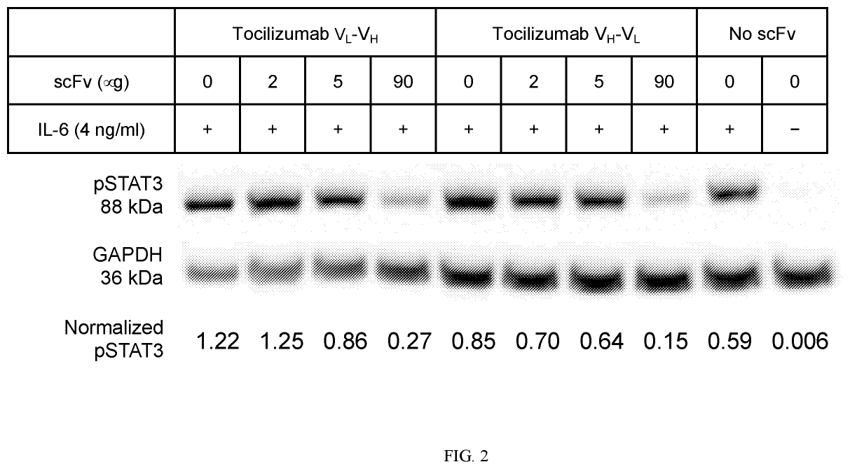

To further narrow the panel of scFvs to identify candidates with therapeutic potential, the inventors evaluated the ability of the IL-6Rα and TNF-α scFvs to inhibit their target cytokines. HEK293T cells were engineered to secrete each scFv, which was subsequently harvested and concentrated from the culture supernatant. The effect of IL-6Rα scFvs on IL-6 signaling was quantified by measuring the expression of phosphorylated STAT3 (pSTAT3), a transcription activator in the IL-6 signaling pathway, in primary human CD4⁺ T cells stimulated with exogenous IL-6 with and without a concentrated dose of scFv. Western blot results showed that scFv addition decreased the extent of STAT3 phosphorylation triggered IL-6 stimulation, with both versions of the scFv (V_{H}-V_{L} and V_{L}-V_{H}) showing similar IL-6 signaling inhibition efficacy, each in a dose-dependent manner (FIG. 2). These results indicate that the tocilizumab-based scFvs are effective in modulating IL-6 signaling.

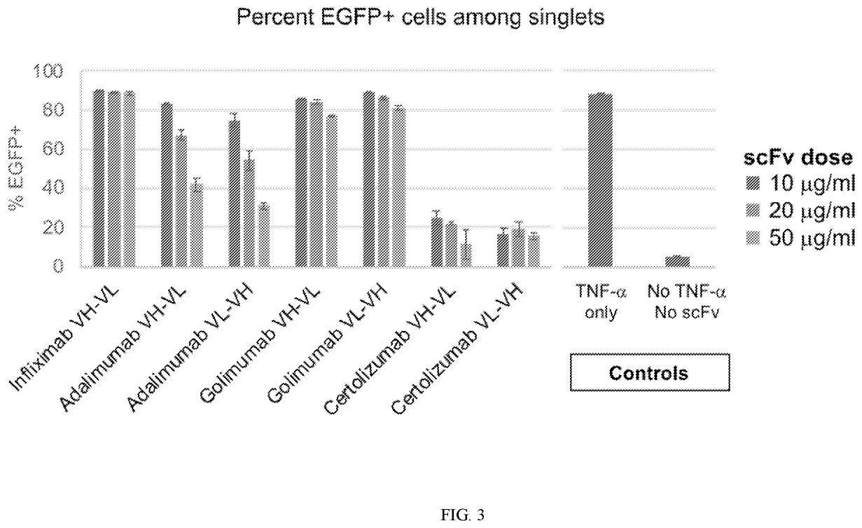

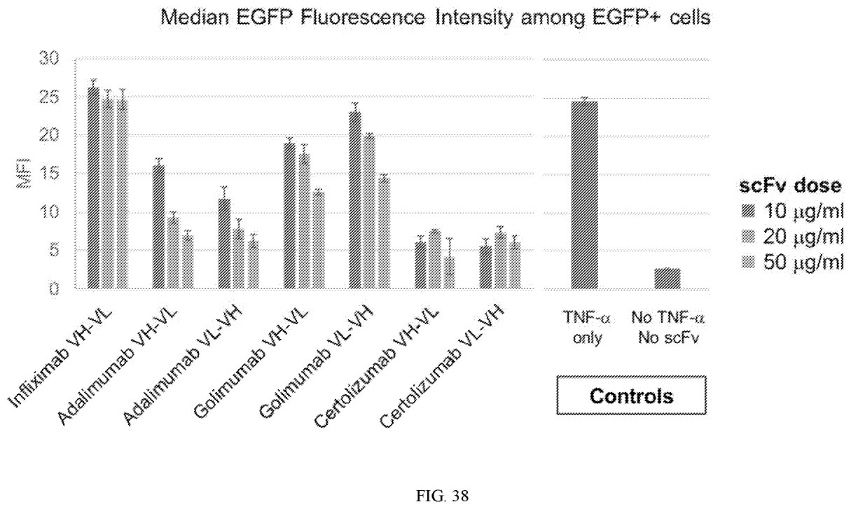

The effect of anti-TNF-α scFvs on TNF-α signaling was assessed by quantifying NFκB signaling upon TNF-α stimulation in a Jurkat cell line that expresses enhanced green fluorescent protein (EGFP) from an NFκB-responsive promoter. Varying concentrations of TNF-α scFv were added to Jurkat cells stimulated with exogenous TNF-α, and the NFκB signaling intensity was measured by EGFP expression after a 24-hour incubation. Cells treated with either orientation of adalimumab- and certolizumab-derived scFvs showed dose-dependent reduction in EGFP expression compared to untreated cells, demonstrating the scFvs' efficacy in inhibiting TNF-α signaling. However, treatment with infliximab- and golimumab-derived scFvs showed limited effects on EGFP expression, leading to the elimination of these candidates from further consideration (FIG. 3, FIG. 38).

3. Cytokine Antagonists can be Efficiently Co-Expressed with CD19 CAR and do not Alter CD4/CD8 Ratio in Primary Human T Cells

Based on the above results, scFvs derived from tocilizumab, emapalumab, adalimumab, and certolizumab were selected for further characterization. Given that little difference was observed between V_{H}-V_{L} and V_{L}-V_{H} orientations, the inventors chose to proceed with the V_{L}-V_{H} designs (except emapalumab scFv, which was used in V_{H}-V_{L} format due to the poor expression of its V_{L}-V_{H} design).

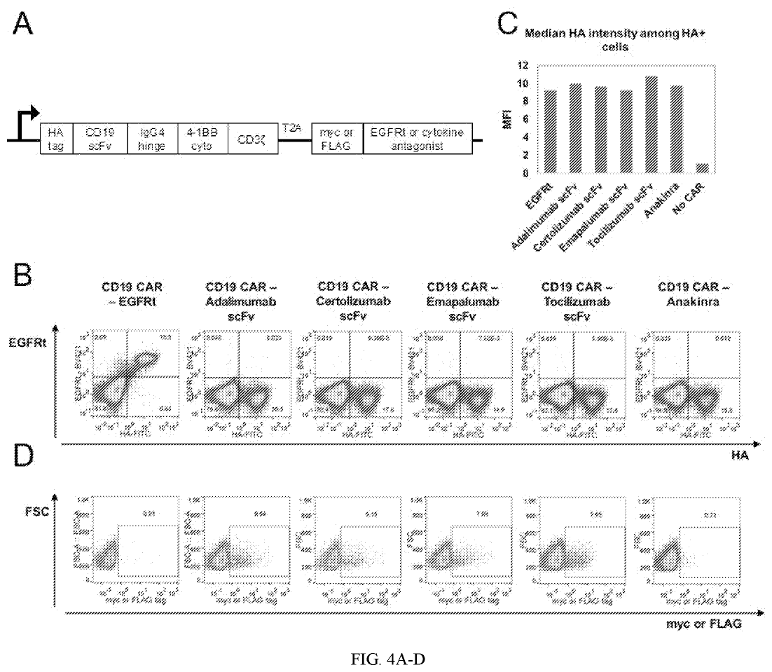

The inventors next constructed bicistronic expression vectors encoding a second-generation CD19 CAR connected to each scFv or anakinra via a “self-cleaving” 2A sequence (FIG. 4A). The CAR, scFv, and anakinra were each labeled with an N-terminal tag to enable antibody staining and subsequent analysis by flow cytometry. The inventors term these biscistronic designs “self-modulating CAR constructs,” as the scFvs are expected to modulate the activities—particularly cytokine production levels—of the CAR-T cells that express these designs.

HEK293T cells transfected with the panel of self-modulating CAR constructs were surface-stained for CAR expression and intracellularly stained for scFv expression. Flow cytometry results indicated that the CAR was effectively expressed in all cases, and co-expression of cytokine agonists (scFv or anakinra) did not impact CAR surface expression levels. (FIG. 4B, 4C). Intracellular staining further confirmed the production of scFv proteins, as well as the relatively low expression of anakinra (FIG. 4D), which is consistent with previously described western-blot results shown in in FIG. 1. Taken together, these observations suggest that anakinra may not be the most effective choice for cytokine regulation due to its low expression levels, which may have been a result of its known short half-life (36, 37). However, given that previous publications had indicated that anakinra administration can mediate the severity of CRS in animal models (13, 14), the inventors chose to retain this construct as a comparison to the scFv-based cytokine antagonists developed in this study.

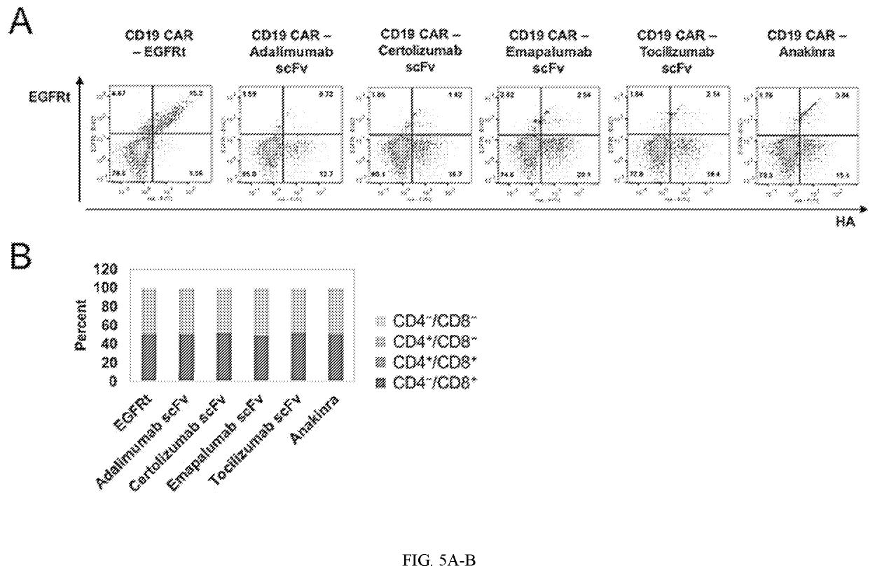

Next, the inventors transduced the panel of self-modulating CAR constructs into primary human T cells and confirmed comparable CAR expression levels across the panel (FIG. 5A). In addition, the CD4/CD8 ratio in T cell culture was unaltered in all the self-modulating CAR T cells (FIG. 5B), indicating that constitutive secretion of cytokine antagonists did not lead to unanticipated changes in T-cell subtype differentiation or the relative growth rate of CD4⁺ vs. CD8⁺ T cells. Taken together, these results suggest that the added production of cytokine antagonists does not exert a significant cost on T cells that would affect their CAR expression level or intrinsic physiology.

4. Secretion of Cytokine Antagonists Reduces Cytokine Production by Both Engineered T Cells and Nearby Immune Cells

The inventors next evaluated the efficacy of different cytokine antagonists in controlling cytokine production by utilizing the Isoplexis technology (20, 21). In brief, Isoplexis is a chip-based assay that enables measurements of cytokine production at a single-cell level. This unique setup allows us to co-incubate T cells with tumor cells and other immune cell types, and interrogate the identity and amount of cytokines produced by each cell type. Armed with this capability, the inventors set out to investigate whether the secretion of cytokine antagonists by CAR-T cells would influence cytokine production by not only the engineered T cells but also nearby immune cells, including unmodified CD4 T cells found among peripheral blood mononuclear cells (PBMC-derived CD4+ T cells) and monocytes (which are believed to be the main producer of key CRS-related cytokines such as IL-6 (23).

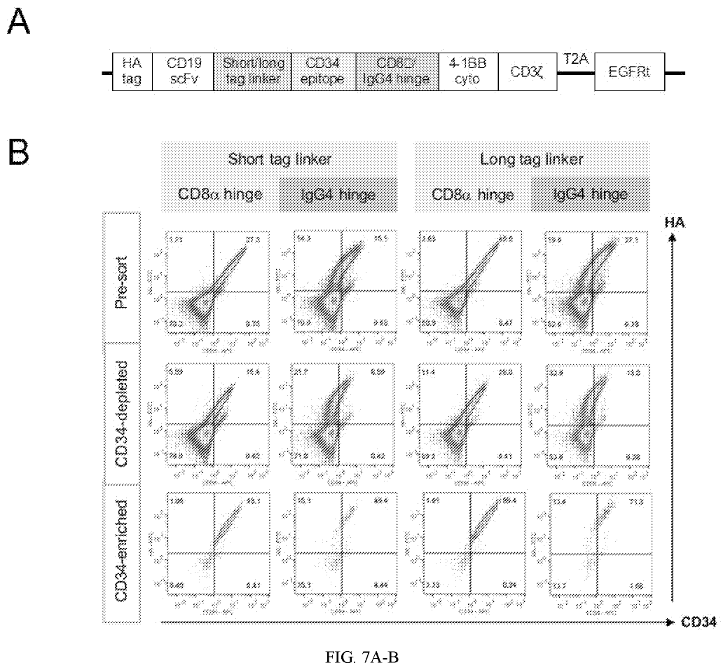

Given inherent cell-to-cell variabilities, it is essential that at least 500 cells of each cell type of interest are quantified to generate reliable data through the Isoplexis assay. This requirement, in turn, necessitates the removal of untransduced T cells in order to avoid measuring cytokines from non-CAR-T cells. Theoretically, CAR-T cells can be enriched by the N-terminal HA-tag attached to the CD19 CAR. However, it had been observed in our laboratory that cell sorting performed with antibodies bound to N-terminal HA-tags of CARs generally yielded low-quality T cells, potentially due to suboptimal CAR signaling triggered by the antibody and consequent exhaustion of the CAR-T cells. To avoid premature exhaustion of T cells, the cubi-CAR strategy developed by Valton et al. (22) was implemented in this study. In short, Valton et al. engineered B cell maturation antigen (BCMA) CAR to contain a CD34 epitope (Supplementary Table 3.S2), which enabled the enrichment of CAR-T cells without triggering T-cell activation. However, the structure of the BCMA CAR is different from that of the CD19 CAR in our study, and it had not been demonstrated whether the CD34 epitope strategy would work in other CAR constructs. To identify a CD19 CAR design that can incorporate the CD34 epitope without undermining CAR signaling ability, a panel of four CD19 CAR constructs were built to test different combinations of linker sequences and extracellular spacers in the CAR (FIG. 7A). Specifically, the CD34 epitope was connected to the C-terminus of the CD19 scFv via either a long or a short tag linker, and the CD34 epitope was followed by either the CD8a hinge used by Valton et al. or the IgG4 hinge used in our original CD19 CAR construct. An HA tag was further attached to the N-terminus of the CAR, such that staining efficiency for the HA tag and the CD34 epitope could be directly compared. Primary human T cells transduced with the panel of CAR constructs were assessed for CAR expression by flow cytometry. HA staining results confirmed that the CD19 CAR was efficiently expressed in all designs, regardless of the length of tag linker or the identity of the extracellular spacer. However, the CD34 epitope could not be efficiently bound by antibodies when coupled with the IgG4 hinge, resulting in reduced cell-sorting efficiency (FIG. 7B). Based on these results, the CAR construct containing the CD34 epitope with a long linker sequence and the CD8a hinge was chosen for subsequent experiments.

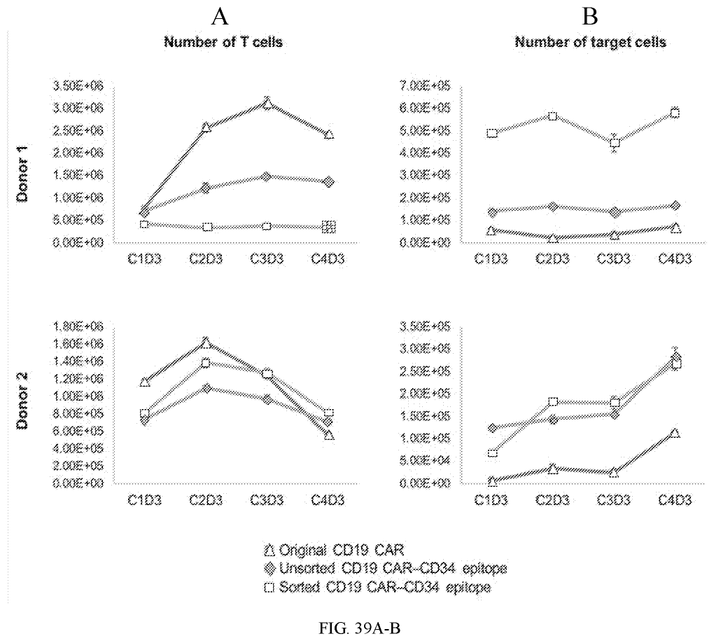

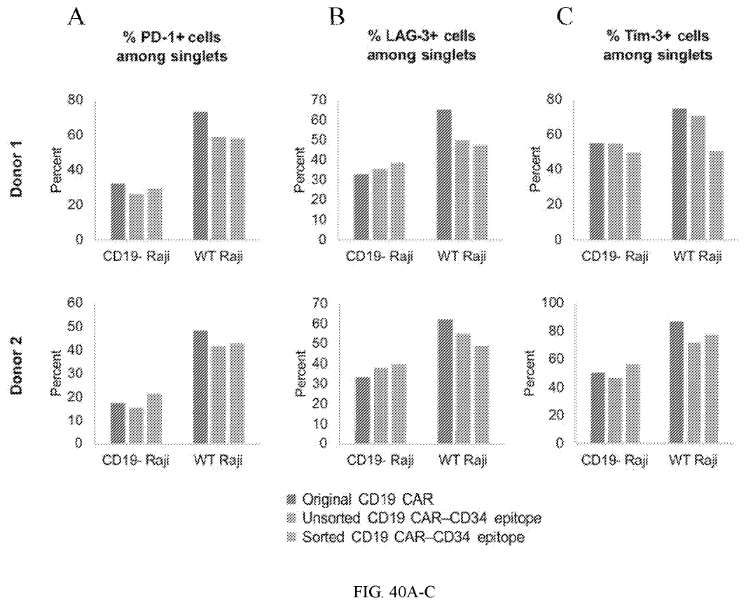

Although enriching cells by CD34 epitope should not stimulate T cells and cause premature exhaustion, it is unclear whether incorporating CD34 epitope would affect the functionality of the CD19 CAR due to the structural change of the protein. In a repeated antigen challenge assay, T cells from two healthy donors encoding original CD19 CAR or CD19 CAR with CD34 epitope (sorted and unsorted) were co-cultured with wildtype Raji cells and evaluated for cell number every three days. CD19 CAR with CD34 epitope showed inferior cytotoxicity and proliferation compare to original CD19 CAR, but the effect of enrichment process on the functionality of T cells is inconsistent between two donors (FIG. 39). The inventors further examined exhaustion-marker expression on CD19 CAR T cells in different test conditions in order to see whether incorporation of CD34 epitope leads to early exhaustion of T cells. Interestingly, CD19 CAR with CD34 epitope actually showed lower levels of PD-1, LAG-3 and Tim-3 expression (FIG. 40) compared to CD19 CAR without CD34 epitope. More experiments will be needed to understand how the incorporation of CD34 epitope, tag linker and CD8a hinge affect CD19 CART cell function. But the inventors decided to continue the Isoplexis experiment due to the lack of alternative method to purify transduced population.

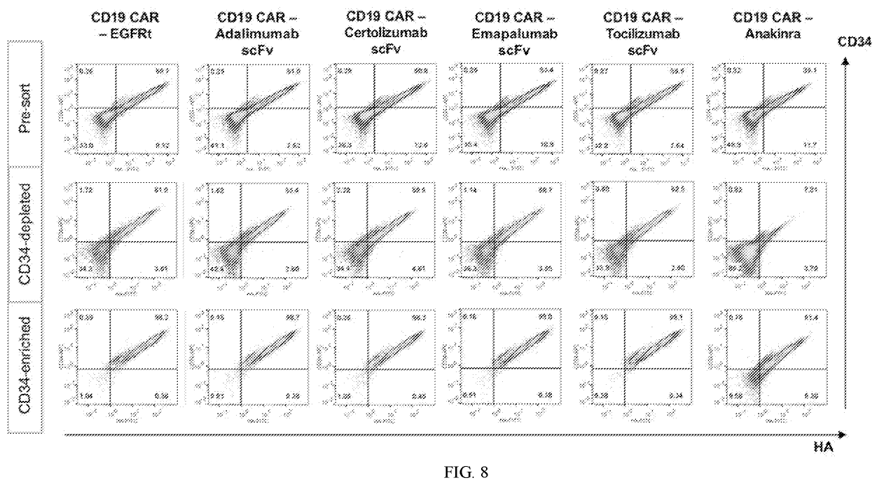

After identifying the proper method for CD34 epitope incorporation in to the CAR construct, a panel of self-modulating CD19 CARs with CD34 epitope and CD8a hinge was cloned. Primary human T cells transduced with the panel of CD34-epitope-CD19 CAR constructs showed efficient staining of both the N-terminal HA tag and the embedded CD34 epitope (FIG. 8). All the T cells were enriched by CD34 epitope expression and cryopreserved for Isoplexis experiment.

A co-culture of 5 cell types was set up to investigate the effect of cytokine antagonist secretion on T cells and nearby cells: (1) CD4⁺ CD19 CAR-T cells, (2) CD8⁺ CD19 CAR-T cells, (3) CD19⁺ K562 cells, (4) PBMC-derived CD4⁺ T cells and (5) PBMC-derived monocytes. Cells were incubated at a 1.5:0.5:2:1:1 ratio, respectively, for 21 hours prior to sample harvest for Isoplexis analysis. This multifaceted co-incubation environment was designed to recapitulate the complexity of CRS observed in vivo while maintaining the precise control afforded by in vitro assays. (See Methods sections for details of assay setup and sample preparation.)

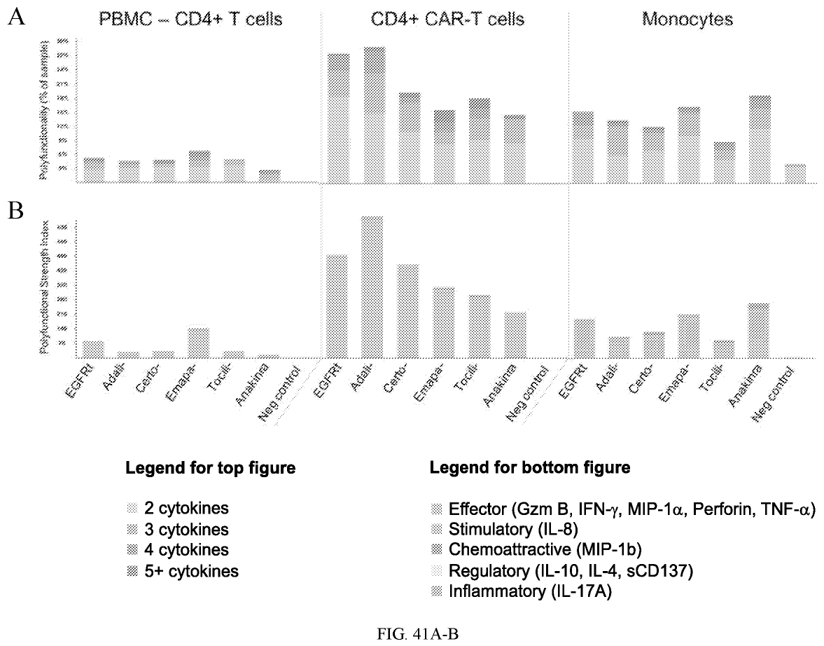

Results of Isoplexis analysis are shown as a measurement of polyfunctionality (i.e., percentage of cells that produce 2 or more cytokines) or polyfunctional strength index (PSI; PSI=(% polyfunctional cells in sample) Σ₋ (i=1)\hat{ }n [symbol] [symbol] MFI of secreted protein i of the polyfunctional cells [symbol] ). Using T cells expressing the CD19 CAR-T2A-EGFRt construct as reference, the inventors observed a reduction in both polyfunctionality and PSI in CD19 CAR-T cells that secreted certolizumab scFv, emapalumab scFv, tocilizumab scFv and anakinra (FIG. 41). Among PBMC-derived CD4⁺ T cells, a reduction in polyfunctionality and PSI was observed in samples that contained each of the cytokine antagonists tested except emapalumab scFv. Similarly, among monocytes, a reduction in polyfunctionality and PSI was observed in samples that contained each of the cytokine antagonists except emapalumab scFv and anakinra. These results suggest that scFvs derived from adalimumab, certolizumab, and tocilizumab were able to modulate cytokine production by not only engineered T cells but also nearby, unmodified T cells and monocytes. This ability to influence cytokine production “in trans” may be critical in the ability to modulate CRS, which is a systemic response resulting from the cytokine production of multiple cell types. It was interesting to note that emapalumab scFv was able to work in cis but not in trans. A potential explanation is that blocking IFN-γ, a critical cytokine for T-cell activation, from a certain population (CAR-T cells) may trigger a feedback loop that promote other populations to produce more cytokines to maintain a stimulating environment for T-cell function. On the contrary, adalimumab scFv was able to work in trans but not in cis. These different behavior in cytokine regulation demonstrated the different working mechanism of different cytokine antagonists. Exploring the working mechanism of different self-modulators could broaden our understanding on immune-regulation in CRS and help the development for novel CRS treatment. However, the exploration of working mechanism is not covered in this work. Based on the results obtained from Isoplexis analysis, the inventors further narrowed our panel of cytokine antagonists to certolizumab scFv, tocilizumab scFv, and anakinra in the follow-up in vivo experiment to verify their ability to mediate CRS (Example 3).

5. Self-Modulating CAR-T Cells Maintain Robust T-Cell Proliferation and Cytotoxicity Upon Antigen Stimulation

Although CRS is a potentially severe side effect of adoptive T-cell therapy, cytokine production constitutes an important arm of T-cell-mediated anti-tumor function. In proposing to modulate cytokine production by CAR-T cells, the inventors also need to ensure that the engineered cells retain essential anti-tumor effector functions. To this end, a repeated antigen challenge assay was performed to evaluate the impact of cytokine antagonist expression on the proliferation and cytotoxicity of T cells. Specifically, engineered CD19 CAR-T cells and CD19⁺ Raji target cells were co-incubated at a 1:1 effector-to-target (E:T) ratio at the start of the experiment (challenge 1 day 0, C1D0). Every 2 days, 10 μl of the co-culture sample was harvested and the number of T cells and Raji cells were quantified by flow cytometry. The remaining culture was re-challenged with fresh Raji target cells at a 1:1 E:T ratio, where the calculation is based on the assumption that the total number of T cells remained the same as on C1D0, minus the amount removed for cell counting.

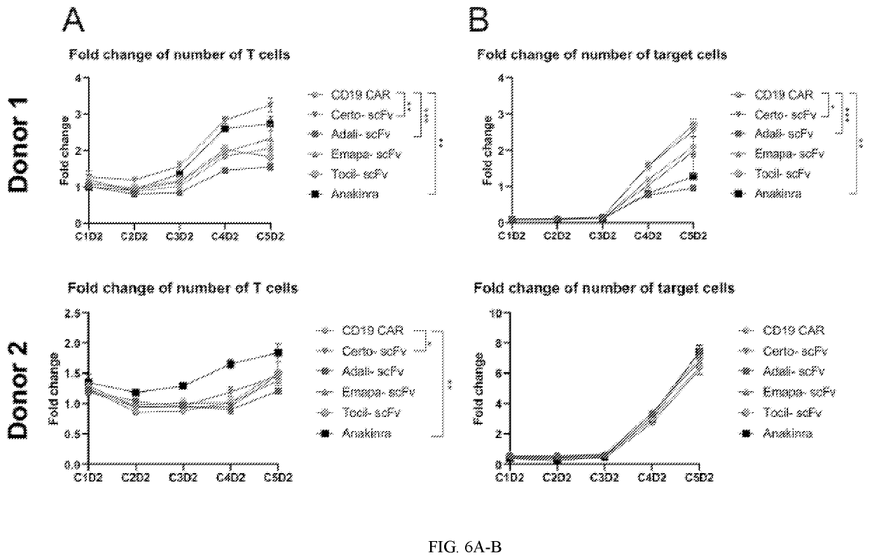

Results showed that all samples tested achieved efficient target-cell clearance and robust T-cell proliferation in the first three challenges, regardless of whether the CAR-T cells secreted cytokine antagonists (FIG. 6). Interestingly, CD19 CAR T cells expressing anakinra were able to reach a higher fold change in T-cell number compared to other conditions. Statistically significant difference in target-cell killing and T-cell proliferation when comparing self-modulating T cells with CD19-CAR T cells was observed in some donors but not others. Thus, the inventors conclude that the impact of different scFvs on T-cell proliferation and cytotoxicity may be donor-dependent. Since all the self-modulating CAR-T cells showed comparable behavior in the first three challenges, the effector function of CAR-T cells was not significantly affected by the expression of cytokine antagonists.

B. Discussion

CRS is a frequently observed toxicity in adoptive-T cell therapy and severe CRS cases have led to multiple patient deaths (2, 24-29). Although current CRS treatment agents such as corticosteroids and tocilizumab are effective for most patients, the lack of new treatment agent poses a risk to patients who are not responsive to either treatment. To improve the safety of adoptive-T cell therapy, a panel of cytokine antagonists was developed and evaluated in vitro for their potential as CRS treatment agents. The choice of cytokines to target was made based on the cytokine expression pattern of CRS patients and the availability of amino acid sequences of clinically approved proteins that target these cytokines. Instead of directly using monoclonal antibodies, cytokine antagonists were implemented in scFv format in order to minimize DNA footprint and maximize transfection and transduction efficiency. Because the ultimate goal of this study is to incorporate different cytokine antagonists into the previously developed “self-modulating T cells” system by co-expressing CAR and cytokine antagonists as “self-modulators” at the same time (Example 1), smaller plasmid size can potentially facilitate the manufacturing process.

In this study, a panel of self-modulators targeting IL-1, IL-6, IFN-γ and TNF-α signaling were generated from the sequences of clinically approved protein therapeutics. Interestingly, not all of scFvs tested demonstrated efficient secretion or blocking efficacy when expressed in scFv format in human cells. With some preliminary screening of the secretion and functionality of different cytokine antagonists (FIGS. 1-3, FIG. 38), five cytokine antagonists (adalimumab, certolizumab, emepalumab and tocilizumab scFvs, plus anakinra) were selected for incorporation into the self-modulating T-cells system. The inventors observed that each of the self-modulators could be co-expressed with CD19 CAR and the secretion of cytokine antagonists did not alter the ratio of CD4⁺ and CD8⁺ T cells in the culture.

When co-incubated with wildtype Raji cells, CD19 CAR T cells were able to clear target cells and proliferate regardless of the expression of different cytokine antagonists. The inventors hypothesized that T cells with the expression of cytokine antagonists maintain comparable effector function due to cytokine redundancy or compensatory feedback loops to stimulate T cells (38). It will be worthwhile to look at a comprehensive panel of cytokine expression to understand how the cytokine antagonist modulate cytokine expression while maintaining T-cell effector function. Based on results from repeated antigen challenge assays, it is possible that the effector function of T cells was regulated in a donor-dependent manner, which may be worth exploring in order to develop personalized CRS treatment or personalized immune response boost.

To obtain a purer population of CAR-T cells for Isoplexis assay, the CD34-epitope tag (22) was incorporated into the CD19 CAR construct. Cytokine antagonists-expressing CD19 CARs with CD34-epitope, long tag linker and CD8a hinge were enriched by cell sorting based on CD34 epitope staining. Although enriching cells by CD34 epitope should not stimulate T cells and cause premature exhaustion according to prior reposts (22), the inventors observed inferior T-cell proliferation and cytotoxicity in CD19 CAR-T cells with CD34 epitope in a repeated antigen challenge experiment. Based on the previous characterization, CD19 CAR has better effector function when the CD19 scFv is more proximal to the T-cell membrane (30), and different hinge sequences may also affect the functionality of CAR (31). It is possible that the additional length introduced by the CD34 epitope and tag linker, or the replacement of the IgG4 hinge with CD8a hinge, dampened the efficacy of CD19 CAR. Although the incorporation of CD34 epitope functions well in the context of a BCMA CAR (22), further optimization is needed to implement this strategy to CD19 CAR and other types of CAR.

Isoplexis results demonstrated that self-modulators can down-regulate the expression of inflammatory cytokines and lower the PSI in engineered T cells as well as nearby, unmodified T cells and monocytes. It is important to see self-modulator's effect on monocytes because it has been reported that monocytes are the major producer of multiple cytokines in CRS patients (23). It was also interesting to observe that emapalumab scFv appears to dampen cytokine production by CAR-T cells but promote cytokine production by non-CAR-T cells. On the contrary, adalimumab scFv appears to lower the PSI of non-CAR-T cells and monocytes but increase the PSI of CAR-T cells. Results from additional donors will be needed to confirm these results. By comparing results from different donors, the inventors would be able to see whether the different behavior from different cytokine antagonists were donor-dependent or universal across donors. If this cytokine expression pattern is consistent across donors, the cytokine expression pattern can be used to identify new therapies for CRS and/or autoimmune diseases caused by abnormal cytokine production. If the regulation of cytokine is indeed donor-dependent, evaluating the immune response of cytokine expression prior to adoptive T-cell therapy may serve as a guide to personalized CRS treatment.

In this study, the inventors demonstrated that the panel of five cytokine derived from adalimumab, certolizumab, emapalumab, tocilizumab and anakinra, can be co-expressed properly with CD19 CAR in primary human T cells. The expression of cytokine antagonists does not alter the effector function of T cells and is able to control the cytokine expression in CAR-T cells and surrounding cells such as non-CAR-T cells and monocytes. This work tested multiple cytokine antagonists as potential new treatments for CRS without dampening the effector function of T cells. Further work is needed to evaluate the efficacy of these self-modulators as CRS treatment in an in vivo setting.

C. Methods

1. Plasmid Construction

Light and heavy variable fragments of monoclonal antibodies connected via an 18-amoni acid linker and anakinra (Supplementary Table 3.S1) were synthesized as gBlocks (Integrated DNA) and cloned into the epHIV7 vector (32, 33), downstream of the EF1α promoter by standard molecular cloning methods. A second-generation CD19 CAR containing 4-1BB co-stimulatory domain was constructed as previously described (34), and bicistronic plasmids expressing an HA-tagged CD19 CAR fused to EGFRt, scFvs or anakinra via a T2A self-cleaving peptide were constructed in the epHIV7 backbone by isothermal DNA assembly. For the purpose of retrovirus production, all the CD19 CAR-T2A-EGFRt/scFvs/anakinra constructs were also transferred to the MSCV vector by standard molecular cloning methods. The MSCV vector was a generous gift from Dr. Steven Feldman (National Cancer Institute).

CD34 epitope with long tag linker and CD8a hinge was synthesized as gBlocks (Integrated DNA) and cloned into the CD19 CAR-T2A-EGFRt construct on MSCV vector by standard molecular cloning methods. Constructs with short tag linker were cloned afterwards by isothermal DNA assembly. The CD34 epitope-long tag linker-CD8a hinge sequence was cloned onto CD19 CAR-T2A-scFvs/anakinra constructs by standard cloning methods.

2. Cell Line Maintenance

Wild-type Raji cells were obtained from ATCC in 2003 and cultured in RPMI-1640 (Lonza) with 10% heat-inactivated FBS (HI-FBS; Life Technologies). Human embryonic kidney (HEK)293T cells were obtained from ATCC in 2011 and cultured in DMEM (HyClone) with 10% HI-FBS. Cell lines were routinely checked for Mycoplasma contamination using MycoAlert PLUS Mycoplasma Detection Kit (Lonza). NFκB EGFP reporter Jurkat cells were a generous gift from Dr. Xin Lin (35) (MD Anderson) and were cultured in RPMI-1640 with 10% HI-FBS. Cell lines received from ATCC were certified by ATCC and no further authentication was performed on these cell lines.

3. Primary Human T Cell Generation and Characterization

Primary human CD4⁺, CD8⁺ and pan T cells as well as peripheral blood mononuclear cells (PBMCs) were isolated from healthy donor blood obtained from UCLA Blood and Platelet Center using antibody cocktails (RosetteSep kits) followed by Ficoll-Paque (GE Healthcare Life Sciences) density-gradient separation. Isolated cells were stimulated with CD3/CD28 Dynabeads (Life Technologies) at a 1:3 bead:cell ratio. Two days after Dynabeads stimulation, cells were transduced with untitered retrovirus. Dynabeads were removed on day 7. All T cells were cultured in RPMI-1640 supplemented with 10% HI-FBS and fed with 50 U/ml IL-2 (Life Technologies) and 1 ng/ml IL-15 (Miltenyi Biotec) every two days.

Subtypes of T cells were distinguished by staining cells with CD4-PE (Biolegend; cat: 300508) and CD8-Viogreen (Miltenyi Biotec; cat: 130-113-164) and assessed by flow cytometry. CD19 CAR expression was distinguished by staining of HA tag antibody (Miltenyi Biotec; cat: 130-092-256) and assessed by flow cytometry.

Primary human T cells incorporated with CD34 epitope were enriched for CD34 expression by magnetic bead-based sorting (Miltenyi Biotec). Specifically, cells were stained with anti-CD34-APC (R&D systems; cat: FAB7227A; clone: QBEnd10) followed by staining with anti-APC microbeads (Miltenyi Biotec, cat: 130-090-855) before loading labeled cells onto magnetic column for enrichment.

Exhaustion-marker expression was quantified by surface antibody staining of PD-1-PE-vio770 (Miltenyi Blotec; cat: 130-099-878), LAG-3-APC (eBioscience; cat: 17-2239-42) and Tim-3-BV421 (Biolegend; cat: 345008).

4. Retrovirus Production

Retroviruses were produced by transfecting HEK293T cells seeded in 10 cm² dishes with retroviral packaging constructs and expression vector using polyethyleneimine (PEI) (Polysciences). Viral supernatant was harvested 48 and 72 h after transfection. Supernatant was filtered through 45 μm filter (VWR) to remove cell debris and either used for transduction directly or stored at −80° C.

5. scFv Production in HEK293T Cells

HEK293T cells were seeded in 10 cm² dishes and transiently transfected with plasmids encoding different scFvs using PEI. Cells were cultured in serum-free media for 24 h starting 24 h after transfection. Culture supernatant was harvested and concentrated using Amicon Centrifugal filter units (10,000 NMWL, EMD Millipore).

6. Western Blot

For HEK293T cell-secreted scFv western blots, scFv samples were prepared as described in the scFv production section. Membranes for scFv western blots were processed with SNAP i.d. protein detection system (EMD Millipore) following manufacturer's protocol. Sample-containing membrane was blocked with 0.5% non-fat milk in TBS-T buffer, then incubated in TBS-T buffer containing anti-FLAG antibody (Sigma Aldrich; cat: F1804-200UG) or anti-myc epitope tag antibody (Thermo Fisher Scientific; cat: MA1-21316).

For pSTAT3 western blot of untransduced cells, 1.5×10⁶ cells/ml/well of primary human CD4+ T cells were seeded in 24-well plates and incubated with indicated amounts of scFvs for 3 h. Indicated wells were subsequently treated with 4 ng/ml of human recombinant IL-6 (Biolegend) for another 30 minutes before cell harvest. Cell pellets were lysed in 1×RIPA buffer (1% Igepal CA-630, 0.1% SDS, 0.5% sodium deoxycholate) supplemented with protease and phosphatase inhibitor cocktail tablets (Fisher Scientific). Samples were run on Bolt 4-12% Bis-Tris Plus gels (Life Technologies) then transferred to nitrocellulous membranes (Life Technologies). Membranes were blocked with 5% BSA (Amresco) in TBS-T buffer (50 mM Tris, 150 mM NaCl, 0.05% Tween 20) for 1 h at room temperature, then incubated in TBS-T buffer containing pSTAT3 (Y705) antibody (Biolegend; cat: 651002) and 5% BSA overnight at 4° C. GAPDH (Sigma Aldrich; cat: G9292) was stained as loading control for all the samples. Anti-mouse secondary antibody conjugated to horseradish peroxidase (HRP; Jackson ImmunoResearch; cat: 115-035-062) was used for both western blots. Images of western blots were visualized using SuperSignal West Pico Chemiluminescent Substrate (Thermo Scientific).

7. Intracellular Staining to Confirm the Expression of Different scFvs and Anakinra

To verify the expression of different scFvs and anakinra in HEK293T cells, 5×10⁴ cells/S00 μl/well were seeded in 24-well plate and transfected with plasmid encoding anakinra using PEI. Cells were cultured for 8 h after transfection then supplemented with 5 μg/ml of Brefeldin A (Biolegend) and 2 μg/ml of monensin (Biolegend) for 20 h. Cells were fixed with 1.5% formaldehyde; permeabilized with ice-cold methanol; stained with antibody binding to FLAG (Miltenyi Biotec; cat: 130-101-571) or myc epitope tag (Miltenyi Biotec; cat: 130-099-616); and assessed by flow cytometry.

8. TNF-α scFv Testing with NFκB EGFP Reporter Jurkat Cell Line

NFκB EGFP reporter Jurkat cells were seeded at 3×10⁴ cells/200 μl/well in 96-well plate with the presence of designated concentration of TNF-α scFvs and incubated overnight a 37° C. 10 ng/ml of recombinant human TNF-α (Miltenyi Biotec; cat: 130-094-015) were added into designated wells after overnight incubation with TNF-α scFvs. Reporter induction was evaluated by measuring EGFP signal on flow cytometer after the 24 h incubation with human TNF-α at 37° C.

9. Repeated Antigen Challenge Assay

Primary human T cells express CD19-CAR-T2A-EGFRt/scFvs/anakinra constructs were seeded at 5×10⁵ cells/ml/well in a 24-well plate and co-incubated with WT Raji cells at 1:1 effector-to-target ratio. Seeding of primary human T cells was determined by the number of CARP cells (5×10⁵ CARP cells) and normalized the total cell number with T cells expressing EGFRt (no CD19 CAR) to make the number of total human T cells consistent among different conditions. Number of T cells and remaining target cells were evaluated by flow cytometry every two days. 5×10⁵ of fresh WT Raji cells were added to co-culture every two days after cell counting.

10. Isoplexis Experiment

This particular experiment was performed by personnel at Isoplexis due to the availability of IsoLight machine. Primary human T cells expressing CD19 CAR-CD34 epitope-EGFRt/scFvs/anakinra were enriched for CD34-epitope expression and froze down at day 12. Frozen T cells along with donor-matched PBMC were shipped overnight to Isoplexis site for experiment setup. Five cell types were included in this co-culture experiment: (1) CD4⁺ CD19 CAR-T cells, (2) CD8⁺ CD19 CAR-T cells, (3) CD19⁺ K562 cells, (4) PBMC-derived CD4⁺ T cells and (5) PBMC-derived monocytes. Cells were incubated at 1.5:0.5:2:1:1 ratio accordingly.

CD4⁺ and CD8⁺ CD19 CAR T cells were derived from CD34-enriched CD19 CAR-T cells by performing a CD8-enrichment using anti-CD8 beads to separate CD4⁺ and CD8⁺ populations. PBMC-derived monocytes were generated by collecting adherent cells from PBMC culture after a 24-h incubation in complete RPMI at 37° C. PBMC-derived CD4⁺ T cells were generated by enriching CD4⁺ cells in PBMC with CD4⁺ T cell isolation kit (Miltenyi Biotec; cat: 130-096-533). CAR-T cells were labeled with CFSE and PBMC-derived CD4+ T cells were labeled with BrilliantViolet dye in order to distinguish cells from different sources.

All the cells were co-incubated at 37° C., 5% CO2 for 21 hours. After incubation, suspension cells were enriched for CD4 expression for CD4⁺ CAR-T cells and PBMC-derived CD4⁺ cells analysis in one chip per condition. Adherent cells were harvested for monocyte analysis in separate chip per condition. Collected cells were loaded onto chips, and cytokine secretions were analyzed on the IsoLight machine.

### Statistical Analysis

Except for western blot, Isoplexis and qualitative analysis of scFvs/anakinra analysis on cells, a minimum of triplicates was chosen to allow calculation of statistics. Statistical significance was analyzed by using two-tailed, unpaired, homoscedastic Student's t-test.

D. Supplementary Tables

SUPPLEMENTARY TABLE 3.S1
Amino acid sequence of cytokine antagonists

| | |
|---|---|
| Linker sequence | GSTSGSGKPGSGEGSTKG |
| for scFv | (SEQ ID NO: 14) |
| Tocilizumab V_{L} | DIQMTQSPSSLSASVGDRVTITCRASQD |
| | ISSYLNWYQQKPGKAPKLLIYYTSRLHS |
| | GVPSRFSGSGSGTDFTFTISSLQPEDIA |
| | TYYCQQGNTLPYTFGQGTKVEIK (SEQ |
| | ID NO: 1) |
| Tocilizumab V_{H} | EVQLQESGPGLVRPSQTLSLTCTVSGYS |
| | ITSDHAWSWVRQPPGRGLEWIGYISYSG |
| | ITTYNPSLKSRVTMLRDTSKNQFSLRLS |
| | SVTAADTAVYYCARSLARTTAMDYWGQG |
| | SLVTVSS (SEQ ID NO: 2) |
| Infliximab V_{L} | DILLTQSPAILSVSPGERVSFSCRASQF |
| | VGSSIHWYQQRTNGSPRLLIKYASESMS |
| | GIPSRFSGSGSGTDFTLSINTVESEDIA |
| | DYYCQQSHSWPFTFGSGTNLEVK (SEQ |
| | ID NO: 3) |
| Infliximab V_{H} | EVKLEESGGGLVQPGGSMKLSCVASGFI |
| | FSNHWMNWVRQSPEKGLEWVAEIRSKSI |
| | NSATHYAESVKGRFTISRDDSKSAVYLQ |
| | MTDLRTEDTGVYYCSRNYYGSTYDYWGQ |
| | GTTLTVSS (SEQ ID NO: 4) |
| Adalimumab V_{L} | DIQMTQSPSSLSASVGDRVTITCRASQG |
| | IRNYLAWYQQKPGKAPKLLIYAASTLQS |
| | GVPSRFSGSGSGTDFTLTISSLQPEDVA |
| | TYYCQRYNRAPYTFGQGTKVEIK (SEQ |
| | ID NO: 5) |
| Adalimumab V_{H} | EVQLVESGGGLVQPGRSLRLSCAASGFT |
| | FDDYAMHWVRQAPGKGLEWVSAITWNSG |
| | HIDYADSVEGRFTISRDNAKNSLYLQMN |
| | SLRAEDTAVYYCAKVSYLSTASSLDYWG |
| | QGTLVTVSS (SEQ ID NO: 6) |
| Golimumab V_{L} | EIVLTQSPATLSLSPGERATLSCRASQS |
| | VYSYLAWYQQKPGQAPRLLIYDASNRAT |
| | GIPARFSGSGSGTDFTLTISSLEPEDFA |
| | VYYCQQRSNWPPFTFGPGTKVDIK |
| | (SEQ ID NO: 7) |
| Golimumab V_{H} | QVQLVESGGGVVQPGRSLRLSCAASGFI |
| | FSSYAMHWVRQAPGNGLEWVAFMSYDGS |
| | NKKYADSVKGRFTISRDNSKNTLYLQMN |
| | SLRAEDTAVYYCARDRGIAAGGNYYYYG |
| | MDVWGQGTTVTVSS |
| | (SEQ ID NO: 8) |
| Certolizumab V_{L} | DIQMTQSPSSLSASVGDRVTITCKASQN |
| | VGTNVAWYQQKPGKAPKALIYSASFLYS |
| | GVPYRFSGSGSGTDFTLTISSLQPEDFA |
| | TYYCQQYNIYPLTFGQGTKVEIK (SEQ |
| | ID NO: 9) |
| Certolizumab V_{H} | EVQLVESGGGLVQPGGSLRLSCAASGYV |
| | FTDYGMNWVRQAPGKGLEWMGWINTYIG |
| | EPIYADSVKGRFTFSLDTSKSTAYLQMN |
| | SLRAEDTAVYYCARGYRSYAMDYWGQGT |
| | LVTVSS (SEQ ID NO: 10) |
| Emapalumab V_{L} | NFMLTQPHSVSESPGKTVTISCTRSSGS |
| | IASNYVQWYQQRPGSSPTTVIYEDNQRP |
| | SGVPDRFSGSIDSSSNSASLTISGLKTE |
| | DEADYYCQSYDGSNRWMFGGGTKLTVL |
| | (SEQ ID NO: 11) |
| Emapalumab V_{H} | EVQLLESGGGLVQPGGSLRLSCAASGFT |
| | FSSYAMSWVRQAPGKGLEWVSAISGSGG |
| | STYYADSVKGRFTISRDNSKNTLYLQMN |
| | SLRAEDTAVYYCAKDGSSGWYVPHWFDP |
| | WGQGTLVTVSS (SEQ ID NO: 12) |
| Anakinra | MRPSGRKSSKMQAFRIWDVNQKTFYLRN |
| | NQLVAGYLQGPNVNLEEKIDVVPIEPHA |
| | LFLGIHGGKMCLSCVKSGDETRLQLEAV |
| | NITDLSENRKQDKRFAFIRSDSGPTTSF |
| | ESAACPGWFLCTAMEADQPVSLTNMPDE |
| | GVMVTKFYFQEDE (SEQ ID NO: 13) |

SUPPLEMENTARY TABLE 3.S2
Amino acid sequence of CD34 epitope and tag linkers

| | |
|---|---|
| CD34 epitope | ELPTQGTFSNVSTNVS (SEQ ID NO: 129) |
| Long tag linker | SGGGGS (SEQ ID NO: 130) |
| Short tag linker | SGGGGSGGGGS (SEQ ID NO: 131) |

E. Reference

The following references and the publications referred to throughout the specification, to the extent that they provide exemplary procedural or other details supplementary to those set forth herein, are specifically incorporated herein by reference.
1. Davila, M. L. et al. Efficacy and toxicity management of 19-28z CART cell therapy in B cell acute lymphoblastic leukemia. Sci. Transl. Med. 6, 1-13 (2014).
2. Maude, S. L. et al. Chimeric Antigen Receptor T Cells for Sustained Remissions in Leukemia. N. Engl. J. Med. 371, 1507-1517 (2014).
3. Locke, F. L. et al. Phase 1 Results of ZUMA-1: A Multicenter Study of KTE-C19 Anti-CD19 CAR T Cell Therapy in Refractory Aggressive Lymphoma. Mol. Ther. 25, 285-295 (2017).
4. Park, J. H. et al. Long-Term Follow-up of CD19 CAR Therapy in Acute Lymphoblastic Leukemia Jae. new engl J. Med. Orig. 378, 449-459 (2018).
5. Neelapu, S. S. et al. Axicabtagene Ciloleucel CAR T-Cell Therapy in Refractory Large B-Cell Lymphoma. N. Engl. J. Med. NEJMoa1707447 (2017). doi:10.1056/NEJMoa1707447
6. R. A., G. et al. Intent-to-treat leukemia remission by CD19 CAR T cells of defined formulation and dose in children and young adults. Blood 129, 3322-3331 (2017).
7. Lee, D. W. et al. Current concepts in the diagnosis and management of cytokine release syndrome. Blood 124, 188-196 (2014).
8. Brudno, J. N. & Kochenderfer, J. N. Toxicities of chimeric antigen receptor T cells: recognition and management. Blood 127, 3321-3331 (2016).
9. Porter, D. L. et al. Chimeric antigen receptor T cells persist and induce sustained remissions in relapsed refractory chronic lymphocytic leukemia. Sci. Transl. Med. 7, 1-12 (2015).
10. Lee, D. W. et al. T cells expressing CD19 chimeric antigen receptors for acute lymphoblastic leukaemia in children and young adults: A phase 1 dose-escalation trial. Lancet 385, 517-528 (2015).
11. Teachey, D. T. et al. Identification of predictive biomarkers for cytokine release syndrome after chimeric antigen receptor T-cell therapy for acute lymphoblastic leukemia. Cancer Discov. 6, 664-679 (2016).
12. Hay, K. A. et al. Kinetics and biomarkers of severe cytokine release syndrome after CD19 chimeric antigen receptor-modified T-cell therapy. Blood 130, 2295-2306 (2017).
13. Norelli, M. et al. Monocyte-derived IL-1 and IL-6 are differentially required for cytokine-release syndrome and neurotoxicity due to CAR T cells. (2018).
14. Giavridis, T. et al. CAR T cell-induced cytokine release syndrome is mediated by macrophages and abated by IL-1 blockade letter. Nat. Med. 24, 731-738 (2018).
15. Sterner, R. M. et al. GM-CSF inhibition reduces cytokine release syndrome and neuroinflammation but enhances CAR-T cell function in xenografts. Blood blood-2018-10-881722 (2018). doi:10.1182/blood-2018-10-881722
16. Lee, D. W. et al. Current concepts in the diagnosis and management of cytokine release syndrome. Blood 124, 188-195 (2014).
17. De Cesaris, P. et al. Tumor necrosis factor-α induces interleukin-6 production and integrin ligand expression by distinct transduction pathways. J. Biol. Chem. 273, 7566-7571 (1998).
18. Gust, J. et al. Endothelial Activation and Blood-Brain Barrier Disruption in Neurotoxicity after Adoptive Immunotherapy with CD19 CAR-T Cells. Cancer Discov. (2017). doi:10.1158/2159-8290.CD-17-0698
19. Santomasso, B. D. et al. Clinical and biological correlates of neurotoxicity associated with car t-cell therapy in patients with B-cell acute lymphoblastic leukemia. Cancer Discov. 8, 958-971 (2018).
20. Ma, C. et al. Multifunctional T-cell analyses to study response and progression in adoptive cell transfer immunotherapy. Cancer Discov. 3, 418-429 (2013).
21. Lu, Y. et al. Highly multiplexed profiling of single-cell effector functions reveals deep functional heterogeneity in response to pathogenic ligands. Proc. Natl. Acad. Sci. 112, E607-E615 (2015).
22. Valton, J. et al. A Versatile Safeguard for Chimeric Antigen Receptor T-Cell Immunotherapies. Sci. Rep. 8, 1-8 (2018).
23. Singh, N. et al. Monocyte lineage-derived IL-6 does not affect chimeric antigen receptor T-cell function. Cytotherapy 19, 867-880 (2017).
24. Brentjens, R., Yeh, R., Bernal, Y., Riviere, I. & Sadelain, M. Treatment of chronic lymphocytic leukemia with genetically targeted autologous T cells: case report of an unforeseen adverse event in a phase I clinical trial. Mol. Ther. 18, 666-668 (2010).
25. Kochenderfer, J. N. et al. B-cell depletion and remissions of malignancy along with cytokine-associated toxicity in a clinical trial of anti-CD19 chimeric-antigen-receptor-transduced T cells. Blood 119, 2709-2721 (2012).
26. Morgan, R. A. et al. Case report of a serious adverse event following the administration of T cells transduced with a chimeric antigen receptor recognizing ERBB2. Mol. Ther. 18, 843-851 (2010).
27. Grupp, S. A. et al. Chimeric antigen receptor-modified T cells for acute lymphoid leukemia. N. Engl. J. Med. 368, 1509-18 (2013).
28. Porter, D. L., Levine, B. L., Kalos, M., Bagg, A. & June, C. H. Chimeric Antigen Receptor-Modified T Cells in Chronic Lymphoid Leukemia. N. Engl. J. Med. 365, 725-733 (2011).
29. Kalos, M. et al. T Cells with Chimeric Antigen Receptors Have Potent Antitumor Effects and Can Establish Memory in Patients with Advanced Leukemia. Sci. Transl. Med. 73, 1-11 (2011).
30. Zah, E., Lin, M., Silva-benedict, A., Jensen, M. C. & Chen, Y. Y. T Cells Expressing CD19/CD20 Bispecific Chimeric Antigen Receptors Prevent Antigen Escape by Malignant B Cells. Cancer Immunol. Res. 4, 498-508 (2016).
31. Gianpietro Dotti, Stephen Gottschalk, Barbara Savoldo, and M. K. B. Design and Development of Therapies using Chimeric Antigen. Immunol Rev.; 257(1) 580, 6269-6274 (2014).
32. June, C. H. Adoptive T cell therapy for cancer in the clinic. J. Clin. Invest. 117, 1466-1476 (2007).
33. Brentjens, R. J. et al. Safety and persistence of adoptively transferred autologous CD19-targeted T cells in patients with relapsed or chemotherapy refractory B-cell leukemias. Blood 118, 4817-4828 (2011).
34. Kochenderfer, J. N. et al. Chemotherapy-refractory diffuse large B-cell lymphoma and indolent B-cell malignancies can be effectively treated with autologous T cells expressing an anti-CD19 chimeric antigen receptor. J. Clin. Oncol. 33, 540-549 (2015).
35. Wang, D. et al. A requirement for CARMAI in TCR-induced NF-kB activation. Nat. Immunol. 3, 830-835 (2002).
36. Yang, B. B. et al. Pharmacokinetics of anakinra in subjects with different levels of renal function. Clin. Pharmacol. Ther. 74, 85-94 (2003).
37. So, A. et al. The role of IL-1 in gout: from bench to bedside. Rheumatology, 57, i12-i19 (2018).
38. Ozaki, K. & Leonard, W. J. Cytokine and cytokine receptor pleiotropy and redundancy. J. Biol. Chem. 277, 29355-29358 (2002).

### Example 3—Developing In Vivo Model for Self-Modulating T Cells and CRS

Based on the reported cases in clinical trials, cytokine concentrations in CRS patients are highly variable and donor-dependent, such that no quantitative thresholds based on cytokine levels can be used to define CRS onset. In the absence of a quantitative definition of CRS occurrence that is translatable into an in vitro experiment setting, demonstration of the efficacy of CRS treatment in an in vivo setting is necessary. In this example, the inventors report the development of multiple in vivo models in an attempt to recapitulate human CRS in mice. The possibility of generating T cells from fetal tissues or cord blood was also evaluated. Lastly, the ability of different self-modulators to prevent CRS-associated adverse events was evaluated in a humanized NSG-SGM3 mouse model. This work evaluated the limitation and efficacy of different in vivo models and demonstrated the applicability of the self-modulating T cell system in an in vivo setting.

Due to the intrinsic difference between human and experimental animals, CRS and neurotoxicity were not observed in preclinical murine and non-human primate models for CAR-T cell therapy (7-12, 16). Instead, CRS induced by adoptive T-cell transfer was first reported as an unforeseen toxicity (1), with subsequent clinical reports providing accumulating knowledge of cell-therapy-induced CRS (1-3, 18-20). However, despite some progress in the identification of predictive biomarkers (2, 3) and the engineering of CAR-T cells with suicide switches (4-6), the development of in vivo models that can reliably recapitulate human CRS symptoms triggered by the adoptive transfer of CAR-T cells has not been fruitful until the past two years.

In 2014, A hu-SCID model which utilizes human peripheral blood mononuclear cells (PBMC)-humanized NOD/SCID/γ_{c}^{-/-} (NSG) mice was reported to demonstrate CRS caused by monoclonal antibodies such as OKT-3 or Campath-1H (7). Although this study showed up-regulation of CRS-associated human cytokines, it remains unclear whether this model could be used to recapitulate CRS triggered by adoptive T-cell transfer, as the mechanism of CRS caused by monoclonal antibody and adoptively transferred T cells can be very different. CRS involving murine cytokines has been observed in syngeneic models with immune-competent mice and murine CAR-T cells (8-10). However, recapitulation of CRS symptoms involving human cytokines has proven much more difficult. An in vivo model that uses human CAR-T cells to target endogenous antigens in SCID-beige mice was reported to exhibit some degree of CRS (11), but the setup of this model may not be applicable to every target because of limited expression of human antigens in mice and minimal cross-talk between human and murine antigens. It has also been shown that CRS may be observable by engrafting SCID-beige mice with high tumor burden followed by high-dose T-cell injection (12). However, due to the absence of some signature cytokines involved in human CRS, such as interleukin (IL)-6, it is uncertain whether this model can fully recapitulate human CRS.

Recently, it has been reported that the involvement of myeloid-lineage cells such as monocyte and macrophage are essential for the occurrence of CRS (12-16). In response, multiple in vivo models for CRS have been developed in NSG^{TgCMV-IL3, CSF2, KITLG}1Eav/MloySzJ (NSG-SGM3) mice, which transgenically express human IL-3, SCF, and GM-CSF to support myeloid cell engraftment (16, 17). A mouse model reported the occurrence of CRS in sub-lethally irradiated, high tumor burden-bearing NSG-SGM3 mice upon the treatment of human CD19 CAR-T cells (17). However, due to the absence of human myeloid cells, some signature cytokines for human CRS, such as IL-6, were still missing in this model. A more comprehensive in vivo model involved the humanization of NSG-SGM3 mice with CD34⁺ cells derived from human cord blood (16). Splenocytes were harvested from humanized mice to generate CAR-T cells, which were adoptively transferred into additional tumor-bearing, donor-matched humanized mice. Using this model, researchers reported the observation of multiple signature cytokines in human CRS as well as the occurrence of neurotoxicity in the humanized animals (16). However, this highly complex model requires a long preparation time, and is subject to limitations in the supply of human hematopoietic stem and progenitor cells (HSPCs) generated from cord blood samples.

Here, the inventors report the development of multiple murine models in an attempt to recapitulate human CRS and evaluate our self-modulating T-cell system in vivo. The inventors began with a PBMC-humanized NSG model, which showed up-regulated human inflammatory cytokines but lack of clinical symptoms and human IL-6 production. The inventors next evaluated the possibility of generating human T cells directly from fetal tissues or cord blood as an alternative to harvesting human T cells from humanized mouse spleen, but found that T cells generated from fetal tissues showed inferior proliferation and cord blood yielded limited HSPCs per sample. Lastly, the inventors explore the use of HSPC and human T cells from two different donors in a humanized NSG-SGM3 mouse model, and successfully used this model to demonstrate the efficacy of the self-modulating T-cell system. T-cell-secreted self-modulators were able to significantly lessen CRS-associated weight loss, clinical symptoms, and mortality in mice treated with adoptive T-cell therapy. The tumor-clearing efficacy of CAR-T cells was not hindered by the expression of self-modulators. Altogether, this example describes the development of multiple in vivo models in order to recapitulate human CRS caused by adoptively transferred T cells, demonstrating the feasibility and potential efficacy of the self-modulating T-cell system for use in adoptive T-cell therapy for cancer.

A. Results

1. PBMC-Humanized NSG Mice Experience Weight Loss Upon Adoptive T-Cell Transfer Due to Potential CRS

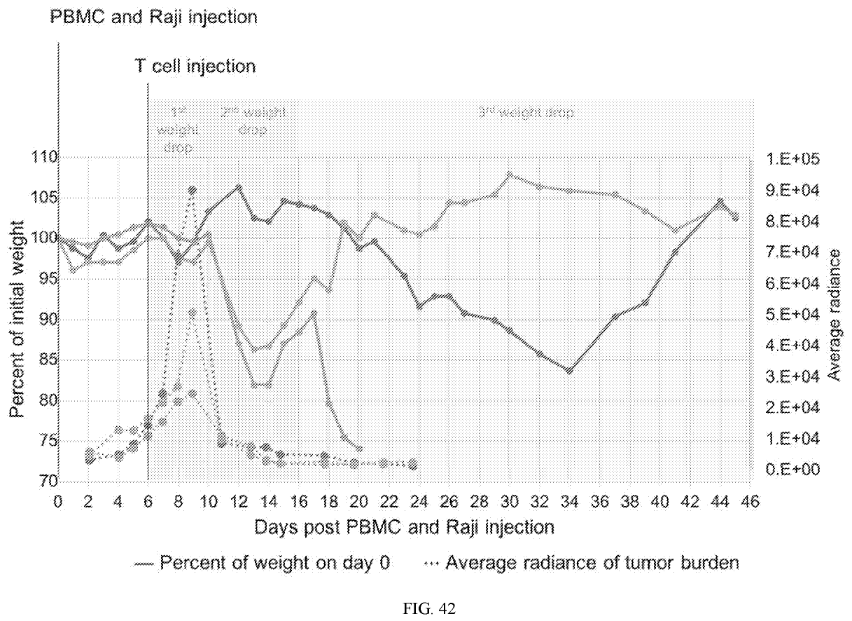

At the time of this study, there was no murine model that can fully recapitulate CRS with human T cells in immune-compromised mice. In addition, most of the existing models did not report the presence of human IL-6, which is a key cytokine for CRS and one of the targets for our self-modulator constructs. To develop a CRS murine model that can recapitulate human CRS in immune-deficient mice, the inventors devised an in vivo study adapted from a previously published model using monoclonal antibodies to induce CRS in PBMC-humanized NSG mice (7). NSG mice were injected with 2×10⁷ human peripheral blood mononuclear cells (PBMCs) and 5×10⁵ firefly luciferase-expressing CD19⁺ Raji lymphoma cells on day 0. Upon verification of tumor engraftment on day 6, mice were treated with 1×10⁷ donor-matched human CD4⁺ T cells transduced with CD19 CAR. The incorporation of PBMCs was expected to provide diverse cell types that better resemble the in vivo environment in humans. Animal weight was monitored daily, and three different periods of weight-drop were observed after T-cell injection (FIG. 42). The first weight drop coincided with the decreasing tumor signal, and the elimination of tumor cells and/or inflammatory response triggered through T-cell activation may have contributed to the slight weight loss observed. After tumor clearance, 2 out of 3 animals experienced a second weight loss of varying severity between day 10 and day 16, followed by a final weight drop after day 16. Due to the presence of high-dose human PBMC, the final weight drop is likely caused by graft-versus-host disease (GvHD). The inventors hypothesize the cause of second weight loss is CRS caused by expanded T-cell population after tumor clearance. However, cytokine measurement and cellular information in the blood sample would be needed to confirm this hypothesis. Unfortunately, blood sample was not analyzed in this animal study due to the absence of CRS-associated symptoms.

2. Modified PBMC-Humanized NSG Mice Model Exhibited Up-Regulation of Human IFN-γ and GM-CSF but No Human IL-6

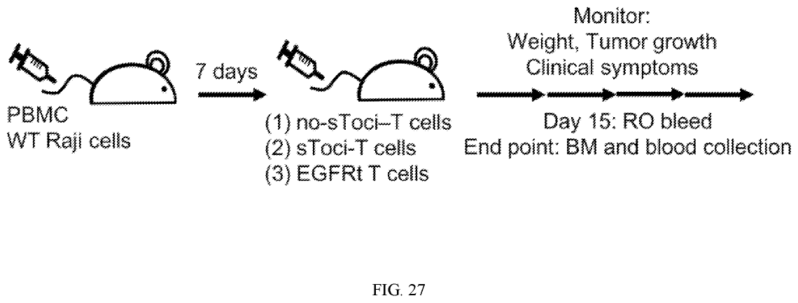

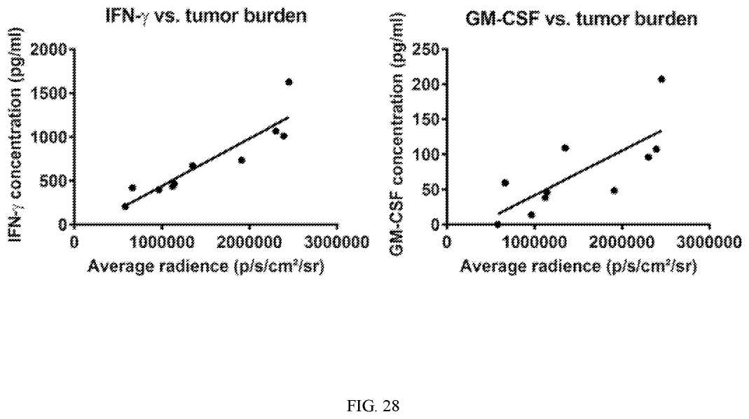

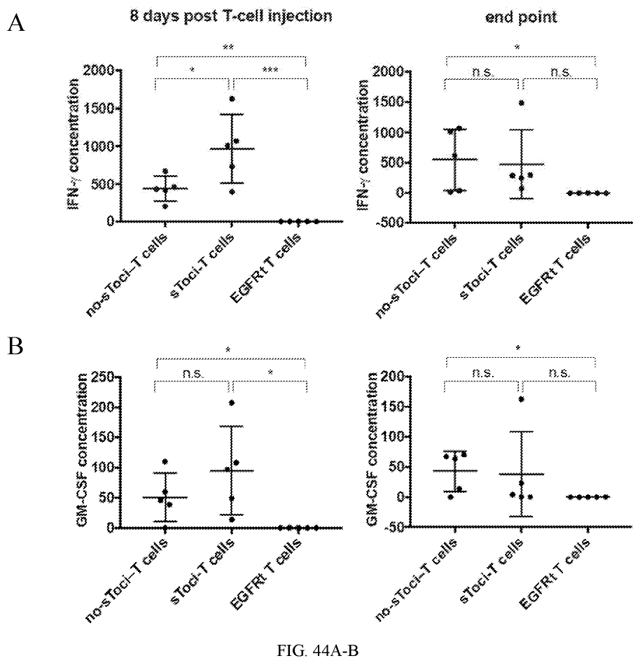

The relatively rapid onset of GvHD in the mouse model described above resulted in a very narrow window during which CRS may be observed. To test the efficacy of self-modulating T cells in PBMC-humanized NSG mouse model while delaying the occurrence of GvHD, the inventors tested NSG mice humanized with a reduced dose of PBMCs (2×10⁶ cells) and subsequently treated with a panel of T cells transduced with (1) CD19 CAR, (2) CD19 CAR with sToci expression and (3) truncated epidermal growth factor receptor (EGFRt) as the negative control. NSG mice were engrafted with 2×10⁶ human PBMC and 5×10⁵ CD19⁺, ffluc-expressing Raji lymphoma cells 7 days before treatment with 2×10⁶ donor-matched CD4⁺ T cells (FIG. 27). When analyzing the plasma content of blood samples collected 8-days post-T-cell injection, the expression of human IFN-γ and GM-CSF were detected. Consistent with clinical observation in human patients (18-20), there was a strong correlation between cytokine concentration and tumor burden (FIG. 28). CD19 CAR-T cells, regardless of sToci expression, showed up-regulated human IFN-γ and GM-CSF expression compared to T cells expressing EGFRt only, indicating cytokine production was triggered by antigen-specific T-cell responses (FIG. 44). However, the expression of sToci was unable to control the cytokine expression in PBMC-humanized NSG mice. Surprisingly, an increase in IFN-γ was observed in sToci-T cells relative to no-sToci-T cells 8-days post-T-cell infusion (FIG. 44A). However, this difference had disappeared by the time of animal sacrifice, at 28-days post-T-cell injection. Previous studies have shown that treatment with tocilizumab can lead to a short-term increase in IL-6, likely due to compensatory effects triggered by feedback loops in IL-6 signaling and production (21). Similar behavior may have resulted in the elevated IFN-γ at the earlier time point, but further investigation would be needed to elucidate this phenomenon fully.

In contrast to IFN-γ and GM-CSF, no human IL-2, IL-4, IL-6, IL-8, IL-10, or TNF-α was present at detectable levels in any of the animal groups. The absence of IL-6 production precludes proper evaluation of the anti-IL-6Rα sToci. Several studies that were published after this in vivo study had been completed shed light on the mechanism of CRS and underscored the importance of myeloid cells in CRS (14, 16, 22). In particular, a publication by Morelli et al. reported the up-regulation of human IL-6 with a humanized NSG-SGM3 mouse model (16), which offer a potential means to test our self-modulator that blocks human IL-6 signaling.

3. Fetal Liver, Spleen or Cord Blood Generate Limited Number of T Cells

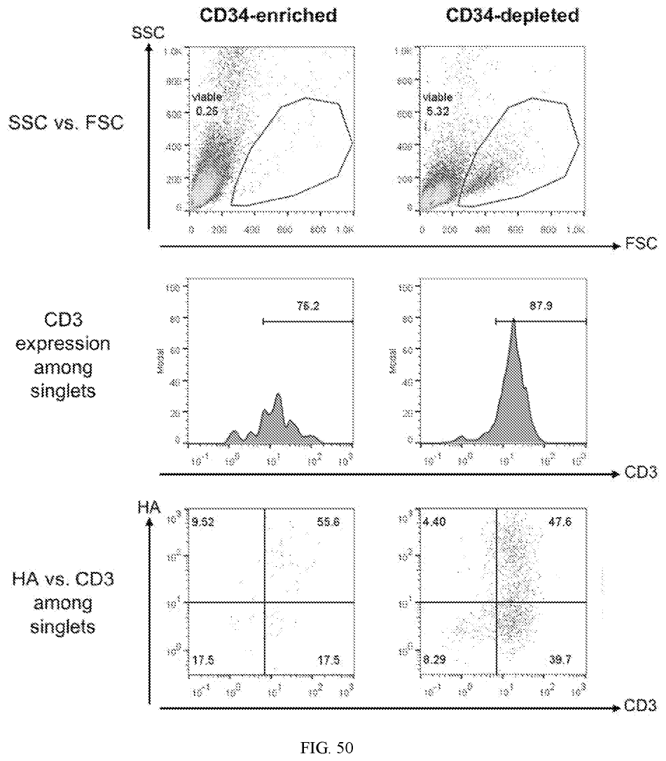

Inspired by the in vivo model generated by Norelli et al. (16), the inventors sought to develop a humanized NSG-SGM3 mouse model that uses hematopoietic stem and progenitor cells (HSPCs) and T cells from the same donor in order to avoid the occurrence of GvHD that may interfere with the observation of CRS. Because of the availability of tissue samples, the inventors first evaluated the possibility of using human fetal liver as the source of both HSPCs and T cells. To obtain CD34⁺ HSPCs for humanization, mononuclear cells isolated from human fetal liver tissue were enriched for CD34 expression. CD3 expression in both CD34-enriched and CD34-depleted cells were evaluated. Although the CD34-enriched population had a higher percentage of CD3⁺ cells, the intensity of CD3 expression level was substantially lower compared to primary human T cells in culture (FIG. 49). Both populations were stimulated with CD3/CD28 dynabeads and transduced with retrovirus encoding the expression of a HA-tagged CD19 CAR construct. Transduction efficiency and CD3 expression were evaluated 8-days after stimulation, upon dynabeads removal. Surprisingly, despite the initially low CD3 expression, CD34-depleted cells showed a distinct population of cells that were CD3 positive and had about 47% transduction efficiency. However, T cells generated from CD34-depleted cells had low viability (about 5%) and could not expand to a number that was necessary for animal experiments. On the other hand, no viable cells were observed in CD34-enriched cells after 8-days stimulation of dynabeads (FIG. 50).

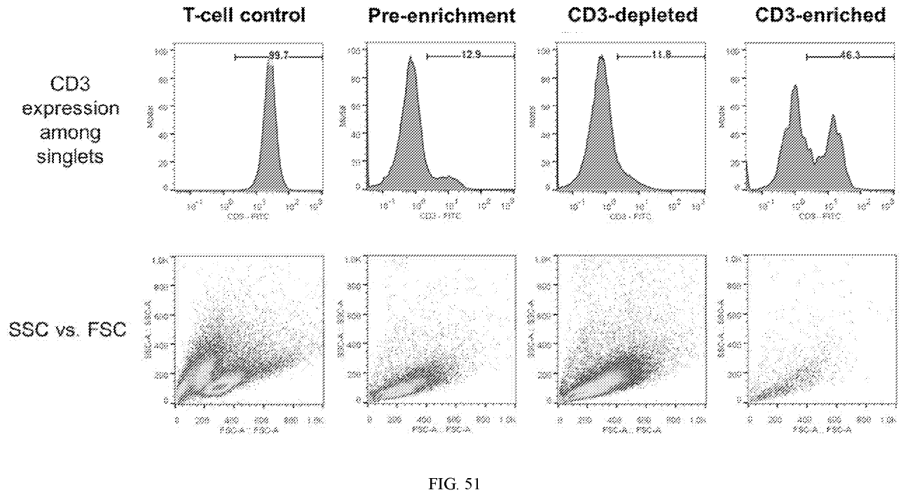

The inventors speculate that the low percentage of T cells or CD3⁺ cells upon stimulation may be the cause of low viability of T cells on day 8. To increase the purity of T cells from the starting population and to potentially improve the fitness of T cell culture, human CD3⁺ cells were enriched from CD34-depleted cells by using bead-based magnetic enrichment before activation with dynabeads. After CD3 enrichment, the percentage of CD3⁺ cells was significantly increased (from 12% to about 46%). However, cells appeared damaged from the enrichment process and led to indistinct populations of viable cells and the inability to expand (FIG. 51).

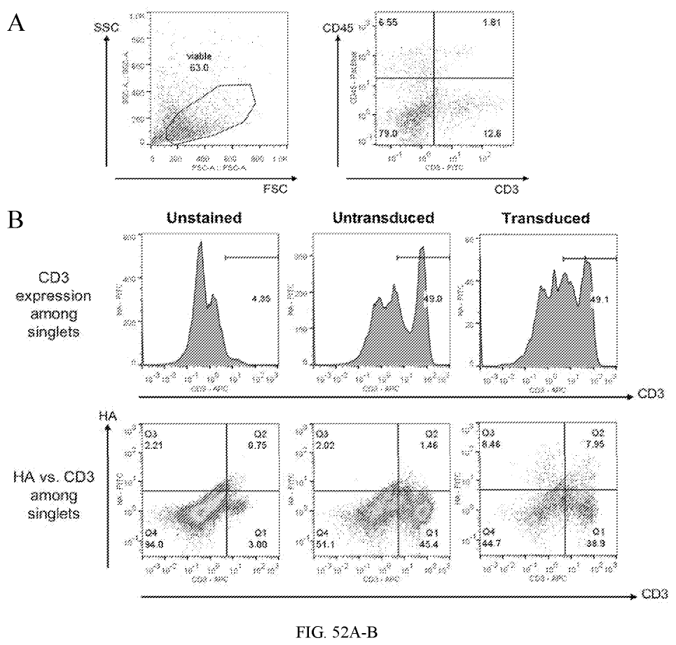

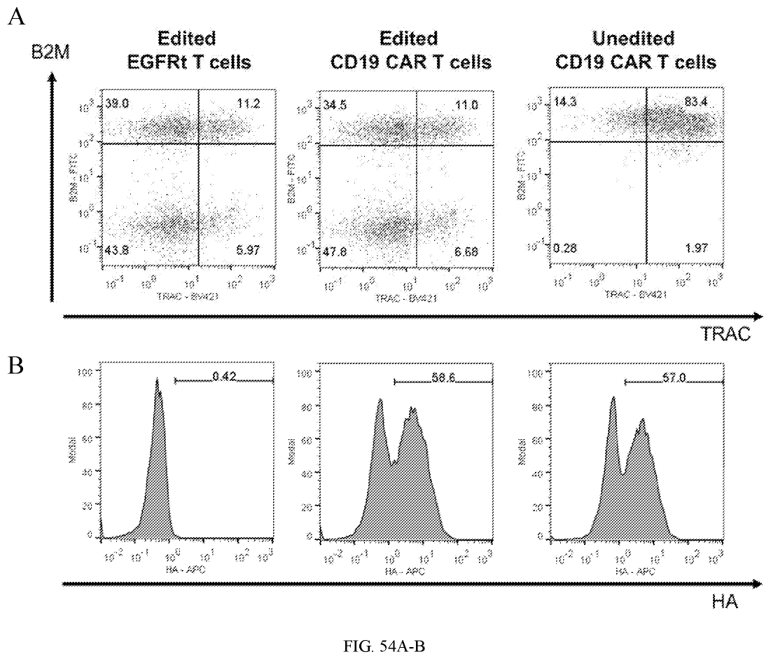

Because of the difficulty experienced in generating T cells from human fetal liver tissue, the inventors next tested the possibility of generating donor-matched HSPCs and T cells from fetal liver and spleen, respectively. Mononuclear cells were generated from donor-matched fetal liver and spleen tissues, with the intention of isolating CD34⁺ HSPCs from fetal liver for mouse humanization and enriching CD3⁺ cells from fetal spleen for T-cell culture. On the day of isolation, most of the cells were viable and had mixed expressions of CD3 and CD45 (FIG. 52A). T cells isolated from fetal spleen were stimulated with CD3/CD28 dynabeads and transduced with retrovirus, and their CD3 expression level and transduction efficiency were re-evaluated upon dynabeads removal 7 days after bead addition. Although a distinct CD3⁺ population was present, there were mixed populations of cells in the culture, and the transduction efficiency was very low (FIG. 54B). The impure T-cell culture eventually led to limited expansion of T cells, which can potentially be solved by additional CD3-enrichment step. However, the number of cells that could be isolated from one fetal spleen sample was too low for fetal spleen to serve as a practical source for T-cell generation (Table 4.1).

TABLE 4.1
Number of cells that can be isolated from tissue samples.

| Cell types | Fetal Liver | Fetal Spleen | Cord blood |
|---|---|---|---|
| CD34⁺ cells | 2 × 10⁷ | N/A | 4.5 × 10⁶ |
| CD34⁻ cells | 1.6 × 10⁸ | 3 × 10⁶ | 1 × 10⁸ |

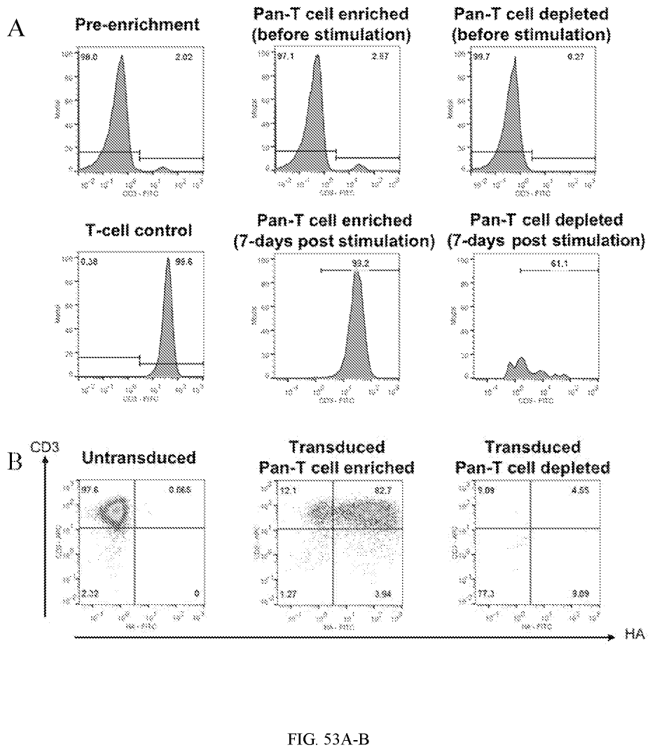

Lastly, the inventors evaluated the possibility of using cord blood as the source for both HSPCs and T cells. Mononuclear cells were isolated from cord blood, followed by CD34-enrichment to obtain HSPCs. CD34-depleted cells were enriched for T cells by bead-based pan-T cell isolation. To our surprise, the CD3 staining performed immediately after T-cell isolation showed very low CD3 expression in all populations (FIG. 53A). Given the uncertain enrichment efficiency, both pan-T cell-enriched and pan-T cell-depleted populations were stimulated with dynabeads and transduced with retrovirus encoding HA-tagged CD19 CAR. As anticipated, the pan-T cell-enriched, but not the pan-T cell-depleted, population expanded after stimulation with anti-CD3/CD28 dynabeads. The purity of T-cell cultures was evaluated again by CD3 staining 7 days later, upon dynabeads removal. At that time, pan-T cell-enriched cells were all CD3⁺ (FIG. 53A). Taken together, these results suggest that pan-T cell enrichment was effective in selecting CD3⁺ cells, and the lack of CD3 staining observed immediately after cell sorting may have been due to the presence of debris or cell types that could not be removed from the cell-isolation process. Another possibility for the suboptimal CD3-enrichment result may be an operator error that resulted in an insufficient amount of enrichment reagent being added to the cell sample during the isolation process. Transduction efficiency was also quantified upon dynabeads removal, and pan-T cell-enriched cells were successfully transduced, with 82.7% of cells being double positive for CD3 and HA-tag staining (FIG. 53B).

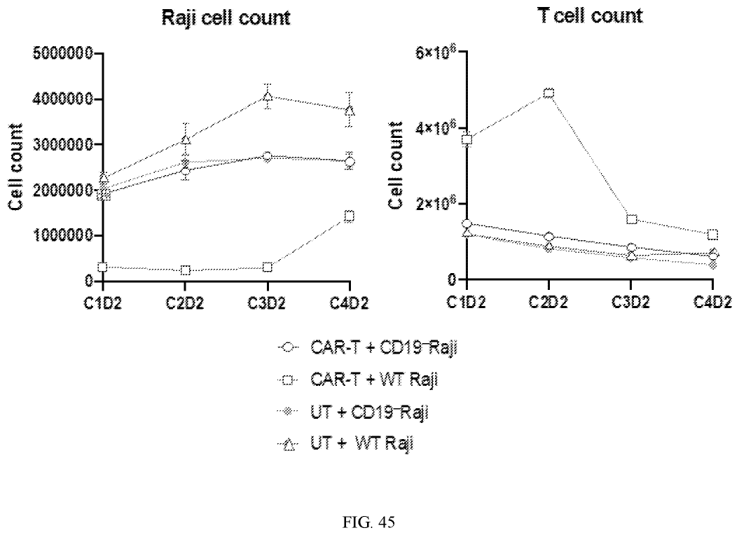

Transduced CAR-T cells were able to expand and proliferate, and their functionality was evaluated in a repeated antigen challenge assay. CAR-T cells and target cells were cultured in 1:1 effector-to-target ratio, supplemented with target cells every two days to test the T cells' functional persistence. Untransduced T cells generated from cord blood did not show unspecific toxicity and were unable to proliferate or kill Raji cells in culture (FIG. 45). In contrast, CD19 CAR-T cells were able to clear target cells and proliferate for up to three challenges. Moreover, no off-target toxicity was observed when CD19 CAR-T cells were co-incubated with CD19⁻ Raji cells (FIG. 45).

Although T cells generated from cord blood showed desired proliferation and T-cell effector function, relatively few HSPCs could be obtained from each cord blood unit, thus limiting the number of animals one could humanize with HSPCs obtained from one cord blood unit (Table 4.1). Also, due to the limited supply and high demand, the inventors could only obtain about one cord blood unit per month on average, which would significantly delay the experimental timeline.

4. TRAC and B2M Edited Cells Delay the Occurrence of GvHD in a Humanized NSG-SGM3 Mouse Model Using Allogeneic HSPCs and T Cells

To move the in vivo evaluation of self-modulating T cells forward, the possibility of using primary human T cells from healthy adult donors in humanized mice receiving HSPCs from fetal liver was evaluated. In this model, HSPCs and T cells originated from different donors. As such, the recipient mice were expected to experience toxicities caused by not only graft-versus-host disease (GvHD) but also graft-versus-graft reactions. The occurrence of multiple immune reactions could obscure the symptoms of CRS and complicate the model. In an attempt to minimize the chance of graft-versus-graft reactions, TRAC and B2M loci in the engineered T cells were knocked out by CRISPR/Cas9 gene editing. Editing at the TRAC locus prevents expression of endogenous T-cell receptor (TCR), thus lowering engineered T cells' ability to attack HSPCs; editing at the B2M locus abolishes proper formation of the major histocompatibility complex MHC class I (MHC-I) molecule, thus preventing the recognition of engineered T cells by HSPC-derived T cells.

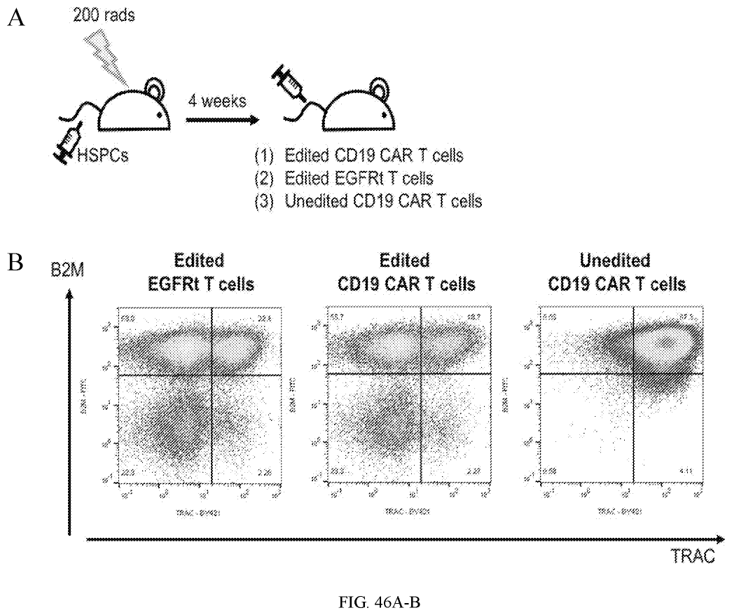

NSG-SGM3, a transgenic mouse strain that expresses human SCF, GM-CSF and IL-3 in the NSG genetic background, was used in this in vivo model to achieve improved engraftment of myeloid-lineage cells, which have been shown to be major producers of cytokines implicated in CRS (14, 23, 24). 6- to 12-weeks-old NSG-SGM3 mice were sub-lethally irradiated prior to humanization with fetal-liver-derived HSPCs delivered via tail-vein injection. Four weeks later, mice confirmed to contain >20% human cells in their peripheral blood were considered humanized and injected with 6×10⁶ of engineered CAR-T cells derived from a healthy adult donor (FIG. 46A).

The engineered T cells were transduced on day 2 and day 3 after dynabeads stimulation and electroporated with ribonucleoprotein (RNP) complexes to knock out TRAC and B2M expression. Engineered cells showed ˜40% TRAC⁻ B2M⁻ population with >50% CAR⁺ cells (FIG. 54). Three T-cell conditions were included in this experiment: (1) TRAC/B2M-knockout (i.e., “edited”) CD19 CAR T cells, (2) edited EGFRt T cells, and (3) unedited CD19 CART cells. Unedited or single-knockout cells were used to normalized edited cells to make sure CD19 CAR and EGFRt conditions have similar compositions of TRAC and B2M expressions (FIG. 46B). The inventors expected unedited CD19 CAR condition to demonstrate the most severe immune reaction reflecting a combination of CRS, graft-versus-graft, and GvHD; followed by edited CD19 CAR condition which would be susceptible to CRS and GvHD; and edited EGFRt condition should show the least severe immune reaction associated with GvHD only.

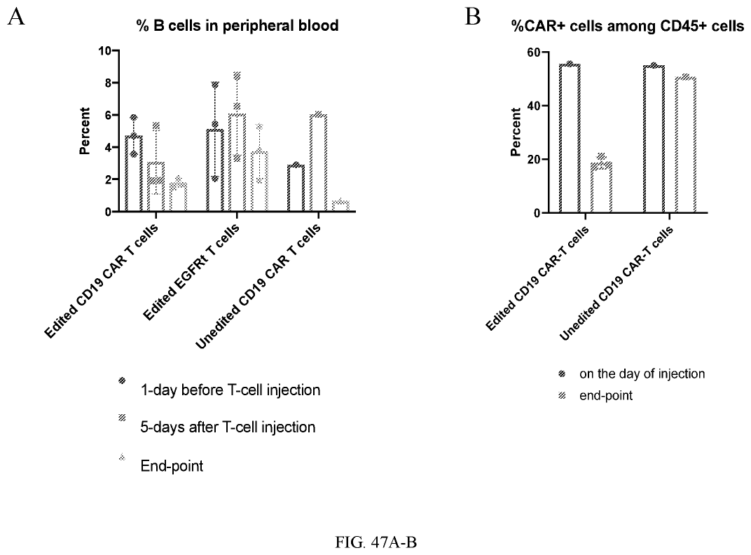

Both edited and unedited CD19 CAR-T cells were able to decrease the percentage of B cells in peripheral blood, indicating the functionality of CD19 CAR in targeting CD19⁺ B cells (FIG. 47A). However, there was a distinct difference in the persistence of edited and unedited CD19 CAR-T cells. Both groups started with ˜55% CAR⁺ cells at the time of T-cell injection, but the % CAR⁺ cells among edited CD19 CAR-T cells dropped below 20% at the endpoint whereas unedited CD19 CAR T cells maintained 50% CAR⁺ cells at the endpoint (FIG. 47B). These results suggest that CAR⁺ cells lacking TCR and/or MHC-I expression may have inferior persistence in vivo, which could significantly reduce their ability to induce the robust immune response that is required to trigger CRS. In fact, the inventors did not observe any symptoms suggesting CRS in any of the animals treated in this study. However, the animal treated with unedited T cells began to show weight loss 7 days after T-cell injection, consistent with expectations of GvHD onset. The observation that symptoms consistent with GvHD did not arise until 7 days post T-cell injection suggests that there may be a viable time window before GvHD onset in which to observe CRS, which is expected to occur within 1-4 days of T-cell injection if it occurred at all. The inventors hypothesized that, in addition to humanization with HSPCs, the engraftment of CD19⁺ tumor cells prior to CD19 CAR-T cell injection may further increase the probability of triggering a sufficiently intense immune response to result in CRS.

5. Modified Humanized NSG-SGM3 Mouse Model Showed Toxicities Consistent with CRS that could be Modulated by Cytokine Antagonists Secreted by CAR-T

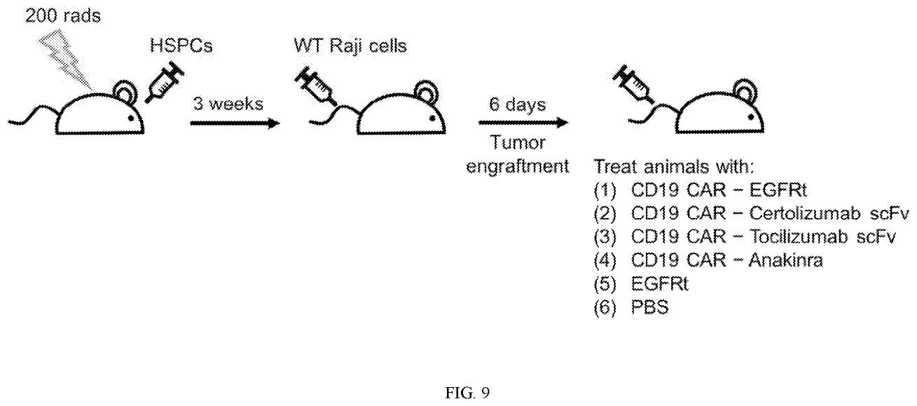

Based on the observations from the previous humanized NSG-SGM3 mouse model, a modified model was developed (FIG. 9). Six- to 8-weeks-old NSG-SGM3 mice were sub-lethally irradiated and injected with 3×10⁵ CD34⁺ HSPCs generated from fetal liver tissue via retro-orbital injection. Three weeks after HSPCs injection, all animals were confirmed to be humanized (i.e., containing more than 20% human cells in peripheral blood). Each animal was given 4.5×10⁶ of EGFP, firefly luciferase (ffluc)-expressing Raji lymphoma cells via tail-vein injection 24 days after HSPC injection. The dose of Raji cells in this study was 9 times higher than

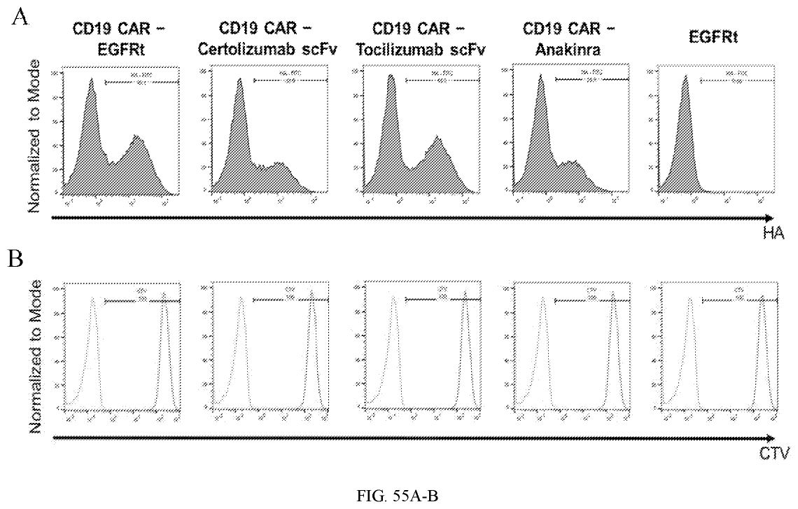

the usual dose used in the laboratory in order to achieve a high tumor burden and overcome the graft-versus-tumor effect that may be exerted by the engrafted HSPCs. Tumor engraftment was monitored by bioluminescence imaging and high tumor burden was observed by day 6. Animals were subsequently treated with T cells encoding CD19 CAR with or without different self-modulators on day 6. Adoptively transferred T cells in this experiment were generated from naïve/memory T cells (CD14⁻, CD25⁻ and CD62L-enriched primary human T cells), which had been shown to have superior T-cell functionality compared to CD3⁺ T cells (unpublished data from previous Chen lab member). Naïve/memory T cells were stimulated with CD3/CD28 dynabeads and transduced with retrovirus encoding: (1) CD19 CAR-EGFRt, (2) CD19 CAR-certolizumab scFv, (3) CD19 CAR-tocilizumab scFv, (4) CD19 CAR-anakinra, or (5) EGFRt. All T-cell lines had similar transduction efficiencies and CAR expression levels on the day of T-cell injection (FIG. 55A). Transduced T cells were labeled with CellTrace Violet (CTV) before T-cell injection to distinguish them from T cells derived from HSPCs that had been used to humanize the murine hosts. Uniform CTV expression levels from all T cell lines were confirmed on the day of T-cell injection (FIG. 55B).

In this mouse model, the inventors expected PBS-treated animals to eventually deteriorate due to tumor progression, whereas animals treated with T cells expressing EGFRt only (i.e., no CAR expression) would be subject to both tumor progression and toxicities due to GvHD. Finally, the inventors expected animals treated with CD19 CAR-T cells to experience acute CRS and, at a slightly later time point, GvHD. To capture rapid changes that are expected to occur in animal health upon T-cell injection, animals were weighed and visually inspected for clinical symptoms (e.g., ruffled hair, hunched posture, low activity, etc.) and general health at least once per day. A clinical score was given to each animal based on a 0-to-5 scale, with 5 being a healthy animal and 0 being an animal that has reached the humane endpoint.

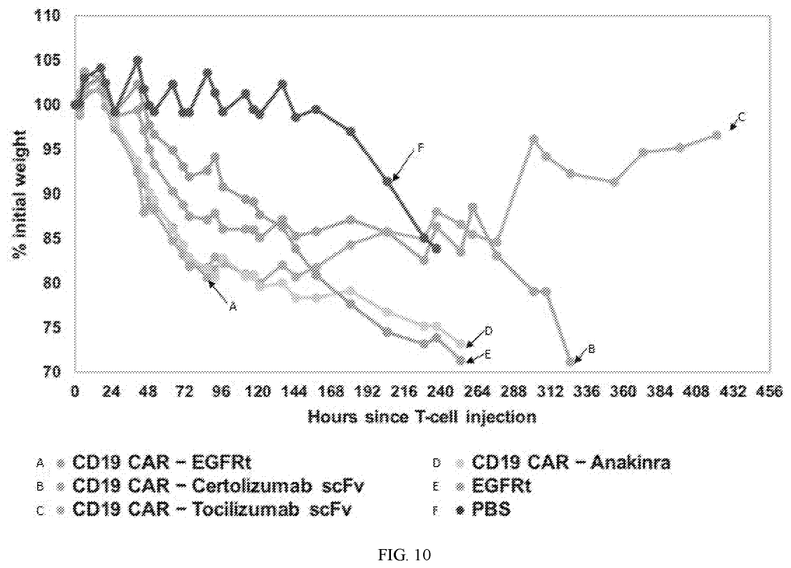

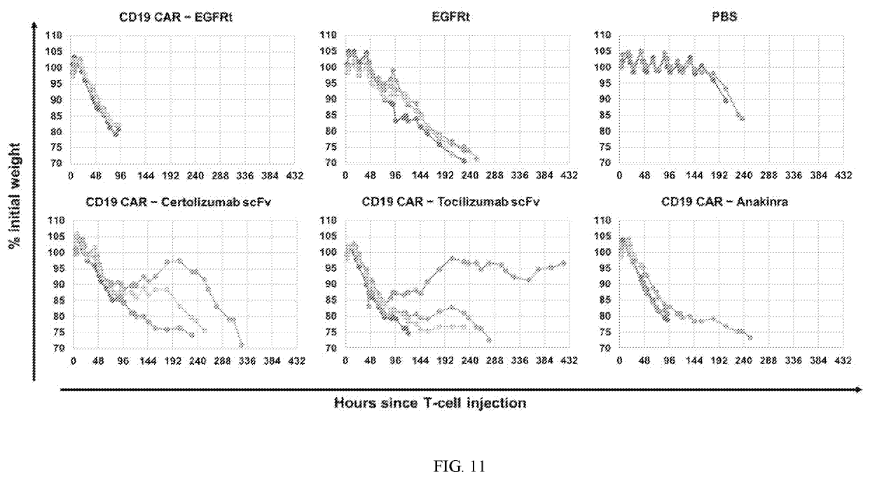

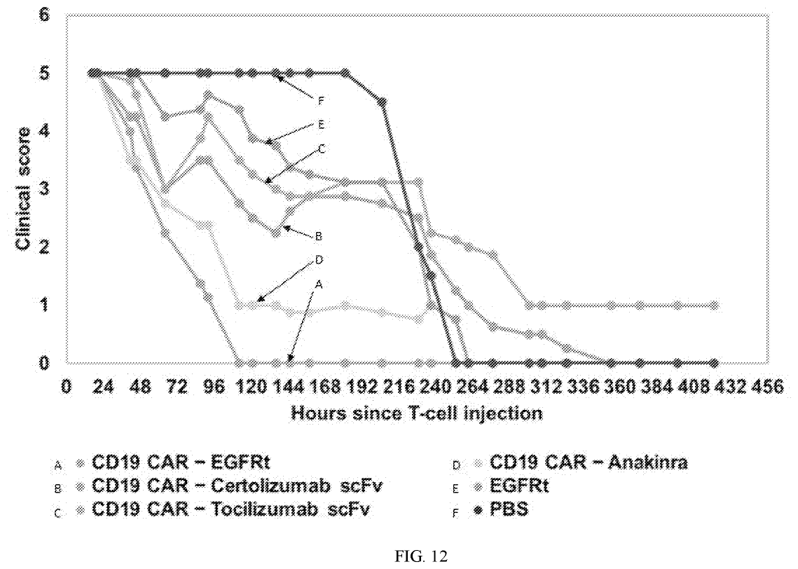

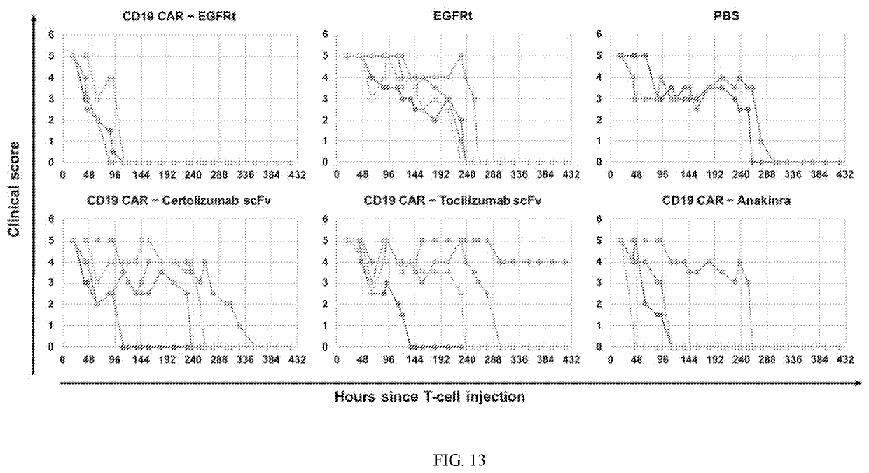

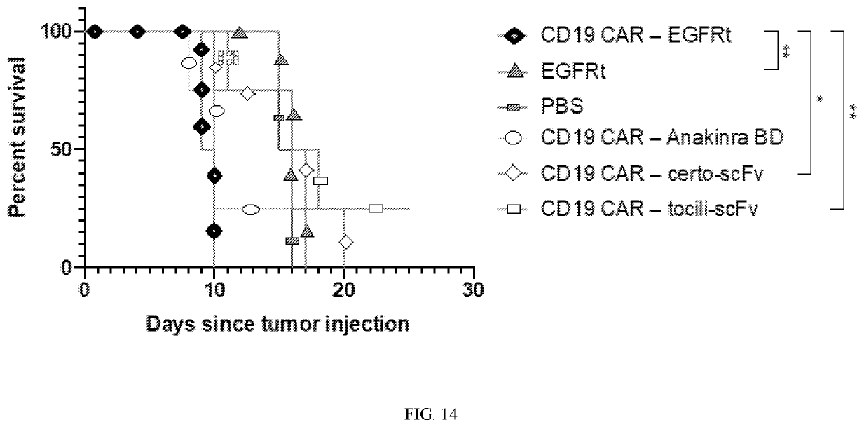

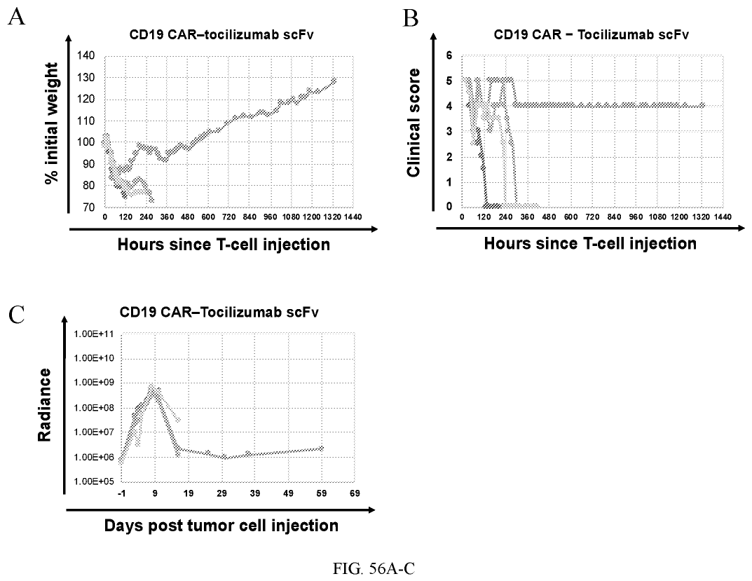

All animals treated with T cells expressing CD19 CAR-EGFRt began exhibiting steep weight loss within 24 hours of T-cell injection, and reached the humane end point within 4 days, consistent with rapid onset of toxicities associated with CRS (FIGS. 10-14 and 56). In comparison, animals treated with EGFRt-expressing T cells did not begin to experience significant weight loss until 48 hours post T-cell injection, whereas animals treated with PBS (i.e., no T cells) showed no significant weight change until 7 days post T-cell injection (FIGS. 10-11). All animals in the EGFRt-T cell and PBS-treated groups reached the humane end point approximately 10 days post T-cell injection, in the presence of high tumor burden (FIG. 14).

Animals treated with T cells that expressed the CD19 CAR together with tocilizumab scFv or anakinra also showed rapid weight loss similar to the animals treated with CD19 CAR-EGFRt T cells (FIGS. 10-11). However, 1 out of 4 animals in the anakinra group survived beyond 10 days post T-cell injection (FIG. 14). Furthermore, 3 out of 4 animals with CD19 CAR-tocilizumab scFv were able to recover 5-days post-T-cell injection, leading to substantially prolonged survival compared to the CD19 CAR-EGFRt group. In fact, 1 out of 4 animals in the tocilizumab group remained tumor free and healthy at the time of this writing, 2 months after T-cell injection (FIGS. 11, 13, 14, and 56).

Animals receiving T cells expressing CD19 CAR-certolizumab scFv showed delayed onset of weight loss compared to those in the CD19 CAR-EGFRt group, but all animals in this group began to exhibit significant weight loss and declining health 10 days after T-cell injection, with all animals reaching the humane end point within 14 days of T-cell injection. (FIGS. 10-14, and 56).

In addition to the quantitative clinical scores, animals in different treatment groups also showed different clinical symptoms at the endpoint (Supplementary Table 4.S1). In the EGFRt and PBS groups, animals exhibited symptoms such as weight loss or hind limb paralysis, which are associated with unspecific illness or high tumor burden. In contrast, animals treated with CD19 CAR-T cells showed signs of potential neurotoxicity (moribund or seizure symptoms) or hypofibrinogenemia (uncontrolled bleeding, bleeding spot at IP injection sites or internal bleeding), which are clinical symptoms associated with CRS. Interestingly, animals receiving CD19 CAR-tocilizumab scFv T cells mostly suffered from weight loss instead of neurotoxicity or hypofibrinogenemia at endpoints, suggesting the presence of tocilizumab scFv may have reduced or prevented CRS-related toxicities (Supplementary Table 4.S1).

Taken together, these results suggest that the secretion of cytokine antagonists such as anakinra and scFvs derived from tocilizumab or certolizumab may have varying degrees of efficacy in tempering CRS-associated toxicities.

6. Expression of Self-Modulators do not Affect the Anti-Tumor Efficacy of CD19 CAR-T Cells In Vivo

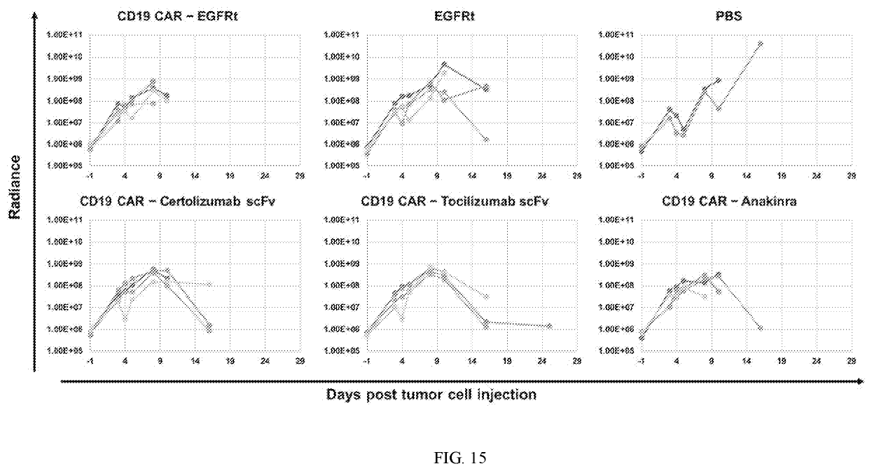

In the animal study described above, tumor burden was monitored via bioluminescence imaging. Although rapid deterioration of animal health limited the number of data points available, it was clear that animals treated with PBS or EGFRt-expressing T cells had poor tumor control (FIG. 15). Animals treated with CD19 CAR-EGFRt T cells also died in the presence of high tumor burden, but this is likely due to the fact that these animals succumbed to CRS-related toxicities before the T cells had the opportunity to eliminate the engrafted tumor. Among animals that were treated with CAR-T cells and survived beyond 8 days post T-cell injection, nearly all showed significant tumor regression and the majority were tumor-free at the time of sacrifice (FIG. 15).

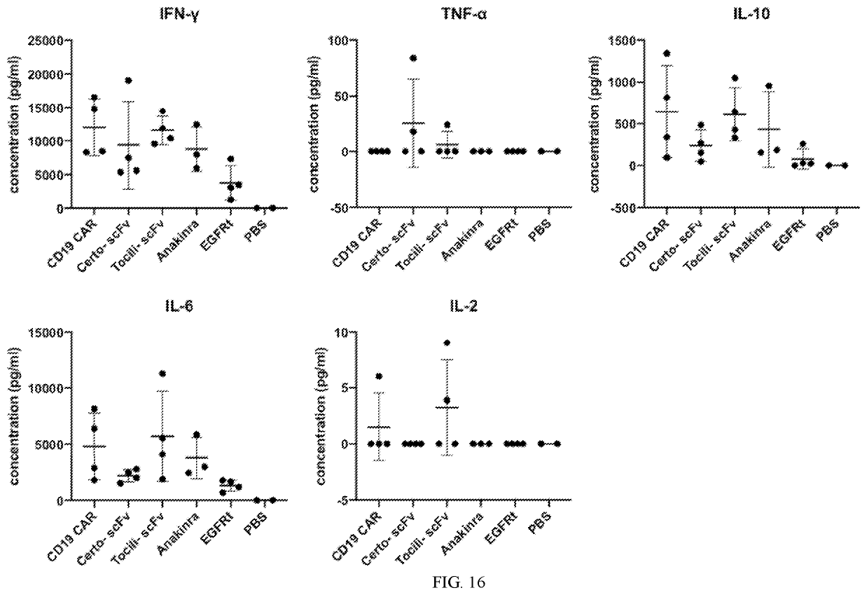

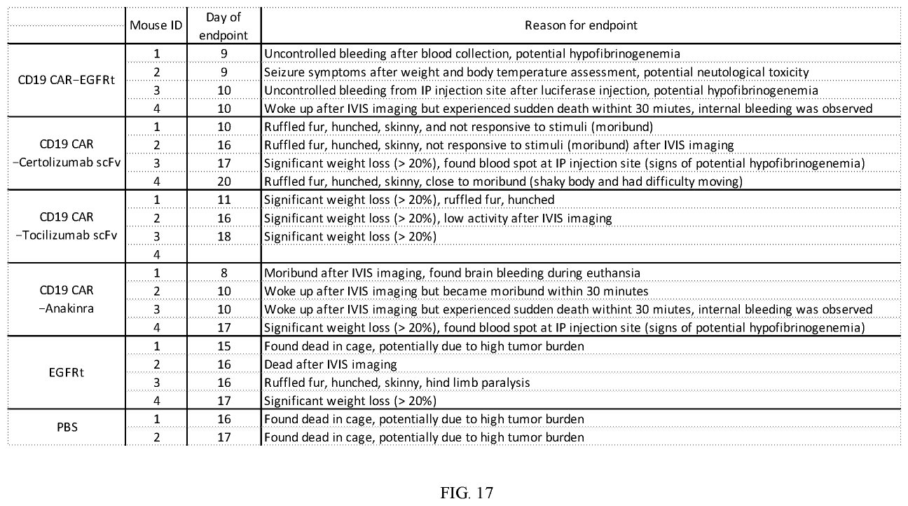

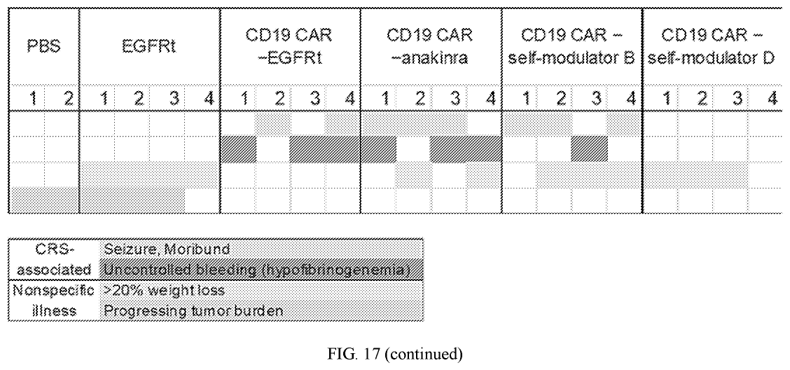

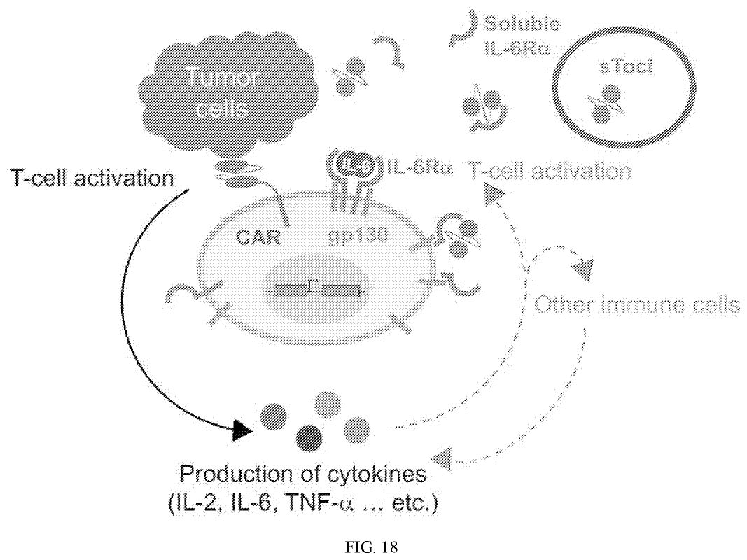

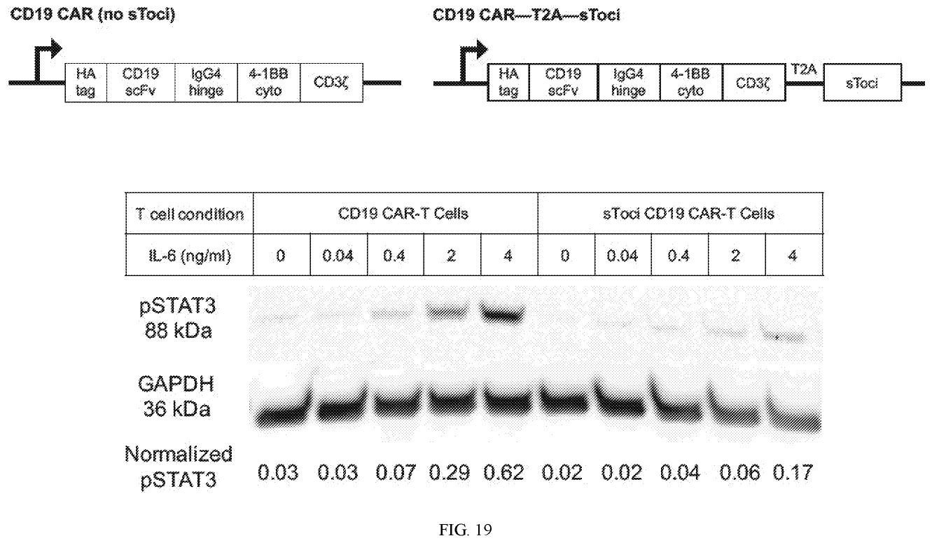

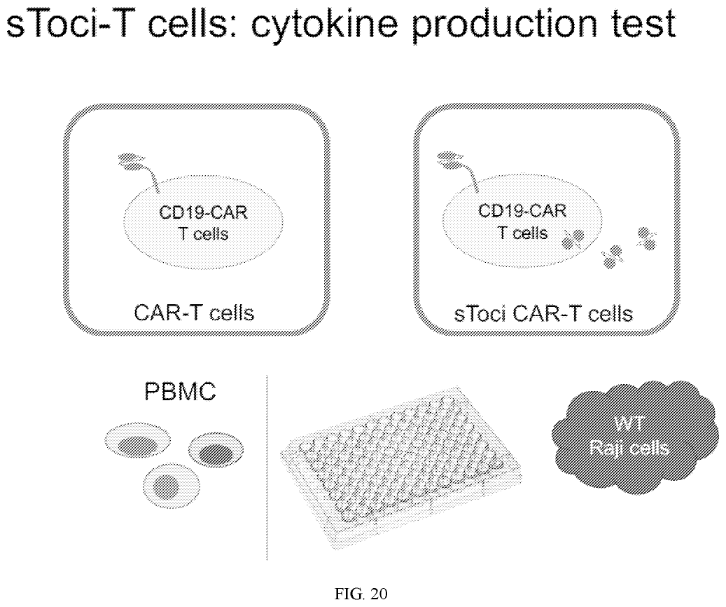

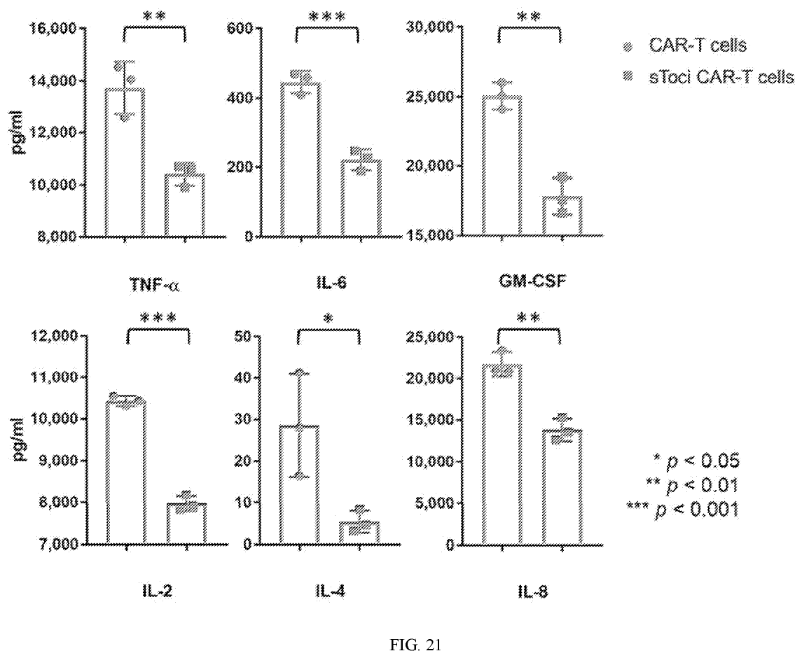

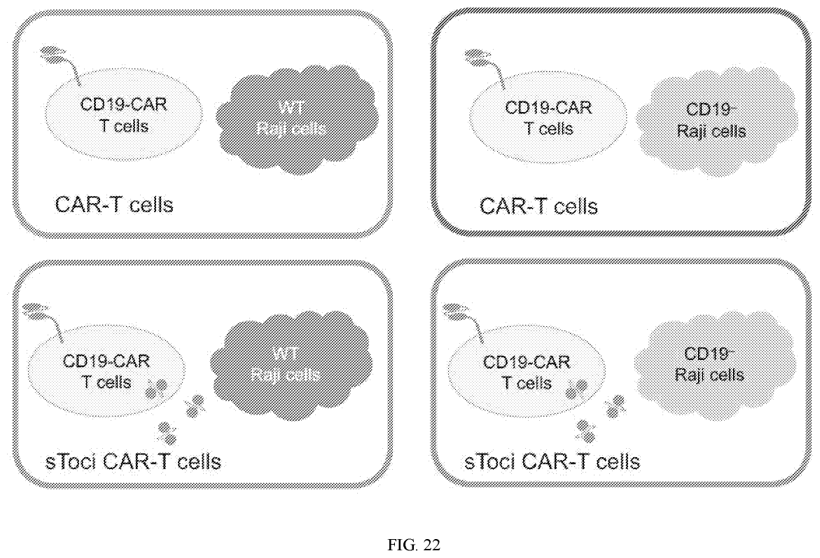

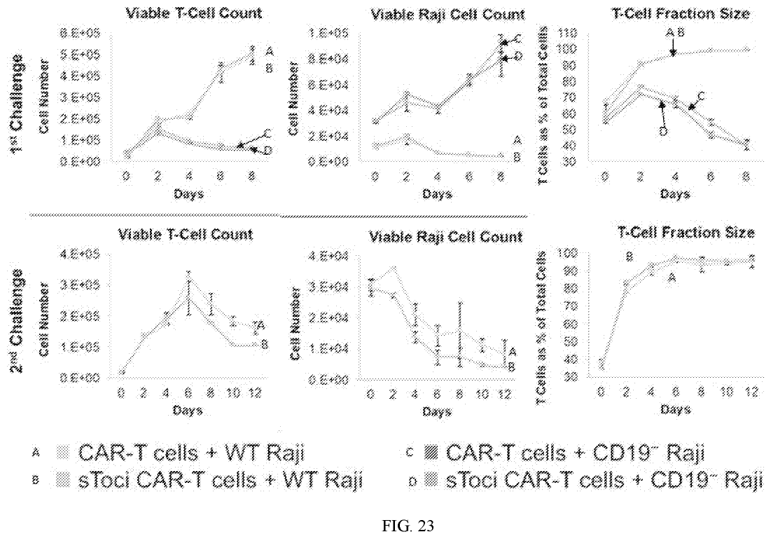

Quantification of serum cytokine levels 3 days after T-cell injection revealed an increase in human IFN-γ, TNF-α, IL-10, IL-6 and IL-2 in animals that were treated with CD19 CAR-T cells, but not in animals treated with PBS. Animals treated with EGFRt-T cells showed slight increases of IFN-γ, IL-10 and IL-6 which may be caused by the graft-versus-graft response. CD19 CAR-certolizumab scFv and CD19 CAR-anakinra treatment groups showed reduced expression of IFN-γ, IL-10, IL-6 and IL-2, compared to the CD19 CAR-EGFRt group, which may be a sign of anakinra and certolizumab scFv's efficacy in mediating CRS. However, based on animal weight, clinical score and endpoint results, the efficacy of CD19 CAR-anakinra T cells in mediating actual CRS clinical symptoms is uncertain. Surprisingly, despite the efficacy of tocilizumab scFv in preventing weight loss and health deterioration, animals in the CD19 CAR-tocilizumab scFv treatment group showed similar levels of cytokine expression as those in the CD19 CAR-EGFRt group, indicating the possibility that blocking IL-6 signaling poses minimal inhibition on the effector function of CAR-T cells and does not alter the in vivo expression of cytokines that were monitored in this study (FIG. 16). The certolizumab scFv group and tocilizumab scFv group had higher levels of TNF-α and IL-6 compare to the CD19 CAR-EGFRt group, respectively, which is likely due to compensatory effects triggered by successful blocking of cytokine signaling (21).

B. Discussion

Due to significant donor-to-donor variation in the serum cytokine levels of CRS patients, definitive threshold in cytokine levels could not be used for CRS diagnosis. In addition, the complexity of CRS cannot be fully recapitulated with in vitro assays because of the involvement of multiple cell types and the intrinsic difference between in vivo environment and in vitro culture. To demonstrate the efficacy of the self-modulating T cell system, the inventors developed multiple murine models in an attempt to recapitulate human CRS in vivo.

Results from PBMC-humanized NSG mouse model demonstrated a positive correlation between tumor burden and expression of human IFN-γ and GM-CSF. However, a lack of CRS-associated clinical symptoms and absence of human IL-6 limited the utility of this model for the study of human CRS.

As an alternative, the inventors evaluated the use of a humanized NSG-SGM3 mouse model reported in a recent publication (16). In the published model, CD34⁺ cells isolated from cord blood were used to humanize NSG-SGM3 mice, and these humanized mice subsequently served as both a source of T cells and the syngeneic recipient of engineered CAR-T cells. Although this model was shown to recapitulate major symptoms of human CRS, including the production of human IL-6, the need to isolate T cells from humanized mice to generate CAR-T cells necessitates a protracted experimental procedure that is both time consuming and highly technically complex. In an attempt to streamline the model while maintaining the ability to trigger CRS in vivo, the inventors evaluated the possibility of generating T cells from fetal tissues and cord blood. Although the inventors found fetal liver to be a plentiful source of CD34⁺ HSPCs, the inventors could not isolate sufficient numbers of viable CD3⁺ T cells from fetal tissue. In contrast, cord blood proved to be a good source of T cells, but not of CD34⁺ cells. Consequently, the inventors chose to pursue a modified mouse model in which CD34⁺ cells and T cells from two different donors would be administered to NSG-SGM3 mice.

An initial pilot study showed that CD19 CAR-T cells derived from a healthy adult donor would trigger GvHD but not CRS in NSG-SGM3 mice humanized with CD34⁺ cells derived from fetal liver. Therefore, a subsequent study was performed with the engraftment of CD19⁺ Raji tumor cells prior to CD19 CAR-T cell infusion. Results from this study provided tantalizing evidence that CD19 CAR-T cells engineered to secrete antagonist proteins that inhibit IL-6Ra, TNF-α, or IL-1R may indeed lessen toxicities associated with CRS without reducing the CAR-T cells' anti-tumor efficacy. However, additional studies with larger animal groups is needed to confirm these findings.

The inventors observed that CD19 CAR-T cells co-expressing the certolizumab scFv was uniquely able to delay animal weight loss associated with CRS. This early action exhibited by the anti-TNF-α certolizumab scFv may be explained by the fact that TNF-α is one of the cytokines that appear early during T-cell activation. In contrast to certolizumab- and tocilizumab-derived scFvs, anakinra was consistently observed to be expressed at low levels in our engineered CAR-T cells (Example 2). This low expression, combined with the known short half-life (2.6 to 6 hours) of anakinra in vivo^{25,26} may explain anakinra's relative lack of efficacy in our in vivo model. However, blockade of IL-1 signaling showed strong protection against CRS in a similar approach reported by Giavridis et al. (12), in which murine (instead of human) IL-1Rα antagonist (IL-1RN) was co-expressed with the CD19 CAR in human T cells. The divergent results obtained in our studies may have been due to differences in the specific mouse models used as well as differences in murine vs. human IL-1 signaling.

It is important to note that CAR-T cells engineered to secrete cytokine antagonists exhibited robust anti-tumor effector function, provided the animals survived the rapid onset of toxicities associated with CRS. A modified model with lower tumor burden and lower T-cell dose may be needed to more thoroughly evaluate the efficacy and persistence of self-modulator-expressing CAR T cells.

Based on the different weight loss patterns observed in animals treated with CAR-T cells that secrete tocilizumab scFv vs. certolizumab scFv vs. anakinra, the inventors hypothesize that self-modulators have different working mechanisms. Interestingly, animals receiving CD19 CAR-tocilizumab scFv T cells showed unaffected cytokine expression 3-days post-T-cell injection, which may be an indication that the tocilizumab scFv mediates CRS through an approach that is independent of the inflammatory cytokines that have been observed (IL-2, IL-6, IL-10, TNF-α, IFN-γ). Understanding of the working mechanism of different self-modulators and how they modulate immune response can broaden our understanding of CRS and support the development of new CRS treatment.

The inventors have demonstrated the development of multiple humanized murine models and evaluated the efficacy of self-modulating T cell systems in vivo. The limitation and expected outcome of different humanized mouse models described in this example can be useful references for future experiments and model development. Results from the humanized NSG-SGM3 model also revealed different behaviors among different self-modulators, and an in-depth exploration of the mechanisms behind each cytokine antagonist could broaden the understanding of CRS and associated immune responses. A fully optimized self-modulating T-cell system can offer a new approach to increase the safety of adoptive T-cell therapy, and utilization of the in vivo model developed in this study may facilitate further understanding of CRS associated with adoptive T-cell therapy for cancer.

C. Methods

1. Primary Human T Cell Generation

Blood from healthy donor that used for T-cell isolation were obtained from the UCLA Blood and Platelet Center. All T cells were cultured in RPMI-1640 supplemented with 10% HI-FBS and fed with 50 U/ml IL-2 (Life Technologies) and 1 ng/ml IL-15 (Miltenyi Biotec) every two days. T-cells in different in vivo models were generated in slightly different protocols due to the purpose of study and the differences were described below.

2. Human CD4⁺ T Cells in PBMC-Humanized NSG Mouse Model

Primary human CD4⁺ T cells as well as peripheral blood mononuclear cells (PBMCs) were isolated using antibody cocktails (RosetteSep kits, STEMCELL Technologies) followed by Ficoll-Paque (GE Healthcare Life Sciences) density-gradient separation. Isolated cells were stimulated with CD3/CD28 Dynabeads at a 1:1 bead:cell ratio. Two days after Dynabeads stimulation, cells were transduced with lentivirus (MOI=3). Dynabeads were removed on day 10 and transduced cells were enriched for CAR expression by magnetic bead-based sorting (Miltenyi Biotec). Enriched cells were then expanded in the presence of irradiated TM-LCL feeder cells (T cell:TM-LCL=1:7). 100 U/ml penicillin and 100 μg/ml streptomycin (Life Technologies) was supplemented once to T-cell expansion cultures at the time of TM-LCL addition.

Primary human CD4⁺ T cells and PBMCs for FIGS. 27, 28, and 44 were isolated as described above. Isolated cells were stimulated with Dynabeads at a 1:3 bead:cell ratio and transduced with untitered retrovirus two days and three days after Dynabeads stimulation. Dynabeads were removed on day 9 and cells were not expanded with TM-LCL feeder cells.

3. CRISPR Edited Human T Cells for Humanized NSG-SGM3 Mouse Model

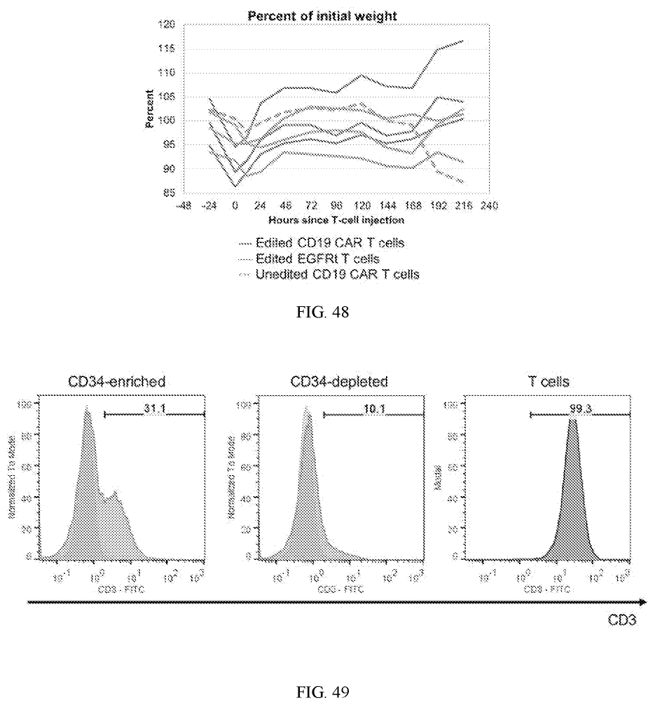

For T cells used in FIGS. 46-48, primary human CD4+ and CD8+ T cells were isolated using antibody cocktail (RosetteSep Kits, STEMCELL Technologies). Isolated cells were stimulated with Dynabeads at a 1:3 bead:cell ratio and transduced with untitered retrovirus two days and three days after Dynabeads stimulation. Dynabeads were removed on day 5 and T cells were electroporated with Cas9-gRNA ribonucleoproteins (RNP) consisted of 300 pmol Cas9 protein and 300 pmol sgRNA. 5×10⁶ cells were mixed with Cas9-RNP and electroporated in Ingenio electroporation solution (Mirus) on Amaxa Nucleofector 2b device (Lonza) with program T-017. Unedited T cells were not electroporated but were in contact with Ingenio electroporation solution. Both edited and unedited T cells were cultured using standard condition (RPMI-1640 supplemented with 10% HI-FBS, fed with 50 U/ml IL-2 (Life Technologies) and 1 ng/ml IL-15 (Miltenyi Biotec) every two days.

4. Naïve Memory Human T Cells for Humanized NSG-SGM3 Mouse Model

For data in FIGS. 9, 10, 12, and 14-16, primary human PBMCs were isolated from whole blood by using Ficoll-Paque density-gradient separation. CD25⁻/CD14⁻/CD62L⁺ naïve memory T cells were subsequently isolated from PBMCs by using magnetic bead-based sorting (Miltenyi) to deplete CD25- and CD14-expressing cells followed by enrichment of CD62L⁺ cells. Isolated cells were stimulated with Dynabeads at a 1:3 bead:cell ratio and transduced with untitered retrovirus two days and three days after Dynabeads stimulation. Dynabeads were magnetically removed on day 7 and T cells were kept in culture in RPMI-1640 supplemented with 10% HI-FBS and fed with 50 U/ml IL-2 (Life Technologies) and 1 ng/ml IL-15 (Miltenyi Biotec) every two days.

5. Processing of Fetal Liver and Fetal Spleen

Fetal liver and spleen were obtained from UCLA Gene and Cellular Therapy Core. Tissues were gently washed with PBS 3 to 4 times followed by dissecting tissues into 3 mm³ pieces in Iscove's Modified Dulbecco's Medium (IMDM) (Thermo Fisher). Gently resuspend tissue and media solution with 16 G needle, 10 ml syringe for 5 to 7 times to homogenize the tissue completely. Incubate homogenized tissue solution in 250 U/ml collagenase (MP Biomedicals), 1200 U/ml hyaluronidase (Sigma-Aldrich), 150 U/ml DNase (Sigma-Aldrich), 1×Pen/strep (Life Technologies) and 1× amphotericin solution (2.5 μg/ml) (HyClone) in IMDM, incubate for 90 minutes. Filter digested tissue suspension through a 100 μm cell strainer followed by mononuclear cell isolation via Ficoll-Paque density-gradient separation. CD34⁺ cells were subsequently enriched after mononuclear cell isolation by performing bead-based magnetic enrichment using human CD34-biotin (Miltenyi) or CD34 MicroBead Kit, UltraPure (Miltenyi). To generate T-cell culture, after CD34-enrichment, T cells in CD34⁻ population were subsequently enriched by using pan T cell isolation kit (Miltenyi) following manufacturer's instruction. Pan T cell enriched cells were then stimulated with CD3/CD28 dynabeads at 3:1 cell:bead ratio followed by transduction with retrovirus 2 and 3 days after stimulation in order to generate T cell culture.

6. Processing of Cord Blood

Cord blood units were obtained from UCLA/CFAR Virology Core Laboratory. Mononuclear cells from cord blood were isolated by diluting cord blood with 3× volume of PBS, followed by Ficoll-Paque density-gradient separation. Mononuclear cells were collected and CD34⁺ hematopoietic stem and progenitor cells were immediately enriched by using CD34 MicroBead Kit, UltraPure (Miltenyi), following manufacturer's instruction. Pan T cell enrichment were performed in CD34⁻ population after CD34-enrichment and pan T cell-enriched cells were subsequently stimulated with CD3/CD28 dynabeads at 3:1 cell:bead ratio to generate T-cell culture. Stimulated cells were transduced with untitered retrovirus 2 and 3 days after stimulation and dynabeads were magnetically removed after 7 days.

7. Repeated Antigen Challenge of T Cells Generated from Cord Blood

T cells generated from cord blood expressing CD19 CAR constructs were seeded at 1×10⁶ cells/well in a 12-well plate with 3 ml of complete RPMI and co-incubated with WT Raji cells at 1:1 effector-to-target ratio. Number of T cells and remaining target cells were evaluated by flow cytometry every two days. 1×10⁶ of fresh WT Raji cells were added to co-culture every two days after cell counting.

8. PBMC-Humanized NSG Mouse Model

All in vivo experiments were approved by the UCLA Institutional Animal Care and Use Committee (IACUC). Six- to eight-week-old NOD/SCID/γ_{c}^{-/-} (NSG) mice were purchased from UCLA Department of Radiation and Oncology. For the study indicated in FIG. 42, three NSG mice were injected with 2×10⁷ peripheral blood mononuclear cells (PBMCs) and 5×10⁵ EGFP⁺, firefly luciferase (ffluc)-expressing Raji cells via tail-vein injection on day 0. On day 10, animals with engrafted tumors were treated with 1×10⁷ donor-matched, CD19 CAR-expressing and CAR-enriched CD4⁺ T cells. Weight of animals were monitored daily, and weight on the day of PBMCs and Raji cells injection was defined as 100% initial weight. Tumor progression was monitored by bioluminescence imaging using an IVIS Lumina III LT imaging System (PerkinElmer). Animals were euthanized at the humane endpoint or at day 45. There was no blinding in the reported animal studies. The sample size n=3 was chosen to generate statistically meaningful data.

Data for the PBMC-humanized NSG mouse study in FIGS. 27, 28, and 44 were generated from the same experiment as indicated in Example 1. In brief, 2×10⁶ PBMCs and 5×10⁵ EGFP⁺, ffluc-expressing Raji cells were injected into NSG mice via tail-vein injection on day 0. Animals with engrafted tumor were treated with 2×10⁶ donor-matched CD4⁺ T cells that were transduced with a truncated epidermal growth factor receptor (EGFRt; negative control) or a CD19 CAR with or without co-expression of single-chain variant of tocilizumab (sToci). Peripheral blood was collected by retro-orbital bleeding on day 15 (8 days post T-cell injection). Animals were sacrificed at the humane endpoint or on day 35 (28 days post T-cell injection), whichever was earlier. At the time of animal sacrifice, a final blood sample was obtained by cardiac puncture. Blood samples were centrifuged at 2000×g at 4° C. for 15 minutes to collect plasma for cytokine measurement using CBA assay kit. Tumor progression was monitored by bioluminescence imaging using an IVIS Lumina III LT imaging System (PerkinElmer). The sample size n=5 was chosen to generate statistically meaningful data. There was no blinding in the reported animal studies.

9. Humanized NSG-SGM3 Mouse Model with CRISPR Edited T Cells

All in vivo experiments were approved by the UCLA Institutional Animal Care and Use Committee (IACUC). Six- to twelve-week-old NSG-SGM3 (NSG^{TgCMV-IL3, CSF2, KITLG}1Eav/MloySzJ) mice were obtained from Jackson Laboratories or breed by UCLA CFAR Humanized Mouse Core Laboratory. For data in FIGS. 46-48, NSG-SGM3 mice were sub-lethally irradiated (200 rads) with a Cesium-137 irradiator and injected with 3×10⁵ human hematopoietic stem and progenitor cells (HSPCs) generated from fetal liver tissues via tail-vein injection. Human Fetal liver tissue was obtained from UCLA Gene and Cellular Therapy Core. The humanization status was confirmed by evaluating the cellular components in peripheral blood, staining the cells with CD3-FITC (Biolegend, clone: UCHT1), CD14-PacBlue (Biolegend, clone: M5E2), CD19-APC (Miltenyi Biotec, clone: LT19), and CD45-PECy7 (Biolegend, clone: HI30) and analyzing on a MACSQuant VYB flow cytometer (Miltenyi Biotec). Animals with more than 20% of total human cells in the peripheral blood were considered as humanized. Once humanization is confirmed in all the animals, 6×10⁶ of unedited T cells transduced with CD19 CAR or edited T cells transduced with truncated epidermal growth factor receptor (EGFRt) or CD19 CAR were injected into animals via tail-vein injection on day 30. Peripheral blood was collected by retro-orbital or submandibular bleeding on day 19 (1-day before T-cell injection), and on day 35 (5-days after T-cell injection). All animals were sacrificed on day 45 (15-days post T-cell injection) due to development of GvHD symptoms in some animals. At the time of animal sacrifice, a final blood sample was obtained by cardiac puncture. Animals were weighted daily per standard animal care and the weight on the day of humanization was defined as 100% initial weight. There was no blinding in the reported animal studies.

10. Humanized NSG-SGM3 Mouse Model with Allogeneic HSPCs and Adoptively Transferred T Cells

Six- to eight-week-old NSG-SGM3 (NSG^{TgCMV-IL3, CSF2, KITLG}1Eav/MloySzJ) mice bred by UCLA CFAR Humanized Mouse Core Laboratory. For data in FIGS. 9, 10, 12, 14, 15, 16, NSG-SGM3 mice were sub-lethally irradiated (200 rads) with a Cesium-137 irradiator and injected with 3×10⁵ human hematopoietic stem and progenitor cells (HSPCs) generated from fetal liver tissues via retro-orbital injection. Human Fetal liver tissue was obtained from UCLA Gene and Cellular Therapy Core. The humanization status was confirmed by evaluating the cellular components in peripheral blood, staining the cells with CD3-FITC (Biolegend, clone: UCHT1), CD14-PacBlue (Biolegend, clone: M5E2), CD19-APC (Miltenyi Biotec, clone: LT19), and CD45-PECy7 (Biolegend, clone: HI30) and analyzing on a MACSQuant VYB flow cytometer (Miltenyi Biotec). Animals with more than 20% of total human cells in the peripheral blood were considered as humanized. Once humanization is confirmed in all the animals (about 3-4 weeks after humanization), 4.5×10⁶ of ffluc-expressing Raji lymphoma cells were injected into animals via tail-vein injection. 6 days after tumor cell injection, 8×10⁶ transduced naïve memory T cells were injected into animals via tail-vein injection. Peripheral blood was collected by retro-orbital or submandibular bleeding on day 5 (1-day before T-cell injection), and on day 9 (3-days after T-cell injection). Weight, body temperature and clinical score of animals were assessed at least twice per day for a week after T-cell injection. Clinical scores of animals were given based on the health condition of animals with 5 being the maximum and 0 being the minimum (Score: 5=healthy animal; 4=normal activity, ruffled hair; 3=ruffled hair, hunched posture, reduced activity but still active when agitated; 2=ruffled hair, hunched posture, low activity; 1=very low activity, not responsive upon agitation, moribund; 0=end point). Tumor progression was monitored by bioluminescence imaging on day −1, day 3, day 4, day 5, day 8, day 10 and day 16. Animals were humanized on humane endpoint.

11. Statistical Analysis

Statistical tests were performed in Excel and GraphPad Prism 8 for Mac. A minimum of triplicate was chosen to allow for calculation of statistics. Statistical significance was analyzed by using two-tailed, unpaired, homoscedastic Student's t-test. Survival curves were evaluated by the log-rank Mantel-Cox text (GraphPad Prism 8).

D. Supplementary Table

Supplementary Table 4.S1: Log of endpoints (days of tumor cell injection as day 0, T-cells
were injected on day 6)

| | | Day of | |
|---|---|---|---|
| | Mouse ID | endpoint | Reason for endpoint |
| CD19 CAR-EGFRt | 1 | 9 | Uncontrolled bleeding after blood collection, potential hypofibrinogenemia |
| | 2 | 9 | Seizure symptoms after weight and body temperature assessment, potential neutological toxicity |
| | 3 | 10 | Uncontrolled bleeding from IP injection site after luciferase injection, potential hypofibrinogenemia |
| | 4 | 10 | Woke up after MS imaging but experienced sudden death withint 30 miutes, internal bleeding was |
| | | | observed |
| CD19 CAR— | 1 | 10 | Ruffled fur, hunched, skinny, and not responsive to stimuli (moribund) |
| Certolizumab scFv | 2 | 16 | Ruffled fur, hunched, skinny, not responsive to stimuli (moribund) after MS imaging |
| | 3 | 17 | Significant weight loss (>20%), found blood spot at IP injection site (signs of potential |
| | | | hypofibrinogenemia) |
| | 4 | 20 | Ruffled fur, hunched, skinny, close to moribund (shaky body and had difficulty moving) |
| CD19 CAR— | 1 | 11 | Significant weight loss (>20%), ruffled fur, hunched |
| Tocilizumab scFv | 2 | 16 | Significant weight loss (>20%), low activity after MS imaging |
| | 3 | 18 | Significant weight loss (>20%) |
| | 4 | | |
| CD19 CAR— | 1 | 8 | Moribund after MS imaging, found brain bleeding during euthansia |
| Anakinra | 2 | 10 | Woke up after MS imaging but became moribund within 30 minutes |
| | 3 | 10 | Woke up after MS imaging but experienced sudden death withint 30 miutes, internal bleeding |
| | | | was observed |
| | 4 | 17 | Significant weight loss (>20%), found blood spot at IP injection site (signs of potential |
| | | | hypofibrinogenemia) |
| EGFRt | 1 | 15 | Found dead in cage, potentially due to high tumor burden |
| | 2 | 16 | Dead after MS imaging |
| | 3 | 16 | Ruffled fur, hunched, skinny, hind limb paralysis |
| | 4 | 17 | Significant weight loss (>20%) |
| PBS | 1 | 16 | Found dead in cage, potentially due to high tumor burden |
| | 2 | 17 | Found dead in cage, potentially due to high tumor burden |

E. Reference

The following references and the publications referred to throughout the specification, to the extent that they provide exemplary procedural or other details supplementary to those set forth herein, are specifically incorporated herein by reference.
1. Brentjens, R., Yeh, R., Bernal, Y., Riviere, I. & Sadelain, M. Treatment of chronic lymphocytic leukemia with genetically targeted autologous T cells: case report of an unforeseen adverse event in a phase I clinical trial. Mol. Ther. 18, 666-668 (2010).
2. Teachey, D. T. et al. Identification of predictive biomarkers for cytokine release syndrome after chimeric antigen receptor T-cell therapy for acute lymphoblastic leukemia. Cancer Discov. 6, 664-679 (2016).
3. Hay, K. A. et al. Kinetics and biomarkers of severe cytokine release syndrome after CD19 chimeric antigen receptor-modified T-cell therapy. Blood 130, 2295-2306 (2017).
4. Diaconu, I. et al. Inducible Caspase-9 Selectively Modulates the Toxicities of CD19-Specific Chimeric Antigen Receptor-Modified T Cells. Mol. Ther. 25, 1-13 (2017).
5. Di Stasi, A. et al. Inducible Apoptosis as a Safety Switch for Adoptive Cell Therapy. N. Engl. J. Med. 365, 1673-1683 (2011).
6. Wang, X. et al. A transgene-encoded cell surface polypeptide for selection, in vivo tracking, and ablation of engineered cells. Blood 118, 1255-1263 (2011).
7. Brady, J. L. et al. Preclinical screening for acute toxicity of therapeutic monoclonal antibodies in a hu-SCID model. Clin. Transl. Immunol. 3, e29 (2014).
8. Sentman, M.-L. et al. Mechanisms of Acute Toxicity in NKG2D Chimeric Antigen Receptor T Cell-Treated Mice. J. Immunol. 197, 4674-4685 (2016).
9. Wang, L. et al. Efficient tumor regression by adoptively transferred CEA-specific CAR-T cells associated with symptoms of mild cytokine release syndrome. Oncoimmunology 5, 1-13 (2016).
10. Pennell, C. A. et al. Human CD19-targeted mouse T-cells induce B-cell aplasia and toxicity in human CD19 transgenic mice. Molecular Therapy (2018). doi:10.1016/j.ymthe.2018.04.006
11. van der Stegen, S. J. C. et al. Preclinical in vivo modeling of cytokine release syndrome induced by ErbB-retargeted human T cells: identifying a window of therapeutic opportunity? J. Immunol. 191, 4589-98 (2013).
12. Giavridis, T. et al. CAR T cell-induced cytokine release syndrome is mediated by macrophages and abated by IL-1 blockade. Nat. Med. 24, 731-738 (2018).
13. Obstfeld, A. E. et al. Cytokine release syndrome associated with chimeric-antigen receptor T-cell therapy; clinicopathological insights. Blood prepublished online Oct. 26 2017 (2017). doi:10.1182/blood-2017-08-802413
14. Singh, N. et al. Monocyte lineage-derived IL-6 does not affect chimeric antigen receptor T-cell function. Cytotherapy 19, 867-880 (2017).
15. Barrett, D. M., Singh, N., Hofmann, T. J., Gershenson, Z. & Grupp, S. A. Interleukin 6 Is Not Made By Chimeric Antigen Receptor T Cells and Does Not Impact Their Function. Blood 128, 654 LP-654 (2016).
16. Norelli, M. et al. Monocyte-derived IL-1 and IL-6 are differentially required for cytokine-release syndrome and neurotoxicity due to CAR T cells. Nat. Med. (2018). doi:10.1038/s41591-018-0036-4
17. Staedtke, V. et al. Disruption of a self-amplifying catecholamine loop reduces cytokine release syndrome. Nature 564, 273-277 (2018).
18. Davila, M. L. et al. Efficacy and toxicity management of 19-28z CAR T cell therapy in B cell acute lymphoblastic leukemia. Sci. Transl. Med. 6, 1-13 (2014).
19. Maude, S. L. et al. Chimeric Antigen Receptor T Cells for Sustained Remissions in Leukemia. N. Engl. J. Med. 371, 1507-1517 (2014).
20. Hay, K. A. et al. Kinetics and biomarkers of severe cytokine release syndrome after CD19 chimeric antigen receptor-modified T-cell therapy. Blood 130, 2295-2306 (2017).
21. Nishimoto, N. et al. Mechanisms and pathologic significances in increase in serum interleukin-6 (IL-6) and soluble IL-6 receptor after administration of an anti-IL-6 receptor antibody, tocilizumab, in patients with rheumatoid arthritis and Castleman disease. Blood 112, 3959-3964 (2008).
22. Giavridis, T. et al. CAR T cell-induced cytokine release syndrome is mediated by macrophages and abated by IL-1 blockade letter. Nat. Med. 24, 731-738 (2018).
23. Obstfeld, A. E. et al. Cytokine release syndrome associated with chimeric-antigen receptor T-cell therapy; clinicopathological insights. Blood 130, blood-2017-08-802413 (2017).
24. Giavridis, T. et al. CAR T cell-induced cytokine release syndrome is mediated by macrophages and abated by IL-1 blockade. Nat. Med. 24, 731-738 (2018).
25. Yang, B. B. et al. Pharmacokinetics of anakinra in subjects with different levels of renal function. Clin. Pharmacol. Ther. 74, 85-94 (2003).
26. So, A. et al. The role of IL-1 in gout: from bench to bedside. Rheumatology, 57, i12-i19 (2018).

### Example 4: Expressing CRS-Mediating Agents in an Inducible Manner

Cytokine release syndrome (CRS) is a common toxicity in patients receiving chimeric antigen receptor (CAR)-T cell therapy. The inventors have demonstrated that the cytokine expression levels can be down-regulated in CAR-T cells by engineering T cells to constitutively express cytokine antagonists (termed “self-modulators”). However, it is unclear whether the constitutive expression of self-modulators would cause any side effect in patients. Thus, to develop smarter CAR-T cells that have the ability to avoid over-stimulation upon antigen stimulation while minimizing potential toxicities associated with the self-modulator design, the inventors developed and tested multiple inducible gene-expression systems to enable regulated production of self-modulators by CD19 CAR-T cells. The incorporation of inducible systems enables CAR-T cells to control their stimulation level when a certain cytokine is present at high levels or when a specific signaling pathway is triggered. This work enables the modulation of T-cell activation with more precise temporal control, which maximizes the efficacy of CAR-T cells and prevents potential side effects caused by the constitutive expression of self-modulators.

The balance of the immune system is finely regulated by multiple components, and the complete scope of immune responses and regulatory mechanisms is yet fully understood and cannot be completely regulated through synthetic biological systems at this time. As such, potentially severe toxicities such as CRS have been observed as side effects associated with therapeutic strategies that seek to leverage and/or disrupt the immune system. Examples 1-3 of this application describes efforts to engineer “self-modulating” CAR-T cells that constitutively secrete cytokine antagonists, so as to minimize the potential for CRS resulting from CAR-T cell therapy. The inventors demonstrated that self-modulating CAR-T cells can down-regulate cytokine production in vitro and lessen CRS-related toxicities in vivo without dampening anti-tumor effector functions. In the systems described so far, CAR-T cells were engineered to constitutively produce cytokine antagonists, and the inventors did not observe any evidence that this additional engineering step exerted substantial metabolic or other noticeable costs to the CAR-T cells' fitness. However, it remains possible that in the human therapeutic context, constitutive secretion of cytokine antagonists may lead to undesirable outcomes such as immune dysregulation or premature exhaustion for the engineered T cells. To address these concerns, the inventors explored multiple strategies to engineer T cells that only activate the self-modulator mechanism in environments of high cytokine concentration or as a consequence of T-cell activation.

Numerous tools in the synthetic biology field have been developed to control gene expression. For example, transcriptional regulatory system has been used to build a variety of regulatory circuits such as oscillators (1, 2), memory circuits)3-5) or biocomputers (6, 7). RNA interference (RNAi) such as short hairpin RNA (shRNA) and microRNA (miRNA) that regulate gene expression at translational level (8, 9), or degradation tag and localization tag that controls the target-target interaction at protein level (10-13) have also been used widely to build complex gene regulatory circuits.

In this example, the inventors aim to control the secretion of self-modulators upon different cues in order to control the response of self-modulators and prevent potential side effects caused by constitutive expression. Because IL-6 is a key cytokine in CRS (14-18), the inventors first developed an IL-6-inducible promoter that controls the secretion of self-modulators at the transcriptional level. When IL-6 signaling is triggered in CAR-T cells, IL-6-inducible promoter drives the expression of self-modulators and prevent CAR-T cells from over-stimulation. To optimize the inducibility and expression level of the inducible promoter, panels of different response elements (RE) and minimal promoters were tested. An additional synthetic transcriptional activator, ZF-STAT3, that preferentially binds to synthetic IL-6-inducible promoter were also incorporated into cells to increase inducibility.

In addition to IL-6, the possibility to induce the expression of self-modulators upon T-cell activation has also been tested by using the nuclear factor of activated T-cells (NFAT)-inducible promoter. NFAT signaling is a major signaling pathway that is activated upon T-cell activation. Although multiple cell types such as monocytes, macrophages are associated with the occurrence of CRS, the T-cell activation is the trigger of the cascade of immune responses that ultimately leads to CRS in patients receiving CAR-T cells. The inventors thus hypothesize that CRS can be prevented or at least ameliorated by triggering the expression of self-modulators upon T-cell activation, and evaluated the effects of linking self-modulator production to NFAT signaling.

Beyond inducible secretion of cytokine antagonists, a negative feedback loop that dampens NFAT signaling in the presence of high IL-6 concentration was also explored as an alternative approach to achieve T-cell self-modulation. To this end, the inventors sought to identify genes that are upregulated upon IL-6 signaling as well as peptide inhibitors for NFAT signaling, with a proposed aim of placing the production of peptide inhibitors under the control of promoters that regulate genes whose expression is linked to IL-6 signaling

Here, the inventors report characterization results on the various strategies mentioned above to control the expression of self-modulators in order to make smarter T cells that can avoid over-stimulation upon antigen stimulation.

A. Results

1. IL-6-Inducible Promoter Comprising the JRE-IL-6 Response Element and YB TATA Minimal Promoter Yields High Fold-Induction in HEK293T Cells

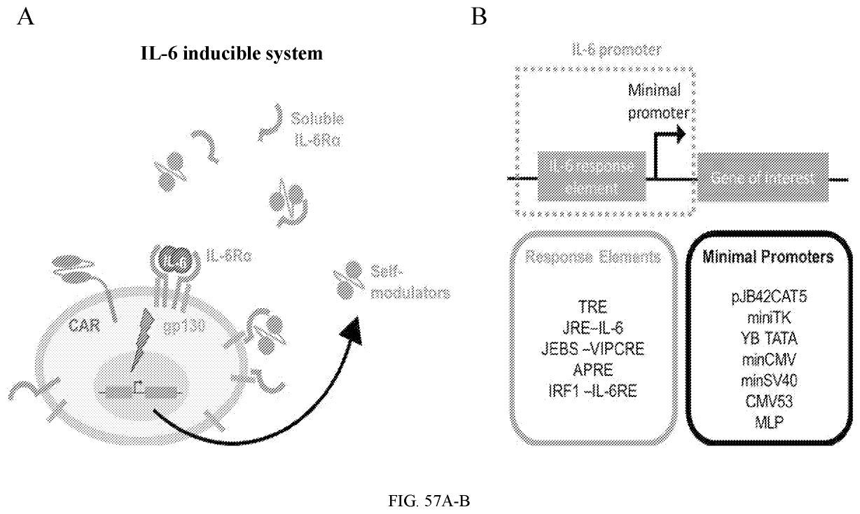

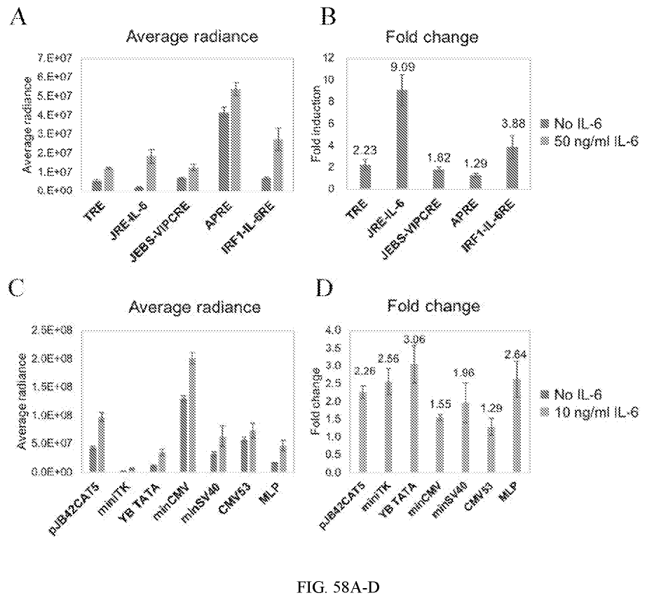

A basic inducible promoter is comprised of one or more response elements (REs) coupled to a minimal promoter (FIG. 57A). To develop an IL-6 inducible promoter, the inventors first evaluated a series of IL-6 REs (TRE (25), JRE-IL-6 (26), JEBS-VIPCRE (26), APRE (27), and IRF1-IL-6RE (28)) and minimal promoters (pJB42CAT5 (29), miniTK (30), YB TATA (31), minCMV (32), minSV40 (33), CMV53 (34) and MLP (35)) (FIG. 57B). Gaussia luciferase (Gluc) was used as an output to quantify gene expression upon IL-6 induction. The panel of IL-6 response elements was first tested by coupling different response elements with miniTK as the minimal promoter. HEK293T cells were transfected with the panel of different IL-6 reporter constructs and induced with 0 or 50 ng/ml IL-6 at approximately 16 hours post transfection. Expression of Gluc was measured after 24 hours of incubation with IL-6. Results from this assay indicated that APRE yielded the highest expression level whereas JRE-IL-6 gave the largest fold change (FIG. 58A, B).

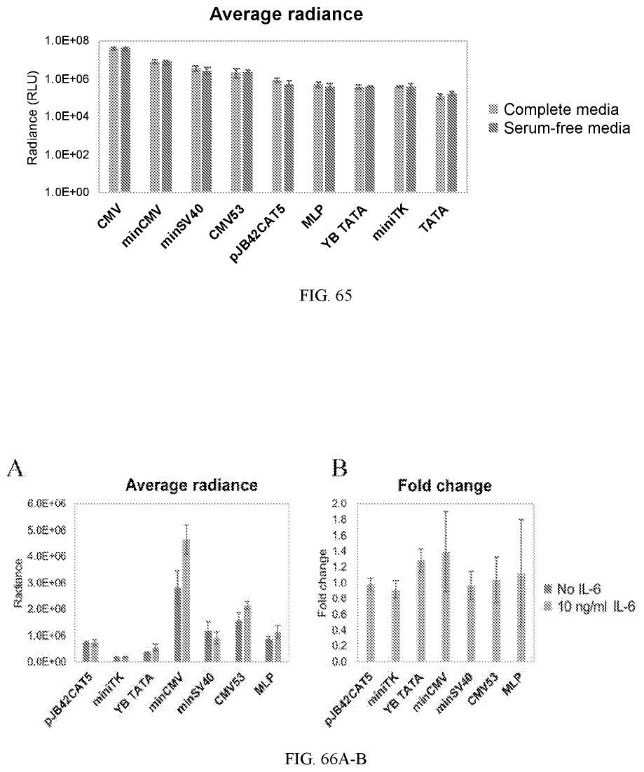

Next, a panel of minimal promoters was characterized for their background expression levels. Minimal promoters driving the expression of Gluc were transfected into HEK293T cells cultured in complete or serum-free media. Gluc activity measurements indicated the minimal promoters' transcriptional activity were not affected by serum, and the panel of minimal promoters provided a range of basal gene expression levels (FIG. 65).

To test the inducibility of different minimal promoters, the inventors coupled JRE-IL-6 with different minimal promoters and transfected the panel of constructs into HEK293T cells. Gluc expression levels and fold changes were evaluated 24 hours after treatment with 0 or 10 ng/ml of IL-6. A lower concentration of IL-6 was used here in order to test the efficacy of IL-6 inducible promoters in a more physiologically relevant concentration of IL-6. Consistent with the result in FIG. 65, minCMV showed the highest expression level among all the minimal promoters tested. However, YB TATA promoter showed the highest fold induction, and the relative performance of all the minimal promoters was consistent with published results when minimal promoters were coupled with hypoxia response elements(36) (FIG. 58C, D). Therefore, the inventors concluded that JRE-IL-6 coupled with YB TATA is the most effective combination in HEK293T cells.

2. IL-6-Inducible Promoters Induce Limited Fold-Induction in Jurkat Cells and Primary Human T Cells

Although the combination of YB TATA and JRE-IL-6 yielded the highest fold change in HEK293T cells, the ultimate goal of this project is to utilize the IL-6 inducible promoter in adoptive T-cell therapy, which uses T cells to produce the output. Thus, the panel of different IL-6 inducible promoters was transfected into Jurkat cells, which is a human T-cell line that is more relevant to the proposed application. However, no significant induction was observed in Jurkat cells and the expression levels were two orders of magnitude lower compared to HEK293T cells (FIG. 66). Possible reasons for low induction and expression levels may be poor transfection efficiency and cell damage caused by the electroporation process used to transfect Jurkat cells.

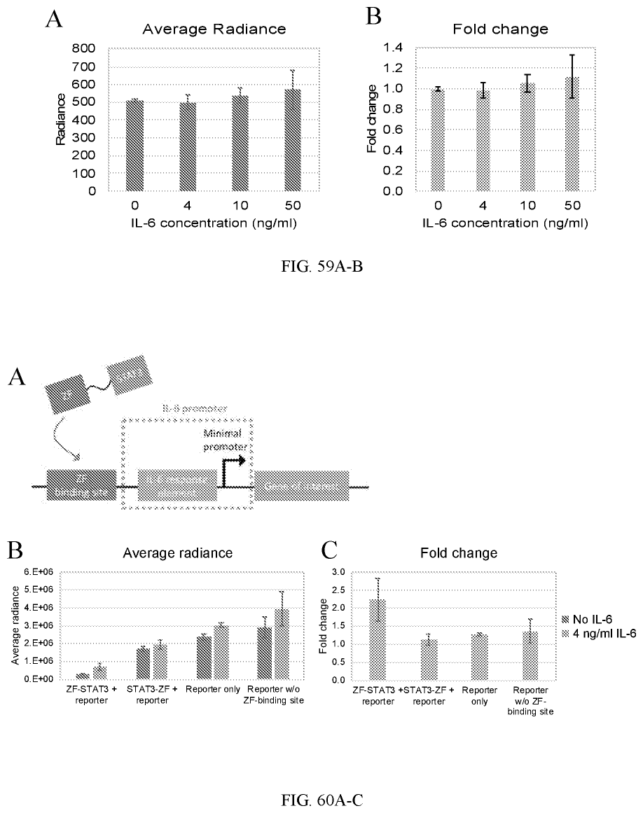

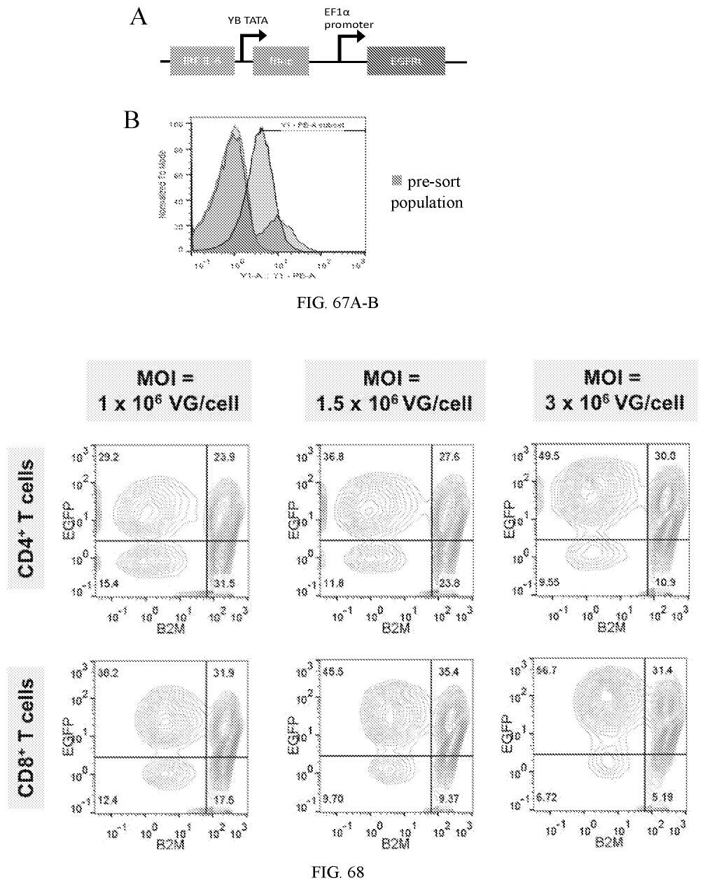

To increase the efficiency of construct integration and test IL-6-inducible promoter in a more relevant platform, lentivirus encoding IL-6 inducible promoter was used to transduce primary human CD4⁺ T cells. IL-6-inducible promoter containing JRE-IL-6 coupled to YB TATA driving the expression of firefly luciferase (ffluc) was chosen based on the expectation that the JRE-IL-6 and YB TATA combination would yield the largest fold change upon IL-6 induction. A constitutive EF1α promoter driving the expression of a truncated epidermal growth factor receptor (EGFRt) was included in the same viral expression vector as the inducible ffLuc expression cassette to allow for the detection and enrichment of transduced cells (FIG. 67A). Primary human T cells were successfully transduced with IL-6-inducible promoter construct albeit with relatively low efficiency, and transduced cells were enriched by magnetic bead-based sorting based on EGFRt expression before IL-6-induction experiment (FIG. 67B). Enriched T cells were induced with 0 to 50 ng/ml of IL-6 for 24 hours before quantifying for ffLuc luminescence. Despite the pure population of transduced T cells used in this experiment, limited inducibility was observed from primary human T cells (FIG. 59). Given the lower gene expression levels of Jurkat cells and primary human T cells relative to HEK293T cells, further optimization is necessary before implementing the IL-6-inducible promoter system in primary T-cell culture.

3. Incorporation of ZF-STAT3 into IL-6-Inducible Promoter Slightly Increases Fold Induction but Lowers Expression Level

One potential cause for the limited inducibility observed in these studies described above was competition between endogenous and transgenic IL-6 response elements for transcription factor binding. To optimize the IL-6-inducible promoter system, the inventors sought to increase available transcription factors by incorporating a zinc finger (ZF)-STAT3 construct that preferentially binds to the transgenic IL-6 response elements. Six ZF binding sites were incorporated upstream of the IL-6-inducible promoter composed of JRE-IL-6 coupled to YB TATA. STAT3, one of the major transcription activators involved in IL-6 signaling, was connected to the ZF via a (G₄S)₄ linker on its N- or C-terminus. The inventors anticipated the transgenic ZF-STAT3 to become phosphorylated upon IL-6 signaling and subsequently translocate to the nucleus, where it would preferentially act on the IL-6 RE on the inducible promoter due to specific recognition between the ZF and the ZF binding site located upstream of the inducible promoter FIG. 60A).

HEK293T cells were transiently transfected with ZF-STAT3 constructs and IL-6 reporters with or without ZF binding sites. Transfected cells were then induced with 4 ng/ml IL-6 for 24 hours before culture supernatant was collected for Gaussia luciferase assay. When ZF-STAT3 (ZF connected to the N-terminus of STAT3) was introduced in the cells, decreased Gluc expression and marginally increased fold-change were observed. One hypothesis for decreased expression level is that unphosphorylated ZF-STAT3 bound to the ZF-binding sites may prevent binding by phosphorylated STAT3 or phosphorylated ZF-STAT3, thereby preventing promoter activation. Interestingly, incorporation of STAT3-ZF (ZF connected to the C-terminus of STAT3) showed no induction upon IL-6 addition, which may be due to improper protein folding. Despite the slightly increased fold-change, the ZF-STAT3 strategy was unlikely to be of great utility in primary human T cells due to its low expression level. Therefore, the inventors chose to examine alternative inducible systems for applications in T-cell therapy.

4. sToci Produced from NFAT-Inducible Promoter Down-Regulates IL-2 and TNF-α Production from Jurkat Cells

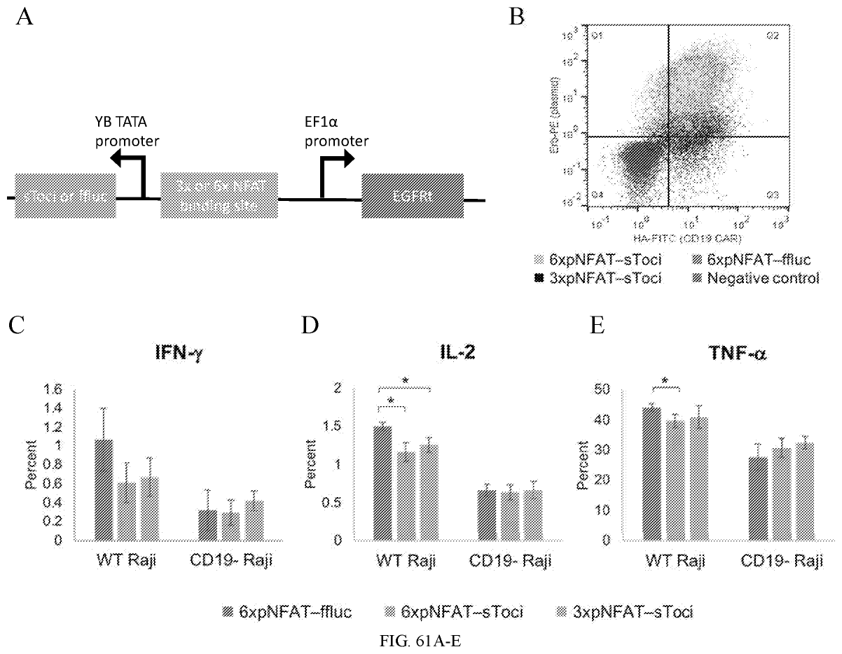

Since T-cell activation is the trigger of CRS, expressing self-modulators upon T-cell activation may be a strategy to prevent CRS at an earlier stage than responding to IL-6 production. NFAT signaling is a key event triggered by T-cell activation (43). NFAT-inducible promoters containing different copies numbers of an NFAT binding site was used to drive the expression of a self-modulator (single-chain variant of tocilizumab, sToci) or ffluc. Constitutively expressed EGFRt was included in the same construct to enable identification of transfected or transduced cells (FIG. 61A). Jurkat cells stably integrated with CD19 CAR were transduced with lentivirus encoding different NFAT reporter constructs, and all the constructs were successfully expressed in Jurkat cells (FIG. 61B).

Transduced Jurkat cells were co-incubated with wildtype or CD19 knock-out Raji cells for 24 hours before analyzing for cytokine expression. Although the difference in IFN-γ expression is statistically insignificant (FIG. 61C), expression of sToci from NFAT-inducible promoters was able to significantly decrease IL-2 and TNF-α production by Jurkat cells (FIGS. 61D, and 61E). With these promising result from Jurkat cells, the inventors next moved on to test NFAT reporter constructs in primary human T cells.

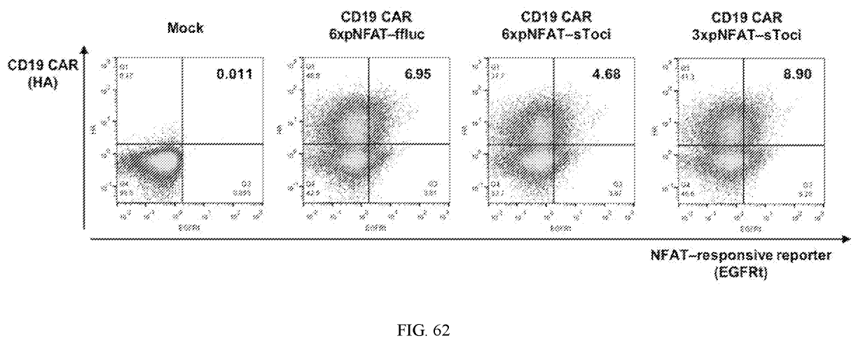

Primary human T cells were transduced with two separate lentiviruses encoding (a) NFAT reporter constructs driving the expression of sToci or ffluc on day 1 and (b) CD19 CAR on day 3. Transduction efficiencies were evaluated on day 9, and less than 10% of the T cells expressed both CD19 CAR and NFAT reporter constructs (FIG. 62). T cells being insufficiently activated on day 1 may have been a reason for poor transduction efficiency of the NFAT reporter construct. In one attempt, cells were able to be enriched twice for CD19 CAR and NFAT reporter expression but were unable to respond to target cells, potentially due to early exhaustion caused by excessive stress caused by cell sorting. After multiple attempts, the inventors concluded that the dual-transduction and enrichment processes cause too much stress on primary human T cells, preventing us from properly evaluating the NFAT reporter strategy. To test the NFAT reporter system, optimization in lentivirus production, transduction, and cell enrichment process will be needed in order to generate healthy primary human T cells for experiments.

5. Alternative Design to Inhibit NFAT Signaling by IL-6-Inducible Production of NFAT Inhibitory Peptides

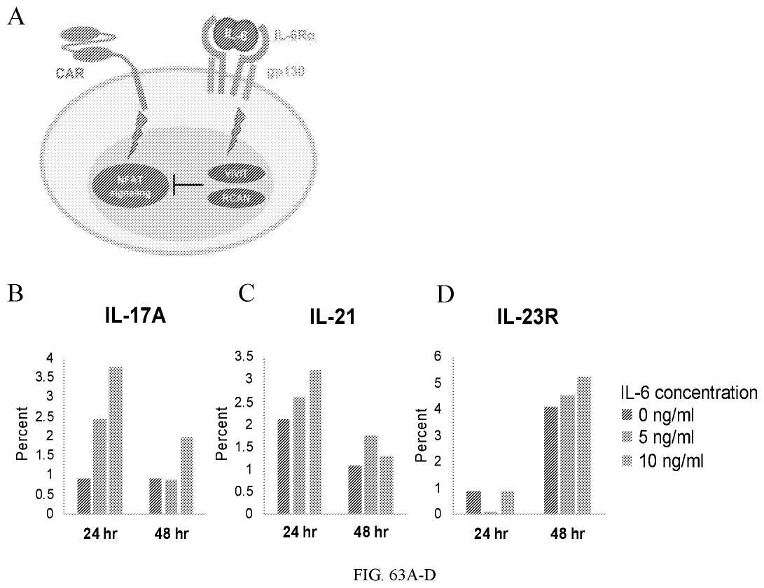

Given the setbacks described above in exploring synthetic transcriptional regulatory systems, an alternative strategy utilizing endogenous IL-6-inducible gene was proposed. IL-6 is considered a key cytokine in CRS, and the inhibition of IL-6 by tocilizumab has been shown to be effective in many patients (15). However, IL-6 itself is unlikely to trigger the full level of toxicity associated with CRS, and the precisely mechanism by which IL-6 signaling inhibition resolves CRS symptoms remains unknown. Nevertheless, given IL-6's apparent role as a key regulator of CRS, the inventors consider the possibility that other cytokines whose production are regulated by IL-6 may contribute to CRS, and that replacing such cytokines with peptides that inhibit NFAT signaling may achieve the dual-purpose of simultaneously lowering the production of harmful cytokines and tampering T-cell activation (FIG. 63A). Specifically, the inventors aimed to identify and subsequently knockout IL-6-induced genes by CRISPR/Cas9-mediated gene editing, followed by homology-directed repair (HDR)-mediated site-specific insertion of VIVIT and RCAN peptides, which have been shown to inhibit NFAT signaling and dampen T-cell activation (21-24).

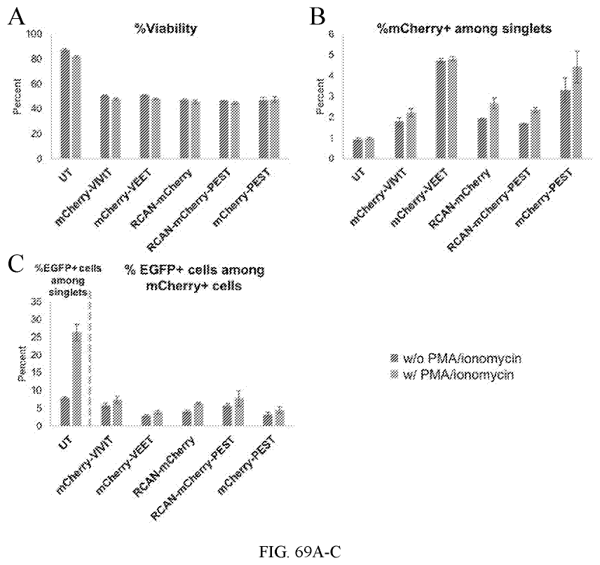

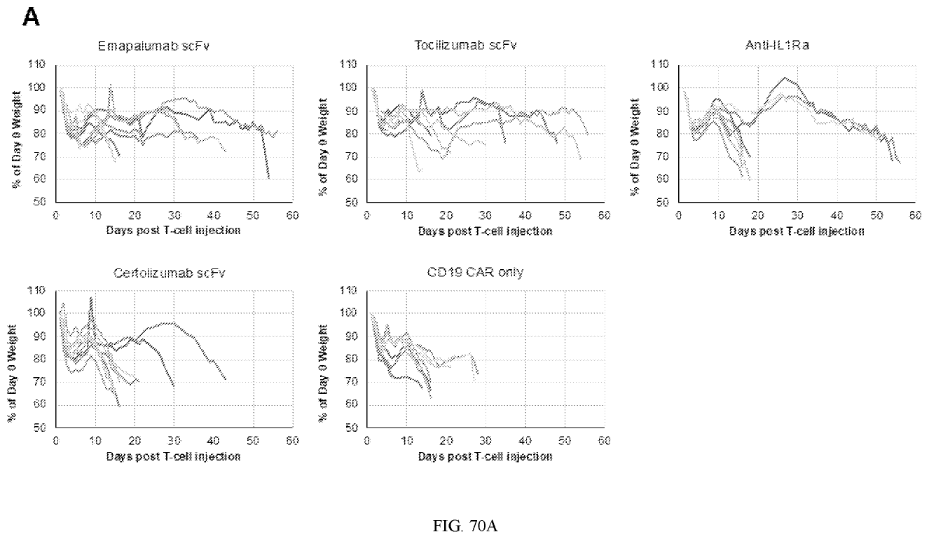

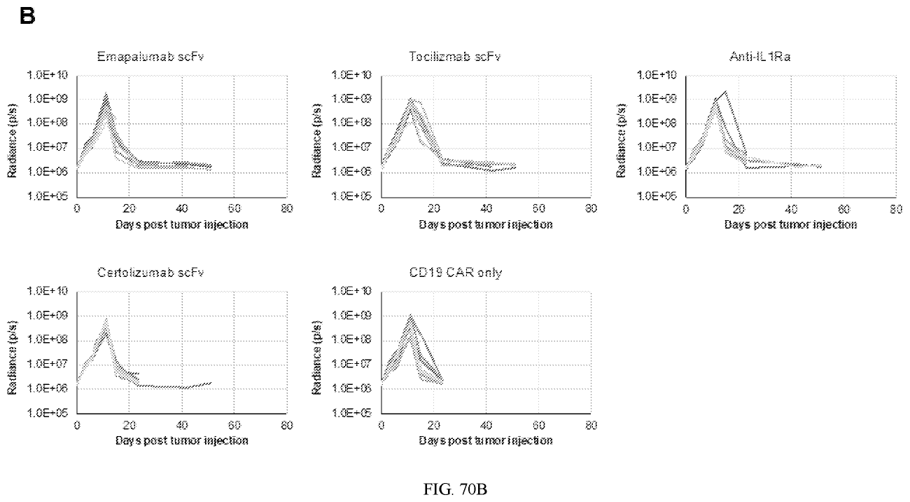

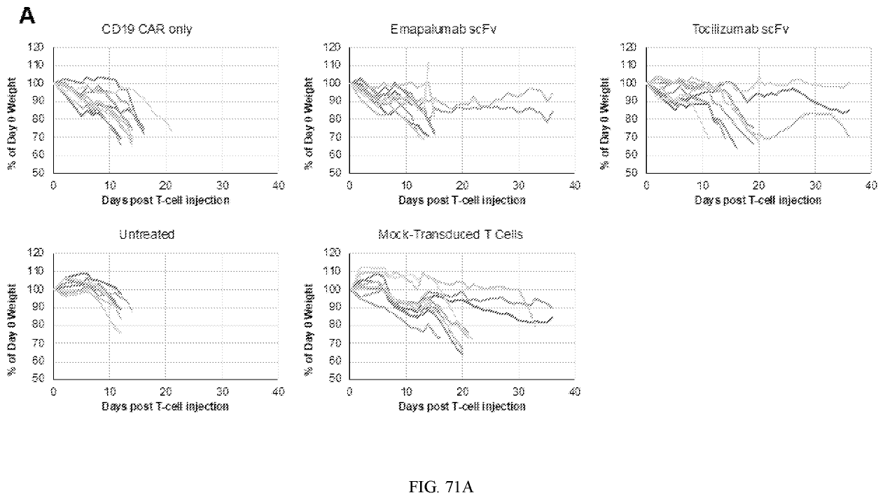

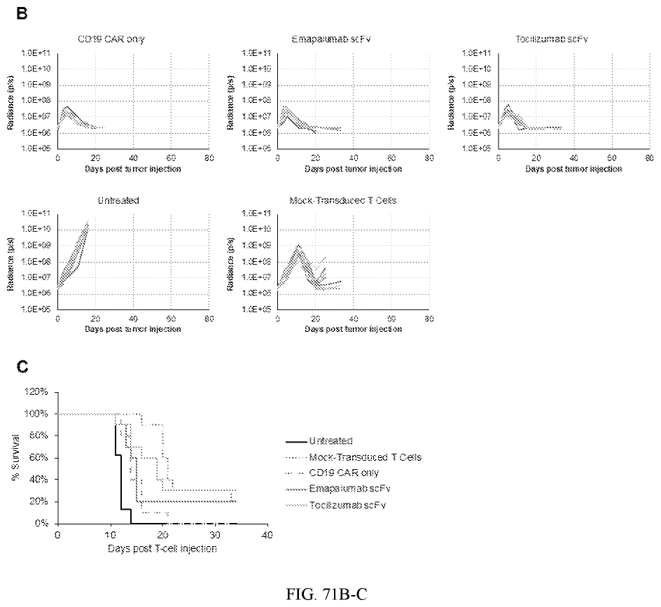

To establish basic feasibility of the CRISPR-HDR protocol in primary T cells, the inventors first designed single-guide RNA (sgRNA) and HDR template to edit the B2M locus that would be used as positive control for constitutive expression. Three days after stimulation, T cells were electroporated with the Cas9/sgRNA ribonucleoprotein (RNP) to knock out B2M gene and transduced with adeno-associated virus (AAV) encoding EGFP flanked by homology arms of B2M locus for HDR. Editing efficiency was checked three days after electroporation, confirming that B2M was efficiently knocked out in both CD4⁺ and CD8⁺ T cells. Furthermore, EGFP expression by edited cells increased with increasing dose of AAV added, confirming proper functions of the sgRNA and HDR template designed (FIG. 68).

The inventors next set out to identify endogenous genes that are induced by IL-6 signaling to serve as potential targets for editing. Three genes—IL-17A (37), IL-21 (38) and IL-23R (39)—that have been reported to be IL-6-responsive were tested by stimulating primary human T cells with 0 to 10 ng/ml of IL-6 for 24 and 48 hours. Expression of IL-17A and IL-21 were evaluated by intracellular staining, and expression of IL-23R was evaluated by surface staining on stimulated T cells. Dose-dependent up-regulation of IL-17A and IL-21 expression were observed at 24 hours. Expression of IL-23R was also observed to be up-regulated in a dose-dependent manner 48 hours after stimulation (FIG. 63B-D). Although up-regulated gene expression was observed, all three genes were expressed at very low levels. The IL-6-inducible genes tested in this experiment have been reported to be mostly produced by Th17 cells, which only represents a small population in the peripheral blood (about 1.2 cells per mm³) (40) and may be a reason for the low expression level observed in the experiment, which used bulk-sorted CD8⁺ T cells. It remains to be determined whether such a low gene expression level would be sufficient to drive an NFAT-signaling modulation system.

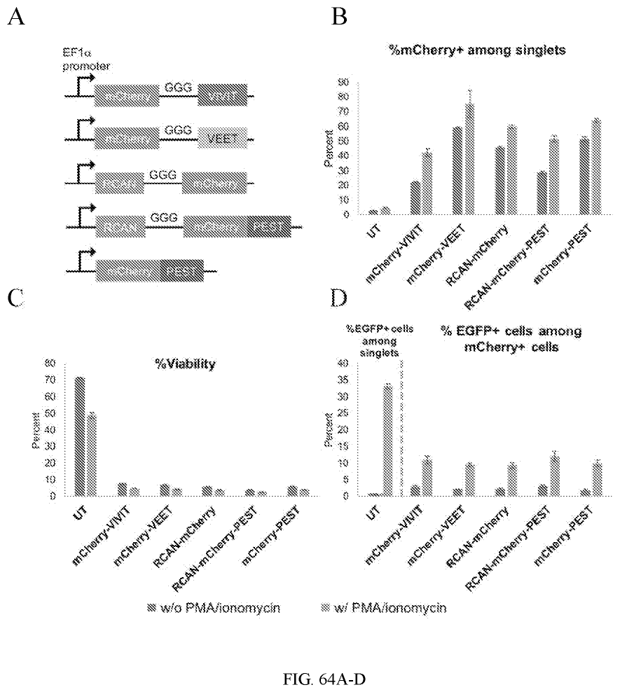

In addition to the identification of IL-6-inducible genes, the efficacy of NFAT-inhibiting peptides (VIVIT and RCAN) were tested in parallel. A panel of VIVIT and RCAN peptides were cloned (FIG. 64A). To allow identification of successfully incorporated construct and maintain the functionality of peptides, mCherry was cloned onto the N-terminus of VIVIT peptide and C-terminus of RCAN peptide. An important consideration in the self-modulator system is that the CRS control mechanism should not negate the therapeutic efficacy of engineered T cells. Therefore, the inventors considered the potential need to limit the half-life of inhibitors of NFAT signaling, so as to prevent undesirable inactivation of T cells. To that end, a PEST sequence was attached to the C-terminus of the RCAN-mCherry construct and compared against un-tagged RCAN-mCherry. Mutated VIVIT peptide (VEET) and mCherry-PEST constructs were included as negative controls.

For the preliminary test, all constructs were expressed under a constitutive EF1α promoter. Constructs were transiently transfected into NFAT reporter Jurkat cells driving the expression of EGFP via electroporation. Twenty-four hours after electroporation, NFAT signaling was induced by the addition of 10 ng/ml PMA and 2 mM ionomycin. Cells were analyzed by flow cytometry for mCherry and EGFP expression 24 hours after PMA and ionomycin addition. All cells showed noticeable mCherry expression, indicating proper transfection efficiency (FIG. 64B). However, cell viability was very low in all electroporated samples (FIG. 64C). In addition, all cells responded to PMA and ionomycin stimulation at a significantly lower degree compared to untransduced cells, suggesting the electroporation process may have damaged the cells and led to suboptimal response towards stimulation (FIG. 64D). No difference in the NFAT-driven EGFP output was observed across samples electroporated with VIVIT, RCAN, or negative-control constructs (FIG. 64D), but it remained unclear whether this was due to a true lack of activity by the peptides or the masking of any activity by the poor health of the electroporated cells. Clearly, alternative strategies for transfection were needed to obtain healthier cells for experimentation.

First, the inventors halved the DNA input in the electroporation process to lower toxicity. Indeed, the viability of transfected Jurkat cells was improved from 10% to ˜40% (FIG. 69A). However, less than 5% of the cells were transfected when the inventors halved the DNA dose, making it difficult to evaluate the efficacy of NFAT-inhibiting peptides (FIG. 69B). Despite the improved cell viability, no up-regulation of EGFP was observed in transfected cells upon PMA and ionomycin stimulation (FIG. 69C), which further indicates that electroporation may have damaged NFAT reporter Jurkat cells in a way that made them incapable of responding to PMA and ionomycin stimulation.

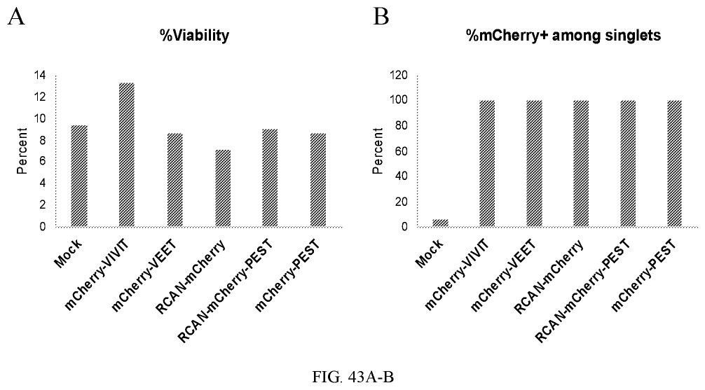

To avoid electroporation as the means of gene transfer, lentiviral constructs encoding the panel of VIVIT and RCAN peptides were generated. Jurkat cells were transduced with lentiviral supernatants by spinfection at 800×g for 90 minutes followed by media change to complete RPMI. Twenty hours after transduction, transduction efficiency and viability of cells were evaluated by flow cytometry. All constructs transduced well but the viability of cells remained very low (FIG. 43). Taken together, the transfection and transduction results suggest the possibility that the VIVIT and RCAN peptides may be inherently toxic to the cells. However, the inventors noted that even cells transfected or transduced with the mCherry-PEST negative-control construct—which should not be toxic-showed low viability. An alternative possibility is that this specific NFAT reporter Jurkat cell line is very sensitive to different culture media and the spinfection in viral supernatant caused irreversible damage to the cells. To further troubleshoot the process and improve the health status of cells, one can potentially try concentrated lentivirus but this study did not explore this possibility.

B. Discussion

In this study, multiple inducible systems were tested to achieve better control of the timing of intervention that could dampen T-cell activation, with the goal of preventing CRS caused by CAR-T cell therapy. First, inducible promoters that respond to IL-6 signaling were developed by testing the combination of different IL-6 REs and minimal promoters. The combination of JRE-IL-6 as RE and YB TATA as minimal promoter showed the highest fold induction upon IL-6 addition in HEK293T cells. However, even this optimized promoter could not achieve robust gene expression or fold-induction or expression levels in primary human T cells. The incorporation of a synthetic transcription activator, ZF-STAT3, was unable to increase gene expression level and fold-induction even in HEK293T cells. Due to the low expression level in primary human T cells, self-modulators expressed by IL-6-inducible promoter may have limited efficacy.

Next, the inventors evaluated the possibility of controlling the expression of self-modulators under an NFAT-inducible promoter, which would be induced upon T-cell activation. In Jurkat cells, an immortalized T-cell line inducibly expressing the self-modulator sToci under the control of an NFAT-inducible promoter was able to down-regulate IL-2 and TNF-α expression upon antigen stimulation. This result demonstrates the possibility of controlling the timing to express self-modulators. However, the inventors were unable to efficiently co-express a CAR and the NFAT-inducible promoter driving the expression of self-modulators in primary human T cells without causing apparent damage to the cells that prevented them from responding to antigen stimulation. An improved manufacturing protocol that enables higher transduction efficiency and less harmful enrichment process will be necessary for this strategy to be useful in primary human T cells. In fact, multiple approaches have been developed in Chen lab after the initial attempt of this strategy with the use of lentivirus. For example, one could use retrovirus to achieve higher transduction efficiency or use the cubi-CAR approach mentioned in Example 2 to enable less stressful enrichment of transduced T cells.

Lastly, a strategy that aimed to utilize endogenous IL-6-responsive genes to drive the expression of NFAT-inhibiting peptides were tested. To ensure proficiency in CRISPR-HDR process, a constitutively expressed gene, B2M, was successfully edited to drive the expression of EGFP. Then the expression levels and fold-induction of three different IL-6-responsive genes (IL-17A, IL-21, and IL-23R) were evaluated in primary human T cells by inducing the expression with different concentrations of IL-6. Although the expression levels of all three genes were up-regulated upon IL-6 addition, the absolute expression levels may be too low to make an impact on mediating CRS. Besides, the efficacy of NFAT-inhibiting peptides (VIVIT and RCAN) were unable to be evaluated in Jurkat cells due to strong toxicity to cells. To further troubleshoot this strategy, alternative IL-6-responsive genes or inducible genes that respond to T-cell activation may be tested. In addition, it is unclear whether the toxicity of NFAT-inhibiting peptides on Jurkat cells came from the electroporation, lentiviral supernatant, or the peptides themselves. Thus, alternative ways for gene integration such as concentrated lentivirus, retrovirus or AAV can be tested.

Here, the inventors evaluated multiple strategies to control the expression of self-modulators at the transcriptional level. Although further improvement such as optimized virus production or improved cell enrichment method can be tested on proposed strategies, the possibility of using different strategies that regulate genes at different levels (such as RNA level or protein level) can also be considered as future plans. It should be noted that inducible expression systems often experience a tradeoff between absolute gene-expression levels and fold-induction ratios—i.e., systems with high fold-induction often have very low basal expression but also relatively low ON-state expression while systems with high absolute expression in the ON state often also have high basal expression and thus low fold-induction. Such a trade-off may ultimately limit the efficacy of self-modulator systems regulated by inducible gene-expression control. In this case, alternative outputs such as NFAT-inhibiting peptides which tamper T-cell activation at an earlier stage may be useful. However, further optimization is still needed to implement NFAT-inhibiting strategy in this system.

C. Methods

1. Plasmid Construction

Sequences of different IL-6 response elements were obtained from previously cited references in the text. The sequence of minCMV promoter was obtained from p5HRE/GFP³² (Addgene); minSV40 was obtained from pGL3-Promoter (Promega); MLP was obtained from pGL4.31 (Promega) and miniTK was obtained from pGluc mini-TK2 (New England Biolabs). Sequences of remaining minimal promoters were obtained from references cited in the text. Sequenced of IL-6 response elements and minimal promoters are listed in Supplementary Table 5.S1 and 5.S2. IL-6-inducible promoter constructs were cloned into pcDNA3.1 (+) backbone (Invitrogen) by standard molecular cloning methods. For lentivirus production, IL-6-inducible promoter with JREIL-6 and YB TATA driving the expression of ffluc was cloned onto epHIV7 vector (41, 42) via standard molecular cloning methods. Sequence of ZF, STAT3 and components on NFAT-inducible promoter were obtained from existing plasmids in Chen lab and their sequences are listed in Supplementary Table 5.S3 and 5.S4. Sequence of NFAT-inhibiting peptides (VIVIT, VEET and RCAN) (Supplementary Table 5.S5) were obtained from previously cited references, synthesized as oligos (Integrated DNA) and cloned into the epHIV7 vector via isothermal DNA assembly. The inventors designed guide RNA to target the first exon of B2M gene (sequence: AGGGUAGGAGAGACUCACGC) and gRNA was synthesized by Synthego. Sequences of B2M gene as homology arms for HDR template were obtained by conducting PCR reactions on genomic DNA isolated from HEK293T cells. Pieces of homology arms and EGFP were cloned onto AAV vector vis isothermal DNA assembly.

2. Cell Line Maintenance

Human embryonic Kidney (HEK)293T, wild-type Raji and Jurkat clone E6-1 cells were obtained from ATCC. EGFP NFAT reporter Jurkat cells were a gift from Dr. Arthur Weiss (University of California, San Francisco). TM-LCL cells were a gift from Dr. Michael Jensen (Seattle Children's Research Institute). Jurkat cells encoded with CD19 CAR were generated in house by transducing parental Jurkat cells with lentivirus followed by magnetic bead-based sorting to enrich the transduced population. HEK293T cells were cultured in DMEM (HyClone) supplemented with 10% heat-inactivated FBS (HI-FBS, Gibco). Wild-type Raji, TM-LCL and Jurkat cells (clone E6-1, EGFP NFAT reporter line and CD19 CAR-expressing line) were cultured in RPMI+10% HI-FBS. All cell lines were regularly tested for Mycoplasma contamination and verified as Mycoplasma free before each experiment.

3. Primary Human T-Cell Generation

Primary human T cells were isolated from healthy donor blood obtained from UCLA Blood and Platelet Center using antibody cocktails (RosetteSep kits, Stemcell Technologies) followed by Ficoll-Paque (GE Healthcare Life Sciences) density-gradient separation. Isolated cells were stimulated with CD3/CD28 Dynabeads (Life Technologies) at a 1:1 bead:cell ratio. Two days after Dynabeads stimulation, cells were transduced with lentivirus (MOI=3). Dynabeads were removed on day 9 or 10. Transduced cells were sorted by magnetic bead-based sorting (Miltenyi Biotec, San Diego, Calif., USA) and expanded in the presence of irradiated TM-LCL feeder cells (T cell:TM-LCL=1:7). All T cells were cultured in RPMI-1640 supplemented with 10% HI-FBS and fed with 50 Um′ IL-2 (Life Technologies) and 1 ng/ml IL-15 (Miltenyi Blotec) every two days. Penicillin-streptomycin (Life Technologies) was supplemented once at 100 U/ml final concentration to T-cell expansion cultures at the time of TM-LCL addition.

4. Cell Transfection and Electroporation

HEK293T cells were seeded at 2.5×10⁴ cells/well in 48-well plate 24-hours before transient transfection. On the day of transfection, 250 ng of total plasmid DNA were transfected using polyethyleneimine (PEI) (Polysciences). Jurkat cells were transiently transfected by using Ingenio electroporation solution (Mirus) on Amaxa Nucleofector 2b device (Lonza), with 5 μg of plasmid DNA and 5×10⁶ cells per electroporation reaction following the manufacturer's protocol.

5. Lentivirus Production

HEK293T cells were seeded in 10 cm² dishes and transfected with lentiviral packaging constructs and expression vector using polyethyleneimine (PEI) (Polysciences). Viral supernatant was harvested 48 and 72 h after transfection. Concentrated lentivirus was generated by PEG-8000 (Bioexpress) treatment and ultracentrifugation and stored at −80° C.

6. AAV Production

HEK293T cells were seeded at 3×10⁶ cells in 10 cm² dish and transfected with AAV packaging constructs and desired construct using polyethyleneimine (PEI) (Polysciences). 72-hours after transfection, cells were harvested and lysed by freeze-thaw method in AAV lysis buffer. Concentrated AAV was generated by iodixanol (StemCell) treatment and ultracentrifugation followed by buffer-exchange with amicon column (Millipore) and stored at 4° C.

7. Gaussia Luciferase Assay

HEK293T cells and Jurkat cells were transiently transfected and incubated at 37° C. for 24 hours before overnight stimulation of different concentrations of IL-6 followed by supernatant collection for assaying. Triplicates were prepared each test condition by seeding HEK293T cells at separate wells before transfection or splitting Jurkat cells at separate wells after electroporation. Luciferase activity was measured by using BioLux Gaussia Luciferase Kit (New England Biolabs) in combination with IVIS Lumina III LT Imaging System (PerkinElmer) or Modulus single-tube luminometer (Turner Biosystems). Background luminescence from mock-transfected cells was subtracted from the raw luminescence values. Fold-induction was calculated by dividing the background-subtracted luminescence signal of induced sample by the average of uninduced samples.

8. Firefly Luciferase Assay

Primary human T cells transduced with IL-6-reporter construct (JRE-IL-6 as response element, YB TATA as minimal promoter) were seeded at 2×10⁵ cells/well in 48-well plate followed by stimulation of different concentrations of IL-6 and incubated at 37° C. for 24 hours. Cells were collected and lysed with culture lysis reagent made in-house (25 mM Tris-HCL, 2 mM dithiothreitol, 2 mM EDTA, 10% glycerol and 1% triton X-100 in MilliQ water) after 24-hour incubation. Lysate was separated from cell debris by a 2-minutes, 12000×g centrifugation. Luciferase activity was measured by mixing 20 μl of lysate with 50 μl of luciferase assay working solution (made in-house: 100 mM Tris-HCL, 5 mM MgCl₂, 250 μM Coenzyme A, 150 μM ATP and 150 μg/ml D-luciferin (Gold Biotech)) and incubated for 10 minutes before luminescence measurement on Modulus single-tube luminometer (Turner Biosystems). Background luminescence from mock-transfected cells was subtracted from the raw luminescence values. Fold-induction was calculated by dividing the background-subtracted luminescence signal of induced sample by the average of uninduced samples.

9. Intracellular Staining

For cytokine production assessment in Jurkat cells containing NFAT-responsive promoter, Jurkat cells with CD19 CAR expression were transduced with lentivirus encoding different NFAT-reporter constructs. After the transduction efficiency has been confirmed, cells were seeded at 1×10⁵ cells/well and co-cultured with target cells at 1 to 1 effector-to-target ratio in the presence of 5 μg/ml Brefeldin A (Biolegend) and 2 μM of Monensin (Biolegend). After overnight incubation, cells were harvested and fixed with 1.5% formaldehyde; permeabilized with ice-cold methanol and stained with anti-IL-2, anti-TNF-α, and anti-IFN-7 antibodies (all from Biolegend). Single-cell cytokine expression was measured on flow cytometer (Miltenyi Biotec).

For the assessment of IL-21 and IL-17A expression, primary human T cells were seeded at 3×10⁵ cells/well in 48-well plate and stimulated with different concentrations of IL-6 for 24 or 48 hours in the presence of 5 μg/ml Brefeldin A (Biolegend) and 2 μM of Monensin (Biolegend). After incubation, cells were harvested and stained as indicated in the previous paragraph but with anti-IL-21 and anti-IL-17A antibodies (all from Biolegend).

10. CRISPR-HDR

Three days after initial stimulation, CD3/CD28 dynabeads were removed from primary human T cells. For each reaction, 5×10⁶ T cells were electroporated with ribonucleoprotein (RNP) complexing mixture (300 pmol of sgRNA mixed with 300 pmol of Cas9 protein) by using Ingenio electroporation solution (Mirus) on Amaxa Nucleofector 2b device (Lonza). Electroporated T cells were seeded into five separate wells in 48-well plate with 500 μl of complete RPMI per well. After a brief recovery, different doses of AAV were added into T-cell culture and incubated for at least two days before analysis on flow cytometer.

11. NFAT Reporter Assays

For NFAT-inhibit peptides testing, 5×10⁶ of EGFP NFAT reporter Jurkat cells were electroporated with 5 μg (or 2.5 μg in the halved dose experiment) of plasmid DNA by using Ingenio electroporation solution (Mirus) on Amaxa Nucleofector 2b device (Lonza). Transfection efficiency was assessed 24-hours after electroporation and cells were split into multiple wells for all the test conditions. Designated cells were then subsequently induced with 10 ng/ml PMA and 2 μM of ionomycin and incubated at 37° C. for 12 hours. After incubation, cells were then analyzed on flow cytometer (Miltenyi Biotec).

12. Statistical Analysis

Except for CRISPR-HDR, expression of endogenous IL-6-responsive gene AND qualitative analysis of transduced cells, a minimum of triplicates was chosen to allow calculation of statistics. Statistical significance was analyzed by using two-tailed, unpaired, homoscedastic Student's t-test.

D. Supplementary Tables

SUPPLEMENTARY TABLE 5.S1
DNA sequences of different IL-6 response elements

| | |
|---|---|
| TRE | GTCGACATTTCCCGTAAATCGTCGA |
| | (SEQ ID NO: 132) |
| JRE-IL-6 | GCGCTTCCTGACAGTGACGCGAGCC |
| | G (SEQ ID NO: 133) |
| JEBS- | GCGCTTCCTGACAGTGACGTCTTTG |
| VIPCRE | (SEQ ID NO: 134) |
| APRE | GCTGTACGGTAAAAGTGAGCTCTTA |
| | CGGGAATGGGAAT |
| | (SEQ ID NO: 135) |
| IRF1-IL-6RE | GCGTGCCGTCATTTCGGGGAAATC |
| | (SEQ ID NO: 136) |

SUPPLEMENTARY TABLE 5.S2
DNA sequences of different minimal promoters

| | |
|---|---|
| pJB42CAT5 | CTGACAAATTCAGTATAAAAGCTTGGGGCTGGGGCC |
| | GAGCACTGGGGACTTTGAGGGTGGCCAGGCCAGCGT |
| | AGGAGGCCAGCGTAGGATCCTGCTGGGAGCGGGGAA |
| | CTGAGGGAAGCGACGCCGAGAAAGCAGGCGTACCAC |
| | GGAGGGAGAGAAAAGCTCCGGAAGCCCAGCAGCG |
| | (SEQ ID NO: 137) |
| miniTK | TTCGCATATTAAGGTGACGCGTGTGGCCTCGAACAC |
| | CGAGCGACCCTGCAGCGACCCGCTTAA |
| | (SEQ ID NO: 138) |
| YB TATA | TCTAGAGGGTATATAATGGGGGCCA |
| | (SEQ ID NO: 139) |
| minCMV | ATCTGGTAGGCGTGTACGGTGGGAGGTCTATATAAG |
| | CAGAGCTCGTTTAGTGAACCGTCAGATC |
| | (SEQ ID NO: 140) |
| minSV40 | TGCATCTCAATTAGTCAGCAACCATAGTCCCGCCCC |
| | TAACTCCGCCCATCCCGCCCCTAACTCCGCCCAGTT |
| | CCGCCCATTCTCCGCCCCATCGCTGACTAATTTTTT |
| | TTATTTATGCAGAGGCCGAGGCCGCCTCGGCCTCTG |
| | AGCTATTCCAGAAGTAGTGAGGAGGCTTTTTTGGAG |
| | GCCTAGGCTTTTGCAAAAAGCTT |
| | (SEQ ID NO: 141) |
| CMV53 | CAACAAAATGTCGTAACAAGGGCGGTAGGCGTGTAC |
| | GGTGGGAGGTCTATATAAGCAGAGCTCGTTTAGTGA |
| | ACCG (SEQ ID NO: 142) |
| MLP | GGGGGGCTATAAAAGGGGGTGGGGGCGTTCGTCCTC |
| | ACTCT (SEQ ID NO: 143) |

SUPPLEMENTARY TABLE 5.S3
Amino acid sequences of ZF and STAT3

| | |
|---|---|
| ZF | PRVRTGSKTPPHERPFQCRICMRNFSDSPTLRRHTRTHT |
| | GEKPFQCRICMRNFSQGANLRRHLRTHTGEKPFQCRIMR |
| | NFSQANTLQRHLKTHTGEK (SEQ ID NO: 144) |
| STAT3 | AQWNQLQQLDTRYLEQLHQLYSDSFPMELRQFLAPWIES |
| | QDWAYAASKESHATLVFHNLLGEIDQQYSRFLQESNVLY |
| | QHNLRRIKQFLQSRYLEKPMEIARIVARCLWEESRLLQT |
| | AATAAQQGGQANHPTAAVVTEKQQMLEQHLQDVRKRVQD |
| | LEQKMKVVENLQDDFDFNYKTLKSQGDMQDLNGNNQSVT |
| | RQKMQQLEQMLTALDQMRRSIVSELAGLLSAMEYVQKTL |
| | TDEELADWKRRQQIACIGGPPNICLDRLENWITSLAESQ |
| | LQTRQQIKKLEELQQKVSYKGDPIVQHRPMLEERIVELF |
| | RNLMKSAFVVERQPCMPMHPDRPLVIKTGVQFTTKVRLL |
| | VKFPELNYQLKIKVCIDKDSGDVAALRGSRKFNILGTNT |
| | KVMNMEESNNGSLSAEFKHLTLREQRCGNGGRANCDASL |
| | IVTEELHLITFETEVYHQGLKIDLETHSLPVVVISNICQ |
| | MPNAWASILWYNMLTNNPKNVNFFTKPPIGTWDQVAEVL |
| | SWQFSSTTKRGLSIEQLTTLAEKLLGPGVNYSGCQITWA |
| | KFCKENMAGKGFSFWVWLDNIIDLVKKYILALWNEGYIM |
| | GFISKERERAILSTKPPGTFLLRFSESSKEGGVTFTWVE |
| | KDISGKTQIQSVEPYTKQQLNNMSFAEIIMGYKIMDATN |
| | ILVSPLVYLYPDIPKEEAFGKYCRPESQEHPEADPGSAA |
| | PYLKTKFICVTPTTCSNTIDLPMSPRTLDSLMQFGNNGE |
| | GAEPSAGGQFESLTFDMELTSECATSPM |
| | (SEQ ID NO: 145) |

SUPPLEMENTARY TABLE 5.S4
DNA sequences of NFAT binding site

| | |
|---|---|
| NFAT | GGAGGAAAAACTGTTTCATACAGAAGGCGT |
| binding site | (SEQ ID NO: 146) |

SUPPLEMENTARY TABLE 5.S5
Amino acid sequences of VIVIT, VEET,
and RACN peptides

| | |
|---|---|
| VIVIT | MAGPHPVIVITGPHEE (SEQ ID NO: 147) |
| VEET | MAGPPHIVEETGPHVI (SEQ ID NO: 148) |
| RCAN | KYELHAATDTTPSVVVHVCES (SEQ ID NO: 149) |

E. References

The following references and the publications referred to throughout the specification, to the extent that they provide exemplary procedural or other details supplementary to those set forth herein, are specifically incorporated herein by reference.
1. Elowitz, M. B. & Leibler, S. A synthetic oscillatory network of transcriptional regulators. Nature 403, 335 (2000).
2. Prindle, A. et al. Rapid and tunable post-translational coupling of genetic circuits. Nature 508, 387 (2014).
3. Siuti, P., Yazbek, J. & Lu, T. K. Synthetic circuits integrating logic and memory in living cells. Nat. Biotechnol. 31, 448-452 (2013).
4. Bonnet, J., Subsoontorn, P. & Endy, D. Rewritable digital data storage in live cells via engineered control of recombination directionality. Proc. Natl. Acad. Sci. 109, 8884 LP-8889 (2012).
5. Ajo-Franklin, C. M. et al. Rational design of memory in eukaryotic cells. Genes Dev. 21, 2271-2276 (2007).
6. Friedland, A. E. et al. Synthetic Gene Networks That Count. Science (80-.). 324, 1199 LP-1202 (2009).
7. Auslander, S., Auslander, D., Muller, M., Wieland, M. & Fussenegger, M. Programmable single-cell mammalian biocomputers. Nature 487, 123 (2012).
8. Fire, A. et al. Potent and specific genetic interference by double-stranded RNA in Caenorhabditis elegans. Nature 391, 806 (1998).
9. Ipsaro, J. J. & Joshua-Tor, L. From guide to target: molecular insights into eukaryotic RNA-interference machinery. Nat. Struct. &Amp; Mol. Biol. 22, 20 (2015).
10. Janse, D. M., Crosas, B., Finley, D. & Church, G. M. Localization to the proteasome is sufficient for degradation. J. Biol. Chem. 279, 21415-21420 (2004).
11. Niopek, D. et al. Engineering light-inducible nuclear localization signals for precise spatiotemporal control of protein dynamics in living cells. Nat. Commun. 5, 4404 (2014).
12. Niopek, D., Wehler, P., Roensch, J., Eils, R. & Di Ventura, B. Optogenetic control of nuclear protein export. Nat. Commun. 7, 10624 (2016).
13. Chen, D., Gibson, E. S. & Kennedy, M. J. A light-triggered protein secretion system. J. Cell Biol. 201, 631 LP-640 (2013).
14. Porter, D. L. et al. Chimeric antigen receptor T cells persist and induce sustained remissions in relapsed refractory chronic lymphocytic leukemia. Sci. Transl. Med. 7, 1-12 (2015).
15. Lee, D. W. et al. T cells expressing CD19 chimeric antigen receptors for acute lymphoblastic leukaemia in children and young adults: A phase 1 dose-escalation trial. Lancet 385, 517-528 (2015).
16. Davila, M. L. et al. Efficacy and toxicity management of 19-28z CAR T cell therapy in B cell acute lymphoblastic leukemia. Sci. Transl. Med. 6, 1-13 (2014).
17. Maude, S. L. et al. Chimeric Antigen Receptor T Cells for Sustained Remissions in Leukemia. N. Engl. J. Med. 371, 1507-1517 (2014).
18. Grupp, S. A. et al. Chimeric antigen receptor-modified T cells for acute lymphoid leukemia. N. Engl. J. Med. 368, 1509-18 (2013).
19. Ran, F. A. et al. In vivo genome editing using Staphylococcus aureus Cas9. Nature 520, 186-191 (2015).
20. Long, C. et al. Postnatal genome editing partially restores dystrophin expression in a mouse model of muscular dystrophy. Science (80-.). 351, 400 LP-403 (2016).
21. Noguchi, H. et al. RCAN-11R peptide provides immunosuppression for fully mismatched islet allografts in mice. Sci. Rep. 7, 1-11 (2017).
22. Kuriyama, M. et al. A cell-permeable NFAT inhibitor peptide prevents pressure-overload cardiac hypertrophy. Chem. Biol. Drug Des. 67, 238-243 (2006).
23. Noguchi, H. et al. A new cell-permeable peptide allows successful allogeneic islet transplantation in mice. Nat. Med. 10, 305-309 (2004).
24. Aramburu, J. et al. Affinity-Driven Peptide Selection of an NFAT Inhibitor More Selective Than Cyclosporin A. Science (80-.). 285, 2129-2134 (1999).
25. Feister, H. a, Auerbach, B. J., Cole, L. a, Krause, B. R. & Karathanasis, S. K. Identification of an IL-6 response element in the human LCAT promoter. J. Lipid Res. 43, 960-970 (2002).
26. Nakajima, K. et al. Identification of a novel interleukin-6 response element containing an Ets-binding site and a CRE-like site in the junB promoter. Mol. Cell. Biol. 13, 3027-41 (1993).
27. Wegenka, U. M., Buschmann, J., Lütticken, C., Heinrich, P. C. & Horn, F. Acute-phase response factor, a nuclear factor binding to acute-phase response elements, is rapidly activated by interleukin-6 at the posttranslational level. Mol. Cell. Biol. 13, 276-288 (1993).
28. Ichiba, M., Nakajima, K., Yamanaka, Y., Kiuchi, N. & Hirano, T. Autoregulation of the Stat3 gene through cooperation with a cAMP-responsive element-binding protein. J. Biol. Chem. 273, 6132-6138 (1998).
29. Nakajima, K. et al. Identification of a novel interleukin-6 response element containing an Ets-binding site and a CRE-like site in the junB promoter. Mol. Cell. Biol. 13, 3027-3041 (1993).
30. McKnight, S. L., Gavis, E. R., Kingsbury, R. & Axel, R. Analysis of transcriptional regulatory signals of the HSV thymidine kinase gene: Identification of an upstream control region. Cell 25, 385-398 (1981).
31. Hansen, J. et al. Transplantation of prokaryotic two-component signaling pathways into mammalian cells. Proc. Natl. Acad. Sci. 111, 15705-15710 (2014).
32. Shibata, T., Giaccia, A. J. & Brown, J. M. Development of a hypoxia-responsive vector for tumor-specific gene therapy. Gene Ther. 7, 493 (2000).
33. Byrne, B. J., Davis, M. S., Yamaguchi, J., Bergsma, D. J. & Subramanian, K. N. Definition of the simian virus 40 early promoter region and demonstration of a host range bias in the enhancement effect of the simian virus 40 72-base-pair repeat. Proc. Natl. Acad. Sci. 80, 721-725 (1983).
34. Mahmoud, L. et al. Green Fluorescent Protein Reporter System with Transcriptional Sequence Heterogeneity for Monitoring the Interferon Response. J. Virol. 85, 9268-9275 (2011).
35. Lewis, E. D. & Manley, J. L. Control of adenovirus late promoter expression in two human cell lines. Mol. Cell. Biol. 5, 2433-2442 (1985).
36. Ede, C., Chen, X., Lin, M. Y. & Chen, Y. Y. Quantitative Analyses of Core Promoters Enable Precise Engineering of Regulated Gene Expression in Mammalian Cells. ACS Synth. Biol. 5, 395-404 (2016).
37. Durant, L. et al. Diverse targets of the transcription factor STAT3 contribute to T cell pathogenicity and homeostasis. Immunity 32, 605-615 (2010).
38. Dienz, O. et al. The induction of antibody production by IL-6 is indirectly mediated by IL-21 produced by CD4+ T cells. J. Exp. Med. 206, 69-78 (2009).
39. Lee, P. W. et al. IL-23R-activated STAT3/STAT4 is essential for Find the latest version: IL-23R-activated STAT3/STAT4 is essential for Th1/Th17-mediated CNS autoimmunity. JCI Insight 2, 1-20 (2017).
40. Jain, P. et al. Th17 and non-th17 interleukin-17-expressing cells in chronic lymphocyticleukemia: Delineation, distribution, and clinical relevance. Haematologica 97, 599-607 (2012).
41. Yam, P. Y. et al. Design of HIV Vectors for Efficient Gene Delivery into Human Hematopoietic Cells. Mol. Ther. 5, 479-484 (2002).
42. Wang, X. et al. A transgene-encoded cell surface polypeptide for selection, in vivo tracking, and ablation of engineered cells. Blood 118, 1255-1263 (2011).
43. Macian, F. NFAT proteins: key regulators of T-cell development and function. Nat. Rev. Immunol. 5, 472-484 (2005).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. All references, cited literature articles, patent publications, and sequences associated with any recited GenBank accession numbers are specifically incorporated herein by reference in their entirety for all purposes.

1. A chimeric tumor necrosis factor alpha (TNF-α) binding polypeptide comprising a heavy chain variable region comprising CDR1, CDR2, and CDR3 attached by a heterologous linker to a light chain variable region comprising CDR4, CDR5, and CDR6; wherein the polypeptide comprises
CDR1, CDR2, and CDR3 of the heavy chain variable region of SEQ ID NO:4 and CDR4, CDR5, and CDR6 of the light chain variable region of SEQ ID NO:3;
CDR1, CDR2, and CDR3 of the heavy chain variable region of SEQ ID NO:6 and CDR4, CDR5, and CDR6 of the light chain variable region of SEQ ID NO:5;
CDR1, CDR2, and CDR3 of the heavy chain variable region of SEQ ID NO:8 and CDR4, CDR5, and CDR6 of the light chain variable region of SEQ ID NO:7; or
CDR1, CDR2, and CDR3 of the heavy chain variable region of SEQ ID NO:10 and CDR4, CDR5, and CDR6 of the light chain variable region of SEQ ID NO:9.

2. The TNF-α binding polypeptide of claim 1, wherein the polypeptide comprises:
a CDR1, CDR2, CDR3, CDR4, CDR5, and CDR6 with at least 80% sequence identity to SEQ ID NOS:97-102, respectively;
a CDR1, CDR2, CDR3, CDR4, CDR5, and CDR6 with at least 80% sequence identity to SEQ ID NOS:103-108, respectively;
a CDR1, CDR2, CDR3, CDR4, CDR5, and CDR6 with at least 80% sequence identity to SEQ ID NOS:109-114, respectively; or
a CDR1, CDR2, CDR3, CDR4, CDR5, and CDR6 with at least 80% sequence identity to SEQ ID NOS:115-120, respectively.

3. The TNF-α binding polypeptide of claim 2, wherein the polypeptide comprises:
a CDR1, CDR2, CDR3, CDR4, CDR5, and CDR6 having a sequence corresponding to SEQ ID NOS:97-102, respectively;
a CDR1, CDR2, CDR3, CDR4, CDR5, and CDR6 having a sequence corresponding to SEQ ID NOS:103-108, respectively;
a CDR1, CDR2, CDR3, CDR4, CDR5, and CDR6 having a sequence corresponding to SEQ ID NOS:109-114, respectively; or
a CDR1, CDR2, CDR3, CDR4, CDR5, and CDR6 having a sequence corresponding to SEQ ID NOS:115-120, respectively.

4. A chimeric interferon gamma (IFN-γ) binding polypeptide comprising a heavy chain variable region comprising CDR1, CDR2, and CDR3 attached by a heterologous linker to a light chain variable region comprising CDR4, CDR5, and CDR6; wherein the polypeptide comprises CDR1, CDR2, and CDR3 of the heavy chain variable region of SEQ ID NO:12 and CDR4, CDR5, and CDR6 of the light chain variable region of SEQ ID NO:11.

5. The IFN-γ binding polypeptide of claim 4, wherein the polypeptide comprises: a CDR1, CDR2, CDR3, CDR4, CDR5, and CDR6 with at least 80% sequence identity to SEQ ID NOS:121-126, respectively.

6. The IFN-γ binding polypeptide of claim 5, wherein the polypeptide comprises: a CDR1, CDR2, CDR3, CDR4, CDR5, and CDR6 having a sequence corresponding to SEQ ID NOS: 121-126, respectively;

7. The chimeric binding polypeptide of any one of claims 1-6, wherein the binding polypeptide is an scFv, (scFv)2, scFvFc, Fab, Fab′, F(ab)2, or a single chain antibody.

8. The chimeric binding polypeptide of any one of claims 1-7, wherein the binding polypeptide is one polypeptide.

9. The chimeric binding polypeptide of claim 8, wherein the one polypeptide is a single chain variable fragment (scFv).

10. The chimeric binding polypeptide of any one of claims 1-9, wherein the heavy chain variable region is on the N-terminal side of the light chain variable region.

11. The chimeric binding polypeptide of any one of claims 1-10, wherein the light chain variable region is on the N-terminal side of the heavy chain variable region.

12. The chimeric binding polypeptide of any one of claims 1-11, wherein the linker comprises the amino acid sequence GSTSGSGKPGSGEGSTKG (SEQ ID NO:14).

13. The chimeric binding polypeptide of any one of claims 1-12, further comprising a leader peptide.

14. The chimeric binding polypeptide of any one of claims 1-13, further comprising an isolation tag.

15. The chimeric binding polypeptide of any one of claim 1 or 7-14, wherein the polypeptide comprises:
the heavy chain variable region of SEQ ID NO:4 and light chain variable region of SEQ ID NO:3;
the heavy chain variable region of SEQ ID NO:6 and the light chain variable region of SEQ ID NO:5;
the heavy chain variable region of SEQ ID NO:8 and the light chain variable region of SEQ ID NO:7; or
the heavy chain variable region of SEQ ID NO:10 and the light chain variable region of SEQ ID NO:9.

16. The chimeric binding polypeptide of any one of claims 4-14, wherein the polypeptide comprises the heavy chain variable region of SEQ ID NO:12 and the light chain variable region of SEQ ID NO:11.

17. The chimeric binding polypeptide of any one of claims 1-16, wherein the polypeptide further comprises a chimeric antigen receptor (CAR).

18. The chimeric binding polypeptide of claim 17, wherein the CAR comprises a CD19 and/or CD20 monospecific or bispecific CAR.

19. The chimeric binding polypeptide of claim 17 or 18, wherein the polypeptide further comprises a cleavage site between i) the CAR and ii) the heavy and light chain variable regions of the chimeric binding polypeptide.

20. The chimeric binding polypeptide of claim 19, wherein the cleavage site comprises a self-cleaving 2A polypeptide.

21. A T cell expressing the chimeric binding polypeptide of any of claims 1-20.

22. The T cell of claim 21, wherein the T cell is reduced in expression of TRAC and/or B2M gene products.

23. The T cell of claim 21 or 22, wherein the endogenous TRAC and/or B2M genes are mutated to reduce or eliminate expression of the TRAC and/or B2M gene products.

24. The T cell of any one of claims 21-23, wherein the T cell further comprises a CAR.

25. The T cell of claim 24, wherein the CAR comprises a CD19 CAR.

26. A nucleic acid molecule encoding the chimeric binding polypeptide of any of claims 1-20.

27. The nucleic acid molecule of claim 26, further comprising a promoter controlling expression of the chimeric binding polypeptide.

28. The nucleic acid molecule of claim 27, wherein the promoter is constitutive.

29. The nucleic acid molecule of claim 27, wherein the promoter is inducible.

30. The nucleic acid molecule of claim 29, wherein the inducible promoter comprises an inducible response element and/or a minimal promoter.

31. The nucleic acid molecule of claim 30, wherein the response element comprises one or more of TRE, JRE-IL-6, JEBS-VIPCRE, APRE, and IRF1-IL-6RE.

32. The nucleic acid molecule of claim 30 or 31, wherein the inducible promoter comprises the minimal promoter: pJB42CAT5, miniTK, YB TATA, minCMV, minSV40, CMV53 or MLP.

33. The nucleic acid molecule of claim 29, wherein the inducible promoter comprises an NFAT-inducible promoter.

34. The nucleic acid molecule of claim 27, wherein the promoter responds positively to at least one cytokine or to T-cell activation.

35. The nucleic acid molecule of claim 34, where the at least one cytokine is IL-6, TNF-α, IFN-γ, IL-1β, IL-2, IL-8, IL-1, or IL-10 or the promoter responds positively to NFAT-1 or NF-κB.

36. An expression construct comprising the nucleic acid molecule of any of claims 26-35.

37. The expression construct of claim 28, wherein the expression construct is a viral vector.

38. The expression construct of claim 37, wherein the viral vector comprises a lenti viral vector.

39. The expression construct of claim 28, wherein the expression construct is a plasmid.

40. A recombinant T cell comprising the nucleic acid expression construct of any of claims 36-39.

41. The T cell of claim 40, wherein the expression construct comprises a cytokine responsive promoter or promoter that increases expression when T cells are activated.

42. The T cell of claim 41, wherein the promoter responds positively to one or more of the following: NFAT-1, NF-κB, IL-6, TNF-α, IFN-γ, IL-1β, IL-2, IL-8, IL-1, and IL-10.

43. A method for reducing the risk of cytokine release syndrome comprising administering to a patient at risk for cytokine release syndrome a composition comprising the chimeric binding polypeptide of any one of claims 1-20 or the T cell of any one of claim 21-25 or 40-42.

44. The method of claim 43, wherein the patient has cancer.

45. The method of claim 44, wherein the method further comprises treating the cancer.

46. The method of claim 45, wherein the cancer comprises lymphoma.

47. The method of any one of claims 43-46, wherein the patient has or will receive adoptive T-cell therapy.

48. The method of claim 47, wherein the patient has or will receive lymphodepletion.

49. The method of claim 43, wherein the patient has an autoimmune disease.

50. The method of any of claims 43-49, the method comprises administering T cells, and wherein the T cells are autologous.

51. The method of any of claims 43-50, further comprising administering to the patient an antihistamine, a corticosteroid, a steroid, acetaminophen, furosemide, and/or intravenous fluids.

52. The method of any of claims 43-51, wherein the patient has one or more symptoms of cytokine release syndrome.

53. The method of any one of claims 43-51, wherein the patient does not have symptoms of cytokine release syndrome.


SEQUENCE LISTING (157 sequences)

SEQ ID NO: 1 - protein, 107 aa, Artificial Sequence [sequence omitted]

SEQ ID NO: 2 - protein, 119 aa, Artificial Sequence
  Description of Artificial Sequence Synthetic polypeptide
EVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSS

SEQ ID NO: 3 - protein, 107 aa, Artificial Sequence
  Description of Artificial Sequence Synthetic polypeptide
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 4 - protein, 120 aa, Artificial Sequence
  Description of Artificial Sequence Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSS

SEQ ID NO: 5 - protein, 107 aa, Artificial Sequence [sequence omitted]

SEQ ID NO: 6 - protein, 121 aa, Artificial Sequence [sequence omitted]

SEQ ID NO: 7 - protein, 108 aa, Artificial Sequence
  Description of Artificial Sequence Synthetic polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIK

SEQ ID NO: 8 - protein, 126 aa, Artificial Sequence
  Description of Artificial Sequence Synthetic polypeptide
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 9 - protein, 107 aa, Artificial Sequence
  Description of Artificial Sequence Synthetic polypeptide
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIK

SEQ ID NO: 10 - protein, 118 aa, Artificial Sequence
  Description of Artificial Sequence Synthetic polypeptide
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSS

SEQ ID NO: 11 - protein, 111 aa, Artificial Sequence
  Description of Artificial Sequence Synthetic polypeptide
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDGSNRWMFGGGTKLTVL

SEQ ID NO: 12 - protein, 123 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGSSGWYVPHWFDPWGQGTLVTVSS

SEQ ID NO: 13 - protein, 153 aa, Artificial Sequence
  Description of Artificial Sequence Synthetic polypeptide
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 14 - protein, 18 aa, Artificial Sequence
  Description of Artificial Sequence Synthetic peptide
GSTSGSGKPGSGEGSTKG

SEQ ID NO: 15 - protein, 246 aa, Artificial Sequence
  Description of Artificial Sequence Synthetic polypeptide
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHVWKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 16 - protein, 245 aa, Artificial Sequence
  Description of Artificial Sequence Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 17 - DNA, 1422 bp, Artificial Sequence
  Description of Artificial Sequence Synthetic polynucleotide
atgctgctgctggtgaccagcctgctgctgtgcgagctgccccaccccgcctttctgctgatccccggcggaagttacccatatgacgttcccgactacgctggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 18 - protein, 20 aa, Artificial Sequence
  Description of Artificial Sequence Synthetic peptide
METDTLLLWVLLLWVPGSTG

SEQ ID NO: 19 - protein, 23 aa, Artificial Sequence
  Description of Artificial Sequence Synthetic peptide
MLLVTSLLLCELPHPAFLLIPDT

SEQ ID NO: 20 - protein, 21 aa, Artificial Sequence
  Description of Artificial Sequence Synthetic peptide
MGTSLLCWMALCLLGADHADG

SEQ ID NO: 21 - protein, 5 aa, Artificial Sequence
  Description of Artificial Sequence Synthetic peptide
DKTHT

SEQ ID NO: 22 - protein, 4 aa, Artificial Sequence
  Description of Artificial Sequence Synthetic peptide
CPPC

SEQ ID NO: 23 - protein, 15 aa, Artificial Sequence
  Description of Artificial Sequence Synthetic peptide
CPEPKSCDTPPPCPR

SEQ ID NO: 24 - protein, 12 aa, Artificial Sequence
  Description of Artificial Sequence Synthetic peptide
ELKTPLGDTTHT

SEQ ID NO: 25 - protein, 10 aa, Artificial Sequence
  Description of Artificial Sequence Synthetic peptide
KSCDKTHTCP

SEQ ID NO: 26 - protein, 7 aa, Artificial Sequence
  Description of Artificial Sequence Synthetic peptide
KCCVDCP

SEQ ID NO: 27 - protein, 7 aa, Artificial Sequence
  Description of Artificial Sequence Synthetic peptide
KYGPPCP

SEQ ID NO: 28 - protein, 15 aa, Homo sapiens
EPKSCDKTHTCPPCP

SEQ ID NO: 29 - protein, 12 aa, Homo sapiens
ERKCCVECPPCP

SEQ ID NO: 30 - protein, 17 aa, Homo sapiens
ELKTPLGDTTHTCPRCP

SEQ ID NO: 31 - protein, 12 aa, Artificial Sequence
  Description of Artificial Sequence Synthetic peptide
SPNMVPHAHHAQ

SEQ ID NO: 32 - protein, 12 aa, Artificial Sequence
  Description of Artificial Sequence Synthetic peptide
ESKYGPPCPPCP

SEQ ID NO: 33 - protein, 12 aa, Artificial Sequence
  Description of Artificial Sequence Synthetic peptide
ESKYGPPCPSCP

SEQ ID NO: 34 - protein, 15 aa, Artificial Sequence
  Description of Artificial Sequence Synthetic peptide
EPKSCDKTYTCPPCP

SEQ ID NO: 35 - protein, 45 aa, Artificial Sequence
  Description of Artificial Sequence Synthetic polypeptide
TTTPAPRPPTPAPTIASQPLSLRPEACRPAAGGAVHTRGLDFACD

SEQ ID NO: 36 - protein, 24 aa, Artificial Sequence
  Description of Artificial Sequence Synthetic peptide
IYIWAPLAGTCGVLLLSLVITLYC

SEQ ID NO: 37 - protein, 23 aa, Artificial Sequence
  Description of Artificial Sequence Synthetic peptide
LGLLVAGVLVLLVSLGVAIHLCC

SEQ ID NO: 38 - protein, 25 aa, Artificial Sequence
  Description of Artificial Sequence Synthetic peptide
ALIVLGGVAGLLLFIGLGIFFCVRC

SEQ ID NO: 39 - protein, 23 aa, Artificial Sequence
  Description of Artificial Sequence Synthetic peptide
LCYLLDGILFIYGVILTALFLRV

SEQ ID NO: 40 - protein, 26 aa, Artificial Sequence
  Description of Artificial Sequence Synthetic peptide
WVLVVVGGVLACYSLLVTVAFIIFWV

SEQ ID NO: 41 - protein, 26 aa, Artificial Sequence
  Description of Artificial Sequence Synthetic peptide
VAAILGLGLVLGLLGPLAILLALYLL

SEQ ID NO: 42 - protein, 24 aa, Artificial Sequence
  Description of Artificial Sequence Synthetic peptide
ALPAALAVISFLLGLGLGVACVLA

SEQ ID NO: 43 - protein, 113 aa, Artificial Sequence
  Description of Artificial Sequence Synthetic polypeptide
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 44 - protein, 112 aa, Artificial Sequence
  Description of Artificial Sequence Synthetic polypeptide
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQRPYYK

SEQ ID NO: 45 - protein, 102 aa, Artificial Sequence
  Description of Artificial Sequence Synthetic polypeptide
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 46 - protein, 101 aa, Artificial Sequence
  Description of Artificial Sequence Synthetic polypeptide
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 47 - protein, 21 aa, Artificial Sequence
  Description of Artificial Sequence Synthetic peptide
ESPYQELQGQRSDVYSDLNTQ

SEQ ID NO: 48 - protein, 86 aa, Artificial Sequence
  Description of Artificial Sequence Synthetic polypeptide
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 49 - protein, 21 aa, Artificial Sequence
  Description of Artificial Sequence Synthetic peptide
DGVYTGLSTRNQETYETLKHE

SEQ ID NO: 50 - protein, 171 aa, Artificial Sequence
  Description of Artificial Sequence Synthetic polypeptide
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 51 - protein, 127 aa, Artificial Sequence
  Description of Artificial Sequence Synthetic polypeptide
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 52 - protein, 21 aa, Artificial Sequence
  Description of Artificial Sequence Synthetic peptide
DQVYQPLRDRDDAQYSHLGGN

SEQ ID NO: 53 - protein, 207 aa, Artificial Sequence
  Description of Artificial Sequence Synthetic polypeptide
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 54 - protein, 21 aa, Artificial Sequence
  Description of Artificial Sequence Synthetic peptide
NPDYEPIRKGQRDLYSGLNQR

SEQ ID NO: 55 - protein, 182 aa, Artificial Sequence
  Description of Artificial Sequence Synthetic polypeptide
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 56 - protein, 21 aa, Artificial Sequence
  Description of Artificial Sequence Synthetic peptide
DQLYQPLKDREDDQYSHLQGN

SEQ ID NO: 57 - protein, 163 aa, Artificial Sequence
  Description of Artificial Sequence Synthetic polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 164 aa, Artificial Sequence
  Description of Artificial Sequence Synthetic polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 59 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 21 aa, Artificial Sequence
  Description of Artificial Sequence Synthetic peptide
NQLYNELNLGRREEYDVLDKR

SEQ ID NO: 61 - protein, 21 aa, Artificial Sequence
  Description of Artificial Sequence Synthetic peptide
DGLYQGLSTATKDTYDALHMQ

SEQ ID NO: 62 - protein, 226 aa, Artificial Sequence
  Description of Artificial Sequence Synthetic polypeptide
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 63 - protein, 188 aa, Artificial Sequence
  Description of Artificial Sequence Synthetic polypeptide
MPGGPGVLQALPATIFLLFLLSAVYLGPGCQALWMHKVPASLMVSLGEDAHFQCPHNSSNNANVTWWRVLHGNYTWPPEFLGPGEDPNEPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 64 - protein, 21 aa, Artificial Sequence
  Description of Artificial Sequence Synthetic peptide
ENLYEGLNLDDCSMYEDISRG

SEQ ID NO: 65 - protein, 20 aa, Artificial Sequence
  Description of Artificial Sequence Synthetic peptide
RPRRSPAQDGKVYINMPGRG

SEQ ID NO: 66 - protein, 20 aa, Artificial Sequence
  Description of Artificial Sequence Synthetic peptide
RPRRSPAQDGKVYINMPGRG

SEQ ID NO: 67 - protein, 68 aa, Artificial Sequence
  Description of Artificial Sequence Synthetic polypeptide
FWVLVVVGGVLACYSLLVTVAFIIFWVRSKRSRLLHSDYMNMTPRRPGPTRKHYQPYAPPRDFAAYRS

SEQ ID NO: 68 - protein, 68 aa, Artificial Sequence; identical to SEQ ID NO: 67
  Description of Artificial Sequence Synthetic polypeptide

SEQ ID NO: 69 - protein, 619 aa, Artificial Sequence
  Description of Artificial Sequence Synthetic polypeptide
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 70 - protein, 9 aa, Artificial Sequence
  Description of Artificial Sequence Synthetic peptide
YPYDVPDYA

SEQ ID NO: 71 - protein, 8 aa, Artificial Sequence
  Description of Artificial Sequence Synthetic peptide
DYKDDDDK

SEQ ID NO: 72 - protein, 10 aa, Artificial Sequence
  Description of Artificial Sequence Synthetic peptide
EQKLISEEDL

SEQ ID NO: 73 - protein, 5 aa, Artificial Sequence
  Description of Artificial Sequence Synthetic peptide
GSGGS

SEQ ID NO: 74 - protein, 4 aa, Artificial Sequence
  Description of Artificial Sequence Synthetic peptide
GGGS

SEQ ID NO: 75 - protein, 4 aa, Artificial Sequence
  Description of Artificial Sequence Synthetic peptide
GGSG

SEQ ID NO: 76 - protein, 5 aa, Artificial Sequence
  Description of Artificial Sequence Synthetic peptide
GGSGG

SEQ ID NO: 77 - protein, 5 aa, Artificial Sequence
  Description of Artificial Sequence Synthetic peptide
GSGSG

SEQ ID NO: 78 - protein, 5 aa, Artificial Sequence
  Description of Artificial Sequence Synthetic peptide
GSGGG

SEQ ID NO: 79 - protein, 5 aa, Artificial Sequence
  Description of Artificial Sequence Synthetic peptide
GGGSG

SEQ ID NO: 80 - protein, 5 aa, Artificial Sequence
  Description of Artificial Sequence Synthetic peptide
GSSSG

SEQ ID NO: 81 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 82 - protein, 44 aa, Artificial Sequence
  Description of Artificial Sequence Synthetic polypeptide
FWVRSKRSRLLHSDYMNMTPRRPGPTRKHYQPYAPPRDFAAYRS

SEQ ID NO: 83 - protein, 35 aa, Artificial Sequence
  Description of Artificial Sequence Synthetic polypeptide
TKKKYSSSVHDPNGEYMFMRAVNTAKKSRLTDVTL

SEQ ID NO: 84 - protein, 37 aa, Artificial Sequence
  Description of Artificial Sequence Synthetic polypeptide
RRDQRLPPDAHKPPGGGSFRTPIQEEQADAHSTLAKI

SEQ ID NO: 85 - protein, 114 aa, Artificial Sequence
  Description of Artificial Sequence Synthetic polypeptide
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 86 - protein, 49 aa, Artificial Sequence
  Description of Artificial Sequence Synthetic polypeptide
HQRRKYRSNKGESPVEPAEPCRYSCPREEEGSTIPIQEDYRKPEPACSP

SEQ ID NO: 87 - protein, 187 aa, Artificial Sequence
  Description of Artificial Sequence Synthetic polypeptide
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 88 - protein, 54 aa, Artificial Sequence
  Description of Artificial Sequence Synthetic polypeptide
HIWQLRSQCMWPRETQLLLEVPPSTEDARSCQFPEEERGERSAEEKGRLGDLWV

SEQ ID NO: 89 - protein, 60 aa, Artificial Sequence
  Description of Artificial Sequence Synthetic polypeptide
CVKRRKPRGDVVKVIVSVQRKRQEAEGEATVIEALQAPPDVTTVAVEETIPSFTGRSPNH

SEQ ID NO: 90 - protein, 22 aa, Artificial Sequence
  Description of Artificial Sequence Synthetic peptide
EGLYNELQKDKMAEAYSEIGMK

SEQ ID NO: 91 - protein, 9 aa, Artificial Sequence
  Description of Artificial Sequence Synthetic peptide
GYSITSDHA

SEQ ID NO: 92 - protein, 7 aa, Artificial Sequence
  Description of Artificial Sequence Synthetic peptide
ISYSGIT

SEQ ID NO: 93 - protein, 12 aa, Artificial Sequence
  Description of Artificial Sequence Synthetic peptide
ARSLARTTAMDY

SEQ ID NO: 94 - protein, 11 aa, Artificial Sequence
  Description of Artificial Sequence Synthetic peptide
RASQDISSYLN

SEQ ID NO: 95 - protein, 7 aa, Artificial Sequence
  Description of Artificial Sequence Synthetic peptide
YTSRLHS

SEQ ID NO: 96 - protein, 9 aa, Artificial Sequence
  Description of Artificial Sequence Synthetic peptide
QQGNTLPYT

SEQ ID NO: 97 - protein, 10 aa, Artificial Sequence
  Description of Artificial Sequence Synthetic peptide
GFIFSNHWMN

SEQ ID NO: 98 - protein, 19 aa, Artificial Sequence
  Description of Artificial Sequence Synthetic peptide
EIRSKSINSATHYAESVKG

SEQ ID NO: 99 - protein, 9 aa, Artificial Sequence
  Description of Artificial Sequence Synthetic peptide
NYYGSTYDY

SEQ ID NO: 100 - protein, 11 aa, Artificial Sequence
  Description of Artificial Sequence Synthetic peptide
RASQFVGSSIH

SEQ ID NO: 101 - protein, 7 aa, Artificial Sequence
  Description of Artificial Sequence Synthetic peptide
YASESMS

SEQ ID NO: 102 - protein, 9 aa, Artificial Sequence
  Description of Artificial Sequence Synthetic peptide
QQSHSWPFT

SEQ ID NO: 103 - protein, 10 aa, Artificial Sequence
  Description of Artificial Sequence Synthetic peptide
GFTFDDYAMH

SEQ ID NO: 104 - protein, 17 aa, Artificial Sequence
  Description of Artificial Sequence Synthetic peptide
AITWNSGHIDYADSVEG

SEQ ID NO: 105 - protein, 9 aa, Artificial Sequence
  Description of Artificial Sequence Synthetic peptide
VSYLSTASS

SEQ ID NO: 106 - protein, 11 aa, Artificial Sequence
  Description of Artificial Sequence Synthetic peptide
RASQGIRNYLA

SEQ ID NO: 107 - protein, 7 aa, Artificial Sequence
  Description of Artificial Sequence Synthetic peptide
AASTLQS

SEQ ID NO: 108 - protein, 9 aa, Artificial Sequence
  Description of Artificial Sequence Synthetic peptide
QRYNRAPYT

SEQ ID NO: 109 - protein, 10 aa, Artificial Sequence
  Description of Artificial Sequence Synthetic peptide
GFIFSSYAMH

SEQ ID NO: 110 - protein, 17 aa, Artificial Sequence
  Description of Artificial Sequence Synthetic peptide
FMSYDGSNKKYADSVKG

SEQ ID NO: 111 - protein, 17 aa, Artificial Sequence
  Description of Artificial Sequence Synthetic peptide
DRGIAAGGNYYYYGMDV

SEQ ID NO: 112 - protein, 11 aa, Artificial Sequence
  Description of Artificial Sequence Synthetic peptide
RASQSVYSYLA

SEQ ID NO: 113 - protein, 7 aa, Artificial Sequence
  Description of Artificial Sequence Synthetic peptide
DASNRAT

SEQ ID NO: 114 - protein, 10 aa, Artificial Sequence
  Description of Artificial Sequence Synthetic peptide
QQRSNWPPFT

SEQ ID NO: 115 - protein, 10 aa, Artificial Sequence
  Description of Artificial Sequence Synthetic peptide
GYVFTDYGMN

SEQ ID NO: 116 - protein, 17 aa, Artificial Sequence
  Description of Artificial Sequence Synthetic peptide
WINTYIGEPIYADSVKG

SEQ ID NO: 117 - protein, 9 aa, Artificial Sequence
  Description of Artificial Sequence Synthetic peptide
GYRSYAMDY

SEQ ID NO: 118 - protein, 11 aa, Artificial Sequence
  Description of Artificial Sequence Synthetic peptide
KASQNVGTNVA

SEQ ID NO: 119 - protein, 7 aa, Artificial Sequence
  Description of Artificial Sequence Synthetic peptide
SASFLYS

SEQ ID NO: 120 - protein, 9 aa, Artificial Sequence
  Description of Artificial Sequence Synthetic peptide
QQYNIYPLT

SEQ ID NO: 121 - protein, 8 aa, Artificial Sequence
  Description of Artificial Sequence Synthetic peptide
GFTFSSYA

SEQ ID NO: 122 - protein, 8 aa, Artificial Sequence
  Description of Artificial Sequence Synthetic peptide
ISGSGGST

SEQ ID NO: 123 - protein, 16 aa, Artificial Sequence
  Description of Artificial Sequence Synthetic peptide
AKDGSSGWYVPHWFDP

SEQ ID NO: 124 - protein, 8 aa, Artificial Sequence
  Description of Artificial Sequence Synthetic peptide
SGSIASNY

SEQ ID NO: 125 - protein, 3 aa, Artificial Sequence
  Description of Artificial Sequence Synthetic peptide
EDN

SEQ ID NO: 126 - protein, 10 aa, Artificial Sequence
  Description of Artificial Sequence Synthetic peptide
QSYDGSNRWM

SEQ ID NO: 127 - protein, 214 aa, Artificial Sequence [sequence omitted]

SEQ ID NO: 128 - protein, 448 aa, Artificial Sequence
  Description of Artificial Sequence Synthetic polypeptide
EVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 16 aa, Artificial Sequence
  Description of Artificial Sequence Synthetic peptide
ELPTQGTFSNVSTNVS

SEQ ID NO: 130 - protein, 6 aa, Artificial Sequence
  Description of Artificial Sequence Synthetic peptide
SGGGGS

SEQ ID NO: 131 - protein, 11 aa, Artificial Sequence
  Description of Artificial Sequence Synthetic peptide
SGGGGSGGGGS

SEQ ID NO: 132 - DNA, 25 bp, Artificial Sequence
  Description of Artificial Sequence Synthetic oligonucleotide
gtcgacatttcccgtaaatcgtcga

SEQ ID NO: 133 - DNA, 26 bp, Artificial Sequence
  Description of Artificial Sequence Synthetic oligonucleotide
gcgcttcctgacagtgacgcgagccg

SEQ ID NO: 134 - DNA, 25 bp, Artificial Sequence
  Description of Artificial Sequence Synthetic oligonucleotide
gcgcttcctgacagtgacgtctttg

SEQ ID NO: 135 - DNA, 38 bp, Artificial Sequence
  Description of Artificial Sequence Synthetic oligonucleotide
gctgtacggtaaaagtgagctcttacgggaatgggaat

SEQ ID NO: 136 - DNA, 24 bp, Artificial Sequence
  Description of Artificial Sequence Synthetic oligonucleotide
gcgtgccgtcatttcggggaaatc

SEQ ID NO: 137 - DNA, 178 bp, Artificial Sequence
  Description of Artificial Sequence Synthetic polynucleotide
ctgacaaattcagtataaaagcttggggctggggccgagcactggggactttgagggtggccaggccagcgtaggaggccagcgtaggatcctgctgggagcggggaactgagggaagcgacgccgagaaagcaggcgtaccacggagggagagaaaagctccggaagcccagcagcg

SEQ ID NO: 138 - DNA, 63 bp, Artificial Sequence
  Description of Artificial Sequence Synthetic oligonucleotide
ttcgcatattaaggtgacgcgtgtggcctcgaacaccgagcgaccctgcagcgacccgcttaa

SEQ ID NO: 139 - DNA, 25 bp, Artificial Sequence
  Description of Artificial Sequence Synthetic oligonucleotide
tctagagggtatataatgggggcca

SEQ ID NO: 140 - DNA, 64 bp, Artificial Sequence
  Description of Artificial Sequence Synthetic oligonucleotide
atctggtaggcgtgtacggtgggaggtctatataagcagagctcgtttagtgaaccgtcagatc

SEQ ID NO: 141 - DNA, 203 bp, Artificial Sequence
  Description of Artificial Sequence Synthetic polynucleotide
tgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagctt

SEQ ID NO: 142 - DNA, 76 bp, Artificial Sequence
  Description of Artificial Sequence Synthetic oligonucleotide
caacaaaatgtcgtaacaagggcggtaggcgtgtacggtgggaggtctatataagcagagctcgtttagtgaaccg

SEQ ID NO: 143 - DNA, 41 bp, Artificial Sequence
  Description of Artificial Sequence Synthetic oligonucleotide
ggggggctataaaagggggtgggggcgttcgtcctcactct

SEQ ID NO: 144 - protein, 97 aa, Artificial Sequence
  Description of Artificial Sequence Synthetic polypeptide
PRVRTGSKTPPHERPFQCRICMRNFSDSPTLRRHTRTHTGEKPFQCRICMRNFSQGANLRRHLRTHTGEKPFQCRIMRNFSQANTLQRHLKTHTGEK

SEQ ID NO: 145 - protein, 769 aa, Artificial Sequence
  Description of Artificial Sequence Synthetic polypeptide
AQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 146 - DNA, 30 bp, Artificial Sequence
  Description of Artificial Sequence Synthetic oligonucleotide
ggaggaaaaactgtttcatacagaaggcgt

SEQ ID NO: 147 - protein, 16 aa, Artificial Sequence
  Description of Artificial Sequence Synthetic peptide
MAGPHPVIVITGPHEE

SEQ ID NO: 148 - protein, 16 aa, Artificial Sequence
  Description of Artificial Sequence Synthetic peptide
MAGPPHIVEETGPHVI

SEQ ID NO: 149 - protein, 21 aa, Artificial Sequence
  Description of Artificial Sequence Synthetic peptide
KYELHAATDTTPSVVVHVCES

SEQ ID NO: 150 - DNA, 732 bp, Artificial Sequence
  Description of Artificial Sequence Synthetic polynucleotide
gatatacaaatgactcaatcaccttcctcactttccgccagcgtgggtgatcgagtgaccatcacatgccgcgcttcacaggacatctcctcttacctgaactggtaccagcagaaacccgggaaagccccaaagctgttgatctactatacatcaagactgcactctggcgtcccctctaggttcagtggatctggctcaggtactgacttcacgttcactatttccagcctgcagccggaagatattgctacctactattgtcagcagggaaataccctcccatatacctttgggcaggggacaaaggtggaaatcaagggcagcacctccggcagcggcaagcctggcagcggcgagggcagcaccaagggcgaggtccaactgcaggaaagcggtccaggcttggtgagaccatcccagaccctgagcctcacctgtaccgtgtccgggtacagtatcacctccgaccatgcatggtcctgggtgcgccagcctcccggaagaggtctggaatggatcgggtatatctcctactcaggaatcaccacctacaatccctcccttaagtcaagggtgactatgctccgcgatacatccaaaaatcagttctcccttcggttgtcaagtgttacagccgccgacaccgcagtctactactgtgcaaggagcctcgccaggacgactgcaatggattattggggccagggctccctggtgactgtcagcagc

SEQ ID NO: 151 - protein, 244 aa, Artificial Sequence
  Description of Artificial Sequence Synthetic polypeptide
DIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKGSTSGSGKPGSGEGSTKGEVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSS

SEQ ID NO: 152 - DNA, 756 bp, Artificial Sequence
  Description of Artificial Sequence Synthetic polynucleotide
gaagtccaactcctggaaagtgggggcggcctcgtgcaacctggaggttccctgaggttgtcctgcgctgctagcgggttcacgtttagcagctatgcaatgtcatgggttaggcaggcacctggcaagggcctggaatgggtttcagctatctcagggtcagggggtagcacctactatgctgatagtgtgaaaggaaggtttacgatctccagagataattccaagaataccctgtaccttcaaatgaacagtctcagggcggaggatacggcagtatactactgcgccaaggacggttctagcggttggtacgtgccgcattggttcgacccgtggggccaagggactttggtcactgtgtcctctggcagcacgtccggcagcggcaagcctggcagcggcgagggatccaccaagggcaactttatgcttactcagccgcatagcgtatccgaaagcccaggcaaaacggttactatttcatgtaccagatccagcggaagcatagctagtaattacgtgcagtggtaccaacagcgaccaggttcaagtccaactactgtgatttatgaagataatcagaggcccagtggggtcccagatcgcttctctggctcaatagattcttcttctaattccgcaagtcttacaatctctgggttgaagacagaagatgaggcggattattactgtcaatcttatgacggttctaaccgctggatgtttgggggcggaacgaagctgacagtcctt

SEQ ID NO: 153 - protein, 252 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGSSGWYVPHWFDPWGQGTLVTVSSGSTSGSGKPGSGEGSTKGNFMLTQPHSVSESPGKTVTISCTRSSGSIASNYVQWYQQRPGSSPTTVIYEDNQRPSGVPDRFSGSIDSSSNSASLTISGLKTEDEADYYCQSYDGSNRWMFGGGTKLTVL

SEQ ID NO: 154 - DNA, 531 bp, Artificial Sequence
  Description of Artificial Sequence Synthetic polynucleotide
atggaaatctgtcggggccttcgctcacatctgatcacactgctcctgttcctgtttcactccgaaacgatttgtaggccgtccggacggaaatcttcaaaaatgcaagcttttcggatatgggatgtaaaccaaaagacgttctaccttcgcaataatcaactcgtggcggggtatcttcagggtcctaatgttaaccttgaggagaagatagacgtcgtaccaatagagccacatgccctctttcttgggattcatgggggtaaaatgtgtctttcatgtgtaaagtctggcgatgaaacacggcttcagcttgaagctgtaaacattactgaccttagtgagaaccgaaaacaggataagcgatttgcgttcatccggtctgacagcggccctactacctcatttgagtcagcggcgtgtccagggtggtttttgtgtacggctatggaagcagatcagcccgtcagtcttaccaatatgccggatgagggagtcatggtaaccaaattctacttccaggaggatgag

SEQ ID NO: 155 - protein, 177 aa, Artificial Sequence
  Description of Artificial Sequence Synthetic polypeptide
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 156 - protein, 20 aa, Artificial Sequence
  Description of Artificial Sequence Synthetic peptide
GGGGSGGGGSGGGGSGGGGS

SEQ ID NO: 157 - RNA, 20 nt, Artificial Sequence
  Description of Artificial Sequence Synthetic oligonucleotide
aggguaggagagacucacgc